(19) United States
(12) Patent Application Publication
(10) Pub. No.: US 2021/0347858 A1
(43) Pub. Date: Nov. 11, 2021

(54) ALIMENTARY AND SYSTEMIC ANTIVIRAL THERAPEUTICS

(71) Applicant: Firebreak, Inc., Boulder, CO (US)

(72) Inventor: Timothy W. Starzl, Boulder, CO (US)

(21) Appl. No.: 17/221,515

(22) Filed: Apr. 2, 2021

Related U.S. Application Data
(60) Provisional application No. 63/004,974, filed on Apr. 3, 2020.
(60) Provisional application No. 63/047,996, filed on Jul. 3, 2020.
(60) Provisional application No. 63/158,682, filed on Mar. 9, 2021.

(51) Int. Cl. C07K 16/10; C07K 16/40; C07K 14/165; C12N 15/85; A61K 45/06

(52) U.S. Cl. CPC C07K 16/10; C07K 16/40; C07K 14/165; C12N 15/85; C12N 2800/107; C07K 2317/76; C07K 2317/11; C07K 2317/23; A61K 45/06

(57) ABSTRACT

Methods and compositions are provided for rapid preparation of anti-SARS-CoV-2 protein polyclonal IgY antibodies. Compositions comprising the antibodies are useful for decreasing transmission, duration, and/or severity of coronavirus disease 2019 (COVID-19). Lingual and sublingual alimentary traps, oral, parenteral, and inhalable compositions are provided for preventing and treating COVID-19. The compositions can be rapidly tailored for seasonal or even monthly mutations in the SARS-CoV-2 viral antigenic proteins. Improved methods of generating IgY antibodies are also provided.

### CROSS REFERENCES TO RELATED APPLICATIONS

This application claims the benefit under 35 U.S.C. § 119(e) of U.S. provisional application No. 63/004,974, filed Apr. 3, 2020, U.S. provisional application No. 63/047,996, filed Jul. 3, 2020, and U.S. provisional application No. 63/158,682, filed Mar. 9, 2021, the entire contents of each of which are incorporated herein by reference.

### BACKGROUND OF THE DISCLOSURE

### Sequence Listing

The instant application includes a Sequence Listing which has been submitted electronically in ASCI format and is hereby incorporated by reference in its entirety. Said ASCI copy, created on Apr. 2, 2021 is named Sequence-Listing-18659-0001USU1 and is 331 kilobytes (KB) in size.

### FIELD OF THE DISCLOSURE

The disclosure provides topical, oral, injectable, intranasal, and inhalable antiviral compositions comprising coronavirus-specific polyclonal immunoglobulin Y antibodies. Improved methods for preparation of polyclonal IgY antibodies are provided. The disclosure provides compositions and methods for treating and/or preventing COVID-19.

### DESCRIPTION OF THE RELATED ART

Coronavirus disease 2019 (COVID-19) is an infectious disease caused by severe acute respiratory syndrome coronavirus 2 (SARS-CoV-2). The disease was first identified in 2019 in Wuhan, the capital of Hubei, China, and has since spread globally, resulting in the 2019-20 coronavirus pandemic. Common symptoms include fever, cough, and shortness of breath. Muscle pain, sputum production, diarrhea, sore throat are less common. Complete or partial loss of smell and loss of taste are particularly common symptoms of cases with no other symptoms. While the majority of cases result in mild symptoms, some progress to pneumonia, multi-organ failure, and death.

Therapeutics and vaccines are under rapid development to address the SARS-CoV-2 global pandemic. Several antiviral drugs are in clinical trials, and a few vaccines have been approved. Remdesivir is an injectable broad-spectrum antiviral medication that has received emergency use authorization for patients requiring hospitalization. Convalescent plasma from recovered COVID-19 patients has been employed for treating severe COVID-19 infections. However, scalability and safety are issues of concern. The volume of plasma from one recovered COVID-19 patient is only enough to treat one to two COVID-19 patients. Further, the plasma must be tested for several other blood borne pathogens prior to use. Safer, highly scalable alternatives to convalescent plasma are desirable.

One of the greatest concerns for government and health officials at present is the emergence of variants. Of particular concern are the variants from the UK (B.1.1.7.), South Africa (B.1.351) and Brazil (P.1) variants. With such a staggering incidence throughout the world, the SARS-CoV-2 virus has ample opportunity to mutate and diminish the efficacy of current vaccines and therapeutics. Centers for Disease Control and Prevention, 2020, “Emerging SARS-CoV-2 Variants.” Centers for Disease Control and Prevention, www.cdc.gov/coronavirus/2019-ncov/more/science-and-research/scientific-brief-emerging-variants.html.

For instance, South Africa has halted the rollout of the AstraZeneca vaccine due to reports of only 10% efficacy against the South African B.1.351 variant. Booth et al. “South Africa Suspends Oxford-AstraZeneca Vaccine Rollout after Researchers Report ‘Minimal’ Protection against Coronavirus Variant.” The Washington Post, WP Company, 8 Feb. 2021, www.washingtonpost.com/world/europe/astrazeneca-oxford-vaccine-south-african-variant/2021/02/07/e82127f8-6948-11eb-a66e-e27046e9e898_story.html. The variants may be more transmissible, and as seen with the AstraZeneca vaccine, each mutation in the Spike protein increases the risk of current therapeutics being rendered ineffective.

While monoclonal antibodies specific for the SARS-CoV-2 spike protein have their advantages, the specificity to a single epitope is disadvantageous as mutations emerge. Cocktails of human monoclonal antibodies have shown promise, for example, when targeting non-overlapping epitopes on the SARS-CoV-2 spike protein, for example, as in REGN-COV2 (Regeneron Pharmaceuticals, Inc.). Baum et al., Science 10.1126/science.abe2402 (2020). In another example, two monoclonal antibodies derived from patient samples, and directed to different SARS-CoV-2 spike protein epitopes were tested in a Phase 2/3 clinical trial among non-hospitalized patients with mild to moderate COVID-19 illness. Treatment with a combination of bamlanivimab and etesevimab, compared to placebo was associated with a statistically significant reduction in SARS-CoV-2 viral load at day 11; however, no significant difference in viral load reduction was observed for monotherapy with bamlanivimab. Gottlieb et al., 2021 JAMA doi:10.1001/jama.2021.0202. Unfortunately, monoclonal antibodies may not be effective for all variants, and may take time to develop.

Recently, a study of antibody resistance of SARS-CoV-2 variants B.1.351 and B.1.1.7 was published showing the UK variant B.1.1.7 to be refractory to neutralization by most mAbs to the N-terminal domain (NTD) of the spike and relatively resistant to a few mAbs to the receptor-binding-domain (RBD), although not more resistant to convalescent plasma or vacinee sera. However, findings on the B.1.351 found this variant is not only refractory to neutralization by most NTD mAbs but also by multiple individual mAbs to the receptor-binding motif on RBD mostly due to an E484K mutation. Moreover, B.1.351 is markedly more resistant to neutralization by convalescent plasma and vaccine sera. Wang, Penfei et al., 2021, Antibody resistance of SARS-CoV-2 variants B.1.351 and B.1.1.7. Nature https://doi.org/10.1038/s41586-021-03398-2.

A recent study isolated infectious B.1.1.7 and B.1.351 strains from acutely infected individuals and examined sensitivity of the two variants to SARS-CoV-2 antibodies present in sera and nasal swabs from individuals infected with previously circulating strains or who were recently vaccinated, in comparison with a D614G reference virus. Sera from 58 convalescent individuals collected up to 9 months after symptoms, similarly neutralized B.1.1.7 and D614G. In contrast, after 9 months, convalescent sera had a mean sixfold reduction in neutralizing titers, and 40% of the samples lacked any activity against B.1.351. Sera from 19 individuals vaccinated twice with Pfizer Cominarty, longitudinally tested up to 6 weeks after vaccination, were similarly potent against B.1.1.7 but less efficacious against B.1.351, when compared to D614G. Neutralizing titers increased after the second vaccine dose, but remained 14-fold lower against B.1.351. In contrast, sera from convalescent or vaccinated individuals similarly bound the three spike proteins in a flow cytometry-based serological assay. Neutralizing antibodies were rarely detected in nasal swabs from vaccinees. Thus, faster-spreading SARS-CoV-2 variants acquired a partial resistance to neutralizing antibodies generated by natural infection or vaccination, which was most frequently detected in individuals with low antibody levels. Results indicate that B1.351, but not B.1.1.7, may increase the risk of infection in immunized individuals. Planas, D., Bruel, T., Grzelak, L. et al. Sensitivity of infectious SARS-CoV-2 B.1.1.7 and B.1.351 variants to neutralizing antibodies. Nat Med (2021). https://doi.org/10.1038/s41591-021-01318-5.

Everyone may be at risk during the SARS-CoV-2 pandemic, but individuals at the greatest risk of exposure are frontline workers, including those in healthcare, nursing homes, meat packing, food processing and distribution, first response, warehouse operations, the military, and other essential services. These individuals face an elevated risk of becoming infected with or spreading COVID-19 and must take precautions to protect themselves, their families, and their communities. In addition, transmission from asymptomatic individuals has been estimated to account for more than half of all transmission. Johansson et al., SARS-CoV-2 transmission from people without COVID-19 symptoms, JAMA Network Open. 2021; 4 (1):e2035057. doi:10.1001/jamanetworkopen.2020.35057.

The provisional leading cause-of-death rankings for 2020 indicate that COVID-19 was the third leading cause of death in the US behind heart disease and cancer. Ahmad et al., 2021, JAMA published online Mar. 31, 2021.

There is a need for rapid, safe, economical and effective methods and compositions to decrease SARS-CoV-2 transmission, and decrease duration and severity of COVID-19 symptoms, as well as methods for rapid development of new prophylactic and therapeutic polyclonal antibodies recognizing SARS-CoV-2 mutants, variants, and strains.

One method to produce specific polyclonal antibodies is by utilizing the immune system of an avian host. For example, when presented with a target antigen (such as recombinant protein), a chicken's acquired immune system will activate and produce antibodies specific to the target antigen. These antibodies are transferred to the yolk of laid eggs. Müller, Sandra et al. “IgY antibodies in human nutrition for disease prevention.” Nutrition Journal vol. 14 109. 20 Oct. 2015, doi:10.1186/s12937-015-0067-3.

Birds (such as laying-hens) are highly cost-effective as producers of antibodies compared with mammals traditionally used for such production. Avian antibodies have biochemical advantages over mammalian antibodies. Immunologic differences between mammals and birds result in increased sensitivity and decreased background in immunological assays, as well as high specificity and lack of complement immune effects when administered to mammalian subjects. In contrast to mammalian antibodies, avian antibodies do not activate the human complement system through the primary or classical pathway nor will they react with rheumatoid factors, human anti-mouse IgG antibodies, staphylococcal proteins A or G, or bacterial and human Fc receptors. Avian antibodies can however activate the non-inflammatory alternative pathway. Thus avian antibodies offer many advantages over mammalian antibodies.

Fu et al. 2006 describe pathogen-free (SPF) chickens immunized with inactivated SARS coronavirus. Journal of virological methods, 2006, Vol 133, Num 1, pp 112-115.

Palaniyappan et al., 2012 describe SARS-Cov-1 N protein used for IgY production, and use in diagnostic ELISA. Poultry Science 91:636-642, 2012.

Shen et al., 2020 describe anti-SARS-CoV-2 IgY isolated from egg yolks of hens immunized with inactivated SARS-CoV-2. SARS-CoV-2 (20SF014-SARS-CoV-2) was expanded in Vero-E6 cells, collected, and stored at −80° C. until use. Hens were subcutaneously immunized with formaldehyde-inactivated SARS-CoV-2 and Freund's complete adjuvant, boosted twice at 2-3 week interval with the mixture of the inactivated virus and Freund's incomplete adjuvant on both wings (0.5 mL/hen). Three weeks after the final immunization, the eggs were collected, and crude IgY antibodies were extracted from the egg yolks. Virologica Sinica. DOI: 10.1007/s12250-021-00371-1.

Wei et al., 2021 describe neutralizing effect of anti-spike-S1 IgYs on a SARS-CoV-2 pseudovirus, as well as its inhibitory effect on the binding of the coronavirus spike protein mutants to human ACE2. SARS-CoV-2 Spike-S1 was expressed in Sf9 insect cells using the baculovirus/insect cell expression system. International Immunopharmacology, 90 (2021) 107172.

Improved methods for rapid production of polyclonal IgY antibodies for use in antiviral, antibacterial, anti-venom, anti-toxin, anti-virulence factor, anti-adherence factor, anti-prion, or anti-prion-like protein, therapeutic or prophylactic compositions are desirable.

### SUMMARY OF THE INVENTION

The present disclosure provides methods and compositions for rapid preparation and use of polyclonal anti-SARS-CoV-2 antibodies and combinations thereof. In particular, anti-SARS-CoV-2 polyclonal IgY antibodies, serum, egg yolk, and/or whole immune egg have been derived from poultry vaccinated with recombinant proteins, nucleic acids, and/or coronavirus vaccines. Compositions comprising the anti-SARS-CoV-2 polyclonal antibodies are useful for decreasing transmission, duration, and/or severity of coronavirus disease 2019 (COVID-19). Oral, parenteral, and inhalable compositions are provided for preventing and treating COVID-19, including Lingual and sublingual alimentary traps. The polyclonal anti-SARS-CoV-2 IgY antibodies have been demonstrated to specifically bind to SARS-CoV-2 variants, and compositions may be rapidly tailored for seasonal or even monthly mutations in the SARS-CoV-2 viral antigenic proteins. Safer, highly scalable alternatives to COVID-19 convalescent plasma are provided herein.

The disclosure provides an antiviral composition comprising an effective amount of anti-SARS-CoV-2-S-protein-specific immunoglobulin Y (IgY) antibodies, anti-SARS-CoV-2-S-protein RBD domain-specific immunoglobulin Y (IgY) antibodies, and/or anti-SARS-CoV-2N-protein-specific polyclonal IgY antibodies, and a pharmaceutically acceptable carrier. The composition may further include anti-bovine, poultry, porcine, canine, human, ferret, or feline coronavirus-specific polyclonal IgY antibodies.

In some embodiments, the SARS-CoV-2-S-protein-specific immunoglobulin Y (IgY) antibodies bind to one or more, two or more, three or more, five or more, or ten or more known SARS-CoV-2 S-proteins in the NCBI database.

In some embodiments, the SARS-CoV-2-N-protein-specific immunoglobulin Y (IgY) antibodies bind to one or more, two or more, three or more, five or more, or ten or more known SARS-CoV-2N-proteins in the NCBI database.

The disclosure provides compositions and antiviral compositions comprising anti-SARS-CoV-2 IgY antibodies specific for SARS-CoV-2 S1(Spike), S1(RBD), S2, M and/or N protein antigens.

In some embodiments, the anti-SARS-CoV-2-S-protein IgY antibodies specifically bind to SARS-CoV-2-S-protein or a variant thereof. Anti-SARS-CoV-2-S-protein IgY antibodies may specifically bind to SARS-CoV-2-S-protein or a variant thereof comprising an amino acid sequence selected from the group consisting of SEQ ID NOs: 1, 6, 7, 10, 11, 12, 13, 14, 15, 16, 17, 18, 36, 37, 38, 39, 40 or a fragment of at least 10, 20, 30, 40, 50, 60, 70, 80, 100, 150, or 200 contiguous amino acid residues thereof, or a substantially similar protein thereof. In some embodiments, IgY antibodies are provided that specifically bind to a SARS-CoV-2 spike protein or a variant thereof. The SARS-CoV-2 spike protein may comprise an amino acid sequence of SEQ ID NO: 1, or a variant thereof, for example, including one or more, two or more, three or more, four or more, five or more, six or more, seven or more, eight or more, nine or more, 10 or more, 11 or more, 12 or more, 13 or more, 14 or more, or one, two, three, four, five, six, seven, eight, nine, 10, 11, 12, 13, or 14 mutations. In some embodiments, the mutations may include an amino acid residue deletion and or an amino acid residue substitution. The SARS-CoV-2 spike protein may comprise an amino acid sequence of SEQ ID NO: 41, or a variant thereof, for example, including one or more, two or more, three or more, four or more, five or more, six or more, seven or more, eight or more, nine or more, 10 or more, 11 or more, 12 or more, 13 or more, 14 or more, or one, two, three, four, five, six, seven, eight, nine, 10, 11, 12, 13, or 14 mutations. The SARS-CoV-2 spike protein may include an amino acid residue deletion or substitution selected from the group consisting of orfΔ3b, deletion 69-70, M129I, deletion 144, P337S, F338K, V341I, F342L, A344S, A348S, A352S, N354D, S359N, V367F, N379S, A372S, A372T, F377L, K378R, K378N, P384L, T385A, T393P, V395I, D405V, E406Q, R408I, Q409E, Q414A, Q414E, Q414R, K417N, A435S, W436R, N439K, N440K, K444R, V445F, G446V, G446S, P499R, L452R, Y453F, F456L, F456E, K458R, K458Q, E471Q, I472V, G476S, S477N, S477L, S477R, T478I, P479S, N481D, G482S, V483A, V483L, G485S, F486S, F490S, S494P, N501Y, V503F, Y505C, Y508H, A520S, A520V, P521S, P521R, A522V, A522S, A570D, D614G, P681H, R683A, R685A, I692V, T716I, F817P, A829T, A892P, A899P, A942P, S982A, K986P, V987P, and/or D1118H. In some embodiments, a SARS-CoV-2 S protein variant may comprise a D614G, N439K, and/or Y453F mutation. In some embodiments, a SARS-CoV-2 S protein variant may comprise a mutation selected from the group consisting of K417N, E484K, N501Y, S477N, L452R, and D614G.

In some embodiments, the compositions of the disclosure comprise anti-SARS-CoV-2-S-protein RBD IgY antibodies that bind to SARS-CoV-2-S-protein RBD domain comprising the amino acid sequence of (YP_009724390.1) (Arg319-Phe541) (SEQ ID NO: 36). In some embodiments, the compositions of the disclosure comprise anti-SARS-CoV-2-S-protein RBD IgY antibodies that bind to SARS-CoV-2-S-protein RBD domain comprising the amino acid sequence of the amino acid sequence of (YP_009724390.1) (Arg319-Phe541(N501Y))) (SEQ ID NO: 37). In some embodiments, the compositions of the disclosure comprise anti-SARS-CoV-2-S-protein RBD IgY antibodies that bind to SARS-CoV-2-S-protein RBD domain comprising the amino acid sequence of (YP_009724390.1) (Arg319-Phe541(E484K))) (SEQ ID NO: 38). In some embodiments, the compositions of the disclosure comprise anti-SARS-CoV-2-S-protein RBD IgY antibodies that bind to SARS-CoV-2-S-protein RBD domain comprising the amino acid sequence of (YP_009724390.1) (Arg319-Phe541(K417N))) (SEQ ID NO: 39). In some embodiments, the compositions of the disclosure comprise anti-SARS-CoV-2-S1-protein IgY antibodies that bind to SARS-CoV-2-S1-protein comprising the amino acid sequence of (YP_009724390.1) (Met1-Arg685(K417N, E484K, N501Y, D614G)) (SEQ ID NO: 40).

In some embodiments, the polyclonal antibodies and or antiviral compositions may include anti-SARS-CoV-2-N-protein polyclonal antibodies. The anti-SARS-CoV-2N-protein polyclonal antibodies may specifically bind to SARS-CoV-2-N-protein comprising an amino acid sequence selected from the group consisting of SEQ ID NO: 4, 8, and 9, or a fragment of at least 10, 20, 30, 40, 50, 60, 70, 80, 100, 150, or 200 contiguous amino acid residues thereof, or a substantially similar protein thereof.

The polyclonal antibodies and antiviral compositions may further include SARS-CoV-2 envelope protein-specific IgY antibodies. In some embodiments, the anti-SARS-CoV-2 envelope protein IgY specifically binds to SARS-CoV-2 envelope protein comprising the amino acid sequence of SEQ ID NO: 3, or a fragment of at least 10, 20, 30, 40, or 50 contiguous amino acid residues thereof, or a substantially similar protein thereof.

The antiviral composition may further include a flavoring, sweetener, stabilizer, pH regulator, preservative, antibody matrix, or vitamin. The composition may be in the form of a lozenge, troche, gel, film, liquid, powder, capsule, tablet, caplet, or fluid. In some embodiments, the specific IgY antibodies are in the form of immune egg, egg yolk, dried immune egg, concentrated IgY, isolated IgY, or purified IgY. In some embodiments, the IgY antibodies are in a purified or concentrated form.

A method is provided for reducing viral replication in a cell comprising treating a coronavirus-infected cell with an effective amount of a composition comprising an effective amount of the composition according to claim 1.

A method is provided for the treatment or prophylaxis of a viral infection in a subject in need thereof comprising administering to the subject a composition comprising a therapeutically effective amount of a mixture of SARS-CoV-2-S-protein-specific IgY antibodies and SARS-CoV-2N-protein-specific polyclonal IgY antibodies, and a pharmaceutically acceptable carrier.

In some embodiments, the viral infection is a coronavirus infection. The coronavirus infection may be COVID-19.

In some embodiments, the composition further comprises anti-bovine coronavirus-specific polyclonal IgY antibodies, anti-avian coronavirus polyclonal IgY antibodies, anti-porcine coronavirus polyclonal IgY antibodies, anti-canine coronavirus polyclonal IgY antibodies, anti-ferret coronavirus polyclonal IgY antibodies, or anti-feline coronavirus polyclonal IgY antibodies.

A method is provided for reducing severity or duration of symptoms of a coronavirus infection in a subject in need thereof, comprising administering a composition comprising a mixture of SARS-CoV-2-S-protein-specific IgY and N-protein-specific polyclonal IgY antibodies and a pharmaceutically acceptable carrier. The symptoms may be selected from the group consisting of fever, cough, muscle aches, lethargy, diarrhea, vomiting, stomachache, shortness of breath, muscle pain, sputum production, diarrhea, sore throat, complete or partial loss of smell, and loss of taste

A vaccine composition is provided for production of polyclonal antibodies, the composition comprising a recombinant SARS-CoV-2-S-protein, and a recombinant SARS-CoV-2N-protein, and a carrier, optionally comprising an adjuvant. The recombinant SARS-CoV-2-S-protein may comprise an amino acid sequence selected from the group consisting of SEQ ID NO: 1, 6, 7, 10, 11, 12, 13, 14, 15, 16, 17, 18, 36, 37, 38, 39, 40, 41 or a fragment of at least 10, 20, 30, 40, 50, 60, 70, 80, 100, 150, or 200 contiguous amino acid residues thereof, or a substantially similar protein thereof. The recombinant SARS-CoV-2-N-protein may comprise an amino acid sequence selected from the group consisting of SEQ ID NO: 4, 8, and 9, or a fragment of at least 10, 20, 30, 40, 50, 60, 70, 80, 100, 150, or 200 contiguous amino acid residues thereof, or a substantially similar protein thereof.

The vaccine composition may include a recombinant SARS-CoV-2 envelope protein. The recombinant SARS-CoV-2 envelope protein may comprise the amino acid sequence of SEQ ID NO: 3, or a fragment of at least 10, 20, 30, 40, or 50 contiguous amino acid residues thereof, or a substantially similar protein thereof. The vaccine composition may include killed or inactivated coronavirus, optionally where the killed or inactivated coronavirus is a bovine coronavirus.

The disclosure provides an antiviral composition comprising an effective amount of anti-SARS-CoV-2-S-protein immunoglobulin Y (IgY) antibodies, and a pharmaceutically acceptable carrier, optionally, further comprising an effective amount of anti-human ACE-2 IgY antibodies, and/or further comprising an effective amount of anti-SARS-CoV-2N-protein-specific polyclonal IgY antibodies. The anti-SARS-CoV-2-S-protein immunoglobulin Y (IgY) antibodies may bind to a SARS-CoV-2 S protein RBD domain. The disclosure provides an antiviral composition comprising an effective amount of anti-SARS-CoV-2-RBD-protein immunoglobulin Y (IgY) antibodies, and a pharmaceutically acceptable carrier. The disclosure provides an antiviral composition comprising an effective amount of anti-ACE-2-immunoglobulin Y (IgY) antibodies, and a pharmaceutically acceptable carrier.

The disclosure provides an antiviral composition comprising an effective amount of anti-SARS-CoV-2-RBD-protein immunoglobulin Y (IgY) antibodies, an effective amount of anti-human ACE-2 IgY antibodies, and a pharmaceutically acceptable carrier.

In some embodiments, the SARS-CoV-2-S-protein comprises an amino acid sequence selected from the group consisting of SEQ ID NO: 1, 6, 7, 10, 11, 12, 13, 14, 15, 16, 17, 18, 26, 27, 36, 37, 38, 39, 40, 41, 86 or a fragment thereof comprising from 50 to 500, or from 100 to 300, or at least 10, 20, 30, 40, 50, 60, 70, 80, 100, 150, or 200 contiguous amino acid residues thereof, or a substantially similar protein

In some embodiments, the human ACE2 protein comprises an amino acid sequence selected from the group consisting of SEQ ID NO: 28, 35, 42, 43, 77, and 78, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

In some embodiments, the SARS-CoV-2-N-protein comprises an amino acid sequence selected from the group consisting of SEQ ID NO: 4, 8, 9, 24, 25, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

In some embodiments, the SARS-CoV-2-RBD-protein comprises an amino acid sequence selected from the group consisting of SEQ ID NO: 15, 36, 37, 38, 39, 122, 123 or a fragment thereof comprising from 10 to 200, 10 to 100, 20 to 50, or at least 10, 20, 30, 40, 50, 60, 70, 80, 100, 150, or 200 contiguous amino acid residues thereof, or a substantially similar protein thereof.

The antiviral composition according to the disclosure may further comprise anti-SARS-CoV-2 envelope protein-specific IgY antibodies. In some embodiments, the anti-SARS-CoV-2 envelope protein comprises the amino acid sequence of SEQ ID NO: 3, or a fragment comprising from 10 to 75, 20 to 50, or at least 10, 20, 30, 40, or 50 contiguous amino acid residues thereof, or a substantially similar protein thereof.

The antiviral composition according to the disclosure may further comprise anti-TMPRSS2 IgY antibodies.

The antiviral composition according to the disclosure may further comprise a flavoring, sweetener, stabilizer, pH regulator, preservative, antibody matrix, or vitamin.

The IgY antibodies according to the disclosure may be in the form of isolated IgY antibodies, whole immune egg, immune egg yolk, defatted immune egg yolk, whole immune egg powder, immune egg yolk powder, defatted immune egg yolk powder, egg extract, serum, or serum extract. The isolated IgY antibodies may be in a purified and or concentrated form. The IgY antibodies according to the disclosure may comprise polyclonal IgY antibodies. In embodiments, the IgY antibodies according to the disclosure comprise neutralizing polyclonal IgY antibodies.

In some embodiments, the neutralizing anti-SARS-CoV-2-S-protein immunoglobulin Y (IgY) antibodies may be derived from eggs of hens inoculated with an immunogen selected from the group consisting of recombinant SARS-CoV-2 S1-RBD-protein or a fragment thereof, recombinant SARS-CoV-2 S-protein or a fragment thereof, recombinant SARS-CoV-2 S-ECD protein or a fragment thereof, recombinant SARS-CoV-2 S1-protein or a fragment thereof, and recombinant SARS-CoV-2 S2-protein or a fragment thereof.

In some embodiments, the compositions according to the disclosure may comprise anti-bovine coronavirus polyclonal IgY antibodies, anti-avian coronavirus polyclonal IgY antibodies, anti-porcine coronavirus polyclonal IgY antibodies, anti-canine coronavirus polyclonal IgY antibodies, anti-ferret coronavirus polyclonal IgY antibodies, and or anti-feline coronavirus polyclonal IgY antibodies.

The disclosure provides a dosage form comprising a composition according to the disclosure in the form of a spray, mouth spray, nasal spray, inhalation aerosol, lozenge, troche, gel, mucoadhesive gel, film, liquid, powder, capsule, tablet, caplet, mouth wash, mouth rinse, mouth gargle, inhalable powder, suppository, inhalable fluid, and injectable fluid.

In some embodiments, a method of reducing viral replication in a cell is provided comprising treating a coronavirus-infected cell with an effective amount of a composition according to the disclosure.

In some embodiments, a method for the treatment or prevention of a viral infection in a subject in need thereof is provided, comprising administering to or exposing the subject to an effective amount of a composition according to the disclosure. The viral infection may be a SARS-CoV-2 viral infection, optionally wherein the SARS-CoV-2 viral infection is COVID-19.

In some embodiments, a method for reducing severity or duration of symptoms of a coronavirus infection in a subject in need thereof, comprising administering a composition according to the disclosure, optionally wherein the coronavirus infection is COVID-19. The symptoms may be selected from the group consisting of fever, cough, muscle aches, lethargy, diarrhea, vomiting, headache, stomachache, shortness of breath, muscle pain, sputum production, diarrhea, sore throat, complete or partial loss of smell, and complete or partial loss of taste.

In some embodiments, a vaccine composition for production of polyclonal antibodies in a production animal is provided, the vaccine composition comprising a recombinant SARS-CoV-2-S-protein, and optionally an adjuvant. In some embodiments, the vaccine composition may further comprise are combinant protein selected from the group consisting of recombinant human ACE-2 protein and recombinant SARS-CoV-2-N-protein. In some embodiments, the vaccine composition may further comprise a veterinary or human coronavirus vaccine. The vaccine composition may further comprise a live, attenuated, inactivated, or killed coronavirus, optionally wherein the live, attenuated, inactivated, or killed coronavirus is an avian coronavirus, bovine coronavirus, porcine coronavirus, canine coronavirus, human coronavirus, and/or feline coronavirus.

The disclosure provides a kit for preventing or decreasing transmission of a SARS-CoV-2 virus, comprising in at least one container, an antiviral composition according to the disclosure, and optionally at least a second container comprising a diluent, a sheet of instructions, and/or an applicator.

The disclosure provides an antivirotic filter treated with a composition of the disclosure.

The disclosure provides a pharmaceutical composition comprising an effective amount of isolated anti-coronavirus IgY antibodies, immune egg, or immune egg yolk derived from eggs of poultry vaccinated with a coronavirus vaccine, recombinant polynucleotide encoding a SARS-CoV-2 protein, and/or a recombinant SARS-CoV-2 protein, or fragment thereof, and a pharmaceutically acceptable excipient or carrier. The anti-coronavirus IgY antibodies may comprise anti-SARS-CoV-2 coronavirus protein IgY antibodies.

The SARS-CoV-2 protein may be selected from the group consisting of an S-, S1, -S2-, RBD-, S1-S2-ECD, S-RBD, N-, or M-SARS-CoV-2 protein.

A composition of the disclosure may further comprise an effective amount of isolated anti-human ACE2 IgY antibodies, immune egg, or immune egg yolk derived from eggs of poultry vaccinated with a recombinant polynucleotide encoding a human ACE2 protein and/or a recombinant or synthetic human ACE2 protein.

In some embodiments, the composition according to disclosure is appropriate for use in treating, preventing, and or decreasing transmission of a SARS-CoV-2 infection.

In some embodiments, the composition according to disclosure is appropriate for use in the manufacture of a medicament for treating, preventing, and or decreasing transmission of a SARS-CoV-2 infection.

The disclosure provides a method for producing immunoglobulin Y (IgY) polyclonal antibodies comprising identifying a target pathogen and/or target biomolecule; selecting a first immunogen derived from the target pathogen and/or biomolecule; preparing a first inoculant comprising the first immunogen, a first adjuvant, and a first vehicle or carrier; inoculating a host avian with the first inoculant; optionally reinoculating the host avian with the first inoculant or a second inoculant comprising a second immunogen, a second adjuvant, and a second vehicle or carrier; collecting eggs and/or blood from the host avian; and processing the eggs or blood to obtain isolated IgY antibodies. In some embodiments, the second inoculant is prepared comprising selecting a second immunogen derived from the target pathogen or target biomolecule; and preparing the second inoculant comprising the second immunogen, the second adjuvant, and the second vehicle.

In some embodiments, the first, second, and or subsequent immunogens are selected from the group consisting of a fixed, attenuated, or inactivated whole cell immunogen, a protein immunogen, and a plasmid DNA encoding a protein immunogen. In some embodiments, the first and second immunogens are different.

In some embodiments, the first, second, and/or subsequent immunogen is a protein immunogen, wherein the protein immunogen is selected from the group consisting of an isolated protein, synthetic protein, or a recombinant protein. In some embodiments, the first, second, and/or subsequent immunogen is a plasmid DNA immunogen encoding a protein immunogen.

In a specific embodiment, the first immunogen is a protein immunogen selected from the group consisting of an isolated protein, synthetic protein, or a recombinant protein; and the second immunogen is a plasmid DNA immunogen encoding the protein immunogen.

In some embodiments, the target pathogen may be selected from the group consisiting of coronavirus such as SARS-CoV-2 virus, SARS-CoV, MERS, norovirus, zika virus such as PRV ABC59, rhinovirus, herpes virus, influenza virus, smallpox virus, Ebola virus, rotavirus, calicivirus, cytomegalovirus, astrovirus, adenovirus, enteric adenovirus, Staphylococcus aureus, Vibrio cholerae such as Vibrio O1, Vibrio O139, Non-O1 Vibrios, Vibrio parahaemolyticus, Campylobacter jejuni, Salmonella spp. such as Salmonella typhimurium, Salmonella enterica serovar Typhi, bacillus spp. such as Bacillus cereus, Bacillus anthracis, Shigella dystenteriae, Plasmodium falciparum, Plesiomonas shigelloides, Escherichia coli [including (EPEC) enteropathogenic E. coli, (ETEC) enterotoxigenic E. coli, (EaggEC) enteroaggregative E. coli, (EIEC) enteroinvasive E. coli, and (EHEC) haemorrhagic E. coli], Yersinia enterocolitica, Aeromonas hydrophila, Clostridium perfringens, Clostridium difficile, enterohepatic Helicobacter (including Helicobacter pylori), Staphylococcus aureus, Klebsiella spp., Mycobacterium tuberculosis, Streptococcus pyogenes, Salmonella enterica serotypes Paratyphi A and B, Enterobacter spp. such as Enterobacter cloacae or Enterobacter sakazakii, Aeromonas spp. such as A. caviae, A. veronii biovar sobria, Proteus spp. such as P. mirabilis or P. vulgaris, Citrobacter spp. such as C. freundii, Serratia spp. such as S. marcescens, S. rubidaea, Cryptosporidium spp., venom, toxin such as cholera toxin, adhesion element, prion protein, and prion-like protein. In some embodiments, the coronavirus is a SARS-CoV-2 virus.

The target biomolecule may be a human ACE 2 protein, or fragment thereof, or a substantially similar protein.

In some embodiments, the first and/or second adjuvant and/or subsequent adjuvant is selected from the group consisting of Freund's Complete Adjuvant (FCA), Freund's Incomplete Adjuvant, mineral adjuvants, such as aluminum compounds, aluminum hydroxide, ALUM, potassium alum, potassium aluminum sulfate, aluminum hydroxy phosphate sulfate, aluminum phosphate, calcium phosphate hydroxide, bacterial adjuvants such as muramyl dipeptides, flagellin, monophosphoryl lipid A, killed Bordetella pertussis, Mycobacterium bovis, toxoids, lipopolysaccharide, aluminum monostearate, mannide monooleate, vegetable oil, paraffin oil, water, polysorbate 80, polysorbate 20, octoxynol-10, octylphenol ethoxylate, block copolymer, CRL-89-41, squalene, oil in water emulsion comprising squalene, Titermax Classical adjuvant (SIGMA-ALDRICH), lipid based immunostimulant complexes (ISCOMS) mix of cholesterol, dioleoyl phosphatidyl choline, 3-O-desacyl-4′monophosphoryl lipid A, Quillaja saponins, Quil A, Lipid A derivatives, cholera toxin derivatives, diphtheria toxoid, heat shock protein (HSP) derivatives, lipopolysaccharide (LPS) derivatives, synthetic peptide matrixes, GMDP, oil-based adjuvant such as Xtend®III (Grand Laboratories, Inc., Larchwood, Iowa) immunostimulants (U.S. Pat. No. 5,876,735), interleukins such as IL-1, IL-2, IL-6, IL-8, IL-12, IL-15, IL-18, cytokines such as interferon gamma, chGMCSF, Flt3 ligand, class B oligodeoxynucleotide (ODN) CpG, phosphorothioate-linked oligodeoxynucleotide, and a plasmid adjuvant DNA encoding a cytokine, interleukin, or heat shock protein. The plasmid adjuvant may comprise a eukaryotic expression vector and encodes a cytokine, interleukin, or heat shock protein, optionally selected from the group consisting of interferon gamma (IFNγ), heat shock protein from M. tuberculosis (HSP70), interleukin-2 from Gallus gallus (IL-2), IL-6, IL-8, IL-15, chicken granulocyte-macrophage colony stimulating factor (chGMCSF), cytokine Flt3 ligand, CCL19. The eukaryotic expression vector of the plasmid adjuvant may be selected from the group consisting of pCI-neo mammalian expression vector, pVIVO2-mcs vector, pVAX1 vector, pIRES Vector, and a pcDNA 3.1 mammalian expression vector.

In some embodiments, the first and/or second and/or subsequent vehicle or carrier comprises one or more components selected from the group consisting of water, phosphate buffered saline, a physiologic buffer, sodium chloride, sucrose, lactose, trehalose, dextrose, microcrystalline cellulose, potassium phosphate, sodium phosphate, magnesium stearate, sodium bicarbonate, sodium carbonate, 2-phonoxyathanol, protamine sulfate, urea, citric acid, sodium metabisulfite, monosodium glutamate, ethlenediamine tetraacetic acid (EDTA), optionally wherein the vehicle or carrier comprises a preservative. The optional preservative may be selected from the group consisting of neomycin, neomycin sulfate, polymixin B, thimerosal, formaldehyde, quaternary ammonium preservative such as benzalkonium chloride, and phenol.

The protein immunogen may be selected from the group consisting of SARS-CoV-2 RBD-protein, SARS-CoV-2 S-protein, SARS-CoV-2 S2-protein, SARS-CoV-2 S1-protein, SARS-CoV-2N-protein, human ACE2 protein, norovirus capsid protein, Plasmodium falciparum circumsporozoite protein, Cryptosporidium protein such as C. parvum P23, a Clostridium difficile protein FliC, FliD, Cwp84, or Toxin B (TcdB), Staphylococcal protein A, CD20 protein, venom, rhinovirus VP4 protein, influenza VP1 capsid protein, prion protein, prion-like protein, herpes simplex virus glycoprotein gD, herpes simplex virus glycoprotein gD, rotavirus VP4 capsid protein, rotavirus VP7 surface glycoporotein, rotavirus NSP4 viral enterotoxin, zika virus NS-1 protein, Smallpox virus vaccinia complement protein (VCP), Bacillus anchracis lethal factor, Bacillus anchracis edema factor, Bacillus anchracis protective antigen (pagA), Ebola virus glycoprotein, Staphylococcus aureus SpA, cholera toxin subunit A, cholera toxin subunit B, and cholera toxin AB5.

In some embodiments, the host avian may be a chicken.

In some embodiments, the first and/or second and/or subsequent immunogen is a plasmid DNA encoding a protein of the target pathogen or a biomolecule. The plasmid DNA may comprise a eukaryotic expression vector. The eukaryotic expression vector may be selected from the group consisting of pCI-neo mammalian expression vector, pVIVO2-mcs vector, pVAX1 vector, pIRES Vector, and a pcDNA 3.1 Mammalian Expression Vector. The eukaryotic expression vector may be a pCI-neo mammalian expression vector comprising the sequence of SEQ ID NO: 83. The plasmid DNA may encodes a SARS-CoV-2 S-protein, SARS-CoV-2 RBD-protein, and/or human ACE2 protein.

The disclosure provides a method for preparing a plasmid DNA immunogen, comprising a) selecting a target protein amino acid sequence or a DNA sequence encoding the target protein amino acid sequence; b) optimizing the codons of a DNA sequence encoding the amino acid sequence of the target protein for expression in Gallus gallus to obtain a codon-optimized target DNA sequence; and c) cloning the codon-optimized target DNA sequence into a eukaryotic expression vector to obtain the plasmid DNA immunogen. The eukaryotic expression vector may be selected from the group consisting of pCI-neo mammalian expression vector, pVIVO2-mcs vector, pVAX1 vector, pIRES Vector, and a pcDNA 3.1 mammalian expression vector.

The target protein sequence may be selected from the group consisting of a SARS-CoV-2 S-protein, SARS-CoV-2 S1-protein, SARS-CoV-2 RBD-protein, SARS-CoV-2N-protein, human ACE2 protein, norovirus capsid protein, Plasmodium falciparum circumsporozoite protein, Cryptosporidium protein such as C. parvum P23, a Clostridium difficile protein, for example, FliC, FliD, Cwp84, or Toxin B (TcdB), Staphylococcal protein A, CD20 protein, venom, rhinovirus VP4 protein, influenza VP1 capsid protein, prion protein, prion-like protein, herpes simplex virus glycoprotein gD, herpes simplex virus glycoprotein gD, rotavirus VP4 capsid protein, rotavirus VP7 surface glycoporotein, rotavirus NSP4 viral enterotoxin, zika virus NS-1 protein, Smallpox virus vaccinia complement protein (VCP), Bacillus anchracis lethal factor, Bacillus anchracis edema factor, Bacillus anchracis protective antigen (pagA), Ebola virus glycoprotein, Staphylococcus aureus SpA, cholera toxin subunit A, cholera toxin subunit B, or cholera toxin AB5, or a fragment thereof, or substantially similar protein.

The plasmid DNA may encode a SARS-CoV-2-S-protein comprising the amino acid sequence selected from the group consisting of SEQ ID NO: 1, 6, 7, 10, 11, 12, 13, 14, 15, 16, 17, 18, 36, 37, 38, 39, 40, 41, 86, or a fragment thereof comprising from 50 to 1000, or from 100 to 500, or at least 10, 20, 30, 40, 50, 60, 70, 80, 100, 150, or 200 contiguous amino acid residues thereof, or a substantially similar protein.

The plasmid DNA may encode a human ACE2 protein comprising an amino acid sequence of SEQ ID NO: 28, 35, 42, 43, 77, 78, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

The plasmid DNA may encode a SARS-CoV-2-N-protein comprising an amino acid sequence selected from the group consisting of SEQ ID NO: 4, 8, 9, 24, 25, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

The plasmid DNA may encode a SARS-CoV-2-RBD-protein comprising an amino acid sequence selected from the group consisting of SEQ ID NO: 15, 36, 37, 38, 39, 122, 123 or a fragment thereof comprising from 10 to 200, 10 to 100, 20 to 50, or at least 10, 20, 30, 40, 50, 60, 70, 80, 100, 150, or 200 contiguous amino acid residues thereof, or a substantially similar protein thereof.

The disclosure provides an oral composition comprising an effective amount of anti-SARS-CoV-2-S-immunoglobulin Y (IgY) antibodies, an effective amount of anti-human ACE-2 IgY antibodies, and a pharmaceutically acceptable carrier. The anti-SARS-CoV-2-S-immunoglobulin Y (IgY) antibodies may comprise anti-SARS-CoV-2-RBD-immunoglobulin Y (IgY) antibodies. The oral composition may further comprise a flavoring, sweetener, stabilizer, pH regulator, preservative, antibody matrix, or vitamin. The oral composition or dosage form may comprise an enteric coating. The oral composition or dosage form may comprise a mucoadhesive gel.

The disclosure provides a dosage form comprising an oral composition according to the disclosure in the form of a spray, lozenge, troche, gel, mucoadhesive gel, film, liquid, powder, capsule, tablet, caplet, mouth rinse, or powder.

The disclosure provides improved methods for rapid production of polyclonal IgY antibodies for use in antiviral, antibacterial, anti-venom, anti-toxin, anti-virulence factor, anti-adherence factor, anti-prion, or anti-prion-like protein, therapeutic or prophylactic compositions.

### BRIEF DESCRIPTION OF THE DRAWINGS

FIG. 1 shows a DNA ladder used to check the PCR products that were run on agarose gels in FIGS. 2A, 2B, and 2C.

FIGS. 2A-C shows photographs of agarose gels illustrating the results of a PCR screen targeting the E. coli expression plasmids assembles and transformed into E. coli BL21 (DE3).

FIG. 2A is a photograph of a gel showing the results for the plasmid pRAB11_P_{XYL/TET}-N (C-terminus his-tag) using the primers DR_788 and DR_215. Positive bands will be 933 base pairs (bp).

FIG. 2B is a photograph of a gel showing the results for the plasmid pRAB11_P_{XYL/TET}-N (N-terminus his-tag) using the primers DR_215 and DR_216. Positive results have a band at 1911 bp.

FIG. 2C is a photograph of a gel showing the results for the plasmids pRAB11_P_{XYL/TET}-S (N-terminus his-tag) (wells 1-9 on the top row) and pRAB11_P_{XYL/TET}-S (C-terminus his-tag) (all other samples on the gel). The primers DR_776 and DR_771 were used and positive colonies have a band at 191 bp. Positive samples for this PCR are represented by tight bands as shown in the top row far left.

FIG. 3 shows a photograph of an agarose gel of PCR products generated to build the E. coli expression vectors for production of SARS-CoV-2 recombinant proteins. Four separate PCR reactions were run for the pRAB backbone fragment generation (lanes 1-4), one for each expression plasmid being built. The expected size of the pRAB backbone fragments are around 6500 base pairs (lanes 1-4), the expected size of the S gene fragments (lanes 5, 7, 8 and 9) are about 3800 base pairs, the expected size of the N gene fragments (lanes 10 and 11) are about 1290 base pairs. Lane 6 shows a DNA ladder with arrows pointing to 1 kb, 2 kb, 4 kb and 7 kb base pair standards.

FIG. 4A and FIG. 4B show photographs of PCR reactions on a 1% agarose gel that were run to screen for fully assembled plasmids. Colonies that grew on the agar plates following the transformation of the Gibson assembly mixture were picked and whole cell lysate was used as the template for the PCR reaction.

FIG. 4A shows the results from screening for the presence of the S gene using the primers DR_776 and DR_771. Positive bands are 191 base pairs and appear as defined bands such as in lane 1.

FIG. 4B shows the results from screening for the presence of the N gene using the primers DR_788 and DR_215. Positive bands are 933 base pairs and appear as defined bands such as in lane 1.

FIG. 5A shows Dot ELISA performed by applying recombinant SARS-CoV-2 proteins S1 (E. coli expressed), S2 (E. coli expressed), N (E. coli expressed) and S2* (Baculovirus insect cell line expressed glycosylated S2 protein) antigens to nitrocellulose paper at dilutions of 0.25 μg, 0.10 μg, 0.05 μg, 0.01 μg, and 0.001 μg in duplicate, adding primary IgY antibodies from day 12 black egg group dehydrated immune eggs, then adding secondary antibodies goat anti-chicken IgY HRP at 1:10,000. Clear binding activity is seen against each of three major subunits of the virus.

FIG. 5B shows a representative Western blot showing Red flock immune egg IgY binding activity against the subunits of the SARS-CoV-2 virus. Clear IgY binding activity against each of the three major subunits of the SARS-CoV-2 virus including S1 (˜75 kDa), S2 (˜58 kDa), N (˜50 kDa), and S2* glycosylated protein (˜59 kDa) is demonstrated.

FIGS. 6A-E show a Gel and a series of Western Blots, using 1 μg of each of the following antigens: S1 (E. coli expressed), S2 (E. coli expressed), N (E. coli expressed), S (S1+S2 ECD Sino, Baculovirus expressed insect cell glycosylated S protein-His tagged; 134 kDa) and probed with prepared with IgY extracts from various egg conditions as primary antibodies. All primary antibodies from different flocks were loaded at 0.015 mg/mL in 10 mL PBST. The secondary antibodies used were goat anti-chicken IgY (Invitrogen 1:10,000).

FIG. 6A shows Coomasie stained TRIS-Acetate gel for each of SARS-CoV-2 S1, S2, N, and S recombinant proteins.

FIG. 6B shows Western blot of Black flock derived IgY showing prominent binding to S2 and S antigens.

FIG. 6C shows Western blot of Red flock derived IgY showing good binding to each of S1, S2, N, and S antigens.

FIG. 6D shows Western blot of SCOURGUARD inoculated flock derived IgY showing good binding to S2 and S antigens.

FIG. 6E shows Western blot of IgY derived from store bought eggs. Surprisingly, binding to S2 and S proteins was exhibited.

FIGS. 7A-7F show another series of gels and Western Blots prepared with IgY processed at different times, or by different methods from eggs from a single flock (Black flock), using the following SARS-CoV-2 antigens: S1=RayBiotech E. coli expressed S1 protein (Val16-Gln690; N-terminal His tagged ˜75 kDa), S2=RayBiotech E. coli expressed S2 protein (Met697-Pro1213; ˜58 kDa), N=Raybiotech E. coli expressed nucleocapsid N protein (Met1-Ala419; ˜50 kDa), S2*=Sino, Baculovirus expressed insect cell glycosylated S2 protein-His tag (Ser686-Pro1213; 59.37 kDa).

FIG. 7A shows Coomasie stained TRIS-Acetate gel.

FIG. 7B shows Western blot of IgY extract derived from Black flock on 5-13-20, clearly exhibiting binding to S2 and S2*antigens.

FIG. 7C shows Western blot of IgY extract derived from Black flock on 5-22-20, clearly exhibiting binding to S2 and S2*, and some binding to S1 and N antigens.

FIG. 7D shows Western blot IgY extract from dehydrated eggs derived from Black flock, clearly exhibiting binding to S2 and S2*, some binding to S1, and faint binding to N antigens.

FIG. 7E shows binding from dehydrated immune eggs from Black flock, clearly exhibiting binding to S2 and S2*, some faint binding to N antigens.

FIG. 7F shows binding from whole immune eggs from Black flock, exhibiting binding to S2, S2*, and N antigens.

FIGS. 8A and B show blocking dot ELISAs with ACE2 (Acros, not His-Tagged) dotted on nitrocellulose paper at 0.1 ug across all conditions, with the exception of the ACE2 (−) control wells, where PBS was coated. Recombinant SARS-CoV-2 S1+S2 extracellular domain (ECD) protein His-tagged (Sino Biological, Inc.) was then dotted directly onto the ACE2 in the amounts 1.0 ug, 0.1 ug, 0.05 ug, 0.01 ug, or 0 ug (−). The blots were probed with Anti-His HRP antibodies (1:1000).

FIG. 8A shows blocking dot ELISA results where color development can be seen for the dots which were exposed to 1.0, 0.1 and 0.05 ug of recombinant SARS-CoV-2 S1+S2 ECD His-tagged antigen. No color development occurred for the 0.01 ug S1+S2 ECD condition or any of the negative controls.

FIG. 8B shows blocking dot ELISA results where color development can be seen for the dots which were exposed to 1.0, 0.1, 0.05, and 0.01 ug of recombinant SARS-CoV-2 S1+S2 ECD His-tagged antigen. Faint color development can be seen for the (−) ACE2 control where high concentration of 1 ug of S1+S2 was added. No color development occurred for the any of the remaining (−) controls.

FIG. 9A shows ELISA reactivity of IgY isolated from certain immune eggs of Table 1B in binding to SARS-CoV-2 S1 and S2 proteins. Green eggs (S1 protein produced in E. coli) exhibited highest reactivity, followed by IgY isolated from Blue eggs (S2 protein produced in E. coli, denatured), store bought eggs, Red eggs (N protein produced in E. coli) and lastly IgY from Scourguard® eggs. Surprisingly, IgY from store bought eggs exhibited binding to S1 and S2 in the ELISA.

FIG. 9B shows ELISA reactivity of IgY isolated from certain immune eggs of Table 1B in binding to SARS-CoV-2N-protein. Red eggs (N protein produced in E. coli) exhibited highest reactivity, followed by Blue eggs (S2 protein produced in E. coli, denatured), and Green eggs (S1 protein produced in E. coli). Very low reactivity was exhibited by IgY isolated from store bought and Scourguard® eggs.

FIG. 10 shows the analysis of the soluble fraction of E. coli harboring N protein expression plasmids FB_jp2 and FB_jp5. (A, top) a membrane from a western blot that was probed with an anti-his HRP conjugated IgG, and (A, bottom) a membrane that was first probed with an anti-his primary IgG and then a chicken anti-rabbit HRP conjugated IgY. (B) shows a PAGE gel that was run at the same time as the gels used in the western blot, but was stained with coomassie blue. A band produced at the correct size in lane 2 indicates that the FBB_jp2 cultures were able to express the protein compared to N protein positive control in lane 12.

FIG. 11 shows a map of the p020 plasmid for human ACE2 made in the Benchling program.

FIG. 12A shows a photo of a Coomassie stained gel of recombinant human ACE2 with human ACE2 standards and both soluble and insoluble fractions of E. coli cell lysates from FBB_p020 production batches. Lane 1 and 2 shows two lots of soluble fraction, lanes 3 and 4 show 2 lots of solubilized inclusion bodies (SIB) PBS dialyzed, lanes 5, 6, 7 show ACE2 standards loaded at 0.8 ug, 0.4 ug, 0.1 ug, respectively, Lane 8 shows Thermo Scientific Spectra Multicolor Broad Range Protein Ladder with bands at top to bottom ˜260 kDa, ˜140 kDa, ˜100 kDa, ˜70 kDa, ˜50 kDa, ˜40 kDa, ˜35 kDa, ˜25 kDa, ˜15 kDa, and ˜10 kDa. Lane 9 and 10 show soluble fractions, lanes 11 and 12 show SIB PBS dialyzed.

FIG. 12B shows a photo of a Western blot of recombinant human ACE2 using anti-His tag probed membrane. The antibodies used to probe and develop the blot above were: primary=anti-His, secondary=anti-mouse HRP conjugated. Lanes 1-12 are the same as shown for Coomassie stained gel shown in FIG. 12A.

FIG. 12 C shows a Western blot using anti-ACE2 probed membrane. The antibodies used in the Western blot above are; primary=anti-ACE2 (anti-ACE2 polyclonal Antibody (PA5-20045, Invitrogen) developed using immunogen synthetic peptide corresponding to amino acids near N-terminus of human ACE2 purified by antigen affinity chromatography), secondary=anti-rabbit IgG. The lanes in the blots are the same as the Coomassie stained gel shown in FIG. 12A.

FIG. 13A shows a photograph of the Western blot of recombinant ACE2 protein produced according to the disclosure at left compared to 0.1 ug ACE2 standard protein at right used for protein calculation using ImageJ software.

FIG. 13B shows a photograph of the histogram produced by each box in FIG. 13A of Western blots of FIG. 13A using ImageJ software and baseline used for determining peak area for quantitation of recombinant ACE2 proteins.

FIG. 14 shows a graph of percent inhibition of RBD:hACE2 binding versus anti-RBD IgY concentration (mg/mL) in the first three weeks post-inoculation at Day 0, Day 14, and Day 21 compared to negative control extracted non-targeted IgY, by ELISA using ACRO Biosystems EP-105 Kit. A significant increase in titer from Day 0 to Day 21 is observed.

FIG. 15 shows a graph of percent inhibition of RBD:hACE2 binding versus anti-RBD IgY concentration (mg/mL) at Day 28, Day 35, and Day 50 post-inoculation, compared to negative control extracted non-targeted IgY, by ELISA using ACRO Biosystems EP-105 Kit. Note the change in the x-axis from FIG. 14 to FIG. 15 to accommodate the increase in RBD-specific IgY titer. A significant increase in titer occurred from Day 0 until Day 28 when that titer is then maintained through at least day 50, as shown in FIG. 15.

FIG. 16 shows a graph of the percent inhibition of RBD:ACE2 binding vs. anti-RBD IgY concentration (mg/mL) in serum extracted from inoculated chickens at Days 15 and 29 post-inoculation, tested with ELISA using the ACRO Biosystems ELISA EP-105 Kit. A significant increase in serum titer is seen between Day 15 and Day 29.

FIG. 17 shows a graph of ELISA reactivity of anti-RBD IgY isolated from raw egg yolk against coated RBD protein. The IgY antibodies were extracted from inoculated chicken eggs, collected at varying time points following RBD inoculation, compared to IgY antibodies sourced from non-targeted chicken eggs. OD at 450 nm was measured using a UV/V is microplate spectrophotometer and plotted against the concentration of total IgY measured by NanoDrop by A₂₈₀. Each sample was plated in duplicate and the averages and standard deviations were calculated. Eggs collected pre-inoculation (Day 0) and non-targeted IgY antibodies show little to no binding activity. The RBD binding activity for each sample dilution series shows a steady increase in RBD-specific antibody titer from Day 14 to Day 50.

FIG. 18 shows a graph of ELISA reactivity of anti-RBD IgY from serum of inoculated chickens against coated RBD protein. Blood was collected at day 15 and 29 following initial RBD inoculation. Reactivity of serum sourced from non-targeted chicken blood was used as negative control. Samples were run in duplicate. Error bars indicate standard deviation. Serum IgY antibodies exhibited ELISA reactivity similar to the egg yolk IgY. Serum IgY exhibited escalating specific ELISA reactivity to the inoculated target protein (RBD) in vitro from day 15 to day 29.

FIG. 19A shows a graph of ELISA activity of a dilution series of extracted anti-Wuhan strain anti-RBD IgY antibodies binding to SARS-CoV-2 RBD [N501Y] mutant protein comprising amino acid sequence of SEQ ID NO: 37 coated to 96 well plate. Sample IgY is detected using goat-anti-chicken IgY-HRP, followed by TMB substrate. Average OD at 450 nm vs. total IgY concentration is plotted; error bars indicate standard deviation. Each sample was plated in duplicate. Negative control is IgY extracted from eggs of non-immunized (non-targeted) chickens.

FIG. 19B shows a graph of ELISA activity of a dilution series of extracted anti-Wuhan strain anti-RBD IgY antibodies binding to SARS-CoV-2 RBD [E484K] mutant protein coated to 96 well plate. Sample IgY is detected using goat-anti-chicken IgY-HRP, followed by TMB substrate. Average OD at 450 nm vs. total IgY concentration is plotted; error bars indicate standard deviation. Each sample was plated in duplicate. Negative control is IgY extracted from eggs of non-immunized (non-targeted) chickens.

FIG. 19C shows a graph of ELISA activity of a dilution series of extracted anti-Wuhan strain anti-RBD IgY antibodies binding to SARS-CoV-2 RBD [K417N] mutant protein coated to 96 well plate. Sample IgY is detected using goat-anti-chicken IgY-HRP, followed by TMB substrate. Average OD at 450 nm vs. total IgY concentration is plotted; error bars indicate standard deviation. Each sample was plated in duplicate. Negative control is IgY extracted from eggs of non-immunized (non-targeted) chickens.

FIG. 19D shows a graph of ELISA activity of a dilution series of extracted anti-Wuhan strain anti-RBD IgY antibodies binding to SARS-CoV-2 South African Spike S1 Variant [K417N, E484K, N501Y, D614G] protein coated to 96 well plate. Sample IgY is detected using goat-anti-chicken IgY-HRP, followed by TMB substrate. Average OD at 450 nm vs. total IgY concentration is plotted; error bars indicate standard deviation. Each sample was plated in duplicate. Negative control is IgY extracted from eggs of non-immunized (non-targeted) chickens.

FIG. 20A shows a graph of competitive ELISA showing inhibition of ACE2:SARS-CoV-2 RBD [N501Y] mutant binding by anti-Wuhan strain RBD IgY. The percent inhibition averages as compared to positive control wells without IgY antibody, of the anti-RBD IgY and the non-targeted IgY samples inhibiting the RBD [N501Y] Mutant:ACE2 interaction, was tested in the EP-105 ACRO inhibition assay. Error bars indicate standard deviation. Each sample dilution was tested in duplicate. Very low inhibition was exhibited by negative control non-targeted extracted IgY at all tested concentrations. Greater than about 97% inhibition of ACE2:SARS-CoV-2 RBD [N501Y] mutant binding by anti-Wuhan strain RBD IgY was demonstrated at total IgY concentrations above 1 mg/mL.

FIG. 20B shows a graph of competitive ELISA showing inhibition of ACE2:SARS-CoV-2 RBD [E484K] mutant binding by anti-Wuhan strain RBD IgY. The percent inhibition averages as compared to positive control wells without IgY antibody, of the anti-RBD IgY and the non-targeted IgY samples inhibiting the RBD [E484K] Mutant:ACE2 interaction, was tested in the EP-105 ACRO inhibition assay. Error bars indicate standard deviation. Each sample dilution was tested in duplicate. Very low inhibition was exhibited by negative control non-targeted extracted IgY. Greater than about 92% inhibition of ACE2:SARS-CoV-2 RBD [E484K] mutant binding by anti-Wuhan strain RBD IgY was demonstrated at total IgY concentrations above 1 mg/mL.

FIG. 20C shows a graph of competitive ELISA showing inhibition of ACE2:SARS-CoV-2 RBD [K417N]mutant binding by anti-Wuhan strain RBD IgY. The percent inhibition averages as compared to positive control wells without IgY antibody, of the anti-RBD IgY and the non-targeted IgY samples inhibiting the RBD [K417N] Mutant:ACE2 interaction, was tested in the EP-105 ACRO inhibition assay. Error bars indicate standard deviation. Each sample dilution was tested in duplicate. Very low to no inhibition was exhibited by negative control non-targeted extracted IgY. Greater than about 95% inhibition of ACE2:SARS-CoV-2 RBD [K417N] mutant binding by anti-Wuhan strain RBD IgY was demonstrated at total IgY concentrations above 1 mg/mL.

FIG. 20D shows a graph of competitive ELISA showing inhibition of ACE2:SARS-CoV-2 South Africa Spike S1 variant [K417N, E484K, N501Y, D614G] binding by anti-Wuhan strain RBD IgY. The percent inhibition averages as compared to positive control wells without IgY antibody, of the anti-RBD IgY and the non-targeted IgY samples inhibiting the SARS-CoV-2 S1 Variant:ACE2 interaction, was tested in the EP-105 ACRO inhibition assay. Error bars indicate standard deviation. Each sample dilution was tested in duplicate. Very low to no inhibition was exhibited by negative control non-targeted extracted IgY. Greater than about 96% inhibition of ACE2:SARS-CoV-2 S1 variant [K417N, E484K, N501Y, D614G] binding by anti-Wuhan strain RBD IgY was demonstrated at total IgY concentrations above 1 mg/mL.

FIG. 20E shows a graph of ELISA reactivity to coated SARS-CoV-2 RBD [L452R] Mutant in indirect binding assay for IgY antibodies extracted from chicken eggs 50 days after initial Wuhan RBD inoculation as compared to IgY antibodies sourced from non-targeted chicken eggs. OD at 450 nm was measured and plotted against the concentration of total IgY measured by the NanoDrop™ One© Instrument. Each sample was plated in duplicate and the averages and standard deviations are shown.

FIG. 20F shows a graph of ELISA reactivity to coated SARS-CoV-2 RBD [S477N] mutant in an indirect binding assay for anti-RBD IgY antibodies extracted from chicken eggs following Wuhan RBD inoculation as compared to IgY antibodies sourced from non-targeted chicken eggs. OD at 450 nm was measured and plotted against the concentration of total IgY measured by the NanoDrop™ One© Instrument. Each sample was plated in duplicate and the averages and standard deviations are shown.

FIG. 21A shows a graph of competitive ELISA results in GenScript SARS-CoV-2 Surrogate Virus Neutralization Test (sVNT) C-Pass™ Kit (GenScript Biotech Corporation). Samples containing anti-SARS-CoV-2 RBD IgY antibodies in formulated spray dried yolk powder harvested from RBD-inoculated chickens at 28, 35 and 50 days after first inoculation were evaluated. Neutralizing anti-SARS-CoV-2 RBD IgY antibodies in formulated spray dried egg yolk powder harvested at 50 days exhibited >90% percent inhibition of RBD:ACE2 binding, within the GenScript sVNT SARS-CoV-2 assay. By comparison, the spray dried non-targeted IgY and spray dried excipient materials displayed a complete absence of neutralizing antibodies, by the sVNT kit standards. Data points were obtained in duplicate and plotted as avg percent inhibition f standard deviation.

FIG. 21B shows a graph of competitive ELISA results for anti-SARS-CoV-2 RBD IgY antibody samples as ability to inhibit ACE2:SARS-CoV-2 RBD binding in the commercial ACRO EP-105 SARS-CoV-2 inhibition assay. Samples containing anti-RBD IgY in spray dried yolk powder harvested from RBD-inoculated chickens at 28, 35 and 50 days after first inoculation were evaluated. Neutralizing anti-RBD IgY in formulated spray dried egg yolk powder harvested at 50 days exhibited >95% percent inhibition of RBD:ACE2 binding. In comparison, the spray dried non-targeted IgY and spray dried excipient materials displayed little to no RBD:ACE binding inhibition indicating an absence of neutralizing antibodies. Data points were obtained in duplicate and plotted as avg percent inhibition±standard deviation.

FIG. 22 shows a graph of inhibition rate (%) of three samples of anti-SARS-CoV-2 RBD IgY in a commercial Cell-Based Pseudovirus Assay (Sino Biological, Inc.) compared to a positive control antibody. Inhibition rates (%) are plotted against antibody concentrations. Sample 1 refers to affinity purified anti-RBD IgY antibodies. Samples 2 and 3 refer to Hodek extracted IgY antibodies containing ˜5-10% specific anti-RBD IgY antibodies. IC₅₀ values are shown in Table 15. anti-RBD IgY antibodies—whether affinity purified or contained within a total IgY extraction-were capable of neutralizing >99% of the pseudovirus activity in the Sino Biological cell-based neutralization assay.

FIG. 23 shows a graph of reactivity of two different pools of anti-ACE2 IgY in chicken serum against coated Creative Biomart-Sourced ACE2 protein in an indirect ELISA assay format. Two groups of chickens were inoculated with recombinant human ACE2 protein from two different suppliers and blood was collected 17 days after initial inoculation. Serum from uninoculated (non-targeted) chickens was used as a negative control. The OD values were plotted against total IgY concentration (mg/mL) to show a dose-dependence in specific antibody concentration. Both pools of anti-ACE2 IgY serum demonstrated reactivity. The Creative Biotech-ACE2 inoculated chicken serum exhibited higher reactivity.

FIG. 24 shows reactivity of two different pools of anti-ACE2 IgY in chicken serum against coated RayBiotech-Sourced ACE2 Protein in an indirect ELISA assay. Two groups of chickens were inoculated with recombinant human ACE2 protein from two different suppliers and blood was collected 17 days after initial inoculation. Serum from uninoculated (non-targeted) chickens was used as a negative control. The OD values were plotted against total IgY concentration (mg/mL) to show a dose-dependence in specific antibody concentration. Both pools of anti-ACE2 IgY serum demonstrated reactivity. The Creative Biotech-ACE2 inoculated chicken serum exhibited higher reactivity.

FIG. 25 shows a graph of percent inhibition of RBD:ACE2 binding v. anti-ACE2 IgY antibody concentration in serum, in a commercial neutralization ELISA assay format, as compared to serum from uninoculated chickens (non-targeted IgY). The anti-(CBio) ACE2 IgY exhibited greater than 95% inhibition of RBD:ACE2 binding at 2 mg/mL total IgY and the anti-(RBio) ACE2 IgY exhibited greater than 85% inhibition at 5 mg/mL total IgY, 17 days after a single inoculation with the recombinant proteins The negative control exhibited less than 5% inhibition.

FIG. 26 shows a graph of ELISA reactivity of two different pools of anti-ACE2 IgY from chicken serum against coated Creative Biomart-sourced recombinant ACE2 protein in an indirect ELISA assay format. Two groups of chickens were inoculated with recombinant human ACE2 protein from two different suppliers and blood was collected 17 days after initial inoculation. Serum from uninoculated (non-targeted) chickens was used as a negative control. The optical density (OD) (absorbance at 450 nm) values were plotted against total IgY concentration (mg/mL) to show a dose-dependence in specific antibody concentration. The Creative Biotech-ACE2 inoculated chicken serum exhibited higher reactivity.

FIG. 27 shows a graph of ELISA reactivity of two different pools of anti-ACE2 IgY from chicken serum against coated RayBiotech-sourced ACE2 protein in an indirect ELISA assay format. Two groups of chickens were inoculated with recombinant human ACE2 protein from two different suppliers and blood was collected 17 days after initial inoculation. Serum from uninoculated (non-targeted) chickens was used as a negative control. The OD (absorbance at 450 nm) values were plotted against total IgY concentration (mg/mL) to show a dose-dependence in specific antibody concentration. The Creative Biotech-ACE2 inoculated chicken serum exhibited higher reactivity.

FIG. 28 shows a graph of percent inhibition of RBD:ACE2 binding v. anti-ACE2 IgY antibody concentration in two different pools of anti-ACE2 IgY from chicken serum, in a commercial neutralization ELISA assay format. Two groups of chickens were inoculated with recombinant human ACE2 protein from two different suppliers and blood was collected 17 days after initial inoculation. Serum from uninoculated (non-targeted) chickens was used as a negative control. The anti-(CBio) ACE2 IgY exhibited greater than 95% inhibition of RBD:ACE2 binding at 2 mg/mL total IgY and the anti-(RBio) ACE2 IgY exhibited greater than 85% inhibition at 5 mg/mL total IgY. The negative control exhibited less than 5% inhibition.

FIG. 29 shows a graph of ELISA reactivity plotted as OD450 v. total IgY concentration between the coated norovirus capsid protein and anti-norovirus capsid protein IgY antibodies present in serum 23 days post the initial inoculation. Serum from uninoculated (non-targeted) chickens was used as a negative control. Each sample was plated in duplicate and the averages and standard deviations are shown in the graph. Specific reactivity was demonstrated.

FIG. 30 shows a graph of ELISA reactivity of anti-whole cell S. aureus IgY antibodies from serum 35 days post initial inoculation to coated formalin-fixed S. aureus cells. The OD (absorbance at 450 nm) values were plotted against total IgY concentration (mg/mL) to show a dose-dependence in specific antibody concentration. Serum from uninoculated (non-targeted) chickens was used as a negative control. Each sample was plated in duplicate and the averages and standard deviations are shown. Specific reactivity to coated formalin-fixed S. aureus cells was demonstrated.

FIG. 31 shows a graph of ELISA reactivity of anti-Staphylococcal protein A (Spa) IgY from serum against coated Spa 35 days post initial inoculation. The OD (absorbance at 450 nm) values were plotted against total IgY concentration (mg/mL) to show a dose-dependence in specific antibody concentration. Serum from uninoculated (non-targeted) chickens was used as a negative control. Each sample was plated in duplicate and the averages and standard deviations are shown. Specific reactivity to coated Spa protein was demonstrated.

FIG. 32 shows a graph of ELISA reactivity of a mixture of anti-SpA and anti-formalin fixed whole cell S. aureus IgY extracted from raw egg yolks against coated formalin-fixed S. aureus Cells. The OD (absorbance at 450 nm) values were plotted against total IgY concentration (mg/mL) to show a dose-dependence in specific antibody concentration. IgY was extracted from eggs that were harvested 18 days after initial inoculation from a mixture of SpA and whole cell S. aureus inoculated chickens. Extracted IgY from non-targeted chickens was run for comparison. Each sample was plated in duplicate and the averages and standard deviations are shown. Specific reactivity to coated formalin-fixed S. aureus cells was demonstrated.

FIG. 33 shows a graph of ELISA reactivity of a mixture of anti-SpA and anti-formalin fixed whole cell S. aureus IgY extracted from raw egg yolks against coated Spa protein. The OD (absorbance at 450 nm) values were plotted against total IgY concentration (mg/mL) to show a dose-dependence in specific antibody concentration. IgY was extracted from eggs that were harvested 18 days after initial inoculation from a mixture of SpA and whole cell S. aureus inoculated chickens. Extracted IgY from non-targeted chickens was run for comparison. Each sample was plated in duplicate and the averages and standard deviations are shown. Specific reactivity to coated Spa protein was demonstrated.

FIG. 34 shows a graph of ELISA reactivity of anti-choleragen IgY antibodies present in serum 21 days following the initial inoculation to coated choleragen. Serum from uninoculated (non-targeted) chickens was used as a negative control. Each sample was plated in duplicate and the averages and standard deviations were are plotted. Specific IgY reactivity to Choleragen was demonstrated. Inoculation of chickens with choleragen was successful in inducing specific antibody production in the target host.

FIG. 35 shows a graph of ELISA reactivity to coated formalin-fixed V. cholerae cells by anti-whole cell V. cholerae IgY in serum collected at 23 days post first inoculation and anti-choleragen IgY antibodies present in serum 21 days following the initial inoculation. Serum from uninoculated (non-targeted) chickens was used as a negative control. Each sample was plated in duplicate and the averages and standard deviations are plotted in the graph. Results show that anti-whole cell V. cholerae IgY in serum exhibited specific binding to coated fixed V. cholerae cells, but anti-choleragen IgY in serum did not exhibit specific binding to coated V. cholerae whole cells.

FIG. 36 shows a graph of ELISA reactivity of anti-ACE2 IgY antibodies present in serum 27 days post the initial plasmid DNA inoculation (ACE2 DNA+CpG) to coated ACE2 protein (Creative Biomart) v total IgY concentration. Serum from uninoculated (non-targeted) chickens was used as a negative control. Each sample was plated in duplicate and the averages and standard deviations are plotted.

FIG. 37 shows a graph of average percent inhibition of RBD:ACE2 binding by anti-ACE2 IgY from Chicken Serum collected at Day 27 post initial plasmid DNA-based inoculation compared to IgY Extracted from non-targeted chicken serum (sVNT Kit). The percent inhibition of RBD:ACE2 binding by anti-ACE2 IgY antibodies in serum from plasmid DNA-inoculated chickens is plotted v total IgY concentration (mg/mL), as compared to serum from uninoculated chickens (non-targeted IgY), tested within the GenScript surrogate Virus Neutralization Test (sVNT) Kit.

FIG. 38 shows a graph of ELISA reactivity of anti-ACE2 IgY extracted from raw egg yolks against collected at Day 16, 23, 30, and 40 post initial inoculation against coated ACE2 Protein. The optical density at 450 nm (OD)±the standard deviation (std dev) of the extracted IgY dilution series from eggs sampled at 16, 23, 30, and 40 days post inoculation (300 ug plasmid ACE2 DNA+20 ug CpG per chicken per innoculation) against coated ACE2 protein. The plates were read at 450 nm, and the OD values were plotted against total IgY concentration (μg/mL), measured by NanoDrop to show a dose-dependence in specific antibody concentration.

FIG. 39 shows a graph of average percent inhibition of RBD:ACE2 binding by anti-ACE2 IgY antibodies in extracted IgY from raw egg yolks from plasmid DNA inoculated chickens at Day 16, 23, 30 and 40 post initial inoculation (ACE2 DNA+CpG adjuvant), as compared to negative non-targeted IgY control samples, tested within the GenScript surrogate Virus Neutralization Test (sVNT) Kit. The same samples were employed in FIG. 38. At day 30, anti-ACE2 IgY exhibits >80% inhibition of RBD:ACE2 binding. At day 40, anti-ACE2 IgY exhibits >90% inhibition of RBD:ACE2 binding.

FIG. 40 shows a graph of ELISA reactivity plotted as optical density 450 nm (OD)±the standard deviation (std dev) of a chicken serum dilution series from pCI_Neo-ACE2 and plasmid adjuvant co-inoculated chickens plated against ACE2 protein. The plasmid adjuvants are as follows: pCI_Neo-IL2, pCl_Neo-IFNγ, and pCl_Neo-chGMCSF. Serum from uninoculated (non-targeted) chickens was used as a negative control. The plates were read at 450 nm and the OD values were plotted against total IgY concentration (mg/mL), measured by NanoDrop, to show a dose-dependent response.

FIG. 41 shows a graph of ELISA inhibition of RBD:ACE2 Binding by anti-ACE2 IgY Serum collected at day 28 post first inoculation from Chickens co-inoculated with pCl_Neo-ACE2 and Plasmid Adjuvants. The average percent inhibition values and standard deviations of the anti-ACE IgY and negative control samples tested in the GenScript surrogate Virus Neutralization Test (sVNT) Kit. Greater than 80% inhibition was exhibited in anti-ACE2 IgY serum from chickens co-inoculated with either the IL2 plasmid adjuvant or the chGNCSF plasmid adjuvant compared to the IFNgamma plasmid adjuvant.

FIG. 42 shows a eukaryotic expression vector map of pCI-neo mammalian expression vector (GenBank® Accession Number U47120, Promega 1841).

FIG. 43 shows a eukaryotic expression vector map of pVIVO2-mcs vector (Invivogen, pvivo2-mcs).

FIG. 44 shows a eukaryotic expression vector map of pVAX1 vector(ThermoFisher V26020).

FIG. 45 shows a eukaryotic expression vector map of pIRES Vector (Clontech, PT3266-5).

FIG. 46 shows a eukaryotic expression vector map of pcDNA 3.1 Mammalian Expression Vector (ThermoFisher V79020).

### DETAILED DESCRIPTION OF THE INVENTION

The term “patient” or “subject” as used herein refers to an animal, for example a mammal, such as a human, who is the object of treatment. The subject, or patient, may be either male or female.

The term “about” as used herein refers to a numeric range that is +/−10% of the given quantity. For example, the term “about 50%” refers to 45% to 55%, and “about 100 mg” refers to 90 mg to 110 mg.

The term “virus” used herein refers to any of a large group of submicroscopic agents that consist of a segment of DNA or RNA surrounded by a coat of protein. Influenza viruses and enteroviruses are RNA viruses. The virus is a parasite that needs a host cell to replicate. Because viruses are unable to replicate without a host cell, they are not considered living organisms in conventional taxonomic systems. They are described as “live” when they are capable of replicating and causing disease. Accordingly, the term “viral activity” refers to any state of being active or any energetic action or movement or liveliness of a virus. Accordingly, the term “viral replication” refers to any process by which genetic materials, a single-celled organism, or a virus reproduces or makes a copy of itself.

The term “infection” as used herein refers to the presence of a virus in or on a subject, which if replication of the virus was retarded or of the activity of the virus was reduced, would result in a benefit to the subject. Accordingly, the term “infection” refers to the presence of pathogens at any anatomical site of a human or animal.

The term “antiviral supplement” as used herein includes any composition used specifically for treatment or prophylaxis of viral infections, particularly SARS-CoV-2 virus infections. The compositions of the disclosure can be evaluated in one aspect by various assays. In another aspect, the compositions of the disclosure can be evaluated by retarding the growth and reproduction of viruses in a cell based assay. In another aspect, the compositions of the disclosure can be evaluated by decreased duration of a viral infection in a patient. In another aspect, the compositions of the disclosure can be evaluated by decreased severity of symptoms in a patient.

The term “whole” in reference to, for example, whole egg, or whole egg yolk, refers to non-defatted egg or egg yolk.

As used herein, the term “pharmaceutically acceptable salt” refers to those salts which retain the biological effectiveness and properties of the active ingredient of the biochemical composition, which are not otherwise undesirable. Pharmaceutically acceptable salts include, but are not limited to, salts of sodium, potassium, calcium, magnesium, aluminum and the like.

As used herein, the term “an effective amount” refers to that an amount of a composition of the disclosure that when administered to an individual subject in need thereof, is sufficient to reduce the virus activity and/or growth thereby enhancing the antiviral activity.

As used herein, the term “therapeutically effective amount” refers to an amount of a composition of the disclosure that when administered to a human subject in need thereof, is sufficient to effect treatment or prophylaxis for SARS-CoV-2 virus infection. The amount that is therapeutically effective will depend upon the patient's size and gender, the stage and severity of the infection and the result sought. For a given patient and condition, a therapeutically effective amount can be determined by methods known to those of skill in the art. For example, in reference to the treatment of a influenza virus infection using the compositions of the present invention, a therapeutically effective amount refers to that amount of the composition which has the effect of (1) reducing the shedding of the virus, (2) reducing the duration of the infection, (3) reducing infectivity and/or, (4) reducing the severity (or, preferably, eliminating) one or more other signs or symptoms associated with the infection such as, for example, fever, cough, shortness of breath, fatigue, muscle aches, muscle pain, sputum production, sore throat, complete or partial loss of smell, complete or partial loss of taste, nausea, vomiting, diarrhea, expectoration chest pain, breathlessness, perspiration, hypoxia (having low oxygen saturation), severely breathless, for example, not able to speak a complete sentence, positive SARS-CoV-2 PCR test, elevated high-sensitivity C-reactive protein, elevated interleukin-6, elevated D-dimer, leukopenia (reduced number of white blood cells), leukocytosis (elevated white blood cells), elevated lactate dehydrogenase, elevated aminotransferase, elevated aspartate aminotransferase, elevated bilirubin. Such an effective dose will generally depend on the factors described above. A prophylactically effective dose is one that reduces the likelihood of contacting an SARS-CoV-2019 virus infection. A prophylactically effective dose is from about 20% to about 100%, preferably from about 40% to about 60%, of a therapeutically effective dose.

The term “neutralizing antibody” refers to an IgY antibody capable of rendering live SARS-CoV-2 coronavirus unable to bind to human ACE2. In some embodiments, this may be determined by any suitable method known in the art.

In one aspect, the administration is oral administration. Generally, a therapeutically effective dose is not less than about 10% and not more than about 200% of the amounts of individual ingredients listed in Table 1. In certain aspects, a therapeutically effective dose for treatment of a viral infection is from about 50% to about 150%, or from about 80% to about 120%, or about 100% identical with the list of components in Table 1. In another aspect, a therapeutically effective dose for treatment of a viral infection in a subject in need thereof is administered every 4 to 6 waking hours, or from about two to six times per day. In a further aspect, a therapeutically effective dose for prophylaxis of a viral infection in a subject in need thereof is administered every 8 to 12 waking hours, or from about one to three times per day.

The term, “pharmaceutically acceptable carrier” as used herein means a pharmaceutically-acceptable material, composition or vehicle, such as a liquid or solid filler, diluent, excipient, solvent or encapsulating material, involved in carrying or transporting the compositions of the invention from one organ, or portion of the body, to another organ, or portion of the body without affecting its biological effect. Each carrier should be “acceptable” in the sense of being compatible with the other ingredients of the composition and not injurious to the subject.

The term “specifically binds,” or “binds specifically to”, means that an antibody or antigen-binding fragment thereof forms a complex with an antigen that is relatively stable under physiologic conditions. Screening for specific binding of polyclonal antibodies may be accomplished using an ELISA method, or the like.

Enzyme-linked Immunosorbent Assays (ELISAs) may be performed in direct antigen-antibody binding, inhibition format or competition format. For example, one or a combination of the recombinant SARS-CoV-2 proteins of the disclosure or commercial proteins may be coated to an ELISA plate, followed by washing and blocking steps, a solution of the IgY antibodies of interest may be added and incubated. Following wash, a solution of a suitable labeled secondary antibody may be added. For example an HRP-labeled secondary antibody anti-IgY secondary antibody-horseradish peroxidase (HRP) labeled reagent may be employed, followed by Steptavidin for colorimetric detection. In another ELISA format S1-S2-ECD recombinant protein is coated to the plates, followed by IgY sample, then ACE2, followed by secondary antiACE2-HRP and streptavidin reagents. In another ELISA format, plates are coated with ACE2, then IgY and S1-S2-ECD are mixed and added to wells, followed by anti-ACE2-HRP secondary antibodies, streptavidin, etc. However, any suitable ELISA method may be employed.

The term “substantial identity” or “substantially identical,” when referring to a nucleotide or fragment thereof, indicates that, when optimally aligned with appropriate nucleotide insertions or deletions with another nucleotide (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleotide molecule having substantial identity to a reference nucleotide molecule may, in certain instances, encode a polypeptide having the same or substantially similar amino acid sequence as the polypeptide encoded by the reference nucleotide molecule.

The term “derived from” when made in reference to a nucleotide or amino acid sequence refers to a modified sequence having at least 50% of the contiguous reference nucleotide or amino acid sequence respectively, wherein the modified sequence causes the synthetic microorganism to exhibit a similar desirable attribute as the reference sequence of a genetic element such as promoter, cell death gene, antitoxin gene, virulence block, or nanofactory, including upregulation or downregulation in response to a change in state, or the ability to express a toxin, antitoxin, or nanofactory product, or a substantially similar sequence, the ability to transcribe an antisense RNA antitoxin, or the ability to prevent or diminish horizontal gene transfer of genetic material from the undesirable microorganism.

The term “derived from” in reference to a nucleotide sequence also includes a modified sequence that has been codon optimized for a particular microorganism to express a substantially similar amino acid sequence to that encoded by the reference nucleotide sequence. The term “derived from” when made in reference to a microorganism, refers to a target microorganism that is subjected to a molecular modification to obtain a synthetic microorganism.

The term “substantial similarity” or “substantially similar” as applied to polypeptides means that two peptide or protein sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions.

The term “conservative amino acid substitution” refers to wherein one amino acid residue is substituted by another amino acid residue having a side chain (R group) with similar chemical properties, such as charge or hydrophobicity. In general, a conservative amino acid substitution will not substantially change the functional properties of the, e.g., toxin or antitoxin protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Polypeptide sequences may be compared using FASTA using default or recommended parameters, a program in GCG Version 6.1. FASTA (e.g., FASTA2 and FASTA3) provides alignments and percent sequence identity of the regions of the best overlap between the query and search sequences (see, e.g., Pearson, W. R., Methods Mol Biol 132: 185-219 (2000), herein incorporated by reference). Another preferred algorithm when comparing a sequence of the disclosure to a database containing a large number of sequences from different organisms is the computer program BLAST, especially BLASTP or TBLASTN, using default parameters. See, e.g., Altschul et al., J Mol Biol 215:403-410 (1990) and Altschul et al., Nucleic Acids Res 25:3389-402 (1997).

Unless otherwise indicated, nucleotide sequences provided herein are presented in the 5′-3′ direction.

All pronouns are intended to be given their broadest meaning. Unless stated otherwise, female pronouns encompass the male, male pronouns encompass the female, singular pronouns encompass the plural, and plural pronouns encompass the singular.

The term “systemic administration” refers to a route of administration into the circulatory system so that the entire body is affected. Systemic administration can take place through enteral administration (absorption through the gastrointestinal tract, e.g. oral administration) or parenteral administration (e.g., injection, infusion, or implantation). For example, the antibodies may be administered intramuscularly, subcutaneously, or intravenously. Antibodies for use in systemic administration may be partially purified, purified and 0.2 micron filtered by any appropriate method known in the art. For example, antibodies may be at least partially purified by precipitation methods. Standards for production of polyclonal antibodies may be found in, for example, EMEA, Guidance of Production and Quality Control of Animal Immunoglobulins and Immunosera for Human Use, 2002, which is incorporated herein by reference.

The term “topical administration” refers to application to a localized area of the body or to the surface of a body part regardless of the location of the effect. Typical sites for topical administration include sites on the skin or mucous membranes. In some embodiments, topical route of administration includes enteral administration of medications or compositions.

The term “including” as used herein is non-limiting in scope, such that additional elements are contemplated as being possible in addition to those listed; this term may be read in any instance as “including, but not limited to.”

The terms “prevention”, “prevent”, “preventing”, “prophylaxis” and as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

### Coronaviruses

Coronaviruses are enveloped viruses with a positive-sense, single-stranded RNA genome belonging to the Coronaviridae family. The viruses can be classified into four genera, namely alpha, beta, gamma and deltaCoVs (Woo et al., 2009, Coronavirus diversity, phylogeny and interspecies jumping, Exp Biol Med 234 (10), pp. 1117-1127).

SARS-CoV-2 represents the seventh coronavirus that is known to cause human disease. Previously identified human CoVs that cause human disease include the alphaCoV hCoV-NL63 and hCoV-229E and the betaCoVs HCoV-OC43, HKU1, severe acute respiratory syndrome coronavirus (SARS-CoV), and Middle East respiratory syndrome coronavirus (MERS-CoV). Both alphaCoVs and the betaCoVs HCoV-OC43 and HKU1 cause self-limiting common cold-like illnesses. However, SARS-CoV and MERS-CoV infection can result in life threatening disease and have pandemic potential.

The SARS-CoV-2 virus belongs to the 2B group of the betacoronavirus family, which is the same family as SARS-CoV and MERS-CoV and has 70% similarity in genetic sequence to SARS. These viruses are given the name corona (Latin for crown) because they possess a crown-like coat (club-shaped glycoprotein spikes protruding from its surface). These spikes allow the viruses to bind to certain receptors on our cells.

The structure of SARS-CoV-2 includes a spike protein, which includes two regions, S1 and S2, where S1 includes a host cell receptor binding domain and S2 is for membrane fusion. The spike protein is a target for neutralizing with antibodies and vaccines. It been reported that SARS-CoV-2 can infect the human respiratory epithelial cells 100-1000 times more than previous coronavirus strains and does so by interacting with human Angiotensin Converting Enzyme 2 (ACE2) receptors.

The Nucleocapsid Protein is the most abundant protein in SARS-CoV-2. The N-protein is a highly immunogenic phosphoprotein and rarely changes. The N protein of SARS-CoV-2 is often used as a marker in diagnostic assays. There is also the hemagglutinin-esterase dimer, a membrane glycoprotein, an envelope protein, and RNA. https://www.rapidmicrobiology.com/test-method/testing-for-the-wuhan-coronavirus-a-k-a-covid-19-sars-cov-2-and-2019-ncov.

The SARS-CoV-2 includes a coronavirus M matrix glycoprotein. One M protein sequence isolated from a coronavirus disease 19 patient in Shanghai may be found in GenBank: comprising amino acid sequence accession QI157163.1; madsngtity eelkklleqw nlvigflflt wicllqfaya nrnrflyiik liflwllwpv tlacfvlaav yrinwitggi aiamaclvgl mwlsyfiasf rlfartrsmw sfnpetnill nvplhgtilt rplleselvi gavilrghlr iaghhlgrcd ikdlpkeitv atsrtlsyyk lgasqrvagd sgfaaysryr ignyklntdh ssssdniall vq. The virus has been designated SARS-CoV-2 by the Coronavirus Study Group (CSG) of the International Committee on Taxonomy of Viruses. The CSG formally recognizes this virus as a sister to severe acute respiratory syndrome coronaviruses (SARS-CoVs) of the species Severe acute respiratory syndrome-related coronavirus and designates it as Severe Acute Respiratory Syndrome Coronavirus 2 (SARS-CoV-2). Infection by the virus can cause respiratory symptoms, fever, and fatigue. But in severe cases, especially people with compromised immune systems, the virus can cause severe acute respiratory syndrome (SARS), organ failure and even death.

The structure of SARS-CoV-2 (also known as 2019-nCoV) has been investigated using cryo-electron microscopy (cryo-EM). Wrapp et al., 2020, Cryo-EM structure of the 2019-nCoV spike in prefusion conformation. Science 367, 1260-1263 (2020) 13 Mar. 2020. Wrapp et al. provide biophysical and structural evidence that the 2019-nCoV S protein binds angiotensin-converting enzyme 2 (ACE2) with higher affinity than does severe acute respiratory syndrome (SARS)-CoV S. Additionally, Wrapp et al. tested several published SARS-CoV RBD-specific monoclonal antibodies S230, m396, and 8OR (each at 1 μM) using biolayer interferometry (BLI) and found that they do not have appreciable binding to 2019-nCoV S, suggesting that antibody cross-reactivity may be limited between the two receptor binding domains (RBDs).

Coronaviruses are dynamic and are continuously mutating. Viruses can change in two different ways: antigenic drift and antigenic shift. For example, viruses are constantly changing by antigenic drift, but antigenic shift may occur only occasionally. Antigenic shift confers a major antigenic change, and is a specific case of reassortment or viral shift that confers a phenotypic change. For example, two or more different strains of a virus, or strain of two or more different viruses, may combine to form a new subtype having a mixture of surface antigens of the two or more original strains. Antigenic drift refers to small, gradual changes that occur through point mutations in the genes that encode the main surface proteins. These point mutations occur unpredictably and result in minor changes to these surface proteins. Importantly, antigenic drift can produce new virus strains that may not be recognized by antibodies to earlier coronavirus strains.

### SARS-CoV-2

SARS-CoV-2 is the seventh coronavirus known to infect humans; SARS-CoV, MERSCoV and SARS-CoV-2 can cause severe disease, whereas HKU1, NL63, OC43 and 229E are associated with mild symptoms. Andersen et al. 2020.

Recently, the carboxypeptidase angiotensin converting enzyme 2 (ACE2) was reported as an entry receptor for SARS-CoV-2. The lungs are among the organs most affected by COVID-19 because the virus accesses host cells via ACE2, which is most abundant in the type II alveolar cells of the lungs. The virus uses a special surface glycoprotein called a “spike” (peplomer) to connect to ACE2 and enter the host cell. Recent reports demonstrating that 2019-nCoV S and SARS-CoV S share the same functional host cell receptor, angiotensin-converting enzyme 2 (ACE2). Zhou et al., Nature (2020).

ACE2 has also been known to be the entry point for other severe acute respiratory syndrome (SARS) coronaviruses. ACE2 plays a key balancing role in the renin-angiotensin system (RAS). RAS activity is intrinsically high in the lung. Pulmonary ACE2 appears to have a role in regulating the balance of circulating Angiotensin II/Angiotensin 1-7 levels. Ang II induces pulmonary vasoconstriction in response to hypoxia, which is important in preventing shunting in patients with pneumonia or lung injury. In acute respiratory distress syndrome (ARDS), the RAS appears crucial in maintaining oxygenation. In ARDS models, ACE2 knockout mice displayed more severe symptoms of the disease compared to wild-type mice, while overexpression appears protective. Tikellis et al., Int J Peptides, 2012, Article ID 256294.

Wang et al. identified the S1 C-terminal domain (SARS-CoV-2-CTD) as the key region in SARS-CoV-2 that interacts with the hACE2 using immunostaining and flow cytometry assays, and solved a 2.5 Å crystal structure of SARS-CoV125 2-CTD in complex with hACE2, which reveals a receptor-binding mode similar overall to that observed for the SARS-CoV RBD (SARS-RBD). However, SARS-CoV-2-CTD forms more atomic interactions with hACE2 than the SARS-RBD, which correlates with data showing higher affinity for receptor binding. Wang et al. 2020 Cell preprint, DOI: 10.1016/j.cell.2020.03.045. Wang et al. also found that a panel of monoclonal antibodies (mAbs), as well as murine polyclonal antisera against SARS-S1/RBD were unable to bind to the SARS-CoV-2 S protein, indicating notable differences in antigenicity between SARS-CoV and SARS-CoV-2, suggesting that the previously developed SARS-RBD based vaccine candidates are unlikely to be of any clinical benefit for SARS-CoV-2 prophylaxis.

In another study, Walls et al. demonstrates that SARS-CoV S murine polyclonal antibodies potently inhibited SARSCoV-2 S mediated entry into cells, indicating that cross-neutralizing antibodies targeting conserved S epitopes can be elicited upon vaccination. Walls et al., Structure, Function, and Antigenicity of the SARS-CoV-2 Spike Glycoprotein, Cell (2020), https://doi.org/10.1016/j.cell.2020.02.058. Walls et al. surmise most of these Abs target the highly conserved S2 subunit (including the fusion peptide region) based on its structural similarity across SARS-CoV-2 and SARS-CoV, the lack of cross-reactivity of several SB-directed Abs (Tian et al., 2020 bioRxiv preprint; Wrapp et al., 2020), and previous reports showing that sera from SARS-CoV-infected individuals target this region (Zhang et al., 2004). Walls et al. note that most SARS-CoV neutralizing Abs isolated to date target the SB domain and that several of them recognize the RBM and prevent receptor engagement.

### Transmission

Transmission of SARS-CoV-2 may occur through direct contact with an infected individual, touching of an affected surface, or through proximity to an infected individual, for example, from person to person through coughing or sneezing. People may also become infected by contact with contaminated surfaces or objects, then by touching their mouth, nose or eyes. An infected adult may be able to infect others beginning at least about one day before symptoms develop and several days after becoming ill.

Time from exposure to onset of symptoms is generally between two and fourteen days, with an average of five days. The standard method of diagnosis is by reverse transcription polymerase chain reaction (rRT-PCR) from a nasopharyngeal swab. The infection can also be diagnosed from a combination of symptoms, risk factors and a chest CT scan showing features of pneumonia.

Recommended measures to prevent infection include frequent hand washing, social distancing (maintaining physical distance from others, especially from those with symptoms), covering coughs and sneezes with a tissue or inner elbow, and keeping unwashed hands away from the face. The use of masks is recommended by some national health authorities for those who suspect they have the virus and their caregivers, but not for the general public, although simple cloth masks may be used by those who desire them.

Current Centers for Disease Control (CDC) guidelines recommend maintaining a minimum of 6 feet (2 meters) between individuals to maintain social distancing and minimize transmission of SARS-CoV-2. However, a human sneeze can eject a turbulent gas cloud of 23-27 feet (7-8 meters) potentially containing respiratory pathogen emissions. Bourouiba, JAMA Insights, Mar. 26, 2020. doi:10.1001/jama.2020.4756. Clearly additional methods of preventing transmission are desirable.

In order to maintain social distancing and limit spread of SARS-CoV-2 infection, many governments have instituted work from home or shelter in place orders, all but shutting down certain segments of society considered as non-essential services. The full economic impact is yet to be determined.

### Epidemiological Features and Clinical Course

There is currently no vaccine or approved specific antiviral treatment for COVID-19. Management involves treatment of symptoms, supportive care, isolation, and experimental measures. Management of SARS-CoV-2 infection typically involves treatment of symptoms, supportive care, isolation, and experimental measures.

A descriptive case series of the first 18 patients diagnosed with polymerase-chain reaction (PCR)-confirmed SARS-CoV-2 infection in Singapore was published providing epidemiological features and clinical course. Young et al., JAMA, published online Mar. 3, 2020, corrected version Mar. 20, 2020, doi:10.1001/jama.2020.3204. Among the first 18 patients diagnosed with SARS-CoV-2 infection in Singapore, clinical presentation was frequently a mild respiratory tract infection. Some patients requiring supplemental oxygen had variable clinical outcomes with variable clinical outcomes following treatment with an anti-retroviral agent. Specifically, among the 18 patients, the median age was 47 yrs, including 9 women (50%). Clinical presentation was an upper respiratory tract infection in 12 (67%) and viral shedding from nasopharynx was prolonged for 7 days or longer among 15 (83%). Six individuals required supplemental oxygen, of these, 2 required intensive care. There were no deaths. Virus was detectable in stool in (4/8 (50%) and blood (1/12 [8%] by PCR but not urine. Five individuals requiring supplemental oxygen were treated with lopinavir-ritonavir. For 3 of the 5 patients fever resolved and supplemental oxygen requirement was reduced within 3 days, whereas 2 deteriorated with progressive respiratory failure. Four of the 5 patients treated with lopinavir-ritonavir developed nausea, vomiting, and/or diarrhea, and 3 developed abnormal liver function test results. Signs and symptoms on presentation, n=18, included fever 13 (72%), cough 15 (93%), shortness of breath 2 (11%), rhinorrhea 1 (6%), sore throat 11 (61%), and diarrhea 3 (17%). Vital signs included median (range) temperature (° C.) 37.7 (36.1-39.6), respiratory rate, breaths/min 18 (16-21), pulse oximeter 02 saturation, % 98 (95-100), systolic blood pressure, mm Hg 131 (103-167), and heart rate, /min 97 (75-118). Abnormal chest radiograph No. (%) 6 (33%). Duration of symptoms (range) for fever, 4 days (0-15), any symptoms, 13 days (0-24).

### Symptoms

According to the CDC, reported illnesses have ranged from mild symptoms to severe illness and death for confirmed coronavirus disease 2019 (COVID-19) cases. Symptoms including fever, cough, and/or shortness of breath may appear 2-14 days after exposure. Emergency warning signs include trouble breathing, persistent pain or pressure in the chest, new confusion or inability to arouse, and bluish lips or face.

Symptoms of SARS-CoV-infection may include fever, cough, fatigue, shortness of breath, dry cough, sore throat, muscle aches, runny and/or stuffy nose. Gastrointestinal symptoms such as nausea, vomiting and diarrhea can also occur.

Symptoms of COVID-19 can include fever, cough, shortness of breath, fatigue, muscle aches, muscle pain, sputum production, diarrhea, sore throat, confusion, lethargy, and complete or partial loss of smell and loss of taste. Potential gastrointestinal manifestations of COVID-19 have been reported including nausea, vomiting, diarrhea, and abnormal liver function tests. SARS-CoV-2 has been detected in patient stool although at this point it is unclear if there is a fecal-oral route of infection.

Some coronavirus symptoms are mild. Upper respiratory tract infection (URTI) may include fever, dry cough, sore throat, sneezing, runny nose. These symptoms may be seen with the common cold or seasonal flu. Some patients after developing upper respiratory tract infection may complicate to lower respiratory tract infection (LRTI), pneumonia. This may include a high fever with chills, cough, expectoration chest pain, breathlessness, perspiration. A chest x-ray may show pneumonia in part of the lung. Patients exhibiting these symptoms should be admitted to the hospital for treatment as soon possible.

Acute Respiratory Distress Syndrome (ARDS) is a severe type of pneumonia where the patient is hypoxic having low oxygen saturation, severely breathless, for example, not able to speak a complete sentence. These types of patients usually need intensive care unit (ICU) and ventilator management. ARDS can complicate into multiple organ dysfunction syndrome (MODS) where other body organs may be affected including kidney, heart, liver, and brain. Once the patient has progressed to MODS, prognosis is extremely poor, and ultimately may result in death.

In one observational study, methylprednisolone treatment was associated with improved outcomes among patients with ARDS. Wu C et al. Risk factors associated with acute respiratory distress syndrome and death in patients with coronavirus disease 2019 pneumonia in Wuhan, China. JAMA Intern Med 2020 Mar. 13; [e-pub]. (https://doi.org/10.1001/jamainternmed.2020.0994). In a cohort of 201 patients early in the COVID-19 epidemic, many patients had elevated inflammatory and coagulation markers associated with poor prognosis in other studies. Although the numbers of patients tested varied, laboratory abnormalities were common, including elevated lactate dehydrogenase in 194 (98%), elevated high-sensitivity C-reactive protein in 166 (85.6%), elevated interleukin-6 in 60 (48.8%), and elevated D-dimer in 44 (23.3%). Age ≥65 years, neutrophilia, and organ or coagulation dysfunction were associated with ARDS and death. Among those with ARDS, treatment with methylprednisolone was associated with significantly better outcomes: 23 of 50 (46%) methylprednisolone recipients died compared with 21 of 34 (61.8%) nonrecipients (hazard ratio, 0.38).

Although corticosteroids appeared to be beneficial in this cohort, this has not been a consistent finding. Kaul, D., Risk factors for ARDS and progression to death among COVID-19 patients, Summary and Comment, NEJM Journal Watch, Infectious Diseases, Mar. 23, 2020 citing Zhou et al. Lancet 2020; 395: 1054-62. In addition, WHO guidelines do not recommend adjunctive corticosteroids outside of a clinical trial.

### Testing and Diagnosis

Several molecular-based detection kits made available for SARS-CoV-2 are real-time reverse transcriptase PCR assays. This is a test where RNA of the virus is detected. Many of the kits contain three assays, each assay targeting a different gene in the virus, so if the virus does mutate the chances of all three targets mutating is low. However if one, or two, of these assays, is positive, then the result must be recorded as inconclusive. These targets are the Orf1 gene (human RNA polymerase protein), the N-gene (the nucleocapsid protein) and the E-gene (envelope protein). There are some kits which target the S-gene (spike protein) but these are limited. An increasing number of the products have received Emergency Use Authorisation (EUA) from the FDA and have received CE-marking for sale in Europe, with still some products only available for Research Use Only (RUO).

Various patient samples may be tested for virus by SARS-CoV-2 RNA polymerase chain reaction (PCR) technique. According to CDC guidelines, swabs with synthetic fibers and plastic shafts should be used for nasopharyngeal collections when 2019-nCoV is suspected. It's important not to use calcium alginate swabs or those with wooden shafts as they may contain materials that interfere with test results. For upper respiratory specimens, the CDC is recommending nasopharyngeal washes/aspirates. nasal aspirates or to collect both a nasopharyngeal swab AND an oropharyngeal swab made of synthetic fiber with plastic applicators. Once the specimens are collected, it is recommended that the swabs are placed in 2-3 ml of viral transport media.

Patient samples may be evaluated by RT-PCR. For example, the “Centers for Disease Control and Prevention (CDC) 2019-Novel Coronavirus (2019-nCoV) Real-Time Reverse Transcriptase (RT)-PCR Diagnostic Panel” is intended for use with Applied Biosystems 7500 Rast DX Real-Time PCR instrument. This test may be used for upper and lower respiratory samples to be performed by laboratories designated by CDC as qualified and in the US certified under Clinical Laboratories Improvement Amendments (CLIA) to perform high complexity tests. Cobas® SARS-CoV-2 Test (Roche, Basel, Switzerland) is a real-time PCR test intended for qualitative detection of nucleic acids from nasopharyngeal and oropharyngeal swab samples. Cobas® SARS-CoV-2 Test is a single well dual target assay for both specific detection of SARS-CoV-2 and pan-sarbecovirus detection for the sarbecovirus subgenus family that includes SARS-CoV-2.

Various research tests are commercially available. For example, RealStar® SARS CoV-2 RT-PCR Kit 1.0 RUO (Research Use Only) (Altona Diagnostics GmbH, Hamburg, Germany) based on real time PCR technology is a dual target assay to rapidly screen lineage B-betacoronaviruses and confirm the SARS-CoV-specific RNA in one reaction.

Immunoassays may be employed to test for SARS-CoV-2-specific antibodies. Commercial assays to detect COVID-19 include a dual ELISA test to detect specific IgA and IgG against the virus in the blood of infected patients. There is also an automated fluorescent assay system to measure quantitatively or semi-quantitatively the concentration of the target analyte, which can be the viral antigen or IgM/IgG. Point of Care assays that test for COVID-19 are currently in development, and are awaiting approval, for example, in the form of EUA (Emergency Use Authorization) or CE-IVD certification. There has been one test given FDA-EUA, the PerkinElmer New Coronavirus Nucleic Acid Detection Kit is a probe-based PCR assay using fluorescent-labeled probes specific to the 2019 coronavirus (SARS-CoV-2) open reading frame lab and nucleocapsid protein genes. http://ir.perkinelmer.com/news-releases/news-release-details/fda-provides-emergency-use-authorization-perkinelmer-covid-19It has a limit of detection (LoD) of 20 copies/mL using a 400 μL sample. These kits need to be used on a small analyzer.

Serologic blood tests that detect coronavirus-specific antibodies from past infection may also be employed. Such tests are available commercially, for example WONDFO® SARS-CoV-2 Antibody Test (lateral flow method) (Wondfo®, Guangzhou, China) may be used for the diagnosis of coronavirus disease (COVID-19), with a result in 15 minutes and detection of both IgG and IgM antibody of SARS-CoV-2. FINECARE™ SARS-VoV-2 Antibody Test (Wondfo®, Guangzhou, China) can be performed on FINECARE™ Analyzers. Detection for both IgG and IgM antibodies of SARS-CoV-2 is possible. Using with FINECARE™ series, multiple parameters can be detected simultaneously including CRP, PCT, SAA, IL-6, Myo, cTn I, cTn T, and D-dimer.

### Prevention and Treatment

Vaccines are being deployed for preventing SARS-CoV-2 infection. One problem with coronavirus vaccine development is that by the time the vaccine is evaluated for safety and efficacy, and receives regulatory approval, the virus may have mutated such that the efficacy of the vaccine is diminished.

No specific therapeutic treatments for COVID-19 are currently available so medical management involves supportive measures. Some drugs developed for other indications have shown efficacy in vitro including antiviral drug remdesivir and monoclonal antibody cocktails.

Clinical trials of remdesivir are underway (NCT04280705). Remdesivir was given on a compassionate use basis to the first COVID-19 case in the US. Holshue et al., NEJM 2020; 382:929-936.

One open label clinical study, single group assignment, is evaluating efficacy and safety of corticosteroids in COVID-19 by administering Methylprednisolone 1 mg/kg/day ivgtt for 7 days. C linicalTrials.gov Identifier: NCT04273321.

Some COVID-19 patients have been treated with plasma from convalescent patients. Shen et al., 2020, Treatment of 5 critically ill patients with COVID-19 with convalescent plasma, JAMA published online Mar. 27, 2020. A preliminary study of 5 severely ill patients with coronavirus disease 2019 (COVID-19) who were treated in the Shenzhen Third People's Hospital, China, using plasma from recovered individuals. All patients had severe respiratory failure and were receiving mechanical ventilation; 1 needed extracorporeal membrane oxygenation (ECMO) and 2 had bacterial and/or fungal pneumonia. Four patients without coexisting diseases received convalescent plasma around hospital day 20, and a patient with hypertension and mitral valve insufficiency received the plasma transfusion at day 10. The donor plasma had demonstrable IgG and IgM anti-SARS-CoV-19 antibodies and neutralized the virus in in vitro cultures. The 5 patients in Shen et al. were also receiving antiviral treatment, primarily with lopinavir/ritonavir and interferon, and methylprednisolone. Nevertheless the use of convalescent plasma may have contributed to their recovery because the clinical status of all patients had improvement approximately 1 week after transfusion, as evidenced by normalization of body temperature as well as improvements in Sequential Organ Failure Assessment scores and PAO2/FIO2 ratio. In addition, the patients' neutralizing antibody titers increased and respiratory samples tested negative for SARS-CoV-2 between 1 and 12 days after transfusion.

Nevertheless regulatory barriers exist that currently limit the use of pathogen reduction technology for convalescent plasma collections or that require several month inventory holds on H-Ig pharmaceuticals. Roback 2020 JAMA. Convalescent plasma to treat COVID-19 possibilities and challenges. Published online Mar. 27, 2020.

An open label, single group assignment, clinical trial using hyperimmune plasma from donors recovered from new coronavirus 2019 as therapy for critical patients with COVID-19 recently started, sponsored by Foundation IRCCS San Matteo Hospital. Clinical Trials.gov. NCT04321421. Briefly, apheresis from recovered donors will be performed with a cell separator device, with 500-600 mL of plasma obtained from each donor. Donors are males, age 18 yrs or more, evaluated for transmissible diseases according to the italian law. Adjunctive tests will be for hepatitis A virus, hepatitis B virus and Parvovirus B-19. All donors will be tested for the Covid-19 neutralizing titer. Each plasma bag obtained from plasmapheresis will be immediately divided in two units and frozen according to the national standards and stored separately. 250-300 mL of convalescent plasma will be used to treat each of the recruited patients at most 3 times over 5 days.

Coronaviruses are dynamic and are continuously mutating. Viruses can change in two different ways: antigenic drift and antigenic shift. For example, viruses are constantly changing by antigenic drift, but antigenic shift may occur only occasionally. Antigenic shift confers a major antigenic change, and is a specific case of reassortment or viral shift that confers a phenotypic change. For example, two or more different strains of a virus, or strain of two or more different viruses, may combine to form a new subtype having a mixture of surface antigens of the two or more original strains. Antigenic drift refers to small, gradual changes that occur through point mutations in the genes that encode the main surface proteins. These point mutations occur unpredictably and result in minor changes to these surface proteins. Importantly, antigenic drift can produce new virus strains that may not be recognized by antibodies to earlier coronavirus strains.

Methods and compositions are provided herein to allow rapid development of new prophylactic and therapeutic polyclonal antibodies recognizing SARS-CoV-2 mutants, variants, and strains.

The present disclosure provides anti-SARS-CoV-2 RBD polyclonal IgY antibodies exhibiting comparable IC₅₀ to that of a reference monoclonal antibody in a cell-based Covid-19 pseudovirus neutralization assay (example 25), as well as broad spectrum activity v. several SARS-CoV-2 Spike and RBD variants (example 23).

Several mutations in SARS-CoV-2 have already been recognized. The term “mutation” refers to the actual change in sequence. For example, D614G is an aspartic acid-to-glycine substitution at position 614 of the spike glycoprotein. Genomes that differ in sequence are often called variants. Two variants can differ by one mutation or many. A variant is a strain when it has a demonstrably different phenotype (e.g., a difference in antigenicity, transmissibility, or virulence). For example, various mutations of SARS-CoV-2 spike glycoprotein have been recognized in China, the United Kingdom, the Netherlands, Denmark, and South Africa. Lauring et al., Genetic variants of SARS-CoV-2-What do they mean?, JAMA, Published online Jan. 6, 2021. doi:10.1001/jama.2020.27124.

For example, the D614G mutation in the spike glycoprotein was recognized in in early March 2020 in Europe, North America and Asia, and it has been reported that the 614G viruses may spread more efficiently. Outbreaks of SARS-CoV-2 began to emerge in mink farms in the Netherlands and Denmark in late spring and early summer 2020. Many SARS-CoV-2 sequences from the Netherlands and Danish outbreaks had an Y453F mutation in the receptor binding domain of the spike glycoprotein. Several individuals in the Danish outbreak had a variant termed cluster 5 which had 3 additional mutations in the spike (del69_70, I692V, and M1229I). An investigation of human convalescent samples suggested a reduction in neutralization activity against cluster 5 viruses. Lineage B.1.1.7 (also called 501Y.V1) is a phylogenetic cluster spreading in southeastern England having 17 lineage-defining mutations which is said to be spreading more quickly than other lineages. Eight of the lineage B.1.1.7 mutations are in the spike glycoprotein, including N501Y in the receptor binding domain, deletion 69_70, and P681H in the furin cleavage site. Additional mutations include D614G which may cause a moderately increased transmissibility, N439K which may be an “escape mutant” for some neutralizing antibodies, and Y453F, which may be an escape mutant for some neutralizing antibodies. The 501Y spike variants are predicted to have higher affinity for human ACE2, and a different variant also with a N501Y mutation is spreading rapidly in South Africa. Lauring et al., Genetic variants of SARS-CoV-2-What do they mean?, JAMA, Published online Jan. 6, 2021. doi:10.1001/jama.2020.27124.

For example, the D614G mutation in the spike glycoprotein was recognized in in early March 2020 in Europe, North America and Asia, and it has been reported that the 614G viruses may spread more efficiently. Outbreaks of SARS-CoV-2 began to emerge in mink farms in the Netherlands and Denmark in late spring and early summer 2020. Many SARS-CoV-2 sequences from the Netherlands and Danish outbreaks had an Y453F mutation in the receptor binding domain of the spike glycoprotein. Several individuals in the Danish outbreak had a variant termed cluster 5 which had 3 additional mutations in the spike (del69_70, I692V, and M1229I). An investigation of human convalescent samples suggested a reduction in neutralization activity against cluster 5 viruses. Lineage B.1.1.7 (also called 501Y.V1) is a phylogenetic cluster spreading in southeastern England having 17 lineage-defining mutations which is said to be spreading more quickly than other lineages. Eight of the lineage B.1.1.7 mutations are in the spike glycoprotein, including N501Y in the receptor binding domain, deletion 69_70, and P681H in the furin cleavage site. The 501Y spike variants are predicted to have higher affinity for human ACE2, and a different variant also with a N501Y mutation is spreading rapidly in South Africa. Lauring et al., Genetic variants of SARS-CoV-2-What do they mean?, JAMA, Published online Jan. 6, 2021. doi:10.1001/jama.2020.27124.

The recombinant proteins, peptides, fragments of the present disclosure may include one or more, two or more, three or more, four or more, five or more, six or more, or seven or more amino acid mutations compared to SEQ ID NO: 1 or SEQ ID NO: 41. The SARS-CoV-2 spike protein may include an amino acid residue deletion or substitution selected from the group consisting of orfΔ3b, deletion 69-70, M129I, deletion 144, P337S, F338K, V341I, F342L, A344S, A348S, A352S, N354D, S359N, V367F, N379S, A372S, A372T, F377L, K378R, K378N, P384L, T385A, T393P, V395I, D405V, E406Q, R408L, Q409E, Q414A, Q414E, Q414R, K417N, A435S, W436R, N439K, N440K, K444R, V445F, G446V, G446S, P499R, L452R, Y453F, F456L, F456E, K458R, K458Q, E471Q, I472V, G476S, S477N, S477L, S477R, T478I, P479S, N481D, G482S, V483A, V483I, G485S, F486S, F490S, S494P, N501Y, V503F, Y505C, Y508H, A520S, A520V, P521S, P521R, A522V, A522S, A570D, D614G, P681H, R683A, R685A, I692V, T716I, F817P, A829T, A892P, A899P, A942P, S982A, K986P, V987P, and/or D1118H. In some embodiments, the mutations in the spike protein comprising SEQ ID NO: 1 or a fragment thereof may include orfΔ3b, deletion 69-70, deletion 144, K417N, Y453F, E484K, N501Y, A570D, D614G, P681H, I692V, T716I, A829T, S982A, D1118, and/or M129I. In some embodiments, mutants of SEQ ID NO: 1 may comprise deletion 69-70, deletion 144, N501Y, A570D, D614G, P681H, T716I, S982A, and/or D1118, or otherwise according to SARS-CoV-2 VUI 202012/01 recently recognized in the United Kingdom. GSAID EpiCoV database. The nucleic acids of the disclosure encoding the recombinant proteins, peptides, or fragments of the present disclosure may include one or more, two or more, three or more, four or more, five or more, six or more, or seven or more nucleic acid mutations encoding variants of SEQ ID NO: 1 selected from the group consisting of orfΔ3b, deletion 69-70, deletion 144, Y453F, N501Y, A570D, D614G, P681H, I692V, T716I, A829T, S982A, D1118, and/or M129I.

In the present disclosure, methods and compositions are provided for treating, preventing and/or preventing transmission of COVID-19.

In some embodiments, a vaccine or inoculum is provided comprising one or more coronavirus recombinant proteins for production of polyclonal immunoglobulin. The coronavirus recombinant proteins may comprise one or more recombinant coronavirus structural proteins. The structural proteins may be derived from a human coronavirus, or a coronavirus infecting cows, goats, sheep, rabbit, horse, pigs, and the like, as well as reptiles, as a source of the antibodies associated with the sequences.

The vaccine or inoculum of the disclosure comprises one or more, two or more, three or more, four or more, or five or more recombinant SARS-CoV-2 proteins, fragments thereof, or substantially similar proteins.

Recombinant SARS-CoV-2 Proteins

Recombinant SARS-CoV2 structural proteins or fragments thereof may be derived from NCBI GenBank assession numbers MN908947.3. Severe acute respiratory syndrome coronavirus 2 isolate Wuhan-Hu-1 complete genome. Wu, F., et al., A new coronavirus associated with human respiratory disease in China, Nature 579 (7798), 265-269 (2020). Several recombinant SARS-CoV-2 proteins and fragments are also commercially available.

The recombinant proteins may comprise full length protein (1-1273) or a fragment of at least 10, 20, 30, 40, 50, 60, 70, 80, 100, 150, or 200 contiguous amino acid residues of SARS-CoV-2 structural protein, surface glycoprotein QHD43416.1 (SEQ ID NO: 1), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, surface glycoprotein NCBI protein id QHD43419.1 (SEQ ID NO: 2), or a substantially similar protein.

The recombinant proteins may comprise full length protein or a fragment of at least 10, 20, 30, 40, 50, 60, or 70 contiguous amino acid residues of SARS-CoV-2 structural protein, E protein, envelope protein NCBI protein ID QHD43418.1 (SEQ ID NO: 3), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, nucleocapsid phosphoprotein QHD43423.2. (SEQ ID NO: 4), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, membrane glycoprotein NCBI protein ID QHD43419.1 (SEQ ID NO: 5), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 His-tagged (N terminus) S protein (SEQ ID NO: 6), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 His-tagged (C terminus) S protein (SEQ ID NO: 7), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 His-tagged (N terminus) N protein (SEQ ID NO: 8), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 His-tagged (C terminus) N protein (SEQ ID NO: 9), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 S1-subunit; 1-685 (SEQ ID NO: 10), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 S2-subunit 686-1273 (SEQ ID NO: 11), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 S protein-ectodomain 327-531 (SEQ ID NO: 12), or a substantially similar protein.

The recombinant proteins may comprise full length protein or a fragment of at least 10, 15, or 20 contiguous amino acid residues of SARS-CoV-2 structural protein, SARS-CoV-2 S1-S2 furin cleavage site; 671-692 (SEQ ID NO: 13), or a substantially similar protein.

The recombinant proteins may comprise full length protein or a fragment of at least 10, 15, 20, or 25 contiguous amino acid residues of SARS-CoV-2 structural protein, SARS-CoV-2 S1-S2 polybasic cleavage site domain 667-694 (SEQ ID NO: 14), or a substantially similar protein.

The recombinant proteins may comprise full length protein or a fragment of at least 10, 20, 30, 40, or 50 contiguous amino acid residues of SARS-CoV-2 structural protein, SARS-CoV-2; S1-Receptor Bonding Domain (RBD); 451-509 (SEQ ID NO: 15), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2; S1 N-terminal domain, NTD protein (SEQ ID NO: 16), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2 S1 C-terminal domain, CTD protein (SEQ ID NO: 17), or a substantially similar protein.

The recombinant proteins may comprise full length protein or a fragment of at least 10, 20, 30, 40, 50, 60, 70, 80, 100, 150, or 200 contiguous amino acid residues of SARS-CoV-2 structural protein, SARS-CoV-2-S1 protein; 20-685 (SEQ ID NO: 18), or a substantially similar protein.

The recombinant proteins may comprise full length protein (1-1273) or a fragment of at least 10, 20, 30, 40, 50, 60, 70, 80, 100, 150, or 200 contiguous amino acid residues of SARS-CoV-2 structural protein, surface glycoprotein QHD43416.1 (SEQ ID NO: 41), or a substantially similar protein.

The recombinant proteins may comprise full length protein or a fragment of at least a SARS-CoV-2 S-protein and a SARS-CoV-2N-protein, or substantially similar proteins. In some embodiments, the recombinant proteins may include full length protein or a fragment of at least 10, 20, 30, 40, 50, 60, 70, 80, 100, 150, or 200 contiguous amino acid residues of SARS-CoV-2 surface glycoprotein (S protein) OHD43416.1 (SEQ ID NO: 1) and full length or at least 10, 20, 30, 40, 50, 60, 70, 80, 100, 150, or 200 contiguous amino acid residues of SARS-CoV-2 nucleocapsid phosphoprotein (N protein) OHD43423.2 (SEQ ID NO: 4).

The ACE2 recombinant proteins may comprise full length protein or a fragment of at least 10, 20, 30, 40, 50, 60, 70, 80, 100, 150, or 200 contiguous amino acid residues of human ACE2 protein or a substantially similar protein. In some embodiments, the human ACE2 protein comprises the amino acid sequence of SEQ ID NO: 28 or SEQ ID NO: 35.

The recombinant proteins of the disclosure may be utilized in an inoculum or vaccine for generation of polyclonal antibodies. In some embodiments, the recombinant proteins of the disclosure may be utilized in an inoculum or vaccine for generation of immunoglobulin Y polyclonal antibodies.

An inoculum or vaccine is provided comprising one or more, two or more, three or more, four or more, or five or more recombinant coronavirus proteins of the disclosure. The inoculum or vaccine may be used for vaccination of an animal for production of polyclonal antibodies. In some embodiments, the recombinant proteins of the disclosure may be utilized in an inoculum or vaccine for generation of immunoglobulin Y polyclonal antibodies.

Vaccines comprising recombinant SARS-CoV-2 structural proteins are provided for use in inoculating a production animal for producing polyclonal antibodies. The production animal may be a hen chicken for production of immune eggs comprising anti-coronavirus specific IgY antibodies.

IgY is a preferred polyclonal antibody type because it has a long record of safety and efficacy for ingestion, ex-vivo, and systemic use, and critically because IgY antibodies are very scalable (to billions of doses) and are very low-cost to produce (from a few cents per dose). IgY enables affordable and accessible prophylactic and therapeutic tools for managing the COVID-19 pandemic.

The recombinant proteins may comprise the amino acid sequence of one or more, two or more, three or more, four or more, or five or more of SEQ ID NO: 1, 2, 3, 4, 5, 6, 7, 8, 9, 10, 11, 12, 13, 14, 15, 16, 17, 18, 36, 37, 38, 39, 40, and or 41 or a fragment thereof, or a substantially similar protein. In some embodiments, the recombinant proteins may include one or more recombinant coronavirus spike proteins (S-proteins) or fragments thereof and one or more recombinant coronavirus nucleocapsid proteins (N-protein) or fragments thereof.

Optionally, recombinant proteins may be concentrated and/or purified. Recombinant proteins may be concentrated and/or purified by any known method. For example, recombinant proteins may optionally be concentrated or purified by a method comprising protein precipitation, size exclusion chromatograph (SEC), affinity chromatography such as a Nickel column, ion-exchange chromatography, and/or reverse-phase chromatography prior to formulation.

The vaccine or inoculum may include a killed or inactivated coronavirus. The killed or inactivated coronavirus may be any appropriate coronavirus including a human or animal coronavirus.

Veterinary coronaviruses are described in Tizard et al., 2020 Vaccine, 38 (33), 5123-5130. However, Tizard states that none of the existing domestic animal vaccines are likely to be in any way protective against SARS-1, MERS or SARS2. Nor do most of the domestic animal diseases closely resemble the acute, lethal pneumonic diseases of animals. The human vaccines will inevitably have to be developed independently.

In the present invention, the vaccine or inoculum for providing anti-coronavirus antibodies may include a veterinary coronavirus vaccine. The veterinary coronavirus vaccines may be any known veterinary coronavirus vaccine known in the art. In some embodiments, the veterinary coronavirus vaccine may be a poultry, porcine, or bovine coronavirus vaccine.

In some embodiments, the veterinary coronavirus vaccine may be a poultry coronavirus vaccine. The poultry coronavirus vaccine may be a poultry Infectious Bronchitis vaccine. For example, the infectious bronchitis poultry vaccine may be a Bronchitis vaccine, Arkansas Type, Live Virus (e.g. Poulvac® IB Ark, Zoetis US); Salmonella enteritidis Bacterin-Newcastle-Bronchitis Vaccine, Mass. Type, Killed Virus (e.g Poulvac® SE-ND-IB, Zoetis US); Newcastle-Bronchitis Vaccine, Mass Type, Killed Virus (e.g AviPro® 201 ND-IB, Elanco); ND and IB (Mass and Ark Types), and Salmonella enteritidis (e.g. AviPro® 329 ND-IB2-SE4, Elanco); Inactivated vaccine containing Newcastle disease (B1 type, LaSota strain), Infectious Bronchitis (Mass type), and Mycoplasma gallisepticum (e.g. AviPro® 304 ND-IB-MG, Elanco); Attenuated live vaccine with Newcastle disease (B1 type, B1 strain) and infectious bronchitis virus (Mass. and Conn. type) (e.g. AviPro® ND IB Polyblanco, Elanco); attenuated live vaccine with Newcastle disease virus (B1 type, LaSota strain) and infectious bronchitis virus (Mass type) (e.g., AviPro® ND IB Sohol, Elanco); live attenuated vaccine against IB (Mass type) via eye drop or spray admin (e.g., Nobilis® IB MA5, MSD Animal Health, UK).

In some embodiments, the veterinary coronavirus vaccine may be any infectious bronchitis vaccine known in the art. For example the infectious bronchitis vaccine may be, Ark Type, Live Virus (e.g. Biomune Company); Ark Type, Live Virus (e.g. Zoetis, Biomune Company); Ark Type, Live Virus (e.g. Boehringer Ingelheim Animal Health USA Inc., Lohmann Animal Health International); Ark Type, Live Virus (e.g. Boehringer Ingelheim Animal Health USA Inc., Intervet Inc.); Conn Type, Live Virus (e.g. Boehringer Ingelheim Animal Health USA Inc.); Delware Type, Modified Live Virus (e.g. Intervet Inc.); Georgia Type, Live Virus (e.g. Intervet Inc., Zoetis, Biomune Company); Georgia Type, Live Virus (e.g. Intervet Inc., Zoetis, Biomune Company); Mass & Ark Types, Live Virus (e.g. Intervet Inc., Zoetis, Biomune Company); Mass & Ark Types, Live Virus (e.g. Biomune Company); Mass & Conn Types, Live Virus (e.g. Boehringer Ingelheim Animal Health USA Inc., Zoetis, Biomune Company); Mass & Conn Types, Live Virus (e.g. Biomune Company); Mass & Conn Types, Live Virus (e.g. Intervet Inc.); Mass Type, Killed Virus (e.g. Zoetis, Biomune Company, Mass Type, Live Virus (e.g. Zoetis, Biomune Company); Mass Type, Live Virus (e.g. Zoetis, Biomune Company); Mass Type, Live Virus (e.g. Intervet Inc.); Mass Type, Live Virus (e.g. Boehringer Ingelheim Animal Health USA Inc.); Mass Type, Live Virus (e.g. Boehringer Ingelheim Animal Health USA Inc.); Mass Type, Live Virus (e.g. Boehringer Ingelheim Animal Health USA Inc.).

In some embodiments, the veterinary coronavirus vaccine may be a Bursal Disease-Newcastle Disease-Bronchitis Vaccine, B1 Type, B1 Strain, Mass & Conn Types, Live Virus (e.g. Boehringer Ingelheim Animal Health USA Inc.); Mass Type, Killed Virus (e.g. Zoetis); Standard & Variant, Mass & Ark Types, Killed Virus (e.g. Boehringer Ingelheim Animal Health USA Inc.).

In some embodiments, the veterinary coronavirus vaccine may be a Bursal Disease-Newcastle Disease-Bronchitis-Reovirus Vaccine, Killed Virus (e.g. Zoetis); Mass Type, Killed Virus (e.g. Biomune Company); Mass Type, Killed Virus (e.g. Zoetis); Mass Type, Killed Virus (e.g. Zoetis, Lohmann Animal Health International); Standard & Variant, Mass & Ark Types, Killed Virus (e.g. Boehringer Ingelheim Animal Health USA Inc.; Lohmann Animal Health International); Standard & Variant, Mass & Ark Types, Killed Virus (e.g. Lohmann Animal Health International); Standard & Variant, Mass Type, Killed Virus (e.g. Intervet Inc., Lohmann Animal Health International, Biomune Company); Standard & Variant, Mass Type, Killed Virus (Boehringer Ingelheim Animal Health USA Inc.); Standard & Variant, Mass Type, Killed Virus (e.g. Zoetis, Biomune Company).

In some embodiments, the veterinary coronavirus vaccine may be a Newcastle-Bronchitis Vaccine, B1 Type, B1 Strain, Mass & Ark Types, Live Virus (e.g. Intervet Inc.); B1 Type, B1 Strain, Mass & Ark Types, Live Virus (e.g. Boehringer Ingelheim Animal Health USA Inc., Zoetis); B1 Type, B1 Strain, Mass & Ark Types, Live Virus (e.g. Boehringer Ingelheim Animal Health USA Inc.); B1 Type, B1 Strain, Mass & Conn Types, Live Virus (e.g. Boehringer Ingelheim Animal Health USA Inc., Zoetis); B1 Type, B1 Strain, Mass & Conn Types, Live Virus (e.g. Lohmann Animal Health International); B1 Type, B1 Strain, Mass & Conn Types, Live Virus (e.g. Boehringer Ingelheim Animal Health USA Inc.); B1 Type, B1 Strain, Mass & Conn Types, Live Virus (e.g Boehringer Ingelheim Animal Health USA Inc.); B1 Type, B1 Strain, Mass & Conn Types, Live Virus (e.g. Intervet Inc.); B1 Type, B1 Strain, Mass Type, Live Virus (e.g. Boehringer Ingelheim Animal Health USA Inc., Zoetis); B1 Type, B1 Strain, Mass Type, Live Virus (e.g. Lohmann Animal Health International); B1 Type, B1 Strain, Mass Type, Live Virus (e.g. Zoetis); B1 Type, B1 Strain, Mass Type, Live Virus (e.g. Boehringer Ingelheim Animal Health USA Inc.); B1 Type, C2 Strain, Mass & Conn Types, Live Virus (e.g. Intervet Inc.); B1 Type, C2 Strain, Mass Type, Live Virus (e.g. Intervet Inc.); B1 Type, LaSota Strain, Mass & Conn Types, Live Virus (e.g. Boehringer Ingelheim Animal Health USA Inc.); B1 Type, LaSota Strain, Mass & Conn Types, Live Virus (e.g. Intervet Inc.); B1 Type, LaSota Strain, Mass Type, Live Virus (e.g. Boehringer Ingelheim Animal Health USA Inc., Zoetis); B1 Type, LaSota Strain, Mass Type, Live Virus (e.g. Boehringer Ingelheim Animal Health USA Inc., Zoetis); B1 Type, LaSota Strain, Mass Type, Live Virus (e.g. Lohmann Animal Health International); Mass & Ark Types, Killed Virus (Boehringer Ingelheim Animal Health USA Inc.); Mass Type, Killed Virus (e.g. Biomune Company); Mass Type, Killed Virus (e.g. Zoetis); Mass Type, Killed Virus (e.g. Lohmann Animal Health International); VG/GA Strain, Mass & Conn Types, Live Virus (e.g. Boehringer Ingelheim Animal Health USA Inc.).

In some embodiments, the veterinary coronavirus vaccine may be a Newcastle Disease-Bronchitis Vaccine-Salmonella Enteritidis Bacterin, Mass & Ark Types, Killed Virus (e.g. Lohmann Animal Health International); Mass & Ark Types, Killed Virus e.g. Lohmann Animal Health International).

In some embodiments, the veterinary coronavirus vaccine may be a Newcastle-Bronchitis Vaccine-Mycoplasma Gallisepticum Bacterin, Mass Type, Killed Virus (e.g. Lohmann Animal Health International).

In some embodiments, the veterinary coronavirus vaccine may be a Newcastle-Bronchitis Vaccine-Salmonella Enteritidis Bacterin, Mass Type, Killed Virus (e.g. Intervet Inc., Zoetis, Biomune Company.

In some embodiments, the veterinary coronavirus vaccine may be a Bronchitis Virus, Mass Type, Killed Virus (e.g. Biomune Company). The veterinary coronavirus vaccine may be administered to poultry by any method known in the art, for example, spray, in drinking water, intranasal, intraocular, or by injection, such as subcutaneous injection, or intramuscular injection.

In some embodiments, the veterinary coronavirus vaccine may be a bovine coronavirus vaccine. The bovine corona virus vaccine may be a live, killed, inactivated or attenuated bovine coronavirus vaccine. The bovine corona virus vaccine may contain a (BCoV) component.

As mentioned above, coronaviruses are currently divided into 4 genera based on partial nucleotide sequences of the RNA-dependent RNA polymerase: alpha (contains 4 subgroups A to D), beta, gamma, and delta. Bovine coronavirus is in the beta A grouping with close relationship to human respiratory coronaviruses. Ellis, John, Canadian Veterinary Journal, February 2019, 60:147-152. In some embodiments, the vaccine or inoculum may include a bovine veterinary coronavirus vaccine comprising a BCoV component. The BCoV component may include inactivated virus, live-attenuated virus, spike protein, for example, a viral vectored, subunit, or viral replicating particles vaccine, or a DNA vaccine, for example, encoding a spike, nucleocapsid, or a membrane protein, where genes encoding antigen are cloned into plasmid expression vector, or incorporated into genome of a carrier cell.

In some embodiments, the BCoV component is a commercial bovine coronavirus vaccine. For example, the BCoV component may be present in any commercial bovine coronavirus vaccine, for example, a SCOURGUARD® vaccine (Zoetis, US). For example, ScourGuard® 4K is for the vaccination of healthy, pregnant cows and heifers as an aid in preventing diarrhea in their calves caused by bovine rotavirus (serotypes G6 and G10), bovine coronavirus, and enterotoxigenic strains of Escherichia coli having the K99 pili adherence factor. ScourGuard 4K contains a liquid preparation of inactivated bovine rotavirus (serotypes G6 and G10) and coronavirus propagated on established cell lines and a K99 E. colibacterin. The bovine coronavirus vaccine may be CALF-GUARD® (Zoetis, US) containing attenuated strains of bovine rotavirus and bovine coronavirus propagated on established cell lines and freeze-dried to preserve stability. The bovine coronavirus vaccine (BCoV) may be SCOUR BOS™ 9 cattle vaccine (Elanco, US) comprising killed bovine rotavirus, coronavirus, as well as Clostridium perfringens type C, and E. coli bacterin toxoid; SCOUR BOS™ 4 (Elanco, US) bovine rotavirus, coronavirus vaccine-killed virus vaccine, e.g. formulated with Xtend™ III adjuvant; GUARDIAN® bovine rotavirus-coronavirus vaccine, killed virus, Clostridium perfringens, types C&D, E. coli bacterin-toxoid vaccine (Intervet/Merck Animal Health, NE, US). The attenuated BCoV vaccine may be, for example, disclosed in U.S. Pat. No. 10,434,168, which is incorporated herein by reference.

The antibody composition may also contain commercial bovine coronavirus antibodies. For example DUAL-FORCE FIRST DEFENSE® bolus (ImmuCell Corp., ME, US) bovine coronavirus-Escherichia coli antibody of bovine origin from hyperimmune colostrum; TRI-SHIELD FIRST DEFENSE GEL® (ImmuCell, ME, US) bovine rotavirus-coronavirus, E. coli antibody of bovine origin may be employed.

The veterinary coronavirus vaccine may be a porcine coronavirus vaccine, for example a porcine epidemic diarrhea virus (PEDV) vaccine, porcine transmissible gastroenteritis (TGEV) vaccine, porcine deltacoronavirus (PDCOV), or other porcine coronavirus vaccine. The porcine coronavirus vaccine may be an inactivated whole virus, live-attenuated whole virus, viral vectored virus for spike protein, subunit vaccine for spike protein, DNA vaccine to spike, nucleocapsid or membrane proteins, viral replicating particles vaccine to spike protein, or recombinant protein vaccine. Porcine coronaviruses are described in Gerdts and Zakhartchouk 2017, Veterinary Microbiology, 206, 45-51, which is incorporated herein by reference.

In some embodiments, the veterinary coronavirus vaccine may be a canine coronavirus vaccine. The canine coronavirus vaccine may be a canine enteric coronavirus vaccine. Canine enteric coronavirus (CCoV) is an alphacoronavirus infecting dogs that is closely related to enteric viruses of cats and pigs. At least two known serotypes exist, as well as novel variants of CCoV containing spike protein N-terminal domains (NTDs) that are closely related to feline and porcine strains. Canine coronaviruses are described in Licitra et al., 2014, Viruses, 6, 3363-3376, which is incorporated herein by reference. Licitra et al., 2014 report there is a high level of naturally occurring mutations occurs among coronaviruses especially within the spike gene, so there is a likelihood of continued emergence of novel CCoVs in the future.

In some embodiments, the veterinary coronavirus vaccine may be a ferret or feline coronavirus (FCoV) vaccine. In some embodiments, the feline coronavirus vaccine is a feline infectious peritonitis (FIP) virus vaccine or a feline enteric virus (FeCV) vaccine. For example, the FIP virus vaccine may be FELOCELL® FIP (Zoetis), modified live virus, which is intended for intranasal vaccination in cats.

One method to produce specific polyclonal antibodies is by utilizing the immune system of an avian host. For example, when presented with a target antigen (such as recombinant protein), a chicken's acquired immune system will activate and produce antibodies specific to the target antigen. These antibodies are transferred to the yolk of laid eggs. Müller, Sandra et al. “IgY antibodies in human nutrition for disease prevention.” Nutrition Journal vol. 14 109. 20 Oct. 2015, doi:10.1186/s12937-015-0067-3.

Birds (such as laying-hens) are highly cost-effective as producers of antibodies compared with mammals traditionally used for such production. Avian antibodies have biochemical advantages over mammalian antibodies. Immunologic differences between mammals and birds result in increased sensitivity and decreased background in immunological assays, as well as high specificity and lack of complement immune effects when administered to mammalian subjects. In contrast to mammalian antibodies, avian antibodies do not activate the human complement system through the primary or classical pathway nor will they react with rheumatoid factors, human anti-mouse IgG antibodies, staphylococcal proteins A or G, or bacterial and human Fc receptors. Avian antibodies can however activate the non-inflammatory alternative pathway. Thus avian antibodies offer many advantages over mammalian antibodies.

Fu et al. 2006 describe pathogen-free (SPF) chickens immunized with inactivated SARS coronavirus. Journal of virological methods, 2006, Vol 133, Num 1, pp 112-115.

Palaniyappan et al., 2012 describe SARS-Cov-1 N protein used for IgY production, and use in diagnostic ELISA. Poultry Science 91:636-642, 2012.

Shen et al., 2020 describe anti-SARS-CoV-2 IgY isolated from egg yolks of hens immunized with inactivated SARS-CoV-2. SARS-CoV-2 (20SF014-SARS-CoV-2) was expanded in Vero-E6 cells, collected, and stored at −80° C. until use. Hens were subcutaneously immunized with formaldehyde-inactivated SARS-CoV-2 and Freund's complete adjuvant, boosted twice at 2-3 week interval with the mixture of the inactivated virus and Freund's incomplete adjuvant on both wings (0.5 mL/hen). Three weeks after the final immunization, the eggs were collected, and crude IgY antibodies were extracted from the egg yolks. Virologica Sinica. DOI: 10.1007/s12250-021-00371-1.

Wei et al., 2021 describe neutralizing effect of anti-spike-S1 IgYs on a SARS-CoV-2 pseudovirus, as well as its inhibitory effect on the binding of the coronavirus spike protein mutants to human ACE2. SARS-CoV-2 Spike-S1 was expressed in Sf9 insect cells using the baculovirus/insect cell expression system. International Immunopharmacology, 90 (2021) 107172.

The present disclosure provides improved methods for rapid production of polyclonal IgY antibodies for use in various antiviral, antibacterial, anti-venom, anti-toxin, anti-virulence factor, anti-adherence factor, anti-prion, or anti-prion-like protein, therapeutic or prophylactic compositions are desirable.

The recombinant protein immunogen may be, for example, an ACE2 protein, such as a human ACE2 protein. The ACE2 protein may comprise the amino acid sequence of SEQ ID NO: 78. Anti-human ACE2 polyclonal antibodies are provided herein. ACE2 is an enzyme found in abundance on the cellular membranes of epithelial cells throughout the body. The anti-ACE2 IgY may be useful alone or in combination with anti-SARS-CoV-2 RBD IgY and/or anti-SARS-CoV-2 S-protein IgY for preparation of compositions for treating or preventing a coronavirus infection, for example, for preventing or decreasing SARS-CoV-2 viral transmission. The isolated biomolecule immunogen may be, for example, a SARS-CoV-2 protein. The SARS-CoV-2 protein may be a SARS-CoV-2 S-protein. The SARS-CoV-2-S-protein may comprise the amino acid sequence of SEQ ID NO: 86, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

Methods for generating anti-ACE2 polyclonal IgY antibodies are provided herein. ACE2 is an enzyme found in abundance on the cellular membranes of epithelial cells throughout the body. Research indicates that the ACE2 enzyme acts as a receptor to which SARS-CoV-2 binds and induces viral infection. To combat viral infection, two strategies (that may be used in combination for additive effect) are proposed: binding the virus at the receptor-binding domain (RBD) of the S1 subunit of the Spike protein and/or binding the docking site of the virus, the ACE2 receptor. Binding the RBD surface protein of the virus prevents the docking of the virus to the receptor by eliminating the “key” in the lock-and-key mechanism. Binding the ACE2 receptor itself also prevents the virus from docking by eliminating the “lock” even if a “key” escapes capture. This strategy promotes competitive inhibition of viral attachment, leaving no site on the virus or the host for infection to initiate. While antibodies specific to ACE2 could potentially cause issues systemically, IgY antibodies cannot pass the gastrointestinal barrier and are therefore of no concern to the systemic system.

The disclosure provides methods for generating anti-ACE2 IgY antibodies and demonstration of neutralizing effects to prevent SARS-CoV-2 RBD:ACE2 binding by anti-ACE2 IgY antibodies in sera after a single inoculation of purified recombinant ACE2 proteins. Recombinant ACE2 proteins were sourced from two suppliers for the first inoculation so that resulting titers could be compared, and one source could be selected for future boosters and production of anti-ACE2 IgY in egg yolk. White Leghorn chickens were inoculated with ACE2 protein in Freund's Complete Adjuvant. The chickens also received subsequent inoculations (boost inoculations). Following inoculation, blood samples were collected and serum was used to qualitatively measure specific antibody titers using antigen-specific indirect binding assays. The chicken serum samples were also tested in a SARS-CoV-2 Surrogate Virus Neutralization Test (sVNT) C-Pass™ Kit (GenScript) to quantitatively determine the neutralizing capacity of the IgY antibodies targeted to ACE2 protein. The specific anti-ACE2 IgY antibodies were identified in sera after a single inoculation. Samples from both ACE2-inoculated chicken groups demonstrated presence of anti-ACE2 IgY antibodies in serum. Further, when tested in the GenScript-sourced SARS-CoV-2 surrogate neutralization assay, anti-ACE2 IgY antibodies were able to neutralize the RBD:ACE2 binding interaction with over 90% efficacy. The recombinant human ACE2 protein may comprise an amino acid sequence of SEQ ID NO: 28, 35, 42, 43, 77 or 78 or a SARS-CoV-2 RBD-binding fragment thereof comprising from 50 to 500, or from 100 to 300, or at least 10, 20, 30, 40, 50, 60, 70, 80, 100, 150, or 200 contiguous amino acid residues thereof, or a substantially similar protein.

The anti-ACE2 IgY may be useful alone or in combination with anti-SARS-CoV-2 RBD IgY for preparation of compositions for treating or preventing a coronavirus infection, for example, for preventing or decreasing SARS-CoV-2 viral transmission.

Methods for production of IgY may comprise administering vaccines or inoculums comprising one or more recombinant human SARS-CoV-2 proteins, recombinant human ACE2 proteins, and optionally a bovine coronavirus vaccine to an appropriate production animal.

In some embodiments, any coronavirus vaccine may be used alone to inoculate poultry for anti-coronavirus immune egg IgY production. For example, the poultry may be inoculated with a commercial veterinary coronavirus vaccine. The coronavirus vaccine may also be used in combination with one or more coronavirus recombinant proteins of the disclosure to inoculate poultry. For example, chickens may be inoculated with both a commercial coronavirus vaccine and one or more coronavirus recombinant proteins. The one or more coronavirus recombinant proteins may be produced in an E. coli or Staphylococcus aureus cell line and whole cells may be used to inoculate chickens. Alternatively, a recombinant cell line comprising plasmids and/or synthetic genes encoding one or more coronavirus antigen proteins may be inactivated or killed using, for example, by mixing with gentamycin prior to inoculation. Alternatively, the recombinant cell comprising one or more genes encoding one or more recombinant proteins may further contain a kill switch, such that the cell will survive and be capable of expressing the one or more recombinant coronavirus proteins in a culture medium, but will autolyze under systemic conditions, for example, after inoculation of chickens to expose recombinant proteins.

### Antibody Production

For the purposes of this disclosure, antibodies may be either monoclonal, polyclonal derived from any animal, fragments, chimeric, humanized or any other form, and antibodies may be of any isotype: for example IgA, IgD, IgE, IgG and IgM (placental mammals), IgY (chicken), or others, may be a bispecific or bifunctional, or multispecific or multifunctional antibody or fragment thereof. In embodiments, the specific binding molecule can be selected from one of three main categories: mammalian monoclonal antibodies, mammalian polyclonal antibodies, and avian polyclonal antibodies; or any fragments derived therefrom that retain the ability to bind to the pathogenic component.

The antibodies of the disclosure may be collected from serum, plasma, colostrum, milk, eggs, or other suitable biologically derived fluid, or from cell culture media, supernatant, etc. The antibodies of the disclosure may be treated in any suitable manner to prepare for formulation and use, including but not limited to separations, plasmapheresis, drying processes, lyophilization, pasteurization, and preservation methods. The antibodies used in this invention may be treated, concentrated, separated, or purified in various ways depending upon their final intended use.

In one embodiment, antibodies are preferably raised in animals by one or more, e.g., by oral, intraocular, inhalable, intranasal, injectable, e.g., subcutaneous (sc), intramuscular (IM), or intraperitoneal (ip) injections, of the relevant antigen and optionally an adjuvant. In one aspect, it may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R N═C═NR, where R and R are different alkyl groups.

In one embodiment, the inoculum may be a recombinant protein—with an adjuvant. The adjuvant may include a PBS and oil adjuvant emulsion (oil in water, 30:70). The final concentration of protein in the emulsion may be ˜0.1-1 mg/mL, 0.1-0.6 mg/mL, about 0.2 mg per ml, 0.3 mg/ml, 0.4 mg/ml, 0.5 mg/ml, or any value in between. The adjuvant may be any suitable adjuvant for use in poultry as known in the art. Squalene (aluminum hydroxide may be present as well).

Animals may be immunized against the antigen, immunogenic conjugates, or derivatives as described herein. In other embodiments, the antibodies may be synthetic or semisynthetic, for example, as are obtained in a phage display library, or prepared as humanized or chimeric antibodies.

Birds (such as laying-hens) are highly cost-effective as producers of antibodies compared with mammals traditionally used for such production. Avian antibodies have biochemical advantages over mammalian antibodies. Immunologic differences between mammals and birds result in increased sensitivity and decreased background in immunological assays; as well as high specificity and lack of complement immune effects when administered to mammalian subjects. In contrast to mammalian antibodies, avian antibodies do not activate the human complement system through the primary or classical pathway nor will they react with rheumatoid factors, human anti-mouse IgG antibodies, staphylococcal proteins A or G, or bacterial and human Fc receptors. Avian antibodies can however activate the non-inflammatory alternative pathway. Thus avian antibodies offer many advantages over mammalian antibodies.

Several advantageous properties and features of IgY compared to mammalian IgG include non-invasive antibody sampling, 50-100 mg IgY per egg, 1500-2500 mg antibody yield per month per animal, specific antibody yield of about 2-10% of total IgY, no protein A protein binding, no interference with mammalian IgG, no interference with rheumatoid factor, no binding to mammalian Fc receptors, no activation of mammalian complement, glycosylation of antibodies, molecular weight of about 167 kD compared to about 160 kD for IgG, and isoelectric point of about pH 6.0-7.5 compared to pH 8.6 for IgG. Carlander et al., Biodrugs 200216(6): 433-437.

In one embodiment, polyclonal antibodies are prepared in eggs of hens inoculated with one of or a mixture of pathogenic components. Pathogenic components may include recombinant coronavirus proteins and/or coronavirus vaccines. Various preparations of specific antigens can also be employed for inoculation. After inoculation, the hen produces eggs containing substantial quantities of specific IgY immunoglobulin in the yolk, as well as small amounts of IgM and IgA immunoglobulins in the albumin. Therefore eggs are an excellent source for large quantities of economically produced, highly specific and stable antibodies. In one embodiment, hen chickens are used to produce avian antibody; however, hen turkeys, ducks, geese, ostriches, etc. may alternatively be used. In one aspect, hens are inoculated by any method known in the art, as described herein. For example, the antigen may be injected intramuscularly (IM) or subcutaneously (SC). One preferred muscle for IM injection in an avian is the breast muscle. Other methods of administration that can be used include subcutaneous injection (SC), intravenous injection (IV), intraperitoneal injection (IP), intradermal, rectal suppository, inhalation aerosol, or oral administration. The inoculant containing an immunogen according to the disclosure may be administered to poultry by any method known in the art, for example, in drinking water, inhalation aerosol, intranasal spray or drop, intraocular spray or drop, or by injection, such as subcutaneous injection, or intramuscular injection.

Plasmid DNA inoculation methods, for example, for administration of plasmid DNA, may include intramuscular injection, subcutaneous injection, DNA tattooing with dermally applied DNA plasmid using a tattoo gun (Hawk Pen, Cheyenne), or Gene Gun, which employs DNA coated gold particles (such as Gold Powder 0.8-1.5 μm from Alfa Aesar) directly into cells using high pressure helium gun (Helios Gene Gun System from Bio Rad).

Heterologous inoculation may be employed, for example, to rapidly increase initial IgY titer, while maintaining good titer over a period of time, by performinginitial protein inoculation of chickens followed by second and subsequent plasmid DNA inoculation encoding same target protein.

Inoculations and booster inoculations may be performed at 1-8 week, 2-6 week, or 3-4 week intervals. In some embodiments, plasmid DNA inoculations and booster inoculations may be performed at 1-3 week or about 2 week intervals. In some embodiments, protein inoculations may be performed at 4-8 week intervals, or 6-8 week intervals.

In some embodiments, IgY is avian IgY. The IgY may be produced in hen chickens. In some embodiments, the IgY is not ostrich IgY. The IgY may be extracted from egg, egg yolk, blood, or serum of immunized birds. The production of avian antibodies may offer advantages over production of mammalian antibodies, including antigen specificity, and lower production costs. The antibody may be a polyclonal antibody or a monoclonal antibody. In some embodiments, the IgY antibody is a polyclonal antibody. In some embodiments, the IgY antibody is not a monoclonal antibody.

The term “ostrich” may be used to define one of the birds that belong to the Struthioniformes. For example, the ostrich may be Struthio camelus. Ostriches can grow up to 9 ft tall and may weigh up to 320 lbs (145 kg). Ostriches may begin mating when they are about 4 years old. Ostriches do not lay eggs throughout the year like chickens do. They have a specific breeding season that may start, e.g., in June/July every year and may lay eggs about every other day until there are at least about 12-15 eggs. Eggs may be laid in a communal pit until enough eggs are in the nest. Ostrich eggs may weigh up to 3 lbs and contain ˜2-4 g IgY. An ostrich may live 50-75 yrs. Ostrich antibodies may be produce by any appropriate method, for example, by the method of U.S. Pat. No. 8,815,244. Immunization of ostriches may require a greater amount of antigen, than used for immunization of chickens. In one method, infant ostriches may be immunized at less than 3 months, and less than 5 months of age. Alternatively, ostriches may be immunized starting at ˜2.5 yrs of age weekly with about 4 mg antigen/0.5 mL per immunization. For example, immunization into dorsal skin of the ostrich may be utilized. Antibodies may be collected from egg yolk after about 6 weeks. In particular, if antibodies are to be collected from sera, ostriches may be preferred due to large volume of blood. Due to the size, difficulty in handling, and value of the ostrich, in some embodiments, IgY production in hen chickens is preferred.

Female chickens may start laying eggs as early as 16-18 weeks of age, or perhaps 20-22 weeks of age, depending on the breed. IgY is produced in egg yolk, so there is no need to bleed animals, considerable amounts of IgY antibodies can be obtained at relatively low cost, the production process may be rapid, IgY may be stored in eggs at 4° C. for extended periods of time, even up to about 1 year. Hen chicken eggs may yield from about 3.5 mg/ml to about 8.4 mg/mL yolk IgY. Amro et al., 2018, Production and purification of IgY antibodies from chicken egg yolk, J Gen Engineer and Biotechnol 16 (2018) 99-103. For example, hen chickens may be inoculated orally, by drinking water, IM, or SC, by inhalation aerosol, etc. starting at about 6 months of age, e.g., at intervals of about 1-2 weeks. In some embodiments, an initial immunization may be followed by 1 or more, 2 or more, 3 or more, 4 or more, 5 or more booster immunizations to maintain antibody titer. The IgY may be isolated from eggs of immunized chickens (immune egg). The immune egg may be whole egg, or egg yolk. The IgY may be isolated and or purified by any appropriate method. For example, IgY may be isolated and or purified from immune egg or yolk by a method comprising an extraction, precipitation and/or a chromatographic method. The chromatographic method may be a low pressure chromatographic method. The chromatographic method may be a gel filtration chromatographic method.

In some embodiments, immune eggs comprising anti-coronavirus IgY antibodies may be generated by inoculating poultry using a vaccine protocol and/or vaccine composition according to the present disclosure. The immunogen for vaccinating chickens may include a recombinant peptide or recombinant protein or nucleotide according to the disclosure. The immunogen may include a human SARS-CoV-2 vaccine. The immunogen may include a coronavirus vaccine. The immunogen may include a veterinary coronavirus vaccine such as an avian infectious bronchitis virus (IBV) vaccine, a bovine coronavirus vaccine, a porcine coronavirus vaccine, an enteric canine coronavirus vaccine, or a feline infectious peritonitis virus (FIPV) vaccine.

In some specific embodiments, a vaccine composition is provided comprising an immunogen such as recombinant SARS-CoV-2 S-protein, RBD protein, and/or N-proteins. The recombinant S-proteins may include a full-length or fragment of a full coronavirus spike protein, spike protein extracellular domain (ECD), S1-protein, receptor binding domain (RBD) protein, S2-protein, and/or N-protein, or a fragment thereof. In some embodiments, the coronavirus protein fragment may comprise from 7 to 150 amino acid residues, 10 to 100 amino acid residues, or from 15 to 75 amino acid residues of the S1-, S2-, RBD- and/or N-proteins. The coronavirus protein fragment amino acid residues may be contiguous amino acid residues or may be discontinuous amino acid residues.

In some embodiments, the chicken may be inoculated with any coronavirus protein, antigen thereof, or fragment thereof. For example, any commercial vaccine may be used to inoculate poultry, for example, comprising a coronavirus antigenic extract, a live, inactivated, attenuated or killed coronavirus vaccine, a coronavirus recombinant protein, fragment thereof, or a coronavirus DNA, or RNA vaccine. Poultry may be vaccinated using any suitable route of administration. The coronavirus vaccine may be a live, inactivated, killed or attenuated vaccine. For example, the virus may be killed by exposure to heat or formaldehyde. The virus may be inactivated by exposure to heat, chemicals or radiation. Split virus vaccines may be produced using a detergent to disrupt the virus. A subunit vaccine may be employed by purifying or isolating antigenic proteins while removing other components necessary for viral replication.

The specific immune state is preferably induced and maintained in the target animal by immunization and repeated booster administrations of an appropriate dosage at fixed time intervals. The time intervals for booster vaccinations are preferably 1-8 week intervals over a period of 1-12 months or more. Dosage may be selected, for example, between about 0.01-5 milligrams of the antigen. In one aspect, the dosage is 0.01 mg to 1.0 mg of antigen per inoculation, preferably 100 mg, 200 mg, 250 mg, 300 mg, 400 mg, 500 mg, or 750 mg antigen per inoculation of a hen chicken. For example, the total number of vaccinations can be selected from 1, 2, 3, 4, 5, or 6 or more in a 12 month period. Typically, a first inoculation is performed on day 1, with booster vaccinations on day 10, and day 20. The hen chicken can be re-vaccinated as needed by monitoring the specific antibody concentration, or titer, in the eggs by, e.g., ELISA. A typical subcutaneous dosage volume for a hen chicken may be selected from between about 0.2 to 1.0 mL, 0.3 to 0.7 mL, or 0.5 mL. However, it is essential that the booster administrations do not lead to immune tolerance. Such processes are well known in the art.

It is possible to use other inoculation maintenance procedures or combination of procedures, such as, for example, intramuscular injection for primary immunization and intravenous injection for booster injections. Further procedures include simultaneously administering microencapsulated and liquid immunogen, or intramuscular injection for primary immunization, and booster dosages by oral administration or parenteral administration by microencapsulation means. Several combinations of primary and booster immunization are known to those skilled in the art.

Improved methods for rapid production of polyclonal IgY antibodies for use in antiviral, antibacterial, antifungal, anti-venom, anti-toxin, anti-virulence factor, anti-adherence factor, anti-prion, or anti-prion-like protein therapeutic or prophylactic compositions.

In embodiments, the immunogen may be a recombinant protein, a whole cell, an inactivated virus, a plasmid DNA, or an isolated biomolecule.

The plasmid DNA immunogen may encode, for example, a recombinant protein as provided herein. In some embodiments, the plasmid DNA encodes a human ACE2 protein, SARS-CoV-2 S protein, or a fragment thereof, or substantially similar protein. In some embodiments, the plasmid DNA encodes a human ACE2 protein comprising the amino acid sequence of SEQ ID NO: 78. In some embodiments, the plasmid DNA encodes a SARS-CoV-2 S protein comprising the amino acid sequence of SEQ ID NO: 86.

The recombinant protein immunogen may be, for example, SARS-CoV-2 S-protein, S1-protein, RBD-protein, or N-protein, human ACE2 protein, norovirus capsid protein, Plasmodium falciparum circumsporozoite protein, Cryptosporidium protein such as C. parvum P23, a Clostridium difficile protein, for example, FliC, FliD, Cwp84, or Toxin B (TcdB), Staphylococcal protein A, CD20 protein, venom, rhinovirus VP4 protein, influenza VP1 capsid protein, prion protein, prion-like protein, herpes simplex virus glycoprotein gD, herpes simplex virus glycoprotein gD, rotavirus VP4 capsid protein, rotavirus VP7 surface glycoporotein, rotavirus NSP4 viral enterotoxin, zika virus NS-1 protein, Smallpox virus vaccinia complement protein (VCP), Bacillus anchracis lethal factor, Bacillus anchracis edema factor, Bacillus anchracis protective antigen (pagA), Ebola virus glycoprotein, Staphylococcus aureus SpA, cholera toxin subunit A, cholera toxin subunit B, or cholera toxin AB5.

The recombinant protein immunogen may be, for example, an ACE2 protein, such as a human ACE2 protein. The ACE2 protein may comprise the amino acid sequence of SEQ ID NO: 78. Anti-human ACE2 polyclonal antibodies are provided herein. ACE2 is an enzyme found in abundance on the cellular membranes of epithelial cells throughout the body. As mentioned herein, the ACE2 enzyme acts as a receptor to which SARS-CoV-2 binds and induces viral infection. To combat viral infection, two strategies (that may be used in combination for additive effect) are proposed: binding the virus at the receptor-binding domain (RBD) of the S1 subunit of the Spike protein and/or binding the docking site of the virus, the ACE2 receptor. Binding the RBD surface protein of the virus prevents the docking of the virus to the receptor by eliminating the “key” in the lock-and-key mechanism. Binding the ACE2 receptor itself also prevents the virus from docking by eliminating the “lock” even if a “key” escapes capture. This strategy promotes competitive inhibition of viral attachment, leaving no site on the virus or the host for infection to initiate. While antibodies specific to ACE2 could potentially cause issues systemically, IgY antibodies cannot pass the gastrointestinal barrier and are therefore of no concern to the systemic system.

The isolated biomolecule immunogen may be, for example, a Staphylococcal Protein A, or a toxin, for example, an exotoxin such as an enterotoxin, or endotoxin lipopolysaccharide, or a fragment thereof.

In some embodiments, methods are provided to rapidly produce a high titer of specific polyclonal antibodies by utilizing the immune system of an avian host. When presented with an antigen (such as whole cells or recombinant proteins), an avian's acquired immune system will activate and produce antibodies specific to the target antigen. These antibodies may be harvested from chicken serum or from the yolk of laid eggs. Müller, Sandra et al., “IgY antibodies in human nutrition for disease prevention.” Nutrition Journal vol. 14 109. 20 Oct. 2015, doi:10.1186/s12937-015-0067-3

The disclosure provides methods for inoculation of avian hosts for the production of IgY antibodies. Immunogens include fixed or inactivated whole cells, isolated or recombinant proteins, and plasmid DNA vaccines that are capable of inducing an immunogenic response and production of IgY antibodies in chickens within approximately one month. The IgY antibodies may be harvested from the inoculated chickens, for example, from egg, egg yolk, blood, or serum. Future implications of these findings could be the production of anti-microbial, anti-virulence factor, and anti-toxin therapeutics for human and animal health. These therapies could be used for the prevention of microbial colonization and derived illnesses from hospital-acquired or community-acquired infections.

Proteins are frequently used as the target inoculum for antibody production in chickens, however formalin-fixed whole cells can also be used to successfully produce IgY antibodies specific to entire cellular bodies. Formalin is a dilute solution of formaldehyde that preserves the cell by creating crosslinking between proteins and inhibits cells infectivity. Thavarajah, Rooban et al. 2012. “Chemical and Physical Basics of Routine Formaldehyde Fixation.” Journal of Oral and Maxillofacial Pathology: JOMFP 16(3):400-405. With the help of an adjuvant, the chicken's immune system will recognize the microbes and their surface proteins as antigen targets, inducing the production of specific antibodies; while the microbes remain incapable of colonization.

Methods are provided herein for generating polyclonal antibodies for use in antiviral, antibacterial, anti-venom, anti-toxin, anti-virulence factor, anti-adherence factor, anti-prion, or anti-prion-like protein, therapeutic or prophylactic compositions.

The viral immunogen may be, for example, a SARS-CoV-2 protein, such as a SARS-CoV-2 spike protein, S1-protein, S2-protein, RBD-protein, norovirus capsid protein, Herpes simplex 1 virus protein such as glycoprotein gD, Herpes simplex 2 virus protein glycoprotein gD, influenza virus capsid protein, such as VP1 capsid protein, rotavirus protein such as, for example, rotavirus VP4 capsid protein, rotavirus VP7 surface glycoprotein, Zika virus protein, such as ZIKV non-structural-1 (NS1) protein, Smallpox virus vaccinia complement protein (VCP), Smallpox virus SPICE protein, Ebola virus glycoprotein, and the like.

The viral immunogen target may be, for example, coronavirus such as a SARS-CoV-2 virus, rotavirus, zika virus such as PRV ABC59, norovirus, rhinovirus, calicivirus, herpes virus, influenza virus, smallpox virus, or an Ebola virus.

The bacterial target may be, for example, Staphylococcus aureus, Mycobacterium tuberculosis, Vibrio cholerae, Clostridium difficile, Clostridium perfringens, Yersinia enterocolitica, Shigella dysenteriae, Escherichia coli, Bacillus cereus, Streptococcus pyogenes, Salmonella enterica serotypes Typhimurium, Typhi, Paratyphi A and B, Klebsiella spp. such as Klebsiella oxytoca or Klebsiella pneumonia, Enterobacter spp. such as Enterobacter cloacae or Enterobacter sakazakii, Aeromonas spp. such as A. hydrophila, A. caviae, A. veronii biovar sobria, Proteus spp. such as P. mirabilis or P. vulgaris, Citrobacter spp. such as C. freundii, and Serratia spp. such as S. marcescens, S. rubidaea,

The immunogen may be a fixed or inactivated whole cell or isolated pathogen selected from the group consisting of: coronavirus, norovirus, rotavirus, calicivirus, enteric adenovirus, cytomegalovirus, astrovirus enteric adenovirus, Campylobacter jejuni, Salmonella, Salmonella typhimurium, Salmonella enterica serovar Typhi, Shigella dystenteriae, Plasmodium falciparum, Plesiomonas shigelloides, Escherichia coli [including (EPEC) enteropathogenic E. coli, (ETEC) enterotoxigenic E. coli, (EaggEC) enteroaggregative E. coli, (EIEC) enteroinvasive E. coli, and (EHEC) haemorrhagic E. coli], Yersinia enterocolitica, Vibrio cholerae O1, Vibrio O139, Non-O1 Vibrios, Vibrio parahaemolyticus, Aeromonas hydrophila, Clostridium perfingens, Clostridium difficile, enterohepatic Helicobacter (including Helicobacter pylori), Staphylococcus aureus, Klebsiella spp., venom, prion protein, prion-like protein.

The immunogen may be a venom protein. The venom protein may be derived from, a snake venom, spider venom, scorpion venom, lizard venom, jellyfish venom, Portuguese man-of-war venom, and the like. The venom protein may be, for example, a Short neurotoxin 2, recombinant venom peptide (Snouted cobra), for example, comprising the amino acid sequence of MICHNQQSSQPPTIKTCPGET NCYKKRWRDHRGTIIERGCGCPSV KKGVGI YCCKTNKCNR (SEQ ID NO: 87). The venom protein may be, for example, a Waglerin-1, recombinant venom peptide (temple pit viper), for example, comprising the amino acid sequence of GGKPDLRPCH PPCHYIPRPKPR (SEQ ID NO: 88). The venom protein may be, for example, a Kunitz-type kappaPI-theraphotoxin-Hsla, recombinant venom peptide, for example, comprising the amino acid sequence of IDTCRLPSDRGRCKASFERWYFNGRTCAKFIYGG CGGNGNK FPTQEACMKRCAKA (SEQ ID NO: 89). The venom protein may be, for example, a African snake venom from Bitis arietans.

The immunogen may be a microbial toxin. The toxin may be may be, for example, a Shiga toxin such as an E. coli Shiga toxin 1 B subunit or Shiga toxin 2 B subunit, Clostridium difficile toxin, such as Cwp84 or C. difficile toxin B, TcdB, Cholera toxin, such as cholera toxin, cholera toxin subunit A, cholera toxin subunit B,

The adherence factor target may be, for example, an adhesin, cadherin, cilia, lectin, pili, fimbrillae, or type 1 fimbriae, or viral adhesion structures. For example, the adherence factor target may be E. coli adherence pili antigens F41, 97P, F19, and or K99.

The prion target may be, for example, a human, cow, sheep, deer, or mouse prion protein (PrP, Prnp). The prion protein may be a human prion protein or peptide fragment thereof. The prion protein may have accession UniProtKB—P04156 (PRIO_HUMAN), and or comprise a sequence of

(SEQ ID NO: 90)
MANLGCWMLVLFVATWSDLGLCKKRPKPGGWNTGGSRYPGQGSPGGNRYP
PQGGGGWGQPHGGGWGQPHGGGWGQPHGGGWGQPHGGGWGQGGGTHSQWN
KPSKPKTNMKHMAGAAAAGAVVGGLGGYMLGSAMSRPIIHFGSDYEDRYY
RENMHRYPNQVYYRPMDEYSNQNNFVHDCVNITIKQHTVTTTTKGENFTE
TDVKMMERVVEQMCITQYERESQAYYQRGSSMVLFSSPPVILLISFLIFL
IVG.

The prion protein may comprise a label or tag such as a His-tag, for example, having amino acid sequence of

(SEQ ID NO: 91)
MGSSHHHHHH SSGLVPRGSH MKKRPKPGGW NTGGSRYPGQ
GSPGGNRYPP QGGGGWGQPH GGGWGQPHGG GWGQPHGGGW
GQPHGGGWGQ GGGTHSQWNK PSKPKTNMKH MAGAAAAGAV
VGGLGGYVLG SAMSRPIIHF GSDYEDRYYR ENMHRYPNQV
YYRPMDEYSN QNNFVHDCVN ITIKQHTVTT TTKGENFTET
DVKMMERVVE QMCITQYERE SQAYYQRGS.

The prion protein may be purchased commercially, or may be produced synthetically or recombinantly by any appropriate recombinant technique known in the art.

The immunogen may be a prion-like protein. The prion-like protein target may be, for example an Abeta or a tau protein. The term “prion-like” is often used to describe several aspects of tau pathology in various tauopathies, like Alzheimer's disease and frontotemporal dementia. True Prions are defined by their ability to induce miss folding of native proteins to perpetuate the pathology. True Prions, like PRNP, are also infectious with the capability to cross species. Since tau has yet to be proven to be infectious it is not considered to be a true prion but instead a “prion-like” protein. Much like true prions, pathological tau aggregates have been shown to have the capacity to induce miss folding of native tau protein. Both misfolding competent and non-mis folding competent species of tau aggregates have been reported, indicating a highly specific mechanism.

The prion-like protein may be amyloid beta. Amyloid beta denotes peptides of from about 36 to about 43 amino acids that are the main component of the amyloid plaques found in the brains of people with Alzheimer's disease. The peptides derive from the amyloid precursor protein, which is cleaved by beta secretase and gamma secretase to yield Aβ. Aβ molecules can aggregate to form flexible soluble oligomers which may exist in several forms. It is now believed that certain misfolded oligomers can induce other Aβ molecules to also take the misfolded oligomeric form, leading to a chain reaction akin to a prion infection. The oligomers are toxic to nerve cells. The other protein implicated in Alzheimer's disease, tau protein, also forms such prion-like misfolded oligomers, and there is some evidence that misfolded Aβ can induce tau to misfold. Amyloid β-Peptide (1-42) (Aβ42) human is a 42-amino acid peptide that plays a key role in the pathogenesis of Alzheimer disease (AD). The main deleterious effects in the pathogenesis are probably regulated by Aβ42, which acts as a repressor or activator of gene transcription causing further synaptic function damage and neuronal degeneration. Aβ42 is regarded as an important role in modulating the function of voltage-gated Ca²⁺- and K⁺-channels of the surface neuronal membranes. Amyloid β-Peptide (1-42) (Aβ42) DAEFRHDSGY EVHHQKLVFF AEDVGSNKGA IIGLMVGGVV IA (SEQ ID NO: 92). Amyloid β-Peptide (1-42) (Aβ42) may be purchased commercially (APExBIO Technology B6057) or may be prepared synthetically or by any appropriate recombinant protein production technique known in the art, for example in E. coli cells.

The prion-like protein may be a tau protein. The tau proteins (or r proteins) are a group of six highly soluble protein isoforms produced by alternative splicing from the gene MAPT (microtubule-associated protein tau). They have roles primarily in maintaining the stability of microtubules in axons and are abundant in the neurons of the central nervous system (CNS). They are less common elsewhere but are also expressed at very low levels in CNS astrocytes and oligodendrocytes. Pathologies and dementias of the nervous system such as Alzheimer's disease and Parkinson's disease are associated with tau proteins that have become hyperphosphorylated insoluble aggregates called neurofibrillary tangles. The tau hypothesis states that excessive or abnormal phosphorylation of tau results in the transformation of normal adult tau into paired-helical-filament (PHF) tau and neurofibrillary tangles (NFTs). The stage of the disease determines NFTs' phosphorylation. In AD, at least 19 amino acids are phosphorylated; pre-NFT phosphorylation occurs at serine 119, 202 and 409, while intra-NFT phosphorylation happens at serine 396 and threonine 231. Through its isoforms and phosphorylation, tau protein interacts with tubulin to stabilize microtubule assembly. All of the six tau isoforms are present in an often hyperphosphorylated state in paired helical filaments (PHFs) in the AD brain. The tau protein may be purchased commercially (e.g., Creative Biomart MAP516-H or MAPT-032H) or may be prepared synthetically or by any appropriate recombinant protein production technique known in the art, for example in E. coli or HEK293 cells.

Repetitive mild traumatic brain injury (TBI) is a central component of contact sports, especially American football, and the concussive force of military blasts. It can lead to chronic traumatic encephalopathy (CTE), a condition characterized by fibrillar tangles of hyperphosphorylated tau. After severe traumatic brain injury, high levels of tau protein in extracellular fluid in the brain are linked to poor outcomes.

The recombinant protein immunogen may be, for example, a norovirus capsid protein. The norovirus capsid protein may comprise the amino acid sequence of SEQ ID NO: 79.

Norovirus (NoV) is known to cause vomiting and diarrhea, and is one of the most prevalent cause of gastroenteritis in the United States. The virus is highly transmissible especially in densely populated locations (e.g., prisons, cruise ships, restaurants, and nursing homes). While the infection is initially acquired through food-borne and water-borne contamination, transmissibility is attributed to the aerosolization of viral particles. People and/or ingestible items in the vicinity of an afflicted individual are at an increased risk of contracting NoV or serving as a vector. Presently, there are no vaccines for the virus or alternative treatments for reducing the viral load from the gastrointestinal system. The virus is typically not persistent and symptoms generally resolve within a few days, however, afflicted individuals are at risk of severe dehydration and death can occur on rare occasion.

Norovirus is classified as a Caliciviridae virus-a single-stranded, positive sense RNA virus. The viral RNA is approximately 7.7 kilobases and codes for three open reading frames (ORF) of which the second ORF encodes for a major capsid protein. Research suggests that the target tissue of the P2 domain of the major capsid protein for NoV is histo-blood group antigen (HBGA), a complex carbohydrate which can be found in red blood cells and intestinal epithelial tissue. An antibody targeting the P2 site may prevent NoV from attaching to HBGA of red blood cells and intestinal epithelial tissue.

Human norovirus is classified into two groups, group 1& group 2. Norwalk virus is the species which belongs to group 1 and was discovered in 1968 at Ohio. Norovirus is a familiar virus which may causes human gastroenteritis with symptoms including vomiting, diarrhea, nausea, and stomach pain. Vomiting or diarrhea may lead to dehydration. Other symptoms may include fever, headache, and body aches. Symptoms may occur 12 to 48 hours after exposure to norovirus. Most people with norovirus infection typically recover within 1 to 3 days. CDC report revealed that there are 19-21 million Americans infected by Nororvirus annually with 800 deaths, 1 in 15 people with infection. Around the world, this virus affects about 267 million people and causes over 200,000 deaths each year; these losses are mostly in less developed countries and in the very young, elderly and immuno-suppressed population, though, most cases are self-limited with a full recovery within just a few days. Norovirus is extremely contagious and can spread from human to human through infected food, water or contaminated surfaces. The outbreaks usually occur from November-April, while the peak is in January. Norovirus is a positive sense RNA virus with 7.5 kb nucleotides, encoding a major structural protein VP1 with 50-55 kDa. The full length of VP1 capsid comprises the internal N-terminal, Hinge, shell (S) and protruding (P) domains. P domain from 225 to 520 forms P1-P2-P1 structure. Moreover, P domain has a receptor binding region which recognizes human histo-blood group antigens (HBGAs). P domain expressed in bacteria can spontaneously form a P dimer as well as a P particle aggregated by 12 P dimers.

Anti-norovirus capsid protein polyclonal IgY antibodies derived from chickens inoculated with a norovirus capsid protein may be used for preparation of a therapeutic composition for treating or preventing a norovirus infection, or for alleviating or reducing severity or duration of symptoms after exposure to norovirus.

The whole cell immunogen may be, for example, a fixed or inactivated whole cell derived from a microorganism. The whole cell immunogen may be, for example, derived from Staphylococcus aureus, Vibrio cholerae, Escherichia coli, Cryptosporidium parvum oocyst, or Mycobacterium tuberculosis, Mycobacterium tuberculosis, Clostridium difficile, Clostridium perfringens, Yersinia enterocolitica, Shigella dysenteriae, Escherichia coli, Bacillus cereus, Streptococcus pyogenes, Salmonella enterica serotypes Typhimurium, Typhi, Paratyphi A and B, Klebsiella spp. such as Klebsiella oxytoca or Klebsiella pneumonia, Enterobacter spp. such as Enterobacter cloacae or Enterobacter sakazakii, Aeromonas spp. such as A. hydrophila, A. caviae, A. veronii biovar sobria, Proteus spp. such as P. mirabilis or P. vulgaris, Citrobacter spp. such as C. freundii, and Serratia spp. such as S. marcescens, or S. rubidaea.

The immunogen may be derived from a whole cell preparation. The whole cell immunogen may be a fixed, inactivated, or attenuated whole cell microorganism. The cell may be fixed, inactivated, or attenuated by any appropriate technique known in the art. Methods of inactivation serve to destroy the pathogens ability to replicate and cause disease, but retain antigenicity for immune system recognition and antibody production.

For example, chemical fixation or inactivation may be performed by exposing the cell or virus particle to a chemical agent, such as an aldehyde (e.g., formaldehyde, glutaraldehyde), an alcohol (methyl alcohol, ethyl alcohol, phenol, acetic acid), oxidizing agent (e.g., osmium tetraoxide, potassium permanganate), or aziridine compounds, metallic compounds, or by exposure to heat or radiation.

Heat inactivation may be performed, for example, by exposing the whole cell preparation may be inactivated by heat, for example by heating to 75-85° C., or ˜80° C. for 5-30, 10-25, or ˜20 min. However, excessive heat inactivation should be avoided because it may denature the protein so the epitope structure may be altered or damaged.

One way to inactivate infectious agents in tissues before histologic analysis is formaldehyde fixation, first characterized in 1893 with the fixation of B. anthracis-infected tissue. Glutaraldehyde, the use of which was described decades later in 1963, may be used to inactivate samples for electron microscopy (EM) analysis. These related aldehydes cross-link primary amines and other reactive groups in proteins, fatty acids, and nucleic acids, thereby halting biochemical reactions and placing cellular structures in permanent stasis resembling structures found in living tissue. Formaldehyde molecules are small and diffuse quickly but may fix tissue slowly. An attractive property of formaldehyde fixation is that it is partially reversible and some denatured antigens can be retrieved to be again recognized by antibodies. In contrast, the larger glutaraldehyde molecules may fix tissues quickly and irreversibly but may not penetrate thick tissues well. For example, fixatives known in the art for electron microscopy may include may include 0.5-10% 4% formaldehyde, 10% formalin (e.g., Sigma), 10% neutral buffered formalin, 10% buffered formalin phosphate (e.g., Fisher Chemical), 4% paraformaldehyde, or 4% paraformaldehyde and 1% glutaraldehyde (Electron Microscopy Sciences) in 0.1 M sodium cacodylate (e.g., Sigma Aldrich) buffer. For example, immediately before its use, a phosphate-buffered saline (PBS), pH 7.4, may be used to dilute 10% formalin to 1% formalin, or used to dilute 16% paraformaldehyde (e.g., Electron Microscopy Sciences) to 4% paraformaldehyde. Chua et al., Emerging Infectious Diseases Vol. 25, No. 5, May 2019, Emerging Infectious Diseases, DOI: 10.3201/eid2505.180928. Other chemical fixatives may include phenol, or an aziridine compound such as binary ethylenimine (BEI). Depending on the chemical reagent employed, inactivation may be performed at room temperature or, for example, at 4° C. For example a BEA solution may be about 0.1M or about 20.5 g/L in 0.175N NaOH. The cell may be fixed or inactivated by treating with 1% formalin for 16-24 h at 4° C. The cell may be fixed or inactivated by exposing to 1-5% phenol in 70% ethanol, for example, at room temperature for at least 10 min. Chedore et al., 2002, J Clin Microbiol, vol. 40, No. 11, p. 4077-4080. Metallic fixatives may include mercuric chloride, mercuric chloride in acetic acid,

Formalin-fixation is a technique which allows for the microbes and their surface proteins to be presented to the immune system while inhibiting cellular activity. The avian's immune system recognizes the microbes and their surface proteins as antigen targets, inducing the production of specific polyclonal antibodies; while the microbes remain incapable of colonization.

In one embodiment, methods are provided for preparing formalin-fixed whole cell inoculation for targeted IgY titer production in avian hosts such as chickens. The inoculum may comprise contain a mixture of phosphate buffered saline (PBS), formalin-fixed cells of interest, and an adjuvant. The adjuvant may be any appropriate adjuvant. For example, the adjuvant may be a Freund's adjuvant, which are oil-based and may act as a nonspecific, immune system stimulator. Freund's Complete adjuvant is typically used for the first inoculation because it contains Mycobacterium which stimulates the immune response. Subsequent injections, referred to as ‘boosters’, use Freund's Incomplete adjuvant which lacks the Mycobacterium and is therefore less likely to cause a host adverse reaction. Briefly, the CFU count for the cells of interest is determined from a cell suspension in PBS. The cells are fixed overnight in a 1% formalin solution, washed in PBS, and resuspended in PBS. Next, the volume of formalin-fixed cells, PBS and Freund's adjuvant for inoculation is calculated. For example, the target cell concentration for Staphylococcus aureus is 1×10⁹ CFU/mL, and 2×10¹⁰ CFU/mL for Vibrio cholerae.^{2,3} The whole cell concentrations may be chosen based on literature values. After the inoculation components are combined, a water-in-oil emulsion may be formed between the aqueous fixed cell solution and Freund's adjuvant by forcibly mixing both liquids between two syringes. After the emulsion is formed, the chickens can be injected, for example, intramuscularly with the mixture.

For example, injection of formalin-fixed S. aureus whole cells into chickens to produce anti-S. aureus IgY has been demonstrated in the literature using various strains of S. aureus. Guimaries et al., 2009, Arch Immunol Ther Exp 57, 377-382. Staphylococcal protein A (SpA) has also been injected into chickens to create anti-SpA IgY. Zhen, Yu-Hong, et al. 2009. Veterinary Microbiology 133(4):317-22.

Staphylococcus aureus is a gram-positive bacterium and an important human pathogen capable of causing a wide range of infectious diseases such as skin infections, bacteremia, endocarditis, pneumonia, and food poisoning. Gnanamani, A. et al., 2017, Frontiers in Staphylococcus aureus, Ch. 1, pp. 1-27, Intech Open Science, http://dx.doi.org/10.5772/67338. S. aureus may be found in the normal human microbial flora of a significant portion of the population, but if it is able to enter the bloodstream or internal tissue, it can cause serious infection. There are many virulence factors associated with S. aureus, including those responsible for host cell attachment and toxin mediated symptoms. One such virulence factor is Staphylococcal protein A (SpA), a S. aureus surface protein which binds to immunoglobulins forcing them into a reverse orientation and thereby preventing phagocytosis by the host immune system. It is one mechanism that allows S. aureus to evade immune response. S. aureus is capable of causing a common hospital-acquired infection or community-acquired infection. There has been an increase in antibiotic resistant strains of S. aureus due to poor antibiotic stewardship and the lack of better alternative therapies. As such, there is a need for other therapies to stem the tide of S. aureus, especially antibiotic resistant strains. Antibodies offer a promising alternative therapeutic with specific target avidity and minimal risk of increasing antimicrobial resistance among the target strains.

The immunogen may be an isolated toxin. For example, an enterotoxin is a toxin specifically affecting cells of the intestinal mucosa, which may cause vomiting and diarrhea. The enterotoxin may be, for example, derived from rotavirus, Bacillus, Clostridium, Escherichia, Staphylococcus, and Vibrio. An enterotoxin is a protein exotoxin released by a microorganism that targets tissues. The enterotoxin may be derived from a viral pathogen. For example, rotavirus NSP4 viral enterotoxin. The enterotoxin may be derived from an enterotoxin-producing bacteria, for example, Clostridium difficile, Clostridium perfringens (Clostridium enterotoxin), Vibrio cholera (cholera toxin), Staphylococcus aureus (Staphylococcal enterotoxin B), Yersinia enterocolitica, Shigella dysenteriae (Shiga toxin), Escherichia coli, for example, enterotoxigenic strains of Escherichia coli producing heat-labile toxin, Bacillus cereus, Streptococcus pyogenes (Streptococcal exotoxins), Salmonella enterica (Salmonella enterotoxin) serotypes Typhimurium, Typhi, Paratyphi A and B, as well as certain Klebsiella spp. such as Klebsiella oxytoca (tilimycin, tilivalline) or Klebsiella pneumonia, Enterobacter spp. such as Enterobacter cloacae or Enterobacter sakazakii, Aeromonas spp. such as A. hydrophila, A. caviae, A. veronii biovar sobria, Proteus spp. such as P. mirabilis or P. vulgaris, Citrobacter spp. such as C. freundii, and Serratia spp. such as S. marcescens or S. rubidaea.

For example, the immunogen may be a cholera toxin, also known as choleragen. Cholera toxin is AB5 multimeric protein complex secreted by the bacterium Vibrio cholerae. Cholera toxin is responsible for the watery diarrhea characteristic of a cholera infection. It is a member of the heat-labile enterotoxin family.

Cholera is caused by Vibrio cholerae bacteria. The bacteria produces a toxin, e.g., in the small intestine known as cholera toxin (also known as choleragen, CTX). Cholera toxin interferes with the normal flow of sodium and chloride. When the bacteria attach to intestinal walls, the body secretes large amounts of water that leads to diarrhea and rapid loss of fluid and salts. Symptoms of cholera may be mild or severe and may include sudden inset of diarrhea, nausea, vomiting, mild to severe dehydration. Dehydration may cause tiredness, moodiness, dry mouth, extreme thirst, reduced urine output, irregular heartbeat, low blood pressure, electrolyte imbalance, muscle cramps, and shock. Children may also experience severe drowsiness, fever, convulsions, or coma. Other complications may include low blood sugar, low potassium levels, kidney failure. Once infected, a subject may shed cholera bacteria in the stool for 7-14 days. Symptoms may develop within 2-3 days after infection.

The immunogen may be derived from Vibrio cholerae which is a gram-negative gammaproteobacteria with serotypes 01 and 0139 responsible for the virulent expression of the cholera toxin, choleragen. Choleragen (cholera toxin) is a hexameric complex with one alpha subunit and 5 beta subunits (an AB5 toxin). Cholera is a serious bacterial disease that typically causes severe diarrhea and dehydration. The disease is typically spread through contaminated water. In severe cases immediate treatment is necessary because death can occur within hours. Choleragen is an enterotoxin and only produced under optimal gene expression conditions released after colonization. Upon entry into the gastrointestinal system, Vibrio cholerae colonizes the small intestines and produces the enterotoxin protein, choleragen. The pentameric beta subunits of the choleragen bind to ganglioside receptors present on the cell membranes of the epithelial cells of the small intestines, after which the alpha subunit enters the cell and induces the adenylate cyclase cascade. The beta subunit of choleragen is responsible for the binding of the ganglioside (GM₁) receptors while the alpha subunit is responsible for the toxigenicity. The ultimate effect of choleragen is the excessive permeability of the cell membrane to sodium, calcium, and chloride ions. As the ions are expelled from the cell membrane, the membrane becomes more permeable to the passage of water molecules. Persistent water loss will cause severe dehydration and even death to the host. For this reason, there is a great need for ingestible therapeutics for those residing in or traveling to regions where access to potable drinking water is limited. It is hypothesized that an ingestible therapeutic could prevent the colonization of V. cholerae in the gastrointestinal system or target the ganglioside receptors to prevent the choleragen mechanism of action if colonization has already occurred.

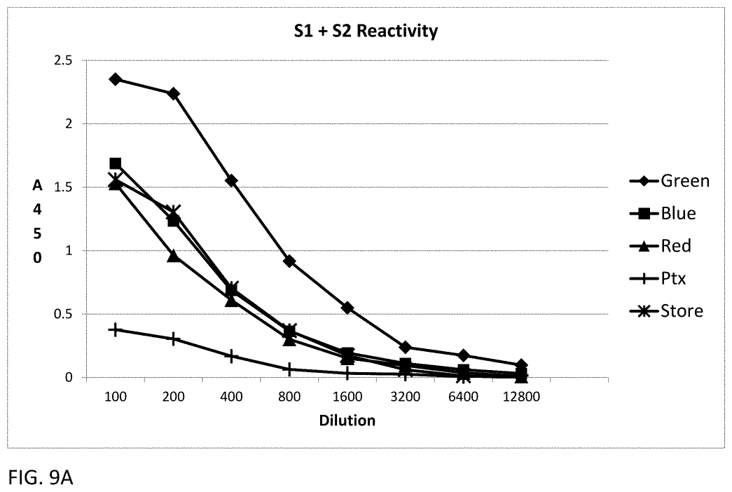

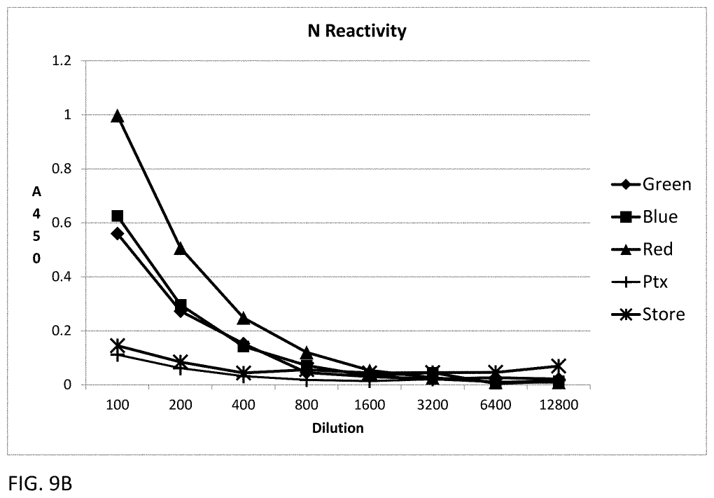

In the present application, hens were inoculated with formalin fixed V. cholerae whole cells and with isolated choleragen. The anti-whole cell V. cholera IgY in serum showed specific ELISA reactivity to coated fixed V. cholera whole cells as shown in FIG. 35; however, anti-choleragen IgY in serum exhibited low binding to coated whole V. cholerae cells. The reactivity of anti-choleragen IgY antibodies to the coated choleragen protein as shown in FIG. 9 indicates the present inoculation technique would allow the production of an anti-choleragen IgY in chicken egg yolk and serum, which may be useful in preparation of compositions to alleviate symptoms caused by colonized V. cholerae. A composition comprising anti-whole cell Vibrio cholerae IgY and anti-choleragen IgY may be useful for treating or preventing a Vibrio cholerae infection, and for alleviating or reducing severity or duration of symptoms due to exposure to cholera toxin.

When cholera toxin beta subunit is fused recombinantly to a particular antigen of interest, it is possible to amplify the polyclonal antibody production against the antigen as well as the subunit. The choleragen toxin subunit can also be expressed with a gene encoding for an antigen or toxin, acting as a genetic adjuvant as well. As demonstrated herein, there is significant anti-choleragen (alpha and beta subunit complex) polyclonal antibody titer and reactivity when inoculated with the complete alpha and beta subunit complex. Future implications could be the coupling to and use of isolated beta pentameric subunits as an adjuvant in compatible ganglioside receptor-utilizing targets.

The immunogen may be a virulence factor which is a biomolecule produced by bacteria, fungi, viruses and protozoa that aid in colonization, immunoevasion of host's immune response, immunosuppression of host's immune response, entry into and out of host cells, or obtain nutrition from the host. Virulence factors may be encoded by mobile genetic elements like plasmids or bacteriophages, and can convert harmless bacteria into dangerous pathogens. Virulence factors that promote host colonization may include adhesins, invasins, and antiphagocytic factors.

### Adjuvants

The immunogen of the disclosure may be combined with an adjuvant for inoculation of chickens. The vaccine for production of polyclonal antibodies may be adjuvanted to enhance the immune response. Adjuvants pertaining to the present disclosure may be grouped according to their origin, be it mineral, bacterial, plant, synthetic, or host product. Adjuvants may be included in the immunization solution/vaccine composition to enhance the specific immune response of the animal. A large number of adjuvants have been described and used for the generation of antibodies in laboratory animals, such as mouse, rats, rabbits and chickens. In such setting the tolerance of side effects is rather high as the main aim is to obtain a strong antibody response.

The vaccine for production of polyclonal antibodies may be adjuvanted to enhance the immune response. Adjuvants pertaining to the present disclosure may be grouped according to their origin, be it mineral, bacterial, plant, synthetic, or host product. The first group under this classification is the mineral adjuvants, such as aluminum compounds. Antigens precipitated with aluminum salts or antigens mixed with or adsorbed to performed aluminum compounds have been used extensively to augment immune responses in animals and humans. In one embodiment, the adjuvant in the immunization composition is from a bacterial origin. Adjuvants with bacterial origins can be purified and synthesized (e.g. muramyl dipeptides, lipid A) and host mediators have been cloned (Interleukin 1 and 2). Known chemical purification of several adjuvants of active components of bacterial origin includes: Bordetella pertussis, Mycobacterium tuberculosis, lipopoly-saccharide, Freund's Complete Adjuvant (FCA) and Freund's Incomplete Adjuvant (Difco Laboratories, Detroit, Mich.) and Merck Adjuvant 65 (Merck and Company, Inc., Rahway, N.J.). In a specific aspect, Freund's Complete Adjuvant and/or Freund's Incomplete Adjuvant may be employed in the immunization compositions of the disclosure. Additionally suitable adjuvants in accordance with the present invention are e.g. Titermax Classical adjuvant (SIGMA-ALDRICH), ISCOMS, Quil A, ALUM, see U.S. Pat. No. 5,554,372, Lipid A derivatives, choleratoxin derivatives, HSP derivatives, LPS derivatives, synthetic peptide matrixes, GMDP, oil-based adjuvant such as Xtend®III (Grand Laboratories, Inc., Larchwood, Iowa) and other as well as combined with immunostimulants (U.S. Pat. No. 5,876,735). B. pertussis is of interest as an adjuvant in the context of the present invention due to its ability to modulate cell-mediated immunity through action on T-lymphocyte populations. Freund's Complete Adjuvant is the standard in most experimental studies. Mineral oil may be added to the vaccination composition in order to protect the antigen from rapid catabolism.

Adjuvants for use with plasmid DNA vaccines may include a Class B oligonucleotide ODN 1826 (Invivogen tlrl-1826)(referred to as CpG). CpG is a family of synthetic oligodeoxynucleotides comprising single stranded DNA containing a cytosine triphosphate deoxynucleotide followed by guanine triphosphate deoxynucleotide, for example, comprising a nucleotide sequence of: TCCATGACGTTCCTGACGTT (SEQ ID NO: 93). This sequence is interpreted, by the host, as a signal of prokaryote invasion and therefore initiates immune system defense mechanisms. CpG has been used to enhance the immune response induced by DNA vaccines

Plasmid DNA adjuvants may be prepared by the methods and eukaryotic expression vectors herein, for example, by cloning to express a selected cytokine. The plasmid adjuvant may be cloned into in a separate plasmid or within the sequence of the plasmid DNA of interest. The plasmid adjuvants used for the DNA-based inoculations herein include separate plasmid adjuvants encoding sequences of the following cytokines: interferon gamma (IFNγ), heat shock protein from M. tuberculosis (HSP70), interleukin-2 from Gallus gallus (IL-2), and chicken granulocyte-macrophage colony stimulating factor (chGMCSF). Other cytokines for plasmid adjuvants include interleukins IL-6, IL-8, IL-15, cytokine Flt3 ligand, CCL19.

### Inoculation Methods

Many other types of materials can be used as adjuvants in immunogenic or immunization compositions according to the present disclosure. They include plant products such as saponin, animal products such as chitin and numerous synthetic chemicals.

In some embodiments, chickens may be vaccinated using isolated and/or purified recombinant viral protein antigens according to the disclosure, or inoculated with killed or live whole-cells of Escherichia coli or Staphylococcus aureus comprising nucleic acid sequences encoding recombinant viral protein antigens of the disclosure, with or without adjuvant. The live whole cells comprising nucleic acid encoding one or more SARS-CoV-2 recombinant proteins may further comprise a kill switch. E. coli and S. aureus kill switch strains contain a further synthetic genetic mutation such that the kill switch strains are unable to grow under systemic in vivo conditions and will autolyze, for example, releasing SARS-CoV-2 recombinant proteins following induction. Kill switched EC and SA strains comprising nucleic acids encoding SARS-CoV-2 S-protein were prepared by a modification of the method of Starzl et al., WO 2019/113096, which is incorporated herein by reference in its entirety.

For example, chickens immunized by the intramuscular route can produce high specific antibody levels in their eggs by day 28 after immunization and continue producing specific antibodies during more than 200 days making antibody preparations available in a short period of time, e.g. less than 4-5 weeks. Eggs contain IgY antibody concentrations of from up to about 50 to about 100 mg per egg. Over 100 mg of purified IgY can be obtained from a single egg. The percentage of antigen specific antibodies in one egg yolk can be up to about 2% to 10%. (daSilva et al., IgY: A promising antibody for use in immunodiagnostic and in immunotherapy. Veterinary Immunol. Immunopath., 135(2010):173-180).

One chicken of a high egg-laying strain can produce around 20 eggs per month. Eggs weigh from about 33 to about 77 grams, with about 10.5% of the whole egg due to shell. The yolk is about 31% of the weight of the whole egg. Upon drying, about 1 kg of dried whole egg powder can be produced from 72 eggs. Therefore, in this calculation, one egg can return about 10-18, 12-16, or about 13.9 g dried whole egg. In another aspect, one immune egg can return from 10 g to about 15 g dried whole egg. In another aspect, the immune eggs of the disclosure are from 40 to 55 mL per egg with about 1-2 mg/mL total IgY per egg. In another aspect, immune eggs of the disclosure contain about 0.01 mg/mL to 0.05 mg/mL specific IgY per egg. Therefore, in one aspect after processing, one dried whole immune egg contains about 80 to 110 mg total IgY and about 6 to 10 mg of total mixed antigen-specific IgY, e.g., from a chicken immunized with, for example a mixed antigen preparation.

It can be determined whether the vaccine has elicited an immune response in the egg-producing animal through a number of methods known to those having skill in the art of immunology. Examples of these include enzyme-linked immunosorbent assays (ELISA), tests for the presence of antibodies to the stimulating antigens, and tests designed to evaluate the ability of immune cells from the host to respond to the antigen. The minimum dosage of immunogen necessary to induce an immune response depends on the vaccination procedure used, including the type of adjuvants and formulation of immunogen(s) used as well as the type of egg-producing animal used as the host.

In one embodiment, hen chickens suitable for the commercial production of eggs are employed in the production of polyclonal antibodies. Any breed of chicken appropriate for egg production can be employed. For example, Rhode Island Reds, White Leghorns, Brown Leghorns, Lohmann Brown hens, sex-linked hybrid crosses, or other breeds suited to large egg size, high volume egg production and ease of handling can be selected. In one aspect, chickens are inoculated as chicks as for standard diseases (e.g. Salmonella, avian influenza, or Newcastle virus etc.). In one aspect, chickens of any age can be inoculated. Hens which are about to reach laying age, about 15-19 weeks for chickens, or any preselected time before or thereafter, are inoculated on a schedule predetermined by the amount and timing of final product to result in a steady continuous production stream. Typically, after a suitable period of isolation and acclimatization of about 2 to 4 weeks, each group will enter into an inoculation program using various antigens or immunization compositions comprising specific antigens to which an antibody is desired.

In one embodiment, the eggs are collected from inoculated chickens and processed as whole eggs. Eggs are stored under refrigeration conditions until enough are collected to prepare a batch. Batches of eggs from predetermined groups of chickens are cracked, the contents are separated from the shells and mixed and preferably pasteurized to eliminate potential contamination from pathogenic microorganisms from the chicken.

In one aspect, the immune egg products are pasteurized. Egg products are processed in sanitary facilities. Shell eggs are processed into immune egg product by automated equipment that removes the shell eggs from flats, washes and sanitizes the shells, breaks the eggs. Optionally, the whites are separated from the yolks. The liquid egg product is optionally filtered, optionally mixed with other ingredients, and is then chilled prior to additional processing. The resulting egg products liquid then receives a lethality treatment such as pasteurization or is heated in the dried form. In the U.S., the 1970 Egg Products Inspection Act (EPIA) requires that all egg products distributed for consumption be pasteurized.

Following pasteurization, the total egg content is dried using standard commercial methods, such as spray drying using ambient or hot air, thermal drying, freeze drying, or lyophilization. In one aspect, an appropriate method of drying the pasteurized liquid egg minimizes damage to the antibodies and molecular components in the egg, resulting in a product that has a high nutrient value and is capable of conferring passive protection.

In some embodiments, the immune egg may be broken, and spray dried, dehydrated, or lyophilized to obtain whole immune egg comprising anti-coronavirus IgY antibodies. Optionally the immune egg yolk may be separated from the egg white albumen. The egg yolk containing the IgY antibodies may be dried by any known means, such as dehydration, spray drying or lyophilization. The IgY antibodies may be further purified and/or isolated by any means known in the art.

In one aspect, the dried egg is tested to determine overall titer or antibody level. Standard test procedures are used, such as ELISA, FIA (fluorescent immunoassay), RIA (radioimmunoassay), or the like. In another aspect, the batch is blended with batches from groups of chickens at other average production levels resulting in a lot containing a standardized amount of antibodies. The dried egg containing specific polyclonal antibodies may be stored in an airtight container at room temperature prior to formulation into the compositions of the disclosure. In embodiments, the dried egg material is used as a whole egg and is not separated out. In embodiments, the whole dried egg material contains at least 5 mg per egg of specific IgY.

In another embodiment, IgY is isolated. The first step in the isolation of IgY is to separate the water-soluble proteins from lipoproteins. Water-soluble proteins constitute 42.4% of the total proteins in egg yolk (Osuga et al., “Egg Proteins: In Food Proteins, J. R. Whitaker and S. R. Tannenbaum eds., AVI Pub. Co., Westport, Conn. (1977)).

Many methods have been used for the isolation and purification of immunoglobulins from egg yolk (Martin et al., Can J. Biochem. Physiol. 35:241 (1957); Martin et al., Can. J. Biochem Physiol. 36:153 (1958); Jensenius et al., J. Immunol. Methods 46:63 (1981); Bade et al., J. Immunol. Methods 72:421 (1984); Polson et al., Immunol. Invest. 14:323 (1985); Hassl et al., J. Immunol. Methods 110:225 (1988)). Hatta et al. (Agric. Biol. Chem. 54:2531 (1990)) used food-grade natural gums (e.g., carrageenan) to remove yolk lipoprotein as a precipitate and to recover IgY in the water-soluble fraction from egg yolk. Methods for recovering antibodies from chicken egg yolk are well known in the art. Several methods can be used for the extraction of IgY from egg yolk, and commercial extraction kits are available (van Regenmortel, M. H. V. (1993). Eggs as protein and antibody factories. In Proceedings of the European Symposium on the Quality of Poultry Meat, pp. 257-263. Tours, France: INRA).

Plasmid DNA Based Inoculations

A plasmid-based expression system may be used to generate specific IgY antibodies according to the disclosure. A plasmid-based expression system may be employed to constitutively express a protein in mammalian cells, specifically chickens, in order to generate an antibody response against a specific protein. In embodiments, a eukaryotic expression vector system is employed that uses a constitutive cytomegalovirus (CMV) promoter and is modeled after a system used by Lee et al. 2006, Clin and Vaccine Immunol 13.3:395-402. The expression vector pCI-Neo was selected because it is a common mammalian expression vector used in various plasmid DNA vaccine studies for chickens.

The target DNA encoding the amino acid sequence of the target protein should be codon optimized to improve gene expression and increase the translational efficiency of the gene. In the process of transcribing DNA into mRNA and translating mRNA into protein, living cells use groupings of three nucleotides, called codons. During protein synthesis, the amino acid that is added to the growing chain of the peptide is determined by the codons. Different codons can code for the same amino acid, so organisms exhibit bias towards certain codons which can lead to an impact in expression. Codon optimization is important for efficiently producing a “non-native” protein within an animal host. The amino acid sequence is retained in codon optimization but the original nucleotide sequence is reassigned based on the codon usage frequency of the animal expressing the protein sequence. In some embodiments, codon optimization leads to increased “non-native” protein expression within the chicken cells.

A method for preparing a plasmid DNA eukaryotic expression vector is provided comprising

a) selecting a target protein amino acid sequence or a DNA sequence encoding the target protein amino acid sequence;
b) optimizing the codons of a DNA sequence encoding the amino acid sequence of the target protein for expression in Gallus gallus to obtain a codon-optimized target DNA sequence; and
c) cloning the codon-optimized target DNA sequence into a eukaryotic expression vector.

The target protein sequence may be selected from, for example, any appropriate bacterial, viral, fungal, or protozoal, protein, toxin, or adhesion element. For example, the target protein sequence may be a SARS-CoV-2 S-protein, S1-protein, RBD-protein, or N-protein, human ACE2 protein, norovirus capsid protein, Plasmodium falciparum circumsporozoite protein, Cryptosporidium protein such as C. parvum P23, a Clostridium difficile protein, for example, FliC, FliD, Cwp84, or Toxin B (TcdB), Staphylococcal protein A, CD20 protein, venom, rhinovirus VP4 protein, influenza VP1 capsid protein, prion protein, prion-like protein, herpes simplex virus glycoprotein gD, herpes simplex virus glycoprotein gD, rotavirus VP4 capsid protein, rotavirus VP7 surface glycoporotein, rotavirus NSP4 viral enterotoxin, zika virus NS-1 protein, Smallpox virus vaccinia complement protein (VCP), Bacillus anchracis lethal factor, Bacillus anchracis edema factor, Bacillus anchracis protective antigen (pagA), Ebola virus glycoprotein, Staphylococcus aureus SpA, cholera toxin subunit A, cholera toxin subunit B, or cholera toxin AB5, or a fragment, or substantially similar protein.

The codons may be optimized for target protein expression in the host species by employing an online tool, for example, such as IDT—https://www.idtdna.com/pages, or Genscript—https://www.genscript.com.

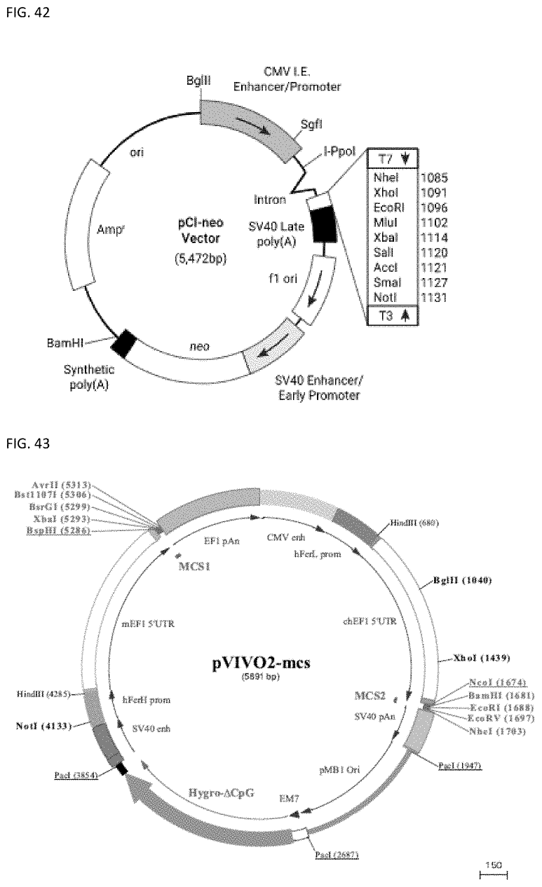

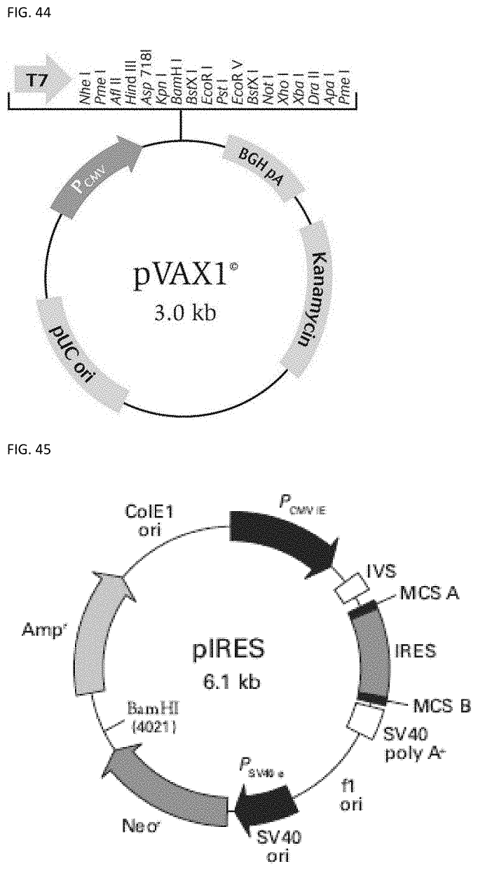

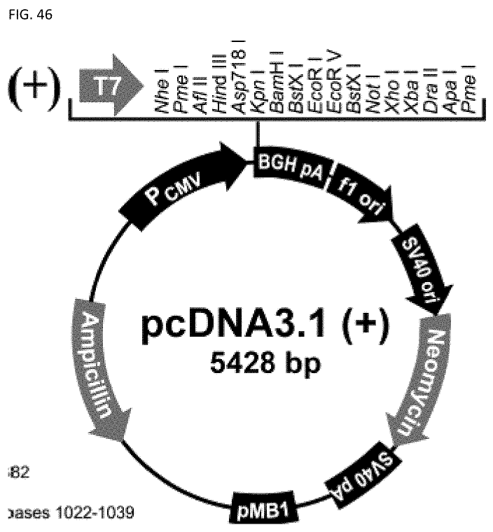

A plasmid DNA eukaryotic expression vector capable of expressing a target protein in a host cell may be employed for preparation of a plasmid DNA vaccine for IgY antibody production. In some embodiments, the eukaryotic expression vector is a pCI-neo eukaryotic expression vector, pVIVO2-mcs expression vector, pVAX1 expression vector, pIRES expression vector, or a pcDNA expression vector, as shown in FIG. 42-FIG. 46, respectively. The expression vector may be purchased commercially, for example, the vector may be selected from the group consisting of a pCI-neo mammalian expression vector (GenBank® Accession Number U47120, Promega 1841), pVIVO2-mcs vector (Invivogen, pvivo2-mcs), pVAX1 vector (ThermoFisher V26020), pIRES Vector (Clontech, PT3266-5), and a pcDNA 3.1 Mammalian Expression Vector (ThermoFisher V79020).

The codon optimized sequence may be pasted into the plasmid sequence in a computer program such as Benchling. Primers may be designed in from about 20 to about 60 bp in length to have at least about 15 bp overlap between the inserted gene and the expression plasmid. In some embodiments, the codon-optimized target DNA sequence is cloned between the T7 promoter sequence and the SV40 terminator sequence in a pCI-neo expression vector to obtain a plasmid DNA eukaryotic expression vector.

The pCI-neo expression vector may comprise the sequence of SEQ ID NO: 83. The pCI-neo plasmid T7 promoter and SV40 terminator sequences are shown in Table 17.

TABLE 17
pCI-neo plasmid Promoter and Terminator Sequences

| Region | Sequence (5′-3′) |
|---|---|
| T7 Promoter | TAATACGACTCACTATAGG |
| | (SEQ ID NO: 84) |
| SV40_PA_Terminator | TGATAAGATACATTGATGAGTTTGGAC |
| | AAACCACAACTAGAATGCAGTGAAAAA |
| | AATGCTTTATTTGTGAAATTTGTGATG |
| | CTATTGCTTTATTTGTAACCATTATAA |
| | GCTGCAATAAAC |
| | (SEQ ID NO: 85) |

The Primers and lyophilized double stranded DNA may be ordered from a commercial source (e.g. GenScript or ICT). Reconstitution of the primers and linear ds DNA and PCR amplification of the expression plasmid backbone is performed. The PCT product is checked on an agarose gel for successful amplification and purity. After PCR is complete 1 uL DpnI restriction endonuclease that cuts methylated DNA is added per 50 uL PCR mixture and incubated at least 1 h at 37° C. If the PCR product is clean band of approximate desired size on the agarose gel, the amplified DNA is purified, e.g. QIAquick PCR purification kit per manufacturers instructions. The linear DNA fragments may be assembled into a circular plasmid using a commercial kit, for example, a Gibson assembly kit per manufacturers protocol. The Gibson assembly mixture may be used to transform electrocompetent E. coli (e.g. Dh5 (NEB C2989K) per manufacturer's instructions. Following electroporation, recover the cells, for example, in SOC media for 1 h at 37° C. at 240 rpm. The cells are recovered and plated on LB agar plates and incubated up to ˜24 h at 37° C. The colonies are screened on transformation plates. One primer is selected that binds to the plasmid backbone and one primer that binds to another piece of DNA used in assembly. About 10 colonies are selected to analyze by PCR. A few positive colonies are picked from the patch plate and cultured overnight in LB broth, then plasmids are purified using for example, a commercial Zyppy Plasmid Mini purification kit per manufacturers instructions. If the extracted plasmid DNA is pure and concentration is sufficient (above 50 ng/uL) the plasmid is paired with primers and sent to a commercial service for DNA sequencing. If sequence is confirmed without error, that clone can be picked and stocked and stored at −80° C. Preparation for large scale purification may include from −80° C. cryostock, a plate is streaked for single colony isolation on a LB carb 100 plate and incubated 18-24 h at 37° C. A single colony is picked to inoculate ˜5 mL LB (carb 100) broth and incubated 9-12 h at 37° C. with shaking. The volume of the culture is scaled up by 1:500 dilutions into 2 L LB broth divided between several 2 L flasks and incubating at 37° C. The plasmid DNA may be extracted using a commercial kit such as PureLink HiPure Expi Plasmid Gigaprep Kit per manufacturers instructions, except, for the final resuspension of the purified pelleted DNA, resuspend in 0.75 mL of molecular biology grade (nuclease free) waterinstead of TE buffer. The concentration of DNA is measured comprising determining A260/A280 using a NanoDrop apparatus. The cloning includes confirming the sequence of the plasmid DNA.

Vaccination

Animals. Any appropriate production animal for production of antibodies such as IgY may be selected. Production animals may be selected from avians such as poultry, rabbits, goats, cattle, pigs, sheep, dogs, and the like. Cattle may include cows for immunization and collection of sera, colostrum or milk containing the desired antibodies. In a particular embodiment, the production animals may include egg-laying poultry. Production animals may be poultry selected from, without limitation, chickens, ducks, geese, turkeys, guinea fowl, ostriches, or emus. In a specific embodiment, the production animals may be chickens.

For production of IgY antibodies, hen chickens may be utilized. For example, Tetra, production Browns, or White Leghorn hen chickens may be used. The hen chickens may be from about 18-30 weeks depending on availability. Animals are kept at room temperature with clean water and laying ration ad libitum.

Serum sample collection. Optionally, serum may be collected before the first inoculation from 20 or so randomly chosen birds (if five per cage, then one from each cage), then on days 7, 21 and 35.

Inoculum. The inoculum may be a recombinant SARS-CoV-2 protein of the disclosure. For example, the recombinant SARS-CoV-2 recombinant protein may be an S-, S1-, S2-, spike ECD, S1-RBD-, or N-protein, or a fragment thereof of the disclosure. The inoculim may alternatively or additionally include a recombinant human ACE2 protein, or a fragment thereof. The recombinant protein(s) may be suspended in a carrier optionally with adjuvant. For example, PBS and oil adjuvant emulsion (oil in water, 30:70). For example, the final concentration of protein in the emulsion may be from 0.1 to 0.4, or 0.2 mg per ml. The adjuvant may be squalene (aluminum hydroxide may be present as well), or another adjuvant according to the disclosure. Plasmid inoculation using a GeneGun is also contemplated. Poly(3-hydroxybutyrate) (PHB) granules, or other biodegradable plastics, including poly-4-hydroxybutyrate(P4HB), polyhydroxyvalerate (PHV), polyhydroxyhexanoate (PHH), poly hydroxyoctanoate (PHO) and their copolymers, as well as dextran or other naturally occurring biopolymer may also be employed as an array of immunogens in the inoculation protocol.

Inoculation schedule. A 0.25 ml volume may be injected into each pectoral muscle of each chicken initially and at each interval (i.e. each animal receives 0.1 mg per inoculation). Other routes of inoculation may include spray, intraocular, intranasal, drinking water, oral, or by subcutaneous injection. The schedule for inoculation may be: initial inoculation on day 0, first booster on day 14, second booster at 28 days. Chickens may be kept an additional 12 months, 6 months, 3 months, or 30 days.

Eggs are to be collected continuously and refrigerated.

### Compositions

The disclosure provides compositions for preventing transmission, decreasing transmission, decreasing infectivity, decreasing severity of symptoms, decreasing duration of symptoms, and/or decreasing number of symptoms of SARS-CoV-2 infection or COVID-19. In one embodiment, the disclosure provides compositions for preventing transmission of and/or preventing and/or treating COVID-19 in a human patient.

Compositions comprising anti-SARS-CoV-2 IgY polyclonal antibodies specific for one or more, two or more, three or more, four or more, or five or more SARS-CoV-2 structural proteins or fragments thereof are provided. The SARS-CoV-2 structural proteins may include full length SARS-CoV-2 spike protein (S-protein) or a fragment thereof and a SARS-CoV-2 nucleocapsid protein (N-protein) or a fragment thereof. In some embodiments, the composition comprises anti-SARS-CoV-2 IgY polyclonal antibodies specific for S1, S2 and N proteins. The compositions may further include anti-bovine coronavirus IgY polyclonal antibodies. The compositions may further include anti-human-ACE IgY polyclonal antibodies. The compositions may further include anti-TMPRSS2 IgY polyclonal antibodies. In a specific embodiment, the composition comprises anti-SARS-CoV-2 IgY polyclonal antibodies specific for S1, S2 and N proteins, and anti-bovine coronavirus IgY polyclonal antibodies.

In some embodiments, the dried, powdered immune egg, egg yolk, isolated or purified IgY may be suspended in a liquid or solid vehicle or diluent. In some embodiments, the vehicle or diluent may be a pharmaceutically acceptable vehicle or diluent. In some embodiments, the dried whole immune egg, egg yolk, isolated or purified IgY may be suspended in a liquid vehicle or diluent. The liquid composition comprising the immune egg, egg yolk, isolated and/or purified IgY may be applied to a subject or surface, for example, via a spray bottle, nebulizer, atomizer, or as a dip, bath, or rinse.

In some embodiments, the antibody compositions of the disclosure may be administered to a subject in need thereof orally, sublingually, oropharyngeally, mouth rinse, gargle, intra nasally, parenterally, rectally, or by inhalation.

Compositions are provided comprising a mixture of anti-SARS-CoV-2 structural protein-specific polyclonal IgY antibodies and a pharmaceutically acceptable carrier, diluent, surfactant, emollient, binder, excipient, isotonicity agent, preservative, stabilizer, antibody matrix, antioxidant, lubricant, sweetening agent, flavoring agent, buffer, thickener, wetting agent, bulking agent, or absorbent.

Pharmaceutically acceptable diluents or carriers for formulating the composition may be selected from the group consisting of water, saline, phosphate buffered saline, phosphate buffered saline with TWEEN 20 (PBST), and/or a solvent. The solvent may be selected from, for example, ethyl alcohol, isopropyl alcohol, caprylyl glycol, toluene, n-butyl alcohol, castor oil, ethylene glycol monoethyl ether, diethylene glycol monobutyl ether, diethylene glycol monoethyl ether, dimethyl sulphoxide, dimethyl formamide and tetrahydrofuran. The carrier or diluent may further comprise one or more surfactants such as i) Anionic surfactants, such as metallic or alkanolamine salts of fatty acids for example sodium laurate and triethanolamine oleate; alkyl benzene sulphones, for example triethanolamine dodecyl benzene sulphonate; alkyl sulphates, for example sodium lauryl sulphate; alkyl ether sulphates, for example sodium lauryl ether sulphate (2 to 8 EO); sulphosuccinates, for example sodium dioctyl sulphonsuccinate; monoglyceride sulphates, for example sodium glyceryl monostearate monosulphate; isothionates, for example sodium isothionate; methyl taurides, for example Igepon T; acylsarcosinates, for example sodium myristyl sarcosinate; acyl peptides, for example Maypons and lamepons; acyl lactylates, polyalkoxylated ether glycollates, for example trideceth-7 carboxylic acid; phosphates, for example sodium dilauryl phosphate; Cationic surfactants, such as amine salts, for example sapamin hydrochloride; quartenary ammonium salts, for example Quaternium 5, Quaternium 31 and Quaternium 18; Amphoteric surfactants, such as imidazol compounds, for example Miranol; N-alkyl amino acids, such as sodium cocaminopropionate and asparagine derivatives; betaines, for example cocamidopropylebetaine; Nonionic surfactants, such as fatty acid alkanolamides, for example oleic ethanolamide; esters or polyalcohols, for example Span; polyglycerol esters, for example that esterified with fatty acids and one or several OH groups; Polyalkoxylated derivatives, for example polyoxy:polyoxyethylene stearate; ethers, for example polyoxyether lauryl ether; ester ethers, for example TWEEN; amine oxides, for example coconut and dodecyl dimethyl amine oxides. In some embodiments, more than one surfactant or solvent is included. The diluent or carrier may be a phosphate buffered saline. For example, a PBS may include 0.137 M NaCl (MW=58.4 g/mol), 0.0027 M KCl (MW=74.55 g/mol), 0.01 M Na2HPO4 (MW=151.96 g/ml), and 0.0018 M KH2PO4 (MW 136.086 g/mol).

The composition may include a buffer to help stabilize the pH. In some embodiments, the pH is between 4.0-8.5, or 4.5-8.0. For example, the pH can be approximately 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0, or any pH value in between. In some embodiments, the pH is from 5.0 to 8.0, 5.5 to 7.8, 6.0 to 7.5, 6.8 to 7.4, or about 7.0, or about 7.4.

Adjustment of the composition to such a pH may be accomplished by adjustment with an acid or base known in the art, by using adequate mixtures of buffer components, or both. The pH may be maintained using any appropriate buffering agent.

Non-limiting examples of buffers can include ACES, acetate, ADA, ammonium hydroxide, AMP (2-amino-2-methyl-1-propanol), AMPD (2-amino-2-methyl-1,3-propanediol), AMPSO, BES, BICINE, bis-tris, BIS-TRIS propane, borate, CABS, cacodylate, CAPS, CAPSO, carbonate (pK1), carbonate (pK2), CHES, citrate (pK1), citrate (pK2), citrate (pK3), DIPSO, EPPS, HEPPS, ethanolamine, formate, glycine (pK1), glycine (pK2), glycylglycine (pK1), glycylglycine (pK2), HEPBS, HEPES, HEPPSO, histidine, hydrazine, imidazole, malate (pK1), malate (pK2), maleate (pK1), maleate (pK2), MES, methylamine, MOBS, MOPS, MOPSO, phosphate (pK1), phosphate (pK2), phosphate (pK3), piperazine (pK1), piperazine (pK2), piperidine, PIPES, POPSO, propionate, pyridine, pyrophosphate, succinate (pK1), succinate (pK2), TABS, TAPS, TAPSO, taurine (AES), TES, tricine, triethanolamine (TEA), and Trizma (tris). The buffering agent may be ammonium phosphate, sodium phosphate, sodium biphosphate, potassium phosphate, potassium biphosphate, meglumine, potassium carbonate, potassium bicarbonate, sodium carbonate, sodium bicarbonate, sodium citrate, sodium dihydrogen citrate, sodium pyrophosphate, sodium succinate, and so forth. The composition may comprise a buffer in an amount of from about 1 mM to about 100 mM.

In some embodiments, the term “buffer” as used herein refers to a pharmaceutically-acceptable buffer. Suitable pharmaceutically-acceptable buffers include but are not limited to phosphate-buffers, histidine-buffers, citrate-buffers, succinate-buffers, acetate-buffers, TRIS-buffers, and the like. Preferred buffers include phosphate buffers, or phosphate-buffered saline. Histidine buffers may include L-histidine or mixtures of L-histidine with L-histidine hydrochloride. The above-mentioned buffers are generally used in an amount of from about 1 mM to about 100 mM, from about 5 mM to about 50 mM, about 10 mM, about 20 mM, about 30 mM, or any value in between.

The pH adjustment can be made with a physiologically acceptable acid, e.g., mineral acid such as HCl, sulfuric acid, phosphoric acid, or nitric acid. The pH adjuster may be any appropriate an organic acid, such as acetic acid, adipic acid, ascorbic acid, benzoic acid, citric acid, galacturonic acid, malic acid, dl-tartaric acid, quinic acid, formic acid, fumaric acid, lactic acid, lactobionic acid, malic acid, maleic acid, malonic acid, propionic acid, and/or succinic acid. The pH adjuster may be an alkalinizing agent such as sodium hydroxide, sodium carbonate, potassium carbonate, sodium bicarbonate, ammonium carbonate, tromethamine, and so forth.

The composition may include a binder may, for example, a gum tragacanth, gum acacia, methyl cellulose, gelatin, polyvinyl pyrrolidone, starch, biofilm component, or any other ingredient of the similar nature alone or in a suitable combination thereof.

Use of biofilm components as a glue or protective matrix is described in U.S. Pat. Nos. 10,086,025; 10,004,771; 9,919,012; 9,717,765; 9,713,631; 9,504,739, each of which is incorporated by reference. Use of biofilms as materials and methods for improving immune responses and skin and/or mucosal barrier functions is described in U.S. Pat. Nos. 10,004,772; and 9,706,778, each of which is incorporated by reference. For example, the compositions may comprise a strain of Lactobacillus fermentum bacterium, or a bioactive extract thereof. In preferred embodiments, extracts of the bacteria are obtained when the bacteria are grown as biofilm. The subject invention also provides compositions comprising L. fermentum bacterium, or bioactive extracts thereof, in a lyophilized, freeze dried, and/or lysate form. In some embodiments, the bacterial strain is Lactobacillus fermentum Qi6, also referred to herein as Lf Qi6. In one embodiment, the subject invention provides an isolated or a biologically pure culture of Lf Qi6. In another embodiment, the subject invention provides a biologically pure culture of Lf Qi6, grown as a biofilm. The pharmaceutical compositions may comprise bioactive extracts of Lf Qi6 biofilm. For example, L. fermentum Qi6 may be grown in MRS media using standard culture methods. Bacteria may be subcultured into 500 ml MRS medium for an additional period, again using proprietary culture methods. Bacteria may be sonicated (Reliance Sonic 550, STERIS Corporation, Mentor, Ohio, USA), centrifuged at 10,000 g, cell pellets dispersed in sterile water, harvested cells lysed (Sonic Ruptor 400, OMNI International, Kennesaw, Ga., USA) and centrifuged again at 10,000 g, and soluble fraction centrifuged (50 kDa Amicon Ultra membrane filter, EMD Millipore Corporation, Darmstadt, Germany, Cat #UFC905008). The resulting fraction may be distributed into 0.5 ml aliquots, flash frozen in liquid nitrogen and stored at −80° C.

The pharmaceutical compositions provided herein may optionally contain a single (unit) dose of probiotic bacteria, or lysate, or extract thereof. Suitable doses of probiotic bacteria (intact, lysed or extracted) may be in the range 104 to 10¹² cfu, e.g., one of 104 to 10¹⁰, 10⁴ to 10, 10⁶ to 10¹², 10⁶ to 10¹⁰, or 106 to 10⁸ cfu. In some embodiments, doses may be administered once or twice daily. In some embodiments, the compositions may comprise, one of at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to 10 about 5%, by weight of the Lf Qi6 extracts.

The abbreviation cfu refers to a “colony forming unit” that is defined as the number of bacterial cells as revealed by microbiological counts on agar plates.

Excipients may include any appropriate pharmaceutical excipient known in the art. Excipients may include, for example, a lactose, mannitol, sorbitol, microcrystalline cellulose, sucrose, sodium citrate, dextrose, dextrose monohydrate, dicalcium phosphate, phosphate buffer, or any other ingredient of the similar nature alone or in a suitable combination thereof.

The composition of the disclosure may contain pharmaceutically acceptable carriers or excipients selected from the group consisting of agar-agar, calcium carbonate, sodium carbonate, silicates, alginic acid, corn starch, potato tapioca starch, primogel or any other ingredient of the similar nature alone or in a suitable combination thereof, lubricants selected from the group consisting of a magnesium stearate, calcium stearate, talc, solid polyethylene glycols, sodium lauryl sulfate or any other ingredient of the similar nature alone; glidants selected from the group consisting of colloidal silicon dioxide or any other ingredient of the similar nature alone or in a suitable combination thereof; a stabilizer selected from the group consisting of such as mannitol, sucrose, trehalose, glycine, arginine, dextran, or combinations thereof, an odorant agent or flavoring selected from the group consisting of peppermint, methyl salicylate, orange flavor, vanilla flavor, or any other pharmaceutically acceptable odorant or flavor alone or in a suitable combination thereof, wetting agents selected from the group consisting of acetyl alcohol, glyceryl monostearate or any other pharmaceutically acceptable wetting agent alone or in a suitable combination thereof, absorbents selected from the group consisting of kaolin, bentonite clay or any other pharmaceutically acceptable absorbents alone or in a suitable combination thereof, retarding agents selected from the group consisting of wax, paraffin, or any other pharmaceutically acceptable retarding agent alone or in a suitable combination thereof.

In some embodiments, the composition comprising one or more coronavirus polyclonal IgY antibodies further comprises an antibody matrix. In some embodiments, the antibody matrix may be selected from the group consisting of natural biopolymer, flavonoid, phospholipids, fatty acid, hydrogel, or a colostrum. The antibody matrix is a protective matrix useful for prolonging antibody stability and or specific ELISA reactivity of the IgY antibodies upon exposure to alimentary canal/digestive tract, for example, in the oral cavity, esophagus, stomach, small intestine, and or large intestine, or simulated gastric fluid. Ingested antibodies may be susceptible to acidic and enzymatic degradation.

The antibody matrix may include a natural biopolymer or enteric compound to slow the release of the antibody in the gastrointestinal tract. Natural biopolymers may include natural polymers that may be produced by the cells of living microorganisms. For example, a natural biopolymer may be selected from alginate, carrageenan, gellan gum, guar gum, gelatin (WO 2009/127519), pectin, collagen, glucans, pullulan, chitosan, starch, polylactic acid (PLA), poly(lactic-co-glycolic acid)(PLGA), poly(acrylic acid)(PAA). Chitosan is a deacetylated derivative of chitin. Chitosan derived from shrimp recently was approved for generally recognized as safe (GRAS) status as a food additive by the US Food and Drug Administration (FDA). Fungal chitosan may be derived from Aspergillus niger. Chitosan is a linear copolymer comprised of randomly repeating glucosamine and N-acetylglucosamine units connected by beta->(1,4) type linkages.

The antibody matrix may include a flavonoid, phospholipids, fatty acids. The phospholipid may be a phosphatidylserine (PS). PS may be derived from soy, fish, krill, sunflower, or bovine sources. Sunflower lecithin is one source of PS that has received GRAS certification. See GRAS Notice 000636, 2016. PS contains a glycerophosphate conjugated with two fatty acids via a phosphodiester linage. The counterion for the phosphate moiety may be a Ca++ ion. Bovine source PS has mainly stearic acid and oleic acids. Plant sources have mainly linoleic acid and oleic acid, fish sources have mainly docosahexaenoic acid (DHA), eicosapentaenoic acid (EPA), and palmitic acid as the main fatty acids. The fatty acid may be caprylic acid, myristic acid, palmitic acid, palmitoleic acid, stearic acid, oleic acid), vaccenic acid, linoleic acid, alpha-linolenic acid, octadecatetraenoic acid, eicosenoic acid, arachidonic acid, eicosapentaenoic acid, erucic acid, cocosapentaenoic acid, docosahexaenoic acid, nervonic acid. The antibody matrix may be a hydrogel. For example, hydrogel microfibers have been generated from ascorbyl palmitate, an amphiphile that is generally recognized as safe (GRAS) by the U.S. Food and Drug Administration. Zhang et al., Sci Transl Med 2015, 7(300): 300ra128. Another GRAS hydrogel may include triglycerol monostearate. pH-sensitive hydrogel synthesis is described by Bellingeri et al., J Food Sci Technol, 2015, 52(5):3117-3122. Hydrogels containing acrylamide and acrylic acid were synthesized and used to encapsulate IgY. Stability of the IgY in simulated gastric fluid was significantly improved. However, the pH sensitive hydrogel retained a significant amount of igY at high pH, when the matrix has negative charges. Hydrogels may also be prepared from chitosan, poly vinylpyrrolidone, polyacrylic acid as polymers, for example, using crosslinkers gitaraldehyde or N,N′-methylene bisacrylamide.

The antibody matrix may be include a colostrum. Colostrum may be used as a protective antibody matrix, for example, as disclosed in Starzl, WO 2012/071346, which is incorporated herein by reference. The colostrum may be obtained from any appropriate mammalian species within 4 days, 3 days, 48 hours, or within 24 hours after giving birth. The colostrum may be a bovine, goat or sheep colostrum. The colostrum may be a non-human colostrum. The colostrum may be a commercially available colostrum such as bovine colostrum, or goat colostrum. The colostrum may be non-defatted colostrum or defatted colostrum. The colostrum may be dried colostrum for example, in a powder form, for example, spray dried or lyophilized. The powdered colostrum may be an instantized colostrum. The colostrum may be a non-hyperimmune colostrum wherein the mammal has not been specifically vaccinated with the target immunogen for the purpose of generating antibodies. In some embodiments, the colostrum may be hyperimmune colostrum, wherein the mammal is immunized with the target immunogen for the purpose of generating immunogen-specific antibodies.

The antibody matrix may include a stabilizing agent selected from the group consisting of a lipid stabilizing agent, a polysaccharide stabilizing agent, a disaccharide stabilizing agent, a sugar alcohol stabilizing agent, or a protein stabilizing agent. The stabilizing agent may be a microencapsulating agent, an acid resistant coating material, and/or enteric coating material.

The stabilizing agent may serve to stabilize the composition with respect to gastrointestinal conditions and/or storage conditions. The stabilizing agent be used as a matrix material that is mixed or blended with the IgY, and/or may be used as a coating material to the matrix composition or the dosage form. Stabilizing agent materials may be selected from the list of excipients provided herein, or as follows, for example, phospholipids (e.g., Lecithin, ADM), lecithin, a medium-chain triglyceride (MCT), an alginic acid, alginate, colostrum fat, ethyl cellulose aqueous dispersion (Surelease®, Colorcon), maize starch, shellac (e.g., dewaxed aqueous based 25%, Marcoat®, Emerson Resources), sodium bicarbonate, pseudolatex, pea starch, hydroxypropyl methyl cellulose (HPMC), water soluble HPMC (e.g., Methocel® F4M or K15M, Dupont), HPMC acetate succinate, maltitol powder (e.g., SweetPearl® (e.g., Roquette), maltodextrin (e.g., Kleptose®, Roquette), disaccharide (e.g., trehalose, Bolise® Treha), casein or a caseinate salt such as calcium caseinate, sodium caseinate, potassium caseinate, magnesium caseinate, whey protein concentrate, milk protein concentrate, sweet whey, non-fat dry milk, xanthan gum (e.g., ADM), polydextrose (e.g., Tate and Lyle, DuPont), prolamine derived from corn, water-insoluble protein isolated from corn (e.g., zein, FloZein), alginate plus ethyl cellulose aqueous dispersion (e.g., Nutrateric®, Colorcon), stearine, glyceryl stearates, hydrogenated vegetable oil, alginate plus ethyl cellulose (e.g., Protect® EN, Sensient), alginate plus maize starch (e.g., Eudraguard Natural, Evonik), hydroxypropyl pea starch (e.g., LyCoat®, Roquette), partially hydrogenated soybean oil, hydrogenated soybean oil, hypromellose acetate succinate (e.g., AQOAT®, Shin-Etsu), aqueous cellulose acetate phthalate polymer (e.g., Aquacoat® CPD, Colorcon), alginate plus acrylic polymer (e.g., Eudraguard® Control, Evonik), or ethyl cellulose aqueous dispersion colloidal (e.g., Aquacoat® ECD, Colorcon).

In some embodiments, the stabilizing lipid may be a phospholipid, lecithin, medium-chain triglyceride, hydrogenated soybean oil, partially hydrogenated soybean oil, stearine, exogenous casein, caseinate, or exogenous colostrum fat. In some embodiments, the matrix stabilizing agent may be a polysaccharide stabilizing agent or disaccharide stabilizing agent. For example, the polysaccharide stabilizing agent may be a starch, alginate, alginic acid, polydextrose, guar gum, xanthan gum, maltodextrin, hydroxypropyl methylcellulose (HPMC; hypromellose), hydroxypropyl cellulose, methylcellulose, hydroxyethyl cellulose, hydroxyethyl methylcellulose, carrageenan, or pectin. The disaccharide stabilizing agent may be trehalose, maltose, sucrose, lactose, lactulose, or cellobiose. The stabilizing disaccharide may be a reducing disaccharide. The stabilizing disaccharide may be a non-reducing disaccharide. In one example, the stabilizing disaccharide may be trehalose. The stabilizing agent may be a sugar alcohol stabilizing agent. The sugar alcohol stabilizing agent may be maltitol, mannitol, xylitol, sorbitol, erythritol. The stabilizing agent may be a stabilizing protein. The stabilizing protein may be a casein, a nutritionally acceptable salt such as calcium caseinate, magnesium caseinate, sodium caseinate, potassium caseinate, whey protein concentrate, whey protein isolate, milk protein concentrate, sweet whey, or non-fat dry milk.

The stabilizing agent may be a microencapsulating agent, acid resistant coating material, and/or enteric coating material. In some embodiments, the microencapsulating agent or enteric coating material may be alginate plus ethyl cellulose aqueous dispersion (e.g., Nutrateric®, Colorcon), alginate plus ethyl cellulose (e.g., Protect® EN, Sensient), alginate plus maize starch (e.g., Eudraguard Natural, Evonik), hydroxypropyl pea starch (e.g., LyCoat®, Roquette), hypromellose acetate succinate (e.g., AQOAT®, Shin-Etsu), aqueous cellulose acetate phthalate polymer (e.g., Aquacoat® CPD, Colorcon), alginate plus acrylic polymer (e.g., Eudraguard® Control, Evonik), or ethyl cellulose aqueous dispersion colloidal (e.g., Aquacoat® ECD, Colorcon).

Stabilizing agents may be present in the composition at 0-60 wt %, 0.5-50 wt %, 5-40 wt %, 1-30 wt %, or 5-25 wt % compared to the total weight of the composition.

The composition may comprise one or more emollients. Non-limiting examples of emollients include stearyl alcohol, mineral oil, dimethicone, petrolatum, glyceryl monoricinoleate, glyceryl mono stearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, sesame oil, coconut oil, arrachis oil, castor oil, acetylated lanolin alcohols, petroleum, mineral oil, butyl myristate, isostearic acid, palmitic acid, isopropyl linoleate, lauryl lactate, myristyl lactate, decyl oleate, and myristyl myristate.

The composition may include a thickener, for example, where the thickener may be selected from hydroxyethylcelluloses (e.g. Natrosol), starch, gums such as gum arabic, kaolin or other clays, hydrated aluminum silicate, fumed silica, carboxyvinyl polymer, sodium carboxymethyl cellulose or other cellulose derivatives, ethylene glycol monostearate and sodium alginates. The composition may include preservatives, antiseptics, pigments or colorants, fragrances, masking agents, and carriers, such as water and lower alkyl, alcohols, such as those disclosed in an incorporated by reference from U.S. Pat. No. 5,525,336 are included in compositions.

The compositions may be provided in ready-to-use form such as a liquid, solution, suspension, cream, lotion, ointment, gel, film, or in a form for further suspension or dilution prior to administration such as a concentrated solution, frozen form, powder, tablet, or troche for dilution or suspension immediately prior to administration. The compositions may also be provided as hard capsules, or soft gelatin capsules, wherein the benign and/or antibody composition is mixed with water or an oil medium, for example, peanut oil, liquid paraffin, or olive oil. Powders and granulates may be prepared using the ingredients mentioned above under tablets and capsules for dissolution in a conventional manner using, e.g., a mixer, a fluid bed apparatus, lyophilization or a spray drying equipment. A dried composition may administered directly or may be for suspension in a carrier. When the composition is in a powder form, the powders may include chalk, talc, fullers earth, colloidal silicon dioxide, sodium polyacrylate, tetra alkyl and/or trialkyl aryl ammonium smectites and chemically modified magnesium aluminum silicate in a carrier. When the composition is in a powder form, the powders may include chalk, talc, fullers earth, colloidal silicon dioxide, sodium polyacrylate, tetra alkyl and/or trialkyl aryl ammonium smectites and chemically modified magnesium aluminum silicate

Compositions are provided comprising a SARS-CoV-2 structural protein-specific polyclonal immunoglobulin, for example IgY, and a pharmaceutically acceptable carrier, diluent, emollient, binder, excipient, lubricant, sweetening agent, flavoring agent, buffer, thickener, wetting agent, or absorbent.

Pharmaceutically acceptable diluents or carriers for formulating the composition may be selected from the group consisting of water, saline, phosphate buffered saline, phosphate buffered saline with TWEEN 20 (PBST), and/or a solvent. The solvent may be selected from, for example, ethyl alcohol, toluene, isopropanol, n-butyl alcohol, castor oil, ethylene glycol monoethyl ether, diethylene glycol monobutyl ether, diethylene glycol monoethyl ether, dimethyl sulphoxide, dimethyl formamide and tetrahydrofuran. The carrier or diluent may further comprise one or more surfactants such as i) Anionic surfactants, such as metallic or alkanolamine salts of fatty acids for example sodium laurate and triethanolamine oleate; alkyl benzene sulphones, for example triethanolamine dodecyl benzene sulphonate; alkyl sulphates, for example sodium lauryl sulphate; alkyl ether sulphates, for example sodium lauryl ether sulphate (2 to 8 EO); sulphosuccinates, for example sodium dioctyl sulphonsuccinate; monoglyceride sulphates, for example sodium glyceryl monostearate monosulphate; isothionates, for example sodium isothionate; methyl taurides, for example Igepon T; acylsarcosinates, for example sodium myristyl sarcosinate; acyl peptides, for example Maypons and lamepons; acyl lactylates, polyalkoxylated ether glycollates, for example trideceth-7 carboxylic acid; phosphates, for example sodium dilauryl phosphate; Cationic surfactants, such as amine salts, for example sapamin hydrochloride; quartenary ammonium salts, for example Quaternium 5, Quaternium 31 and Quaternium 18; Amphoteric surfactants, such as imidazol compounds, for example Miranol; N-alkyl amino acids, such as sodium cocaminopropionate and asparagine derivatives; betaines, for example cocamidopropylebetaine; Nonionic surfactants, such as fatty acid alkanolamides, for example oleic ethanolamide; esters or polyalcohols, for example Span; polyglycerol esters, for example that esterified with fatty acids and one or several OH groups; Polyalkoxylated derivatives, for example polyoxy:polyoxyethylene stearate; ethers, for example polyoxyethe lauryl ether; ester ethers, for example Tween; amine oxides, for example coconut and dodecyl dimethyl amine oxides. In some embodiments, more than one surfactant or solvent is included.

The compositions of the disclosure may further comprise a surfactant in an amount of from about 0.001% to about 1% w/v, preferably from about 0.005 to about 0.05%. The term “surfactant” as used herein denotes a pharmaceutically acceptable surface-active agent. In the composition of the invention, the amount of surfactant is described as a percentage expressed in weight/volume. The most commonly used weight/volume unit is mg/mL. Suitable pharmaceutically acceptable surfactants include but are not limited to nonionic surfactants such as TWEEN™, PLURONICS™, polyethylene glycol (PEG), polyethylen-sorbitan-fatty acid esters, polyethylene-polypropylene glycols, polyoxyethylene-stearates, polyoxyethylene monolauryl ethers, and sodium dodecyl sulphates. Certain polyethylen-sorbitan fatty acid esters are polyethylen(20)-sorbitan-esters (polysorbate 20, sold under the trademark Tween 20™ and polyoxyethylen(20)-sorbitanmonooleate (polysorbate 80 sold under the trademark Tween 80™). Certain polyethylene-polypropylene glycols are those sold under the names Pluronic® F68 or Poloxamer 188™. Certain polyoxyethylene-stearates are those sold under the trademark Myrj™. Certain Polyoxyethylene monolauryl ethers are those sold under the trademark Brij™. When polyethylen-sorbitan-polyethylen(20)-sorbitan-esters (Tween 20™) and polyoxyethylen(20)sorbitanmonooleate (Tween 80™) are used, they are generally used in an amount of about 0.001 to about 1%, preferably of about 0.005 to about 0.1% and still preferably about 0.01% to about 0.04% w/v.

The composition may further comprise an isotonicity agent in an amount of from about 5 mM to about 350 mM. The term “isotonicity agent” as used herein denotes pharmaceutically-acceptable isotonicity agent. Isotonicity agents are used to provide an isotonic composition. An isotonic composition is liquid or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars including but not limited to glucose, sucrose, lactose, trehalose or glycerin; sugar alcohols may include sorbitol or mannitol, and any component from the group of amino acids, sugars, salts and combinations thereof. For example, the composition of the invention may comprise a sugar in an amount of about 25 mM to about 500 mM, or any value in between. Salts may include alkaline metal, alkaline earth metal or ammonium salts of organic acids such as citric acid, tartaric acid or acetic acid, e.g. sodium citrate, sodium tartrate, sodium succinate, or sodium acetate, or of mineral acids such as hydrochloric acid, e.g. sodium chloride. Sugars may include disaccharides may include lactose, trehalose, and sucrose; sugar alcohols may include sorbitol or mannitol. Polysaccharides may include glucose polymers produced by fermentation of sucrose by bacteria, such as the genus Leuconostoc, commercialized as Dextran®, such as Dextran® 40, Dextran® 70 or Dextran® 75, or a highly branched, high mass, hydrophilic polysaccharide such as Ficoll® or aloe vera. Polyvalent alcohols such as polyethylene glycol or polyvinyl alcohol or a combination of two or more of these may be employed.

The compositions of the disclosure may comprise a preservative. Preservatives may be selected from tocopherols, octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and meta-cresol); low molecular weight (less than about 10 residues) polypeptides; proteins, such as serum albumin, gelatin, or immunoglobulins; chelating agents such as EDTA; salt-forming counter-ions such as sodium; metal complexes (e.g. Zn-protein complexes). The preservative may be a tocopherol on the list of FDA's GRAS food preservatives. The tocopherol preservative may be, for example, tocopherol, dioleyl tocopheryl methylsilanol, potassium ascorbyl tocopheryl phosphate, tocophersolan, tocopheryl acetate, tocopheryl linoleate, tocopheryl linoleate/oleate, tocopheryl nicotinate, tocopheryl succinate. The composition may include, for example, 0-2%, 0.05-1.5%, 0.5 to 1%, or about 0.9% v/v or wt/v of a preservative. In one embodiment, the preservative is benzyl alcohol.

The compositions of the disclosure may include a stabilizer and/or antioxidant. The stabilizer may be, for example, an amino acid, for example, arginine, glycine, histidine, or a derivative thereof, imidazole, imidazole-4-acetic acid, for example, as described in U.S. Pat. No. 5,849,704. The stabilizer may be a “sugar alcohol” may be added, for example, mannitol, xylitol, erythritol, threitol, sorbitol, or glycerol. In the present context “disaccharide” is used to designate naturally occurring disaccharides such as sucrose, trehalose, maltose, lactose, sepharose, turanose, laminaribiose, isomaltose, gentiobiose, or melibiose. The antioxidant may be, for example, ascorbic acid, glutathione, methionine, and ethylenediamine tetraacetic acid (EDTA). The optional stabilizer or antioxidant may be in an amount from about 0 to about 20 mg, 0.1 to 10 mg, or 1 to 5 mg per mL of the liquid composition.

Antibody compositions for topical administration may be provided in liquid, solution, suspension, cream, lotion, ointment, gel, or in a solid form such as a powder, tablet, or troche for suspension immediately prior to administration. Compositions comprising SARS-CoV-2 structural protein-specific polyclonal immunoglobulin, for example IgY, are provided for use in the form of a sublingual lozenge, intranasal gel, buccal gel, eye drop, inhalable powder, inhalable solution, injectable solution, suspension, oral film, oral composition, mouth rinse, oral spray, intranasal spray, topical cream or lotion, ointment, gel, topical spray, suppository, intravenous drip, and intravenous fluid compositions.

In some embodiments, the composition may be a face cream, hand cream, face lotion, or hand lotion. The face or hand cream or lotion may be applied topically to the skin, and or used to seal a face mask, gloves, gowns, and the like. The compositions for topical use may also be provided as hard capsules, or soft gelatin capsules, wherein the benign and/or synthetic microorganism is mixed with water or an oil medium, for example, peanut oil, liquid paraffin, or olive oil. Powders and granulates may be prepared using the ingredients mentioned above under tablets and capsules for dissolution in a conventional manner using, e.g., a mixer, a fluid bed apparatus, lyophilization or a spray drying equipment.

The intranasal gel or buccal gel may be a mucoadhesive gel. The IgY antibodies may be embedded in any appropriate mucoadhesive gel or polymer known in the art. Mucoadhesive gels may be prepared using a carbopol (hydroxymethyl cellulose), such as carbopol 940, sodium carboxymethyl cellulose (sodium CMC), hydroxypropyl methyl cellulose (HPMC) (e.g., K4M), polyvinylpyrrolidone, sodium alginate, gelatin, xanthan gum, chitosan, carnauba wax, or hydroxypropyl cellulose, e.g., alone or in mixtures, for example, in about 2 wt % to about 10 wt %, or about 3 wt % to about 5 wt % of the composition. Aslani et al., 2013 Adv Biomed Res 2:21, Fini et al, 2011, Pharmaceutics 3, 665-679.

A dried antibody composition may be in a ready-to-use form to be administered directly or may be for suspension in a carrier prior to administration. When the composition is in a powder form, the powders may include chalk, talc, fullers earth, colloidal silicon dioxide, sodium polyacrylate, tetra alkyl and/or trialkyl aryl ammonium smectites and chemically modified magnesium aluminum silicate.

The antibody composition of the disclosure may include a bulking agent, for example, to aid in spray drying, freeze-drying or lyophilization. For example, the bulking agent of sugar alcohols and disaccharides and mixtures thereof. The ratio of immune egg, immune egg yolk, or isolated and/or purified IgY to sugar alcohol or disaccharide may vary from about 0.005 to about 1.5 on a weight basis. Thus, the amount of disaccharide may be from about 0.67 to about 200 mg per mg of isolated and/or purified IgY, or from about 1.1 to about 50 mg per mg of isolated and/or purified IgY. Alternatively, the disaccharide such as sucrose, mannitol and/or trehalose may be employed at 0-50 mg/mL, 10-30 mg/mL, or 15-25 mg/mL when preparing the solutions for spray drying, freeze drying or lyophilization. Zinc acetate such as Zn(Ac)₂.H₂O, may optionally be added to the composition e.g. containing PBS or histidine buffers at from 0 to 0.5 mg/mL, or 0.05 to 0.2 mg/mL.

Liquid and lyophilised antibody compositions for parenteral administration according to the invention may be prepared as follows.

### Preparation of Liquid Compositions.

Compositions of the disclosure may be prepared by homogenization of solutions of antibodies in the production buffer (e.g. 10 mM phosphate buffered saline, at ˜pH 7.4, or 20 mM histidine buffer at ˜pH 6.0, or 20 mM histidine buffer at ˜pH 6.0 containing 140 mM sodium chloride and 0.01% (w/v) polysorbate 20). Compositions of antibodies can also be prepared by adjusting the protein concentration to the desired concentration by dilution with buffer. Excipients for stabilizing the protein and for tonicity adjustment may be added as required and can be added in dissolved form or alternatively as solid.

Surfactant may be added to the compositions as a stock solution as required. Compositions may be sterile filtered through 0.22 micron filters and aseptically aliquoted into sterile glass vials and closed with rubber stoppers and alucrimp caps. These compositions may be stored at different temperatures for different intervals of time and removed for analysis at predetermined timepoints for stability studies. Compositions may be analyzed 1) by UV spectrophotometry, 2) by Size Exclusion Chromatography (SEC), 3) for visible and subvisible particles, 4) by Ion exchange chromatography (IEC) and 5) by turbidity of the solution.

### Preparation of Lyophilized Compositions

Solutions of antibodies may be prepared as described above for liquid compositions, or manufactured by homogenizing antibody solutions buffer at appropriate pH, optionally containing a sugar and a surfactant. Compositions are sterile filtered through 0.22 micron filters and aseptically aliquoted into sterile glass vials. The vials may be partly closed with rubber stoppers suitable for the use in lyophilization processes and transferred to the drying chamber of the lyophilizer. Any lyophilisation method known in the art is intended to be within the scope of the invention. For example, the lyophilization process may include the cooling of the composition from room temperature to approx 5° C. (pre-cooling) followed by a freezing at −40° C. Antibody compositions dried using the described lyophilisation processes are expected to have conveniently quick reconstitution times of about 2-3 minutes. The lyophilised vials may be stored at different temperatures for different intervals of time. The lyophilised compositions may be reconstituted with the respective volume of water for injection (WFI) prior to use or analysis.

### Stability

The antibody compositions may exhibit storage stability determined as losing less than 30%, 20%, 10% or 5% of original antibody concentration over at least one, two, three months, six months, 12 months 18 months, or 24 months when stored at a frozen, refrigerated, or preferably at room temperature. The antibody compositions should exhibit a good stability upon storage at 2-8° C. and 25° C. with adequate stability with regard to physical endpoints such as aggregation and chemical endpoints such as fragmentation. Stability analysis of compositions of the disclosure may include 1) analysis by UV spectrophotometry, 2) determination of the reconstitution time, 3) analysis by Size Exclusion Chromatography (SEC) 4) by Ion exchange chromatography (IEC), 5) determination of subvisible and visible particles and 6) by turbidity of the solution. Size exclusion chromatography (SEC) may be performed to detect soluble high molecular weight species (aggregates) and low molecular weight hydrolysis products in the compositions. The method may employ a suitable HPLC instrument equipped with a UV detector (detection wavelength 280 nm) and a Zorbax GF-250 column (9.4×250 mm, Agilent); with for example, a 200 mM sodium phosphate pH 7.0 as mobile phase.

Ion Exchange Chromatography (IEC) may be performed to detect chemical degradation products altering the net charge of antibodies in the compositions. The method may employ a suitable HPLC instrument equipped with a UV detector (e.g., detection wavelength 220 and 280 nm) and an appropriate ion exchange solid phase column. The ion exchange column may be a cation exchange or an anion exchange column as appropriate. For example, a weak cation exchange stationary phase may be employed, such as a Dionex ProPac WCX-10 column (4 mm×250 mm). A 10 mM sodium phosphate buffer pH 6.0 in H.sub.2O and 10 mM sodium phosphate buffer pH 6.0+0.75M NaCl may be used as mobile phases A and B, respectively, with a flow rate of 1.0 mL/min.

The UV spectroscopy for determination of the protein concentration may be performed on any UV spectrophotometer, such as a Varian Cary Bio UV spectrophotometer at 280 nm. For the determination of the turbidity, opalescence may be measured in FTU (turbidity units), for example, using a HACH 2100AN turbidimeter at room temperature.

Samples may be analyzed for subvisible particles, for example, by using a HIAC Royco PharmaSpec (HRLD-150), and for visible particles by using a Seidenader V90-T visual inspection instrument.

The composition may be in the form of a spray, fluid, rinse, lozenge, troche, gel, film, liquid, powder, capsule, tablet, caplet, film, ointment, cream, or lotion. The fluid may be, for example, a solution or a suspension.

In one embodiment, the fluid is a spray. The spray may be administered, for example, at an appropriate concentration to any mucosal or dermal surface of a subject. For example, the spray may be administered orally, nasally, or to the oropharyngeal cavity. The spray may alternatively be utilized at an appropriate concentration for treatment of physical surfaces, facility controls, personal protective equipment, or medical devices. For example, the spray may be utilized in the treatment of one or more layers of PPE such as, but not limited to masks, gowns, shields, shoe covers, or gloves. The spray may be used to treat one or more surfaces of medical devices such as oropharyngeal airways. The spray may be used to treat one or more layers of air filters to create an antivirotic filter.

PPEDuring the COVID-19 pandemic many frontline workers have begun using personal protection equipment (PPE) as part of their regular working protocol. Increasingly, this is strongly recommended by public health authorities. Despite the use of ordinary PPE, infection rates among frontline workers continues to rise, and in close confinement environments the increase is alarming.

Compositions of the disclosure may be administered or applied as sprays.

Compositions of the disclosure may be used to treat subjects, personal protective equipment, vehicles, surfaces, air filters, or in other facility controls, for example, in military installations, nursing homes, assisted living, long term care facilities, government facilities, prisons, jails, food processing plants, schools, churches, sports facilities, arenas, day care facilities, nurseries, office buildings, apartment buildings, airlines, trains, light rail, buses, ships, restaurants, grocery stores, retail shops, postal facilities, shipping facilities, transportation industry, livery, homes, hotels, motels, convention halls, and in the hospitality industry. The disclosure provides a biological PPE composition capable of adding a layer of protection for these workers by using anti-SARS-CoV-2 antibodies to trap and neutralize this virus in the oral, nasal, and ex-vivo areas, before it has a chance to infect (intake) or to be spread (shedding). In one embodiment, the biological PPE composition is provided in a spray bottle format and is used to create a full spectrum of protection by coating one or more of nasal passages, mouth and throat, hands, and mask. By simply spraying these areas with biological PPE composition, a layer of specifically targeted polyclonal antibodies is generated, intercepting and disabling SARS-CoV-2 virus that comes into contact with this layer. For example, the spray composition is capable of remaining active in the nose and throat for 8-12 hours. The disclosure provides a biological PPE spray composition that is a safe and effective prophylactic intervention in development that is, safe to use in the nasal passages, in the mouth, the throat, on the skin, and on a protective mask and gown.

In some embodiments, a room temperature stable biological PPE composition is provided. In one embodiment, the spray composition comprises anti-SARS-CoV-2 RBD, S1, S2 and or N IgY antibodies, and optionally anti-ACE2 IgY antibodies.

### Kits

Any of the above-mentioned anti-SARS-CoV-2 polyclonal antibody compositions may be provided in the form of a kit. In some embodiments, a kit comprises a container housing the antibody composition solution or a container housing freeze-dried, dehydrated, or spray dried antibody composition. Kits can optionally include a second container including diluent. Kits may also include one or more antimicrobial agents. Kits can also include instructions for administering and/or applying the composition. In certain embodiments, instructions are provided for mixing the antibody composition with diluent. In some embodiments, a kit further includes an applicator to apply the antibody composition to a subject.

For example, a kit may comprise a composition of the disclosure and one or more applicators or bottles. For example, 12 ml nasal spray bottles, and/or one or more 50 ml mist-pump spray bottles. For example, a single application of the composition may include: (i) intranasal application in a volume of from 10 to 200 microliters, 50 to 150 microliters or about 100 microliters of the spray composition in each nostril; (ii) application to hands of one 50 to 500 microliter, 100 to 200 microliter or about 150 microliters of spray composition on each hand; (iii) application of two 50 to 500 microliter, 100 to 200 microliter or about 150 microliters of composition in the mouth to the back of the throat; (iv) and/or application of two or three 50 to 500 microliter, 100 to 200 microliter or about 150 microliters of spray composition over a protective mask. The spray composition provides active antibody protection over the surfaces involved in the respiratory and contact routes of SARS-CoV-2 transmission. For example, one spray composition kit may enable 30 days or more of full spectrum protection, when used twice per day.

In some embodiments, the composition is administered topically, for example, as a dermal, mucosal, intraoral, intranasal, or oropharyngeal spray or atomized spray. The kit may also include a spray applicator, intranasal spray applicator, or mucosal atomizer device (MAD).

In some embodiments, the containers in the kit may be fitted with, or fitted to, a mucosal atomization device (MAD), laryngo-tracheal mucosal atomization device, bottle atomizer, venturi atomizer, or positive displacement atomizer, for example, for intranasal or oropharyngeal delivery. The kit may include an intubating airway with mucosal atomization and oxygen delivery. One or more syringes may also be included in the kit. For example, a syringe may be employed for administering an appropriate dose of the antibody composition comprising anti-SARS-CoV-2 polyclonal antibodies. Any air, if present, is expelled from syringe. A mucosal atomization device (MAD) may be attached to the syringe via luer lock. The syringe plunger is briskly compressed to create a rapid intranasal mist spray of about 0.05 to 1 mL, 0.1 to 0.5 mL, or 0.2 to 0.4 antibody spray composition per nostril. In some embodiments, the kit comprises one or more single dose containers filled with the composition, a sheet of instructions, and one or more mucosal atomization devices (MADs). In some embodiments, one MAD is pre-fitted to each single dose container. In some embodiments, the kit comprises one or more MADs capable of being fitted to the single dose container(s) prior to use. In some embodiments, the MAD can be fitted to a syringe, or an MAD with syringe can be used in conjunction with a filled vial. Any mucosal atomization device (MAD) capable of being fitted to a syringe, e.g., fitted with a luer lock, can be employed. MADs are available commercially and include LMA/MAD Nasai™ intranasal mucosal atomization device (LMA North America, Inc, San Diego, Calif.), and Wolfe-Tory Mucosal Atomization Device MAD (Wolfe-Tory Medical, Salt Lake City, Utah).

Inventive IgY antibodies provided herein have been demonstrated to actively bind to S1, and S2, RBD regions of SARS-CoV-2, which are sites known to be effective in neutralizing the ability of the COVID-19 virus to infect cells. The estimated 99+% neutralizing titer of S2 antibodies is approximately 30 ug/ml, and the estimated 99+% neutralizing titer of S1 antibodies is approximately 15 ug/ml. The spray composition application will be provided at a concentration of these antibodies higher than, or several times greater than these levels.

In some embodiments, a spray composition is provided comprising about approximately 30 ug/ml-250 ug/ml or more, 50 ug-100 ug/ml, or about 60 ug/ml to about 80 ug/ml anti-SARS-CoV-2 S1, S2 and/or N protein IgY antibodies.

In some embodiments, the inventive spray composition comprising anti-SARS-CoV-2 RBD, S1, S2 and or N protein IgY antibodies may be a food-based product, imparting an advantage in that they cannot be overdosed. Antibodies ingested are used while active and are then digested as protein. They are not introduced systemically and are not immunogenic in this application.

In some embodiments, an oral composition is provided comprising an anti-SARS-CoV-2 RBD, S1, S2 and or N protein IgY antibodies and a pharmaceutically acceptable carrier or excipient. Optionally, the oral composition may include anti-ACE2 IgY antibodies. The oral composition may be in the form of a tablet, capsule, troche, film, gel, or in a powder formulation for suspension. In one aspect, powdered dried immune egg is packaged in an airtight packet. Immediately prior to oral administration, the contents of the packet are suspended, or dissolved, in about a liquid and administered orally. In one aspect, the composition may also be provided in a liquid form for administration. In another aspect, the contents of a single dose packet are dissolved in about 2-3 ounces of water and administered orally. Formulations for oral use may also be prepared as troches, chewable tablets, or as hard gelatin capsules.

Oral compositions are provided comprising anti-SARS-CoV-2 RBD polyclonal IgY antibodies, optionally anti-ACE2 IgY antibodies, and a pharmaceutically acceptable carrier, diluent or excipient as provided herein. For example, an inert solid diluent (e.g., potato starch, lactose, microcrystalline cellulose, calcium carbonate, calcium phosphate, dextrose, or kaolin), may be employed in tablet, capsule or powder form, or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, spray drying equipment, freeze-drying and/or lyophilization equipment. In some embodiments, the diluent or arrier is a commercially available carrier or excipient such as, for example, FIRMAPRESS® pharmaceutical grade excipient powder/dextrose monohydrate which may be blended with coloring, odorant, flavoring, such as a mint flavoring.

Other oral compositions have been found to be effective in reducing upper respiratory tract infections. For example, Pedimune (bovine colostrum, Merck Ltd.) was effective in the prophylactic treatment of recurrent URTIs and diarrhea in reducing not only the episodes but also the hospitalization due to them. Patel et al., Indian J Pediatr. 2006 July; 73 (7):585-91 Pedimun in recurrent respiratory infection and diarrhea—the Indian experience. 605 children (1-8 yrs) having recurrent episodes of upper respiratory tract infections or diarrhea received bovine colostrum (Pedimune) 3 g once daily for 12 weeks. Episodes of URTI and diarrhoea reduced significantly by 91.19% and 86.60% at the end of therapy respectively.

An oral composition is provided comprising anti-SARS-CoV-2 RBD, S1, S2 and/or N protein IgY antibodies, a colostrum, a pharmaceutically acceptable carrier or excipient, and optionally anti-ACE2 IgY antibodies.

### Oral Films and Rapidly Dissolving Oral Capsules

The oral composition may be in the form of an oral fast dissolving film or oral rapidly dissolving capsule. Oral mucosal delivery via sublingual, buccal, and mucosal routes by use of thin films and capsules may be employed. Oral dissolving film is a thin film with an area of 1-20 cm\hat{ }2, depending on loading of antibodies. Antibodies may be loaded up to a single dose of about 30 mg. The rapidly dissolving film or oral capsule may be allowed to dissolve in the mouth to coat the oral cavity and throat with the antibodies. Formulation considerations such as plasticizers may be important factors affecting mechanical properties.

In some embodiments, an example an oral or buccal film formulation may include 5-30 wt % antibodies, 45 wt % water soluble polymer such as HPMC E3, E5 and E15, and K3, methyl cellulose, A-3, A-6 and A-15, pullulan, carboxymethylcellulose, (E.G. CMC cekol 30), polyvinylpyrrolidone, (e.g., PVP K90), pectin, gelatin, sodium alginate, hydroxypropylcellulose, polyvinyl alcohol, maltodextrins; 0-20 wt % plasticizer such as glycerol, dibutylphallate, polyethylene glycol and the like; 3-6 wt % sweetening agents such as saccharin, cyclamate, aspartame and the like; 2-6 wt % saliva stimulating agents such as citric acid, malic acid, lactic acid ascorbic acid and the like; and fillers, colors and flavors q.s. such as FD and C colors, US FDA approved flavors. See Bala 2013, Int J Pharm Investig, 3(2): 67-76. Commercially available film forming compositions may be employed such as XGEL™ film, SOLULEAVES™ film, FOAMBURST™ film, and WAFERTAB™ film.

### Antivirotic Filters and Face Masks

Antivirotic filters and face masks may be treated with IgY antibodies of the disclosure which have the capability of trapping and/or neutralizing coronaviruses. A neutralization titer against a coronavirus antigen may be determined. A base filter material is treated with antibodies of the disclosure. Base filter material may be any suitable filter material such as cellulose, cotton, glass or synthetic fibers. For example, cellulose-based filter material may be, e.g., cellulose nitrate, mixed cellulose esters, cellulose fabric, microfibrillated cellulose. Glass may include spun borosilicate glass fibers, quartz, etc. Synthetic material may include, e.g., polyester, polypropylene, polyacrylic, polyurethane, or silanes. The antivirotic filter may be inserted between an air filter and grill of an air purifier unit. The reactivity against viral antigen may be measured by ELISA. The antibody filter of a specific area (e.g., 4×4 cm) may be inoculated with a virus dispersion (e.g., 10\hat{ }5TCID₅₀/ml×0.5 ml) and left untouched for 10 min, virus may be extracted from filters and the TCID50 evaluated by the method of Kosugi et al., 2008, Development of Antibacterial properties and an Antivorotic multifunctional bio-filter and the Air Purification System Living Space Purifier KPD1000; FujiFilm Lifescience Research Laboratories; UDC 614.712+614.48. The antivirotic filter as an air purifier may be replaced every three to six months, but may be effective for up to one year without exposure to light. The antivirotic filter as a face mask may be employed as an insert to a fabric face mask to entrap coronaviruses. The insert may be removed and replaced prior to washing.

### Mouthwash

In some embodiments, the fluid is a mouth rinse or mouthwash. Without being bound by theory, the anti-coronavirus IgY antibodies are expected to coat the throat and alimentary canal for several hours following mothwash or mouthrinse. For example, retention of IgY in the human oral cavity is documented. Carlander et al., 2002 Biodrugs 16(6): 433-437. Several studies show that viral infections can be prevented with IgY in a dose-dependent manner. For example, the presence of yolk anti-Pseudomonas aeruginosa antibodies in saliva from healthy volunteers over time after 1 or 2 minutes' mouth rinse, performed in the evening, with an aqueous IgY antibody preparation. The test persons rinsed the mouth with 8.0 ml phosphate buffered saline before gargling with the antibody preparation 8 and 24 hours later. Statistical analysis was performed with the Mann-Whitney U test. The antibody titers in the mouth rinses were tested for their specific activity against P. aeruginosa by ELISA. The next morning there were still active antibodies detected in the saliva from 18 of 19 subjects. After 24 hours, active antibodies could be detected in saliva from only a few of the subjects. A 2-minute mouth rinse resulted in higher mean ELISA absorbance values than a 1-minute rinse.

In some embodiments, the antibody composition of the disclosure may be administered to a subject in need thereof orally, sublingually, oropharyngeally, as a mouthwash, mouth rinse, or gargle. The mouth rinse or gargle may include swish and spit or swish and swallow modes of administration.

### Parenteral Compositions

IgY may be safer for use in parenteral applications than polyclonal IgG because IgY does not bind to rheumatoid factor (an inflammatory response marker) in blood (Larsson et al. 1988), IgY does not activate mammalian complement factors (Larsson et al. 1992), IgY does not bind to cell surface Fc receptor (Schmidt et al. 1993), and IgY does not bind to protein A (Kronvall et al. 1974) or protein G (Akerstrom et al. 1985).

The antibody composition of the disclosure may be administered to a subject, for example, according to known parenteral methods, by intravenous administration as a bolus or by continuous infusion over a period of time, by intramuscular, intraperitoneal, intracerobrospinal, subcutaneous, intra-articular, intrasynovial, or intrathecal routes. In some applications, intravenous or subcutaneous administration of the antibodies may be preferred. The antibody compositions for parenteral in vivo administration must be sterile. This may be accomplished by filtration through sterile filtration membranes.

### Oral Compositions

The term “dose” of the composition refers to that amount that provides therapeutic effect in an administration regimen. The liquid composition may be formulated to include anti-coronavirus antibodies in an amount from about 2 mg/mL to about 200 mg/mL, about 5 mg/mL to about 100 mg/mL, or about 10 mg/mL to about 50 mg/mL specific IgY antibodies, or an equivalent amount of dried immune egg, or immune egg yolk. In some embodiments, the liquid composition of the disclosure may comprise the isolated or purified antibodies in an amount of from about 1 to about 150 mg/ml, preferably in an amount of from about 5 to about 100 mg/ml, from about 10 to about 30 mg/ml, or about 25 mg/ml. In some embodiments, the compositions of the disclosure may be prepared containing amounts of anti-coronavirus IgY antibodies at least about 0.01 mg/ml-40 mg/mL, 0.1-25 mg/mL, 0.5-10 mg/ml, or 1-5 mg/mL, based on a liquid ready-to-use composition. In a specific aspect, the composition comprises an equivalent weight amount of dried immune egg product or dried immune egg yolk.

In some embodiments, one dose of an oral or topical composition of the disclosure may contain 0.1 to 15 g, 0.5 to 10 g, 1 to 7 g, or 2 to 5 g, or 0.5 g, 1 g, 2 g, 3 g, 4 g, 5 g, 5 g, 6 g, 7 g, 8 8, 9 g, 10 g, 12 g, 15 g of dried immune egg, or 10 ug to 500 mg, 20 ug to 300 mg, 10 ug to 100 mg, 1 mg to 50 mg, 20 to 60 mg, 10 ug to 500 ug, 20 ug to 300 ug, or 40 ug to 100 ug of the specific antibodies.

The compositions of the disclosure includes a composition comprising anti-coronavirus IgY antibodies and optionally anti-ACE2 IgY antibodies and or anti-TMPRSS2 (transmembrane protease serine 2 serine protease) IgY antibodies. In depth analysis of epithelial cells in the respiratory tree reveals that nasal epithelial cells, specifically goblet/secretory cells and ciliated cells display the highest ACE2 expression of all epithelial cells analyzed by Waradob Sungnak et al., Nature Medicine 2020, DOI: 10.1038/s41591-020-0868-6. In addition, S-protein priming protease TMPRSS2 (transmembrane protease, serine 2) is thought to govern viral entry. In some embodiments, an intranasal spray or gel comprising (i) anti-SARS-CoV-2 S1, S2, and/or N-proteins and optionally (ii) anti-human ACE2 antibodies, to sterically block binding to ACE2 in nasal epithelial cells, and optionally (iii) anti-TMPRSS2 antibodies to block S-protein priming.

Several antiviral supplement formulations comprising various vitamins and minerals are known and some are available commercially. Many of these formulations are made up of dietary supplements as defined under the Federal Food, Drug and Cosmetic Act, Chapter II Section 201, [21 U.S.C. § 321], paragraph ff, and thus may be classified as dietary supplements rather than drugs.

In some embodiments, the composition may be a dietary supplement or a medical food.

In another aspect, the compositions of the disclosure are effective for oral administration in the treatment of a pathogenic infection. In another aspect, the compositions of the disclosure are provided in a powdered, solid form for suspension immediately prior to administration. This has the advantage that the full dose is easily administered and ingested by the subject suffering from the pathogenic infection.

In various embodiments, the composition is administered as a prophylactic or therapeutic composition. In various aspects, the composition includes a pharmaceutically acceptable diluent or carrier. In various aspects, the composition does not include a polymer, copolymer, liposome, hydrogel, or fibrin. In various aspects, the composition does not include microspheres or microcapsules. In various aspects, the composition does not include an immunogen or antigen. The composition of the invention can be administered via oral delivery, nasal deliver, ophthalmic delivery, ocular delivery, mucosal delivery, or a combination thereof.

One embodiment of this invention uses oral administration. It has been demonstrated in both human and animal systems that oral (ingested) administration of antibodies, immunoglobulins, and other biological immune factors can have measurable effects on the course, severity and duration on diseases of, for example, associated with, or influenced by, the gastrointestinal system.

In some embodiments, compositions of the present disclosure are oral compositions. In one embodiment, the oral composition can be in the form of a powder, capsule, tablet, troche, liquid, or caplet. The powder may be utilized in a capsule fill, or sold in a single dose packet meant to mix with a food such as applesauce, or can be an effervescent powder formulation sold in a single dose packet and meant for suspension in a liquid. In one aspect, the capsule, tablet, lozenge is intended for ingestion by swallowing. In another aspect, the tablet, capsule, or lozenge is orally disintegrable. In one aspect, the tablet, capsule, lozenge or troche is a slow release composition. In another aspect, the tablet, lozenge, troche or capsule is an immediate release composition. In another aspect, the oral composition can be a prepackaged liquid drink, wherein the formulation is suspended in a flavored liquid. In preferred aspects, the composition is in the form of a tablet, a capsule, or a powder meant to mix with a food, such as applesauce. Although the compositions of the disclosure are primarily oral forms, other modes of administration such as parenteral forms, or anal suppositories have been contemplated.

The powder, tablet, capsule, and caplet forms of the disclosure may comprise, aside from those components specified above, other various additives, such as vehicle, binder, disintegrating agent, lubricant, thickener, surfactant, osmotic pressure regulator, electrolyte, sweetener, flavoring, perfume, pigment, pH regulator and others appropriately as required.

Specifically, the additives may include starches such as wheat starch, potato starch, corn starch, and dextrin, sugars such as sucrose, glucose, fructose, maltose, xylose, and lactose, sugar alcohols such as sorbitol, mannitol, maltitol, and xylitol, isotransposable glycosides such as coupling sugar and paratinose, vehicles such as calcium phosphate and calcium sulfate, binders and thickeners such as gum tragacanth, gum acacia, starch, sugar, gelatin, gum arabic, dextrin, methyl cellulose, polyvinyl pyrrolidone, polyvinyl alcohol, hydroxy propyl cellulose, xanthan gum, pectin, casein, and alginic acid. The composition may contain a film forming agent or thickening agent such as acrylates/C10-30 alkyl acrylate crosspolymer, polyacrylic acid (Carbomer), sodium polyacrylate, pullulan, siliconized pullulan (e.g., TSPL-30-D5), polyvinyl alcohol/polyethylene glycol graft copolymer (e.g., KOLLICOAT®, BASF), a water soluble film forming polymer such as HPMC E3, E5 and E15, and K3, methyl cellulose (e.g. methyl cellulose A-3, A-6 and A-15), pullulan, carboxymethylcellulose, (E.G. CMC cekol 30), polyvivylpyrrolidone, (e.g., PVP K90), pectin, gelatin, sodium alginate, hydroxypropylcellulose, polyvinyl alcohol, maltodextrins. The composition may contain lubricants such as leucine, isoleucine, valine, sugar-ester, hardening oil, stearic acid, magnesium stearate, talc, and macrogol, disintegrating agents such as avicel, CMC, CMC-Na and CMC-Ca, surfactants such as polysorbate and lecithin, and sweeteners such as sugars, sugar alcohols, aspartame, alitame, other dipeptides, stevia, and saccharin, and they may be used in proper amounts selectively in consideration of the relation with the essential components, property of the composition, manufacturing method, etc.

In another embodiment, compositions of the disclosure can optionally further comprise one or more odorant agent or flavoring agents. The optional odorant agent or flavoring agent may be added to increase patient acceptability and compliance with the recommended dosing schedule. The flavoring agents that may be used include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including, without limitation, lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, artificial vanilla, cinnamon derivatives, and various fruit flavors, whether employed individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. In a specific aspect, the flavoring is spearmint oil. The flavor is optionally present from about 0.1% to about 5% by weight of the antiviral composition.

Tablets or lozenges may be molded tablets or compressed tablets. Tablets or lozenges may be formed by wet granulation, dry granulation, and direct compression. These techniques are known to one of skilled in the art and are described, for example, in the United States Pharmacopeia National Formulary USP XXII, 1990, pp. 1696-1697. Various other vitamins may be added to composition. Tablets may optionally further comprise flavorings or sweeteners. In one aspect, the sweetened, flavored tablet is utilized as a lozenge to be dissolved in the mouth. The compositions of the disclosure can also be prepared in a chewable form or an effervescent form. For effervescent preparations, the manufacturing method in the disclosure is basically same as in the manufacturing method of the usual effervescent preparations such as effervescent tablets. That is, components are weighed, mixed, and prepared directly by the powder compression method, dry or wet granular compression method, etc. For example, a lozenge composition may include isolated anti-coronavirus IgY, immune egg, or immune egg yolk in spray dried, freeze-dried or lyophilized format, and a carrier or excipient, such as calcium carbonate, and optionally additional components such as one or more of a stabilizer such as trehalose and or mannitol, a flow agent such as silicon dioxide, a lubricant such as magnesium stearate, a flavoring such as vanilla or berry flavor, a neutralizing agent such as citric acid and or ascorbic acid, a sweetener such as stevia and or sucralose, and a preservative such as benzyl alcohol.

The isolated anti-coronavirus IgY, immune egg, or immune egg yolk may be derived from eggs of poultry vaccinated with a coronavirus vaccine, such as a poultry, bovine, porcine, canine, human, feline, or ferret coronavirus vaccine, and or a recombinant SARS-CoV-2 recombinant protein such as an S-, S1, -S2-, S1-S2-ECD, S-RBD-, N-, and M-protein as provided herein, and/or a human ACE protein, such as ACE or ACE-2, TMPRSS2 protein; or fragment(s) thereof. In embodiments, the anti-coronavirus IgY may be combined with an anti-human ACE IgY and/or anti-TMPRSS2 IgY. In some embodiments, a lozenge is provided comprising an anti-coronavirus IgY, anti-human ACE IgY, and/or anti-TMPRSS2 IgY, or antigen-binding fragment(s) thereof. The anti-coronavirus IgY may be an anti-SARS-CoV-2 IgY, anti-SARS-CoV IgY, or anti-MERS CoV IgY, or a combination thereof. The anti-SARS-CoV-2 IgY may be an anti-S1 protein IgY, anti-S2 IgY, anti-N-protein IgY, anti-M-protein IgY. The anti-S1-protein IgY may be an anti-S1(spike) protein IgY or anti-S1 (RBD) IgY.

Orally disintegrable tablets and lozenges are described, for example, in U.S. Pat. No. 7,431,942, Shimuzu et al., Nguyen et al., 2011, J Am Dental Assoc 142(8), 943-949, each of which is incorporated herein by reference. Lozenges with a hard candy base can be prepared, for example, by the techniques of U.S. Pat. No. 6,316,008, Godfrey, which is incorporated herein by reference.

A liquid composition may further comprise other nutrients. Such liquid compositions may be prepared as described in U.S. Pat. No. 6,037,375, Sakamoto et al., which is incorporated herein by reference. As particularly preferred food materials, sweeteners such as organic acids and carbohydrates may be used. Organic acid components include citric acid, tartaric acid, malic acid, and succinic acid, and citric acid is particularly preferable. These organic acids are added usually in a range of 100 to 1500 mg/100 ml, preferably 250 to 800 mg/100 ml, and the composition of the material in beverage form can be prepared.

Various sweeteners can be optionally used in the tablet, liquid, capsule, lozenge or troche formulations of the disclosure. Examples of carbohydrates and sweeteners include monosaccharides such as glucose and fructose, disaccharides such as maltose, sucrose, other ordinary sugars, sugar alcohols such as xylitol, sorbitol, glycerin and erythritol, polysaccharides such as dextrin and cyclodextrin, and oligosaccharides such as fructo-oligosaccharide, galacto-oligosaccharide and lacto-sucrose. Of the carbohydrates, as the components not adversely affecting the lipid metabolism, fructose and glycerin are preferred. As oligosaccharide, addition of lacto-sucrose is preferred. A beverage composition of the disclosure can increase bifidobacteria in the body or lower the putrefaction products depending on the blend of the lacto-sucrose, so that the immune system can be intensified further. Other sweeteners include natural sweeteners such as thaumatin, stevia extract, rebaudioside A, glycyrrhizinic acid, etc. and synthetic sweeteners such as saccharin, aspartame, etc. These carbohydrates may be also added as carbohydrate mixture such as isomerized sugar and refined sugar. The sweetener is optionally present from about 0.1% to about 5% by weight of the solid composition. The blending of the carbohydrates may be about 1 to 15 g in 100 ml of the beverage composition of the disclosure, preferably about 3 to 12 g. The content of the oligosaccharide is about 0.5 to 10 g, preferably 1 to 3 g.

The nutrient liquid composition of the disclosure may also comprise, aside from the above, various nutrients, vitamins, minerals (electrolytes) including trace elements, perfumes including synthetic perfumes and natural perfumes, coloring matter, flavors (fruit, vanilla, chocolate, etc.), pectic acid and its salts, alginic acid and its salts, organic acids, thickener as protective colloidal substance, pH regulator, stabilizer, preservative, glycerins, alcohols, and sparkling component for carbonated beverages. In addition, the composition of the disclosure may also contain natural juice or fruit to be presented as fruit drink or vegetable drink. These may be used either alone or in combination of two or more kinds. The blending rate of these additives is not particularly limited, and is generally selected in a range of about 0 to 20 parts by weight to 100 parts by weight of the composition of the disclosure.

The liquid composition of the disclosure may be also prepared in an effervescent form. The effervescent form should contain, aside from the essential components of the disclosure, proper amounts of sodium carbonate and/or sodium hydrogen carbonate and neutralizing agent as foaming components. The neutralizing agent used herein is an acidic compound capable of generating carbon dioxide by neutralizing sodium carbonate or sodium hydrogen carbonate. Such compound includes, for example, L-tartaric acid, citric acid, fumaric acid, ascorbic acid and other organic acid. Preferred ascorbic acid possesses both the action of neutralizing agent and the action of antioxidant.

### Administration

In another embodiment, the disclosure provides a method of reducing the severity and duration of symptoms of COVID-19 in a human by administering the composition, or reducing infectivity. In this embodiment, the compositions are taken at the first signs of viral illness. In one aspect, the composition may administered every two, four, six, or eight waking hours with water until symptoms are resolved. In another aspect, the compositions are administered about every four hours after first symptoms appear. In another aspect, the composition is administered twice a day until symptoms are resolved.

Symptoms may include fever, dry cough, fatigue, lethargy, headache, muscle aches, sore throat, loss of taste, anosmia, loss of smell, runny and/or stuffy nose. Gastrointestinal symptoms may include nausea, vomiting and diarrhea.

In one aspect, the compositions of the disclosure are used to treat patients suffering from a coronavirus pathogenic infections. The compositions and formulations for oral administration can be administered once, twice, three times, or four times a day for two, three, four, five, six, seven, 8, 9, 10, 11, or 12 consecutive days for the treatment of a pathogenic infection. In one aspect, the composition is administered twice per day for five days for the treatment of a pathogenic infection. In another specific aspect, the composition is administered once per day for three consecutive days for the effective treatment in non-neonatal children or adults. In another aspect, the composition may be regularly administered for the prophylaxis of a pathogenic infection.

In the case of a composition for the treatment of a pathogenic infection of a mucosal membrane by topical administration to a mucosal membrane, the composition can be administered two to six times per day for a period of three to 12 days.

In a preferred embodiment, the disclosure provides a composition effective for treating symptoms of a coronavirus infection such as COVID-19. The composition takes advantage of an effective polyclonal antibody production strategy (chicken innoculation, with antibody harvesting through eggs) to generate high specificity antibodies targeted to structural proteins in order to reduce infectivity, decrease duration, or decrease severity of symptoms of COVID-19.

In one aspect, the composition of the disclosure is administered as an adjunct therapy to antiviral, and/or corticosteroid treatment. In another aspect, the composition of the disclosure is administered as an adjunct to antiviral treatment.

In another embodiment, the disclosure provides a method of reducing the severity and duration of symptoms of a coronavirus infection in a human by administering the composition. In this embodiment, the compositions are taken at the first signs of illness. In one aspect, the composition is administered every six waking hours with water until symptoms are resolved. In another aspect, the compositions are administered about every four hours after first symptoms appear. In another aspect, the composition is administered twice a day until symptoms are resolved.

In another embodiment, the composition of the present disclosure reduces coronavirus viral infection of cells.

### EXAMPLES

### Example 1. Recombinant Protein Production-CoV-2 Structural Protein Expression in E. coli

The coronavirus called CoV-2 contains 4 structural proteins. E. coli plasmid expression vectors were prepared that contain the spike protein (S) or the nucleocapsid protein (N) with either a C or N terminal hexa his-tag. The amino acid sequences of the recombinant His-tagged S- and N-proteins with either a C or N terminal his-tag are SEQ ID NO: 7, SEQ ID NO: 6, SEQ ID NO: 9, and SEQ ID NO: 8, respectively.

The DNA sequence for the genes was PCR amplified from plasmids containing either the native cDNA sequence (N gene), or an E. coli codon optimized version of the gene (S gene) purchased from Genscript. The his-tags (6× histidine residues) were added to the genes during the PCR amplification by adding the DNA sequence for the his-tags to the primers used in the PCR amplification process. The residues were added either directly in front of the stop codon or after that start codon for both genes.

The DNA sequences were added to the plasmid backbone pRAB11, and their expression is regulated by the P_{XYL/TET} promoter, which is tightly regulated and can be derepressed by adding anhydrotetracycline (ATc) to the culture media.

The PCR amplified DNA fragments for the viral genes with his-tags was added to a linear PCR amplification of the pRAB11 plasmid backbone using the Gibson assembly kit per the manufacturer's instructions (NEB). Following the assembly reaction, 0.6 μL of the reaction solution was transformed into electrocompetent E. coli BL21 (DE3) cells, recovered at 37° C. for one hour in SOC media and plated on LB carbenicillin (100 μg/mL). Colonies that formed on the plates were screened for the presence of the plasmid by PCR. Liquid cultures were started from the colonies positive for the PCR screening, and the plasmid was purified from the cells and sent to confirm the DNA sequence for the viral genes and the promoter system used on the plasmid.

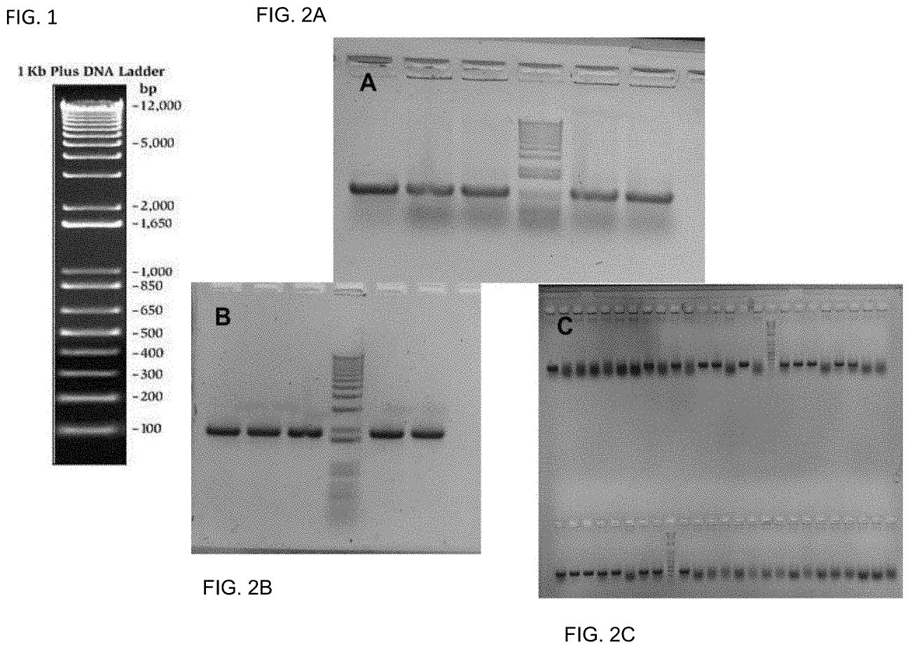

FIG. 1 shows the DNA ladder used to check the PCR products that were run on agarose gels in FIGS. 2A, 2B, and 2C.

FIG. 2 shows the results of a PCR screen to targeting the E. coli expression plasmids assembles and transformed into E. coli BL21 (DE3). FIG. 2 (A) is a photograph of a gel showing the results for the plasmid pRAB11_P_{XYL/TET}-N(C-terminus his-tag) using the primers DR_788 and DR_215. Positive bands will be 933 base pairs (bp). FIG. 2(B) is a photograph of a gel showing the results for the plasmid pRAB11_P_{XYL/TET}-N (N-terminus his-tag) using the primers DR_215 and DR_216. Positive results should have a band at 1911 bp. FIG. 2 (C) is a photograph of a gel showing the results for the plasmids pRAB11_P_{XYL/TET}-S(N-terminus his-tag) (wells 1-9 on the top row) and pRAB11_P_{XYL/TET}-S (C-terminus his-tag) (all other samples on the gel). The primers DR_776 and DR_771 were used and positive colonies should have a band at 191 bp. Positive samples for this PCR are represented by tight bands as shown in the top row far left.

TABLE 1A
DNA Primers used in PCR reactions and their
sequences

| Primer Name | DNA Sequence (5′ → 3′) |
|---|---|
| DR_788 | CAAGAGCAGCATCACCGCCATTGC |
| | (SEQ ID NO: 19) |
| DR_215 | CCGACCTCATTAAGCAGCTCTAATGCGCTG |
| | (SEQ ID NO: 20) |
| DR_216 | GGTGTGAAATACCGCACAGATGCGTAAGG |
| | (SEQ ID NO: 21) |
| DR_776 | GCAATCATTTCATCTGTGAGCAAAGGTG |
| | (SEQ ID NO: 22) |
| DR_771 | GATCCATCAAAACCAAGCAAGAGGTC |
| | (SEQ ID NO: 23) |

Table 1A shows the primer name and single stranded DNA sequence in the 5′ to 3′ direction. The primers were used to screen the fully assembled circularized expression plasmids containing the viral structural proteins.

### Example 2. Vaccine Preparation

Recombinant SARS-CoV-2 S-proteins and N-proteins, prepared by the protocol of example 1, are employed, for example in the vaccination of chickens to obtain egg antibody. The amino acid sequences of the recombinant His-tagged S- or N-proteins are SEQ ID NO: 7, SEQ ID NO: 6, SEQ ID NO: 9, and SEQ ID NO: 8, respectively.

To prepare the vaccine of the disclosure, the recombinant proteins are combined with an adjuvant. A suitable carrier such as PBS and/or a stabilizer according to standard, known in the art methods may be added. Any known adjuvant that enhances the antigenicity of the vaccine may be used, including oil based adjuvants, Freund's incomplete adjuvant, alginate adjuvant, and aluminum hydroxide adjuvant, but preferably an oil based adjuvant such as Xtend˜III. The vaccines are used in the liquid state.

For example, an oil based, adjuvant such as Xtend®III (Grand Laboratories, Inc., Larchwood, Iowa) may be employed. An initial 0.25 ml intramuscular vaccination into one or both pectoral muscles with the recombinant vaccine of this disclosure elicits significant serological responses in vaccinated chickens.

### Example 3. Vaccination Protocols

Chicken Information

Strain: Tetra or production Browns (or as available)

### Gender: Female Poults

Age: 19 weeks or so (currently egg laying)
Use: Egg laying Production

Flock Size and Groups

Example 3A. 100 hen chickens are divided into 4 groups of 25. If extra chickens are available, increase all groups evenly. If fewer chickens are available, reduce all groups evenly.

Group 1: S Protein w/HIS in PBS, 25 Chickens
Group 2: N Protein w/HIS in PBS, 25 Chickens
Group 3: S Protein w/HIS in Scourguard 4kc, 25 Chickens
Group 4: N Protein w/HIS in Scourguard 4kc, 25 Chickens

Example 3B. HyLine Brown hen chickens of about 6 months of age were divided into flocks of at least 25 each. Hens were inoculated with either a recombinant SARS-CoV-2 protein, whole cell E. coli with induced full spike protein (strain FBB_p004; pRAB11_P_{XYL/TET}-CoV_2 S gene with C terminal 6×his-tag, Black flock), and/or a commercial veterinary coronavirus vaccine as shown in Table 1B. Recombinant SARS-CoV-2 proteins were obtained commercially including Nucleocapsid protein (N-protein; Met1-Ala419; ˜50 kDa; N-terminal His-tag; RayBiotech) produced in E. coli (Red flock); Spike protein, S1 subunit (S1-protein; Val16-Gln690; ˜75 kDa; N-terminal His-tagged; RayBiotech) produced in E. coli (denatured) (Green flock); Spike protein, S2 subunit (S2-protein; Met697-Pro1213; ˜58 kDa; N-terminal His tagged; RayBiotech) produced in E. coli (denatured) (Blue flock); S1 subunit protein (RBD), glycosylated (RBD protein; Arg319-Phe541; calculated mass ˜25 kDa, migrates ˜30 kDa glycosylated in SDS-PAGE with DTT, beta-mercaptoethanol reducing conditions; C-terminal His-tagged; RayBiotech) expressed in human embryonic kidney (HEK293) cells (Orange flock); N-protein, glycosylated (Met1-Ala419; calculated mass of ˜47 kDa; migrates ˜55 kDa major band in SDS-PAGE in DTT, beta mercaptoethanol reducing conditions; minor bands at 25-30 kDa cleaved products; C-terminal His tagged; RayBiotech) expressed in HEK293 cells. Unless otherwise specified, hens were inoculated at a dose of 100 micrograms protein in a 0.5 mL volume with either Scourguard®, or Freund's complete adjuvant. Whole E. coli cells expressing full recombinant SARS-CoV-2 spike protein were inoculated at ˜5*10\hat{ }9 cfu in Freunds complete adjuvant or Scourguard®.

TABLE 1B
Flock Inoculations

| # | | | | | | First | | Other |
|---|---|---|---|---|---|---|---|---|
| Chickens | Color | | | | Dose | Inoc | Booster | Vax |
| in Flock | Code | Inoculation | Adjuvant | Dose | Volume | Day | Days | Series |
| 25 | Red | N protein | Scourguard ® | 100 ug | 0.5 mL | 0 | 15 | JY |
| | | produced in | | | | | | |
| | | E. coli | | | | | | |
| 25 | Green | S1 protein | Scourguard ® | 100 ug | 0.5 mL | 0 | 15 | JY |
| | | produced in | | | | | | |
| | | E. coli | | | | | | |
| | | (denatured) | | | | | | |
| 25 | Blue | S2 protein | Scourguard ® | 100 ug | 0.5 mL | 0 | 15 | JY |
| | | produced in | | | | | | |
| | | E. coli | | | | | | |
| | | (denatured) | | | | | | |
| 25 | Black | Whole-cell | Scourguard ® | ~5*10\hat{ }9 | 0.5 mL | 0 | 15, 39 | JY |
| | | E. coli with | | CFU | | | | |
| | | induced full | | | | | | |
| | | spike protein | | | | | | |
| | | (p_004) | | | | | | |
| 25 | Orange | S1 RBD protein | Scourguard ® | 100 ug | 0.5 mL | 0 | | JY |
| | | produced in | | | | | | |
| | | HEK cells | | | | | | |
| | | (glycosylated) | | | | | | |
| 25 | Brown | N protein | Scourguard ® | 100 ug | 0.5 mL | 0 | | JY |
| | | produced in | | | | | | |
| | | HEK cells | | | | | | |
| | | (glycosylated) | | | | | | |
| 25 | Purple | N/A | | | | | | JY |
| 25 | Pink | Whole cell | Freunds | ~5*10\hat{ }9 | 0.5 mL | 0 | | JY |
| | | E coli with | complete | CFU | | | | |
| | | induced full | adjuvant | | | | | |
| | | spike protein | | | | | | |
| | | (p_004) | | | | | | |
| 100 | White | Scourguard ® | | | | | | PTX |
| 200 | Black 2 | Whole-cell | Scourguard ® | ~5*10\hat{ }9 | 0.5 mL | 0 | | PTX |
| | | E. coli with | | CFU | | | | |
| | | induced full | | | | | | |
| | | spike protein | | | | | | |
| | | (p_004) | | | | | | |

Chickens in each flock in Table B also received additional vaccines using either the JY or PTX series. JY series chickens received a series of vaccines including Newcastle-Bronchitis Vaccine (B1 type, B1 strain; of the Massachusetts and Connecticut types; Merial) as a spray at 2 weeks of age; Newcastle-Bronchitis vaccine (B1 type, B1 strain; Massachusetts and Arkansas types; Merial) as a spray at 4 weeks of age; Newcastle-Bronchitis vaccine (B1 type, LaSota strain, Mass type, Holland strain; Merial) as a spray at about 7 and 13 weeks of age. PTX series chickens received a series of vaccines including Newcastle-Bronchitis Vaccine (B1 type, B1 strain; of the Massachusetts and Connecticut types; Merial) in water at 18 days; Newcastle-Bronchitis vaccine (B1 type, B1 strain; Massachusetts and Arkansas types; Merial) in water at 4 weeks; Newcastle-Bronchitis vaccine (B1 type, LaSota strain, Mass type, Holland strain; Merial) as a spray at about 7 weeks, and Salmonella enteritidis Bacterin-Newcastle-Bronchitis vaccine, Massachusetts type, killed vaccine (Poulvac®-SE-ND-IB vaccine; Zoetis) at about 13 and 15 weeks of age by injection. Group Immunogen Preparation

Immunogen preparations for groups 1 and 2 will include 2,500 ug of S (group 1) or N (group 2) will receive protein antigen with N′ HIS label in 12.5 ml PBS. This quantity provides 25 inoculations, each with 100 ug/ml pure S or N protein antigen with N′ HIS label in 0.5 ml of PBS.

Immunogen preparations for groups 3 and 4. In Groups 3 and 4, Scourguard® 4kc must be diluted for use in chickens. The dilution is the standard full bovine dose (2 ml of vaccine) diluted 1:4 in PBS. 2,500 ug of S (group 1) or N (group 2) protein antigen with N′ HIS label, in 12.5 ml diluted Scourguard® 4kc. This quantity provides 25 inoculations, each with 100 ug/ml of pure S or N protein antigen with N′ HIS label, in 0.5 ml of diluted Scourguard 4kc.

Excessive amounts of antigen protein in the injection may potentially induce spontaneous tolerance (at which point no antibodies to the injections will be produced). On the opposite end of the risk spectrum, chronic inflammatory conditions may develop if excessive antigen is utilized. The objective is to find the “sweet spot” that optimizes the balance between sustainability and titer. According to the literature, the range of antigen that should be used is between 25 and 200 μg. Initially, 100 ug per chicken will be employed and will vary the amount as needed. No more than 500 μg should be administered to avoid accidentally inducing tolerance.

### Initial Inoculation Procedure

For all groups, each chicken receives one 0.5 ml intramuscular inoculation of the appropriate group formulation in right breast.

### Long Term Inoculation Schedule

Initial inoculation: right breast inoculation as described above. Booster inoculations will follow similar protocol (details to be determined). A production animal vaccine of 2 ml-5 ml should be diluted to 1:4 for the initial injection, 1:8 for the follow up booster, and 1:16 for the second follow up. Dilution should be done with PBS buffer or sterile saline, then aliquot to 500 μg total volume for injection until revised by dose optimization analysis.

At 2 weeks (+2 weeks): left breast inoculation with 1:8 Scourguard 4kc dilution.

At 4 weeks (+2 weeks): right breast inoculation with 1:16 Scourguard 4kc dilution.

Each hen will receive an ankle band, or have their feathers dyed, according to the group coloring scheme. The groups will be permanently segregated from each other so that the eggs from each group can easily be identified. Upon harvest, a colored sharpie or magic marker should be used to mark the egg with the appropriate group color coding. Each group's eggs are to be collected in a container color coded to the appropriate group. These color-coding guidelines are strictly enforced to minimize mixing of group eggs that may lead to inaccurate testing of titer levels.

Egg Collection

Eggs are harvested and stored in separate containers for each group, labeled by date produced, and stored in a refrigerator daily. Each hen lays approximately one egg per day. E every third day, requiring on-site storage of 200 eggs in a refrigerator, and 100 eggs collected same day not requiring cool storage. The pickup schedule is adjusted based on refrigerator capacity. All eggs will be processed and tested for total and specific IgY titer levels.

It is important to examine the chicken breast prior to any injection to ensure no lesions, rashes, abnormal inflammation or other signs of distress are present. If a chicken is exhibiting any of these signs, do not proceed with injection and instead bring the chicken to the attention of the veterinarian.

Eggs typically exhibit usable titers by, or just following, the second inoculation. Eggs produced prior to the second inoculation should be preserved separately, by color-coded group and date produced, for dose optimization and other analysis or research.

### Example 4. Quantitative Enzyme-Linked Immunosorbent Assay (ELISA) for Egg Powder Specific IgY and Total IgY

The antibody activity of total IgY and specific anti-antigen IgY can be determined using Enzyme-Linked Immunosorbant Assay (ELISA), for example, by a modification of the method of Liou et al., 2011, J. Anim. Vet. Adv., 10(18):2349-2356, as described below.

Microtiter plates are coated with either 100 uL mixed antigen preparation (10 ug per well) or coated with 100 uL rabbit anti-chicken IgY antibody (10 ug/mL, Sigma-Aldrich), for control wells. The plate is incubated overnight at 4° C. After washing with PBS-Tween 20 buffer, plates are blocked with 2% BSA and incubated overnight at 4° C. The wells are then washed with PBS-Tween 20 buffer and once with PBS. Thereafter, diluted IgY or dried egg powder stock (10 mg/mL) is serially diluted with 1% BSA and added to sample wells at 100 uL per well. Wells for standard curve are filled with 100 uL serial dilutions of standard IgY at, e.g., at concentration ranges of, e.g., 0.015-1 ug/mL and incubated overnight at 4° C. After washing with PBS-Tween 20 buffer, 100 uL of alkaline phosphatase-conjugated goat anti-chicken IgY is added to the wells and incubated 2 hours at 37° C. After washing with PBS-Tween 20 buffer, 100 uL disodium p-nitrophenyl phosphate as substrate is added to each well and allowed to react for 10 min at 37° C. The absorbance is measured at 405 nm using a plate reader. The absorbance of standard curves provides a relative measurement of specific anti-antigen IgY concentration.

For measurement of total IgY, each well of the microtiter plate is coated with rabbit anti-chicken IgY antibody (10 ug/mL). After incubation and washing as above, 100 uL of diluted dried egg powder is added and assay is performed as above.

### Example 5. Quantitative Enzyme-Linked Immunosorbent Assay (ELISA) for Egg Powder Specific IgY

Costar half-area high binding assay plates (Corning #3690) are coated with purified 2019-nCoV S-protein or N-protein at 100 ng/well in PBS overnight at 4° C. and blocked with 3% milk powder (w/v) in PBS buffer at 37° C. 3-fold serially diluted immune egg, or purified IgY antibodies are added and incubated for 1.5 h at 37° C. HRP-conjugated rabbit anti-chicken IgY (Sigma-Aldrich) is used for detection. Enzymatic activity is measured with the subsequent addition of substrate ABTS (2,2′-Azinobis [3-ethylbenzothiazoline-6-sulfonic acid]-diammonium salt), and signal reading was carried out at 405 nm.

### Example 6. Plasmid Construction for Recombinant SARS-CoV-2 Proteins

Plasmids were constructed for E. coli expression of the nucleocapsid (N) and spike (S) proteins from the novel coronavirus termed CoV-2. A plasmid based protein expression system available in our lab is a high copy plasmid backbone called pRAB11. The plasmid contains the promoter called P_{Tet} which can be de-repressed (induced) by adding anhydrotetracycline (ATc) to the culture media.

A common technique used in molecular biology to easily purify recombinant proteins is to add 6 adjacent histidine residues to the protein, usually on either end of the amino acid sequence, which is done by adding a DNA sequence coding for the histidine residues to the DNA sequence coding for the expressed protein. If the histidine residues are exposed on the outside of the protein it can be easily purified using immobilized metal affinity chromatography (IMAC), which usually consists of columns packed with a nickel resin. The expression plasmids that are discussed in this report have either a C or N terminus 6×his-tag added to the recombinant proteins.

The genes that code for the viral proteins were added behind the promoter, and the new plasmids were transformed into an E. coli strain that is optimized for protein expression. After the plasmids were verified by sequencing the plasmid DNA the strains were grown up, and ATc was added to culture media to induce expression of the proteins. Several techniques were used to analyze the protein expression prior to the using the bacterial expression system as an antigen for inoculating chickens for the purpose of producing IgY targeted towards the viral proteins.

Table 2 shows the single stranded DNA oligonucleotide sequences used to PCR amplify DNA sequences to generate DNA fragments to create circular plasmids, screening plasmids, and sequencing recombinant genes.

TABLE 2
Primers used in PCRs

| Primer | |
|---|---|
| Name | DNA Sequence (5′ to 3′) |
| DR_782 | GTGTATAAGGTGATGTCTGATAATGGACCCCAAAAT |
| | CAGC |
| | (SEQ ID NO: 44) |
| DR_781 | AGTGGTGATGGTGATGATGGGCCTGAGTTGAGTCAG |
| | CAC |
| | (SEQ ID NO: 45) |
| DR_784 | TCAGGCCCATCATCACCATCACCACTAAGTGACAT |
| | ATAGC |
| | (SEQ ID NO: 46) |
| DR_783 | CATTATCAGACATCACCTTATACACCTCCTCTCT |
| | GCGG |
| | (SEQ ID NO: 47) |
| DR_786 | CATCATCACCATCACCACTCTGATAATGGACCCC |
| | AAAATC |
| | (SEQ ID NO: 48) |
| DR_789 | TGGTGCGGCTATATGTCACTTAGGCCTGAGTTGA |
| | GTCAGC |
| | (SEQ ID NO: 49) |
| DR_765 | AGTGGTGATGGTGATGATGCATCACCTTATACAC |
| | CTCCTC |
| | (SEQ ID NO: 50) |
| DR_790 | TGCTGACTCAACTCAGGCCTAAGTGACATATAGC |
| | CGCACC |
| | (SEQ ID NO: 51) |
| DR_794 | CAGAGAGGAGGTGTATAAGGTGATGTTTGTTTTT |
| | CTTGTTTTATTGCCACTAG |
| | (SEQ ID NO: 52) |
| DR_795 | CACTTAGTGGTGATGGTGATGATGTGTGTAATGT |
| | AATTTGACTCCTTTGAGC |
| | (SEQ ID NO: 53) |
| DR_778 | ACATCATCACCATCACCACTAAGTGACATATAGC |
| | CGCACCAATAAAAATTG |
| | (SEQ ID NO: 54) |
| DR_791 | GCAATAAAACAAGAAAAACAAACATCACCTTATA |
| | CACCTCCTCTCTGC |
| | (SEQ ID NO: 55) |
| DR_797 | AGGTGATGCATCATCACCATCACCACTTTGTTTT |
| | TCTTGTTTTATTGCCACTAGTC |
| | (SEQ ID NO: 56) |
| DR_747 | GGCTATATGTCACTTATGTGTAATGTAATTTGAC |
| | TCCTTTGAGC |
| | (SEQ ID NO: 57) |
| DR_779 | GGTGATGAAGTCAGACAAATCGC |
| | (SEQ ID NO: 58) |
| DR_796 | TACATTACACATAAGTGACATATAGCCGCACCAA |
| | TAAAAATTGATAATAGCTGAGC |
| | (SEQ ID NO: 59) |
| DR_215 | CCGACCTCATTAAGCAGCTCTAATGCGCTG |
| | (SEQ ID NO: 60) |
| DR_216 | GGTGTGAAATACCGCACAGATGCGTAAGG |
| | (SEQ ID NO: 61) |
| DR_788 | CAAGAGCAGCATCACCGCCATTGC |
| | (SEQ ID NO: 62) |
| DR_776 | GCAATCATTTCATCTGTGAGCAAAGGTG |
| | (SEQ ID NO: 63) |
| DR_771 | GATCCATCAAAACCAAGCAAGAGGTC |
| | (SEQ ID NO: 64) |
| DR_767 | CACACGCCTATTAATTTAGTGCGTG |
| | (SEQ ID NO: 65) |
| DR_768 | CGCACTAAATTAATAGGCGTGTGC |
| | (SEQ ID NO: 66) |
| DR_769 | GGTGATGAAGTCAGACAAATCGC |
| | (SEQ ID NO: 67) |
| DR_770 | TCAGGATGTTAACTGCACAGAAGTC |
| | (SEQ ID NO: 68) |
| DR_771 | GATCCATCAAAACCAAGCAAGAGGTC |
| | (SEQ ID NO: 69) |
| DR_772 | CTGAAGTGCAAATTGATAGGTTGATCACAG |
| | (SEQ ID NO: 70) |
| DR_773 | CAACACAGTTTATGATCCTTTGCAACCTG |
| | (SEQ ID NO: 71) |
| DR_774 | CCATCATGACAAATGGCAGGAGCAG |
| | (SEQ ID NO: 72) |
| DR_775 | CCACAAACAGTTGCTGGTGCATGTAG |
| | (SEQ ID NO: 73) |
| DR_776 | GCAATCATTTCATCTGTGAGCAAAGGTG |
| | (SEQ ID NO: 74) |
| DR_777 | CTTAGTGGTGATGGTGATGATGTTTGTATAGTTC |
| | ATCCATGCCATGTG |
| | (SEQ ID NO: 75) |
| DR_778 | ACATCATCACCATCACCACTAAGTGACATATAGC |
| | CGCACCAATAAAAATTG |
| | (SEQ ID NO: 76) |

Table 3 shows one strand of the double stranded DNA sequences used to express the his-tagged CoV-2 recombinant proteins in E. coli. The bolded portion highlights the DNA sequence that codes for the 6×his-tag.

TABLE 3
DNA Sequences for CoV-2 Recombinant Protein Expression

| Gene | DNA Sequence 5′-3′ |
|---|---|
| N gene with | ATGCATCATCACCATCACCACTCTGATAATGGACCCCAAAATCAGCGAAATGCAC |
| N-term 6x | CCCGCATTACGTTTGGTGGACCCTCAGATTCAACTGGCAGTAACCAGAATGGAGAA |
| His-tag | CGCAGTGGGGCGCGATCAAAACAACGTCGGCCCCAAGGTTTACCCAATAATACTG |
| (p001) | CGTCTTGGTTCACCGCTCTCACTCAACATGGCAAGGAAGACCTTAAATTCCCTCGA |
| FBB_DNA_ | GGACAAGGCGTTCCAATTAACACCAATAGCAGTCCAGATGACCAAATTGGCTACTA |
| 012 | CCGAAGAGCTACCAGACGAATTCGTGGTGGTGACGGTAAAATGAAAGATCTCAGT |
| | CCAAGATGGTATTTCTACTACCTAGGAACTGGGCCAGAAGCTGGACTTCCCTATGG |
| | TGCTAACAAAGACGGCATCATATGGGTTGCAACTGAGGGAGCCTTGAATACACCA |
| | AAAGATCACATTGGCACCCGCAATCCTGCTAACAATGCTGCAATCGTGCTACAACT |
| | TCCTCAAGGAACAACATTGCCAAAAGGCTTCTACGCAGAAGGGAGCAGAGGCGGC |
| | AGTCAAGCCTCTTCTCGTTCCTCATCACGTAGTCGCAACAGTTCAAGAAATTCAACT |
| | CCAGGCAGCAGTAGGGGAACTTCTCCTGCTAGAATGGCTGGCAATGGCGGTGATG |
| | CTGCTCTTGCTTTGCTGCTGCTTGACAGATTGAACCAGCTTGAGAGCAAAATGTCTG |
| | GTAAAGGCCAACAACAACAAGGCCAAACTGTCACTAAGAAATCTGCTGCTGAGGC |
| | TTCTAAGAAGCCTCGGCAAAAACGTACTGCCACTAAAGCATACAATGTAACACAA |
| | GCTTTCGGCAGACGTGGTCCAGAACAAACCCAAGGAAATTTTGGGGACCAGGAAC |
| | TAATCAGACAAGGAACTGATTACAAACATTGGCCGCAAATTGCACAATTTGCCCCC |
| | AGCGCTTCAGCGTTCTTCGGAATGTCGCGCATTGGCATGGAAGTCACACCTTCGGG |
| | AACGTGGTTGACCTACACAGGTGCCATCAAATTGGATGACAAAGATCCAAATTTCA |
| | AAGATCAAGTCATTTTGCTGAATAAGCATATTGACGCATACAAAACATTCCCACCA |
| | ACAGAGCCTAAAAAGGACAAAAAGAAGAAGGCTGATGAAACTCAAGCCTTACCGC |
| | AGAGACAGAAGAAACAGCAAACTGTGACTCTTCTTCCTGCTGCAGATTTGGATGAT |
| | TTCTCCAAACAATTGCAACAATCCATGAGCAGTGCTGACTCAACTCAGGCCTAA |
| | (SEQ ID NO: 24) |
| N gene with | ATGTCTGATAATGGACCCCAAAATCAGCGAAATGCACCCCGCATTACGTTTGGTGG |
| C-term 6x | ACCCTCAGATTCAACTGGCAGTAACCAGAATGGAGAACGCAGTGGGGCGCGATCA |
| His-tag | AAACAACGTCGGCCCCAAGGTTTACCCAATAATACTGCGTCTTGGTTCACCGCTCT |
| (p002) | CACTCAACATGGCAAGGAAGACCTTAAATTCCCTCGAGGACAAGGCGTTCCAATTA |
| FBB_DNA_ | ACACCAATAGCAGTCCAGATGACCAAATTGGCTACTACCGAAGAGCTACCAGACG |
| 013 | AATTCGTGGTGGTGACGGTAAAATGAAAGATCTCAGTCCAAGATGGTATTTCTACT |
| | ACCTAGGAACTGGGCCAGAAGCTGGACTTCCCTATGGTGCTAACAAAGACGGCAT |
| | CATATGGGTTGCAACTGAGGGAGCCTTGAATACACCAAAAGATCACATTGGCACCC |
| | GCAATCCTGCTAACAATGCTGCAATCGTGCTACAACTTCCTCAAGGAACAACATTG |
| | CCAAAAGGCTTCTACGCAGAAGGGAGCAGAGGCGGCAGTCAAGCCTCTTCTCGTTC |
| | CTCATCACGTAGTCGCAACAGTTCAAGAAATTCAACTCCAGGCAGCAGTAGGGGA |
| | ACTTCTCCTGCTAGAATGGCTGGCAATGGCGGTGATGCTGCTCTTGCTTTGCTGCTG |
| | CTTGACAGATTGAACCAGCTTGAGAGCAAAATGTCTGGTAAAGGCCAACAACAAC |
| | AAGGCCAAACTGTCACTAAGAAATCTGCTGCTGAGGCTTCTAAGAAGCCTCGGCAA |
| | AAACGTACTGCCACTAAAGCATACAATGTAACACAAGCTTTCGGCAGACGTGGTCC |
| | AGAACAAACCCAAGGAAATTTTGGGGACCAGGAACTAATCAGACAAGGAACTGAT |
| | TACAAACATTGGCCGCAAATTGCACAATTTGCCCCCAGCGCTTCAGCGTTCTTCGG |
| | AATGTCGCGCATTGGCATGGAAGTCACACCTTCGGGAACGTGGTTGACCTACACAG |
| | GTGCCATCAAATTGGATGACAAAGATCCAAATTTCAAAGATCAAGTCATTTTGCTG |
| | AATAAGCATATTGACGCATACAAAACATTCCCACCAACAGAGCCTAAAAAGGACA |
| | AAAAGAAGAAGGCTGATGAAACTCAAGCCTTACCGCAGAGACAGAAGAAACAGC |
| | AAACTGTGACTCTTCTTCCTGCTGCAGATTTGGATGATTTCTCCAAACAATTGCAAC |
| | AATCCATGAGCAGTGCTGACTCAACTCAGGCCCATCATCACCATCACCACTAA |
| | (SEQ ID NO: 25) |
| S gene with | ATGCATCATCACCATCACCACTTTGTTTTTCTTGTTTTATTGCCACTAGTCTCTAGT |
| N-term 6x | CAGTGTGTTAATCTTACAACCAGAACTCAATTACCCCCTGCATACACTAATTCTTTC |
| His-tag | ACACGTGGTGTTTATTACCCTGACAAAGTTTTCAGATCCTCAGTTTTACATTCAACT |
| (p003) | CAGGACTTGTTCTTACCTTTCTTTTCCAATGTTACTTGGTTCCATGCTATACATGTCT |
| FBB_DNA_ | CTGGGACCAATGGTACTAAGAGGTTTGATAACCCTGTCCTACCATTTAATGATGGT |
| 014 | GTTTATTTTGCTTCCACTGAGAAGTCTAACATAATAAGAGGCTGGATTTTTGGTACT |
| | ACTTTAGATTCGAAGACCCAGTCCCTACTTATTGTTAATAACGCTACTAATGTTGTT |
| | ATTAAAGTCTGTGAATTTCAATTTTGTAATGATCCATTTTTGGGTGTTTATTACCAC |
| | AAAAACAACAAAAGTTGGATGGAAAGTGAGTTCAGAGTTTATTCTAGTGCGAATA |
| | ATTGCACTTTTGAATATGTCTCTCAGCCTTTTCTTATGGACCTTGAAGGAAAACAGG |
| | GTAATTTCAAAAATCTTAGGGAATTTGTGTTTAAGAATATTGATGGTTATTTTAAAA |
| | TATATTCTAAGCACACGCCTATTAATTTAGTGCGTGATCTCCCTCAGGGTTTTTCGG |
| | CTTTAGAACCATTGGTAGATTTGCCAATAGGTATTAACATCACTAGGTTTCAAACTT |
| | TACTTGCTTTACATAGAAGTTATTTGACTCCTGGTGATTCTTCTTCAGGTTGGACAG |
| | CTGGTGCTGCAGCTTATTATGTGGGTTATCTTCAACCTAGGACTTTTCTATTAAAAT |
| | ATAATGAAAATGGAACCATTACAGATGCTGTAGACTGTGCACTTGACCCTCTCTCA |
| | GAAACAAAGTGTACGTTGAAATCCTTCACTGTAGAAAAAGGAATCTATCAAACTTC |
| | TAACTTTAGAGTCCAACCAACAGAATCTATTGTTAGATTTCCTAATATTACAAACTT |
| | GTGCCCTTTTGGTGAAGTTTTTAACGCCACCAGATTTGCATCTGTTTATGCTTGGAA |
| | CAGGAAGAGAATCAGCAACTGTGTTGCTGATTATTCTGTCCTATATAATTCCGCAT |
| | CATTTTCCACTTTTAAGTGTTATGGAGTGTCTCCTACTAAATTAAATGATCTCTGCT |
| | TTACTAATGTCTATGCAGATTCATTTGTAATTAGAGGTGATGAAGTCAGACAAATC |
| | GCTCCAGGGCAAACTGGAAAGATTGCTGATTATAATTATAAATTACCAGATGATTT |
| | TACAGGCTGCGTTATAGCTTGGAATTCTAACAATCTTGATTCTAAGGTTGGTGGTA |
| | ATTATAATTACCTGTATAGATTGTTTAGGAAGTCTAATCTCAAACCTTTTGAGAGAG |
| | ATATTTCAACTGAAATCTATCAGGCCGGTAGCACACCTTGTAATGGTGTTGAAGGT |
| | TTTAATTGTTACTTTCCTTTACAATCATATGGTTTCCAACCCACTAATGGTGTTGGTT |
| | ACCAACCATACAGAGTAGTAGTACTTTCTTTTGAACTTCTACATGCACCAGCAACT |
| | GTTTGTGGACCTAAAAAGTCTACTAATTTGGTTAAAAACAAATGTGTCAATTTCAA |
| | CTTCAATGGTTTAACAGGCACAGGTGTTCTTACTGAGTCTAACAAAAAGTTTCTGC |
| | CTTTCCAACAATTTGGCAGAGACATTGCTGACACTACTGATGCTGTCCGTGATCCA |
| | CAGACACTTGAGATTCTTGACATTACACCATGTTCTTTTGGTGGTGTCAGTGTTATA |
| | ACACCAGGAACAAATACTTCTAACCAGGTTGCTGTTCTTTATCAGGATGTTAACTG |
| | CACAGAAGTCCCTGTTGCTATTCATGCAGATCAACTTACTCCTACTTGGCGTGTTTA |
| | TTCTACAGGTTCTAATGTTTTTCAAACACGTGCAGGCTGTTTAATAGGGGCTGAAC |
| | ATGTCAACAACTCATATGAGTGTGACATACCCATTGGTGCAGGTATATGCGCTAGT |
| | TATCAGACTCAGACTAATTCTCCTCGGCGGGCACGTAGTGTAGCTAGTCAATCCAT |
| | CATTGCCTACACTATGTCACTTGGTGCAGAAAATTCAGTTGCTTACTCTAATAACTC |
| | TATTGCCATACCCACAAATTTTACTATTAGTGTTACCACAGAAATTCTACCAGTGTC |
| | TATGACCAAGACATCAGTAGATTGTACAATGTACATTTGTGGTGATTCAACTGAAT |
| | GCAGCAATCTTTTGTTGCAATATGGCAGTTTTTGTACACAATTAAACCGTGCTTTAA |
| | CTGGAATAGCTGTTGAACAAGACAAAAACACCCAAGAAGTTTTTGCACAAGTCAA |
| | ACAAATTTACAAAACACCACCAATTAAAGATTTTGGTGGTTTTAATTTTTCACAAAT |
| | ATTACCAGATCCATCAAAACCAAGCAAGAGGTCATTTATTGAAGATCTACTTTTCA |
| | ACAAAGTGACACTTGCAGATGCTGGCTTCATCAAACAATATGGTGATTGCCTTGGT |
| | GATATTGCTGCTAGAGACCTCATTTGTGCACAAAAGTTTAACGGCCTTACTGTTTTG |
| | CCACCTTTGCTCACAGATGAAATGATTGCTCAATACACTTCTGCACTGTTAGCGGGT |
| | ACAATCACTTCTGGTTGGACCTTTGGTGCAGGTGCTGCATTACAAATACCATTTGCT |
| | ATGCAAATGGCTTATAGGTTTAATGGTATTGGAGTTACACAGAATGTTCTCTATGA |
| | GAACCAAAAATTGATTGCCAACCAATTTAATAGTGCTATTGGCAAAATTCAAGACT |
| | CACTTTCTTCCACAGCAAGTGCACTTGGAAAACTTCAAGATGTGGTCAACCAAAAT |
| | GCACAAGCTTTAAACACGCTTGTTAAACAACTTAGCTCCAATTTTGGTGCAATTTCA |
| | AGTGTTTTAAATGATATCCTTTCACGTCTTGACAAAGTTGAGGCTGAAGTGCAAAT |
| | TGATAGGTTGATCACAGGCAGACTTCAAAGTTTGCAGACATATGTGACTCAACAAT |
| | TAATTAGAGCTGCAGAAATCAGAGCTTCTGCTAATCTTGCTGCTACTAAAATGTCA |
| | GAGTGTGTACTTGGACAATCAAAAAGAGTTGATTTTTGTGGAAAGGGCTATCATCT |
| | TATGTCCTTCCCTCAGTCAGCACCTCATGGTGTAGTCTTCTTGCATGTGACTTATGT |
| | CCCTGCACAAGAAAAGAACTTCACAACTGCTCCTGCCATTTGTCATGATGGAAAAG |
| | CACACTTTCCTCGTGAAGGTGTCTTTGTTTCAAATGGCACACACTGGTTTGTAACAC |
| | AAAGGAATTTTTATGAACCACAAATCATTACTACAGACAACACATTTGTGTCTGGT |
| | AACTGTGATGTTGTAATAGGAATTGTCAACAACACAGTTTATGATCCTTTGCAACC |
| | TGAATTAGACTCATTCAAGGAGGAGTTAGATAAATATTTTAAGAATCATACATCAC |
| | CAGATGTTGATTTAGGTGACATCTCTGGCATTAATGCTTCAGTTGTAAACATTCAAA |
| | AAGAAATTGACCGCCTCAATGAGGTTGCCAAGAATTTAAATGAATCTCTCATCGAT |
| | CTCCAAGAACTTGGAAAGTATGAGCAGTATATAAAATGGCCATGGTACATTTGGCT |
| | AGGTTTTATAGCTGGCTTGATTGCCATAGTAATGGTGACAATTATGCTTTGCTGTAT |
| | GACCAGTTGCTGTAGTTGTCTCAAGGGCTGTTGTTCTTGTGGATCCTGCTGCAAATT |
| | TGATGAAGACGACTCTGAGCCAGTGCTCAAAGGAGTCAAATTACATTACACATAA |
| | (SEQ ID NO: 26) |
| S gene with | ATGTTTGTTTTTCTTGTTTTATTGCCACTAGTCTCTAGTCAGTGTGTTAATCTTACAA |
| C-term 6x | CCAGAACTCAATTACCCCCTGCATACACTAATTCTTTCACACGTGGTGTTTATTACC |
| His-tag | CTGACAAAGTTTTCAGATCCTCAGTTTTACATTCAACTCAGGACTTGTTCTTACCTT |
| (p004) | TCTTTTCCAATGTTACTTGGTTCCATGCTATACATGTCTCTGGGACCAATGGTACTA |
| FBB_DNA_ | AGAGGTTTGATAACCCTGTCCTACCATTTAATGATGGTGTTTATTTTGCTTCCACTG |
| 15 | AGAAGTCTAACATAATAAGAGGCTGGATTTTTGGTACTACTTTAGATTCGAAGACC |
| | CAGTCCCTACTTATTGTTAATAACGCTACTAATGTTGTTATTAAAGTCTGTGAATTT |
| | CAATTTTGTAATGATCCATTTTTGGGTGTTTATTACCACAAAAACAACAAAAGTTG |
| | GATGGAAAGTGAGTTCAGAGTTTATTCTAGTGCGAATAATTGCACTTTTGAATATG |
| | TCTCTCAGCCTTTTCTTATGGACCTTGAAGGAAAACAGGGTAATTTCAAAAATCTTA |
| | GGGAATTTGTGTTTAAGAATATTGATGGTTATTTTAAAATATATTCTAAGCACACGC |
| | CTATTAATTTAGTGCGTGATCTCCCTCAGGGTTTTTCGGCTTTAGAACCATTGGTAG |
| | ATTTGCCAATAGGTATTAACATCACTAGGTTTCAAACTTTACTTGCTTTACATAGAA |
| | GTTATTTGACTCCTGGTGATTCTTCTTCAGGTTGGACAGCTGGTGCTGCAGCTTATT |
| | ATGTGGGTTATCTTCAACCTAGGACTTTTCTATTAAAATATAATGAAAATGGAACC |
| | ATTACAGATGCTGTAGACTGTGCACTTGACCCTCTCTCAGAAACAAAGTGTACGTT |
| | GAAATCCTTCACTGTAGAAAAAGGAATCTATCAAACTTCTAACTTTAGAGTCCAAC |
| | CAACAGAATCTATTGTTAGATTTCCTAATATTACAAACTTGTGCCCTTTTGGTGAAG |
| | TTTTTAACGCCACCAGATTTGCATCTGTTTATGCTTGGAACAGGAAGAGAATCAGC |
| | AACTGTGTTGCTGATTATTCTGTCCTATATAATTCCGCATCATTTTCCACTTTTAAGT |
| | GTTATGGAGTGTCTCCTACTAAATTAAATGATCTCTGCTTTACTAATGTCTATGCAG |
| | ATTCATTTGTAATTAGAGGTGATGAAGTCAGACAAATCGCTCCAGGGCAAACTGGA |
| | AAGATTGCTGATTATAATTATAAATTACCAGATGATTTTACAGGCTGCGTTATAGCT |
| | TGGAATTCTAACAATCTTGATTCTAAGGTTGGTGGTAATTATAATTACCTGTATAGA |
| | TTGTTTAGGAAGTCTAATCTCAAACCTTTTGAGAGAGATATTTCAACTGAAATCTAT |
| | CAGGCCGGTAGCACACCTTGTAATGGTGTTGAAGGTTTTAATTGTTACTTTCCTTTA |
| | CAATCATATGGTTTCCAACCCACTAATGGTGTTGGTTACCAACCATACAGAGTAGT |
| | AGTACTTTCTTTTGAACTTCTACATGCACCAGCAACTGTTTGTGGACCTAAAAAGTC |
| | TACTAATTTGGTTAAAAACAAATGTGTCAATTTCAACTTCAATGGTTTAACAGGCA |
| | CAGGTGTTCTTACTGAGTCTAACAAAAAGTTTCTGCCTTTCCAACAATTTGGCAGA |
| | GACATTGCTGACACTACTGATGCTGTCCGTGATCCACAGACACTTGAGATTCTTGA |
| | CATTACACCATGTTCTTTTGGTGGTGTCAGTGTTATAACACCAGGAACAAATACTTC |
| | TAACCAGGTTGCTGTTCTTTATCAGGATGTTAACTGCACAGAAGTCCCTGTTGCTAT |
| | TCATGCAGATCAACTTACTCCTACTTGGCGTGTTTATTCTACAGGTTCTAATGTTTTT |
| | CAAACACGTGCAGGCTGTTTAATAGGGGCTGAACATGTCAACAACTCATATGAGTG |
| | TGACATACCCATTGGTGCAGGTATATGCGCTAGTTATCAGACTCAGACTAATTCTC |
| | CTCGGCGGGCACGTAGTGTAGCTAGTCAATCCATCATTGCCTACACTATGTCACTT |
| | GGTGCAGAAAATTCAGTTGCTTACTCTAATAACTCTATTGCCATACCCACAAATTTT |
| | ACTATTAGTGTTACCACAGAAATTCTACCAGTGTCTATGACCAAGACATCAGTAGA |
| | TTGTACAATGTACATTTGTGGTGATTCAACTGAATGCAGCAATCTTTTGTTGCAATA |
| | TGGCAGTTTTTGTACACAATTAAACCGTGCTTTAACTGGAATAGCTGTTGAACAAG |
| | ACAAAAACACCCAAGAAGTTTTTGCACAAGTCAAACAAATTTACAAAACACCACC |
| | AATTAAAGATTTTGGTGGTTTTAATTTTTCACAAATATTACCAGATCCATCAAAACC |
| | AAGCAAGAGGTCATTTATTGAAGATCTACTTTTCAACAAAGTGACACTTGCAGATG |
| | CTGGCTTCATCAAACAATATGGTGATTGCCTTGGTGATATTGCTGCTAGAGACCTC |
| | ATTTGTGCACAAAAGTTTAACGGCCTTACTGTTTTGCCACCTTTGCTCACAGATGAA |
| | ATGATTGCTCAATACACTTCTGCACTGTTAGCGGGTACAATCACTTCTGGTTGGACC |
| | TTTGGTGCAGGTGCTGCATTACAAATACCATTTGCTATGCAAATGGCTTATAGGTTT |
| | AATGGTATTGGAGTTACACAGAATGTTCTCTATGAGAACCAAAAATTGATTGCCAA |
| | CCAATTTAATAGTGCTATTGGCAAAATTCAAGACTCACTTTCTTCCACAGCAAGTG |
| | CACTTGGAAAACTTCAAGATGTGGTCAACCAAAATGCACAAGCTTTAAACACGCTT |
| | GTTAAACAACTTAGCTCCAATTTTGGTGCAATTTCAAGTGTTTTAAATGATATCCTT |
| | TCACGTCTTGACAAAGTTGAGGCTGAAGTGCAAATTGATAGGTTGATCACAGGCAG |
| | ACTTCAAAGTTTGCAGACATATGTGACTCAACAATTAATTAGAGCTGCAGAAATCA |
| | GAGCTTCTGCTAATCTTGCTGCTACTAAAATGTCAGAGTGTGTACTTGGACAATCA |
| | AAAAGAGTTGATTTTTGTGGAAAGGGCTATCATCTTATGTCCTTCCCTCAGTCAGCA |
| | CCTCATGGTGTAGTCTTCTTGCATGTGACTTATGTCCCTGCACAAGAAAAGAACTTC |
| | ACAACTGCTCCTGCCATTTGTCATGATGGAAAAGCACACTTTCCTCGTGAAGGTGT |
| | CTTTGTTTCAAATGGCACACACTGGTTTGTAACACAAAGGAATTTTTATGAACCAC |
| | AAATCATTACTACAGACAACACATTTGTGTCTGGTAACTGTGATGTTGTAATAGGA |
| | ATTGTCAACAACACAGTTTATGATCCTTTGCAACCTGAATTAGACTCATTCAAGGA |
| | GGAGTTAGATAAATATTTTAAGAATCATACATCACCAGATGTTGATTTAGGTGACA |
| | TCTCTGGCATTAATGCTTCAGTTGTAAACATTCAAAAAGAAATTGACCGCCTCAAT |
| | GAGGTTGCCAAGAATTTAAATGAATCTCTCATCGATCTCCAAGAACTTGGAAAGTA |
| | TGAGCAGTATATAAAATGGCCATGGTACATTTGGCTAGGTTTTATAGCTGGCTTGA |
| | TTGCCATAGTAATGGTGACAATTATGCTTTGCTGTATGACCAGTTGCTGTAGTTGTC |
| | TCAAGGGCTGTTGTTCTTGTGGATCCTGCTGCAAATTTGATGAAGACGACTCTGAG |
| | CCAGTGCTCAAAGGAGTCAAATTACATTACACACATCATCACCATCACCACTAA |
| | (SEQ ID NO: 27) |

Physical copies of the DNA for the CoV-2 N and S genes were obtained by purchasing the gene sequences from IDT and Genscript. The N gene was purchased from IDT. It is the native gene sequence on a plasmid that is to be used for a standard in a qPCR reaction. An E. coli codon optimized version of the S gene was purchased from Genscript.

The genes were PCR amplified from the plasmid templates and combined with a PCR amplified pRAB11 backbone. The following steps outline the reagents used to create and assemble the plasmids. All PCR steps used Q5 High Fidelity polymerase (NEB). N gene C-terminal 6×his-tag, N gene N-terminal his-tag, S gene C-terminal his tag, and S gene N-terminal his-tag were produced as follows.
1) FBB_p002 pRAB11_P_{Tet}-N gene (C-term 6×his-tag).
   a) N gene—DR_782/DR_781 annealing at @ 70° C. with 40 second extension (1288 base pairs (bp)).
   b) pRAB backbone—DR_784/DR_783 annealing at @ 70° C. with 3 min 45 second extension (6545 bp).
2) FBB_p001 pRAB11_P_{Tet}-N gene (N-term 6×his-tag).
   a) N gene—DR_786/DR_789 annealing at @ 70° C. with 40 second extension (1294 bp).
   b) pRAB backbone—DR_765/DR_790 annealing @ 63° C. with 4.5 min extension (6555 bp)
3) FBB_p004 pRAB11_P_{Tet}-S gene (C-term 6×his-tag).
   a) S gene—DR_794/DR_795 annealing at @ 65° C. with 2 minutes extension (3865 bp).
   b) pRAB backbone—DR_778/DR_791 annealing at @ 68° C. with 4 minute extension (6558 bp).
4) FBB_p003 pRAB11_P_{Tet}-S gene (N-term 6×his-tag)
   a) S gene—DR_797/DR_747 annealing @ 65° C. with 3 minute extension (3858 bp).
   b) pRAB backbone—DR_779/DR_796 annealing at @ 65° C. with 4 minute extension (6546 bp).

All PCR products were analyzed by running the products on a 1% agarose gel, incubated with the restriction endonuclease DpnI (NEB) to remove the methylated DNA used as a template, and purified using Qiagen PCR Purification kit per the manufacturer's instructions. The concentration of the purified PCR product was determined using a NanoDrop™ OneC Microvolume UV-Vis Spectrophotometer (ThermoScientific™). Purified products were used to assemble circular plasmid using the Gibson assembly kit (NEB) per the manufacturer's instructions. Once the Gibson assembly was complete, 0.6 μL of the reaction mixture was used to transform electrocompetent BL21 DE3 (Sigma) E. coli.

Electrocompetent cells plus reaction mixture was placed in a 0.1 mM gap electroporation cuvette and shocked using a Gene pulser II (BioRad) at 1.5 kV and 200Ω resistance. Immediately following the shock step, 1 mL of SOC recovery media (Sigma) was added to the cuvette, and the cells plus media were transferred to a 15 mL culture tube and incubated in a shaking incubator at 37° C. and 250 RPM for one hour, then plated on LB agar plates with 100 μg/mL carbenicillin (Teknova) and incubated for 16 hours at 37° C.

Colonies that formed on the LB carb plates were patched to a fresh LB carb agar plate and screened by PCR for the presence of the plasmid containing either the N or S genes using one primer that binds to the plasmid backbone and one primer that binds to the gene of interest (N), or both primers that bind to the gene of interest (S).
c) N gene colony PCR
   i) DR_215/DR_788 annealing @ 60° C. with 1 minute extension (˜933 bp).
d) S gene colony PCR
   i) DR_776/DR_771 annealing @ 55° C. with 20 seconds extension (191 bp).

The PCR products were run on either a 2% or 1% agarose gel to detect whether a colony was either positive or negative for the presence of a circular plasmid with the recombinant gene of interest. Midori Green (Bulldog Bio) was added to the agarose gel prior to casting the gel and the bands were visualized using a blue LED transilluminator. 4 positive colonies screened were picked and struck out for single colony isolation on new LB carb plates.

High fidelity PCR was performed to generate a PCR product of the promoter region and the gene of interest that could be submitted for Sanger sequencing to confirm the correct DNA sequence on the plasmids contained in the E. coli cells. Primers used were DR_215/DR_216 @ 72° C. with either 2.5 (S) or 1 (N) minute extension time.

PCR products were run on an agarose gel to confirm good amplification, then purified using a Qiagen PCR Cleanup kit per the manufacturer's instructions, and sent to Quintara for sequencing. Sequencing reads from Quintara were aligned to the reference DNA sequence using the DNA sequence alignment tool in Benchling (San Francisco, Calif.). One colony showing a perfect alignment between the sequencing reads from Quintara and the reference strand was picked and stocked in the −80° C. freezer.

CoV-2 Protein Production by E. coli

A small aliquot of sequence confirmed E. coli strains were taken from the ultracold freezer and struck out on LB agar plates containing 100 μg/mL carbenicillin (Teknova) and incubated overnight (12-16 hours) at 37° C.

The following day 3 colonies were picked from a plate and used to inoculate 100 mL of LB broth media (with 100 μg/mL carbenicillin) in a 500 mL baffled shake flask, and incubated at 37° C. until the OD600 reading was at 1. When the OD600 reached a value of 1, 1 μg/mL ATc was added to the culture media to induce the expression of the recombinant viral proteins. The culture flasks were incubated for another 6 hours at room temperature shaking at 250 rpm. Following the 6 hr incubation, the cells were harvested by centrifugation and the pellets frozen at −20° C.

### Results

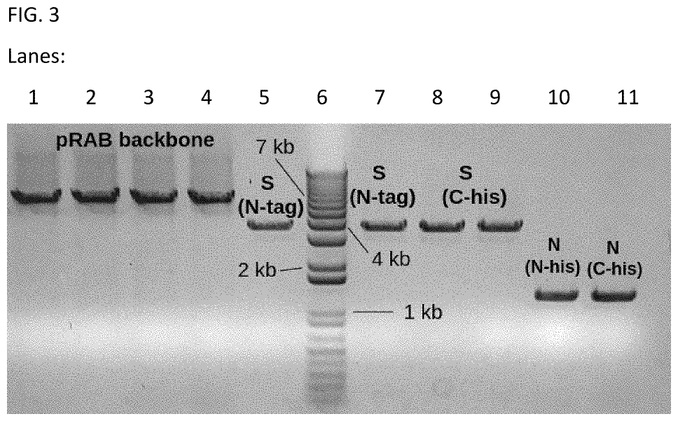

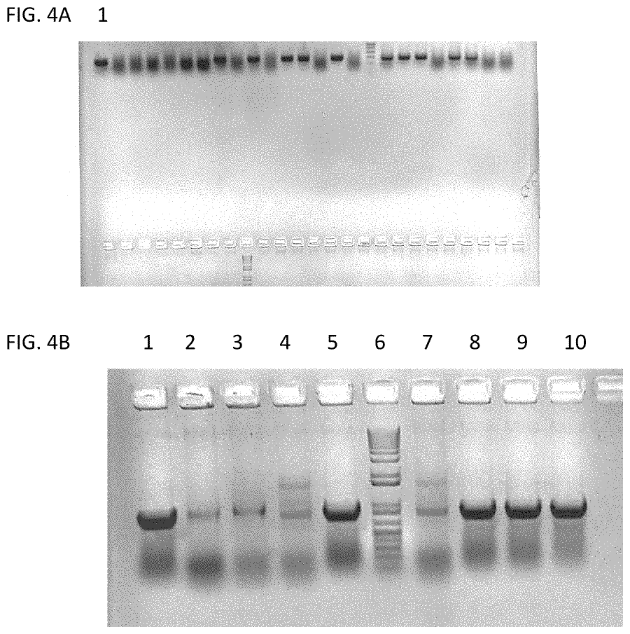

FIG. 3 shows a photograph of an agarose gel of the PCR products generated to build the E. coli expression vectors. Two replicates of the S gene PCR were run in order to generate sufficient quantities of the DNA fragment. Four separate PCR reactions were run for the pRAB backbone fragment generation (lanes 1-4), one for each expression plasmid being built. The expected size of the pRAB backbone fragments are around 6500 base pairs, the expected size of the S gene fragments (lanes 5, 7, 8 and 9) are about 3800 base pairs, the expected size of the N gene fragments (lanes 10 and 11) are about 1290 base pairs. Lane 6 shows a DNA ladder with arrows pointing to 1 kb, 2 kb, 4 kb and 7 kb base pair standards. FIG. 4A and FIG. 4B show photographs of PCR reactions on a 1% agarose gel that were run to screen for fully assembled plasmids. Colonies that grew on the agar plates following the transformation of the Gibson assembly mixture were picked and whole cell lysate was used as the template for the PCR reaction. FIG. 4A shows the results from screening for the presence of the S gene using the primers DR_776 and DR_771. Positive bands are 191 base pairs and appear as defined bands such as in lane 1. FIG. 4B shows the results from screening for the presence of the N gene using the primers DR_788 and DR_215. Positive bands are 933 base pairs and appear as defined bands such as in lane 1. Three colonies that showed positive bands for each construct were grown up to make strain and plasmid freezer stocks, and the plasmid was submitted for sequencing the DNA in the promoter region along with the recombinant gene on the plasmid. Colonies that showed a perfect sequence were kept and stored in the lab's freezers for later expression experiments.

Four new expression vectors were created for the purposes of expressing recombinant CoV-2 proteins in either Staphylococcus aureus (SA) or E. coli. The plasmids contain either the spike (S) gene or the nucleocapsid (N) gene, with either a C or N terminus 6×his-tag. The his tag allows for purification of the recombinant protein from whole cell lysate using immobilized metal affinity chromatography (IMAC), or the ability to identify the protein on a western blot or ELISA using an antibody that binds to the 6×his tag. The plasmids were transformed into BL21(DE3) E. coli cells and stocked in −80° C. freezer.

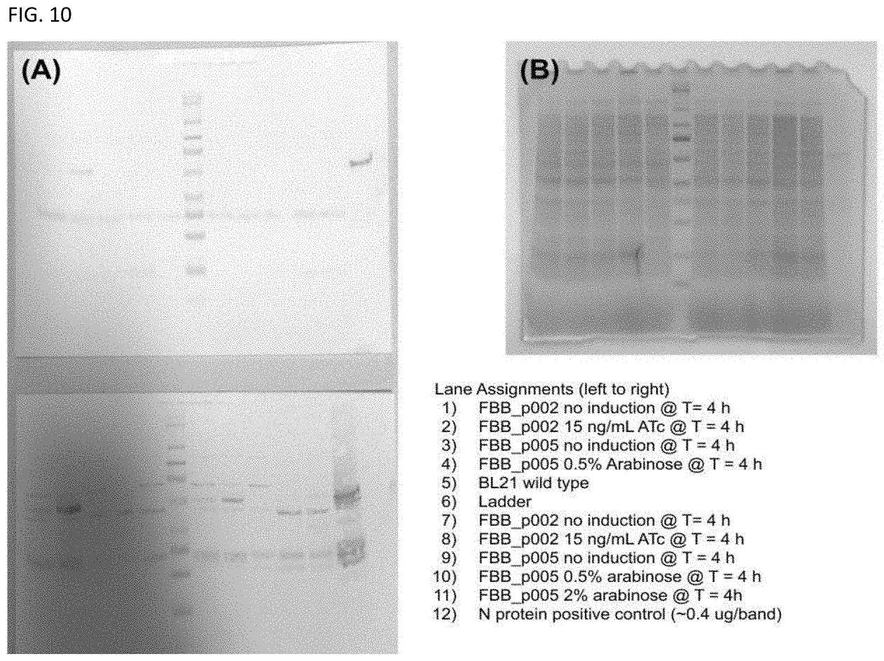

The following protocol was used to express the recombinant proteins in transformed E. coli. A small aliquot of sequence confirmed E. coli strains were taken from the ultracold freezer and struck out on LB agar plates containing 100 μg/mL carbenicillin (Teknova) and incubated overnight (12-16 hours) at 37° C. The following day 3 colonies were picked from a plate and used to inoculate 100 mL of LB broth media (with 100 μg/mL carbenicillin) in a 500 mL baffled shake flask, and incubated at 37° C. until the OD600 reading was at 1. When the OD600 reached a value of 1, 1 μg/mL ATc was added to the culture media to induce the expression of the recombinant viral proteins. The culture flasks were incubated for another 6 hours at room temperature shaking at 250 rpm. Following the 6 hr incubation, the cells were harvested by centrifugation and the pellets frozen at −20° C. Images of gel and blots for N protein are shown in FIG. 10.

FIG. 10 shows the analysis of the soluble fraction of E. coli harboring N protein expression plasmids FB_p002 and FB_p005. (A, top) a membrane from a western blot that was probed with an anti-his HRP conjugated IgG, and (A, bottom) a membrane that was first probed with an anti-his primary IgG and then a chicken anti-rabbit HRP conjugated IgY. (B) shows a PAGE gel that was run at the same time as the gels used in the western blot, but was stained with coomassie blue. A band produced at the correct size in lane 2 indicates that the FBB_jp2 cultures were able to express the protein compared to N protein positive control in lane 12.

There was no band produced at the correct size that would indicate that the FBB_jp5 cultures were able to express the protein. This plasmid FBB_p005 contains the P_{BAD} promoter which is inducible by adding arabinose to the culture. The reason for the lack of protein expression is most likely due to the BL21 strain of E. coli having an intact arabinose metabolism pathway which would lead to the inducer arabinose to be metabolized by the cells rather than inducing the promoter. The FBB_p05 plasmid should be transformed into a strain with this pathway knocked out, such as DC10B.

Staphylococcus aureus strains were also developed. Cultures were set up employing two SA kill switched strains with two versions of full-length S-protein one with a C-terminal His tag and one with an N-terminal His tag, and the same for the N-protein, a Staph aureus 502a WT strain with a His-tagged GFP induced the same way as the other strains, and the two KS stains with no plasmids. Incubation at 37 deg C. Kill switch SA strains contained synthetic genetic mutation such that SA strains were unable to grow under systemic in vivo conditions and will autolyze. Kill switched SA strains were prepared by a modification of the method of Starzl et al., WO 2019/113096, which is incorporated herein by reference in its entirety. Samples were processed and gels were run using Coomassie staining and Western blots using the His-tagged antibody. (data not shown).

### Example 7. Dot ELISAs and Western Blots

Dot ELISAs were performed by applying recombinant SARS-CoV-2 proteins S1 (RayBiotech, E. coli expressed S1), S2 (RayBiotech, E. coli expressed S2), N (RayBiotech, E. coli expressed N protein) and glycosylated S2 protein (Sino, Baculovirus insect cell line expressed S2 protein) antigens to nitrocellulose paper at dilutions of 0.25 μg, 0.10 μg, 0.05 μg, 0.01 μg, and 0.001 μg in duplicate. Eggs identified by flock color coding were according to Table 1B.

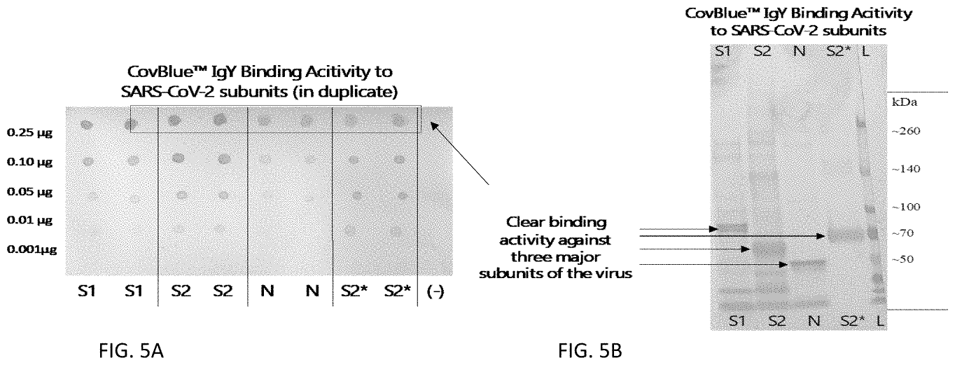

All primary IgY antibodies were from day 12 black egg group eggs. The secondary antibodies used were goat anti-chicken IgY HRP at 1:10,000. Results are shown in FIG. 5A. Clear binding activity is seen against each of three major subunits of the virus. Binding of IgY to S2 and S2* is clearly seen down to 0.01 μg antigen. Binding of IgY to S1 and N is clearly seen down to about 0.05 μg antigen.

A first series of Western blots were prepared using IgY extracts from various egg conditions as primary antibodies. The following SARS-CoV-2 recombinant proteins were employed. S1=RayBiotech E. coli expressed S1 protein (Val16-Gln690; N-terminal His tagged ˜75 kDa), S2=RayBiotech E. coli expressed S2 protein (Met697-Pro1213; ˜58 kDa), N=Raybiotech E. coli expressed nucleocapsid N protein (Met1-Ala419; ˜50 kDa), S2*=Sino, Baculovirus expressed insect cell glycosylated S2 protein-His tag (Ser686-Pro1213; 59.37 kDa). Two gels were loaded with 1 microgram of protein in each lane. Each gel was cut into 3 strips. One strip was Coomassie stained, and the remaining strips were processed as Western blots using several different IgY extracts as primary antibodies including immune egg Black IgY, Black IgY+0.9% benzyl alcohol, Red IgY, Scourguard IgY, and store bought egg IgY. All primary antibodies were added at 0.015 mg/mL in 10 mL PBST. The secondary antibodies were goat anti-chicken IgY (Invitrogen 1:10,000). FIG. 5B shows a representative Western blot showing Red IgY binding activity against the subunits of the COVID-19 virus. Clear IgY binding activity against each of the three major subunits of the SARS-CoV-2 virus including S1, S2, N, and S2* glycosylated protein is demonstrated.

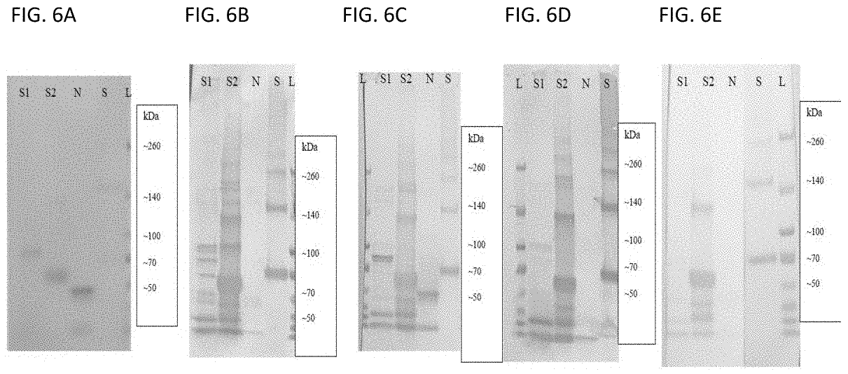

A second series of Western Blots was prepared with IgY extracts from immune eggs from different innoculated flocks, or control store bought eggs, as primary antibodies, using the following antigens: S1=Ray Biotech E. coli expressed S1 protein, S2=RayBiotech E. coli expressed S2 protein, N=Raybiotech E. coli expressed N protein, S=S1+S2 ECD Sino Biological, Inc, Baculovirus expressed insect cell glycosylated S protein (His tagged, 1209 amino acids, ˜134 kDa). Two gels were loaded with 1 μg of protein each lane and each gel was cut into three strips. One strip was Coomasie stained (FIG. 6A), and the remaining strips were processed as Western Blots using several different IgY extracts as primary antibodies. All primary antibodies were loaded at 0.015 mg/mL in 10 mL PBST. The secondary antibodies used were goat anti-chicken IgY (Invitrogen 1:10,000). FIG. 6B shows Black flock derived IgY showing prominent binding to S2 and S antigens. FIG. 6C shows Red flock derived IgY showing good binding to each of S1, S2, N, and S antigens. FIG. 6D shows SCOURGUARD inoculated flock derived IgY showing good binding to S2 and S antigens. FIG. 6E shows IgY derived from store bought eggs; surprisingly binding to S2 and S was exhibited. A further investigation revealed that store bought eggs (Simple truth cage free) came from Opal Foods in Neosho, Mo. and had received veterinary coronavirus vaccines including Newcastle-Bronchitis vaccines of the Mass, Conn, Holland and Ark types (Zoetis).

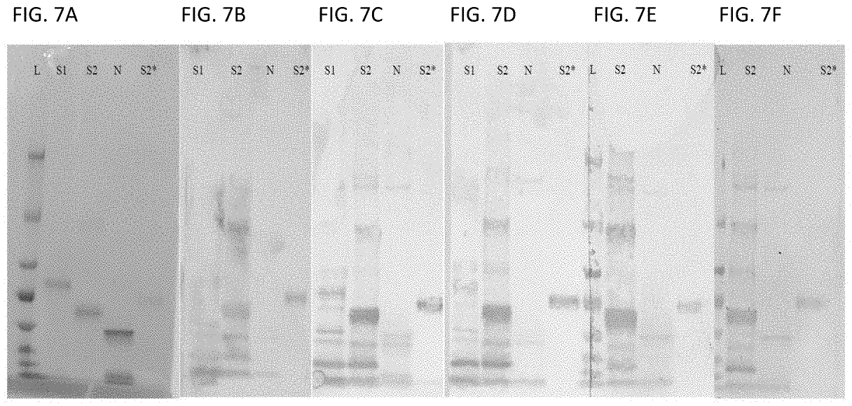

A third series of Western Blots was prepared with IgY processed at different times, or by different methods from eggs from a single flock (Black flock), using the following antigens: S1=Ray Biotech E. coli expressed S1 protein, S2=RayBiotech E. coli expressed S2 protein, N=Raybiotech E. coli expressed N protein (non-glycosylated), S2*=Sino, Baculovirus expressed insect cell glycosylated S2 protein. Two gels were loaded with 1 μg of S1, S2, and N. Baculovirus insect cell S2* was loaded at 0.25 μg. Each lane and each gel was cut into three strips. One strip was Coomasie stained (FIG. 7A), and the remaining strips were processed as Western Blots using several different IgY extract from different processes as primary antibodies. All primary antibodies were loaded at 0.015 mg/mL in 10 mL PBST. The secondary antibodies used were goat anti-chicken IgY (Invitrogen 1:10,000). FIG. 7B shows IgY extract derived from Black flock, clearly exhibiting binding to S2 and S2*. FIG. 7C shows IgY extract derived from Black flock 10 days later than FIG. 7B, clearly exhibiting binding to S2 and S2*, and some binding to S1 and N antigens. FIG. 7D shows IgY extract from dehydrated eggs derived from Black flock, clearly exhibiting binding to S2 and S2*, some binding to S1, and faint binding to N antigens. FIG. 7E shows binding from dehydrated immune eggs from Black flock, clearly exhibiting binding to S2 and S2*, some faint binding to N. FIG. 7F shows binding from whole immune eggs from Black flock, exhibiting binding to S2, S2*, and N antigens. Each of extracted IgY, dehydrated immune eggs, dehydrated extracted immune eggs, and whole immune eggs exhibited binding to the SARS-CoV-2-antigens S1, S2, S2*, and N antigens.

### Example 8. Blocking Dot ELISA Development

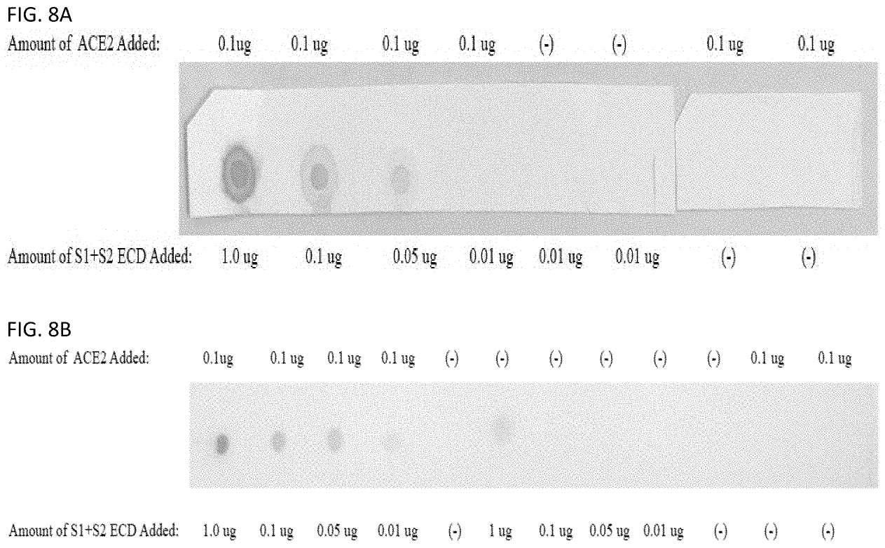

In this example, ACE2 (Acros, not His-Tagged) was coated (dotted) on nitrocellulose paper at the same amount (0.1 ug) across all conditions, with the exception of the ACE2 (−) control wells, where PBS was coated. Recombinant SARS-CoV-2 S1+S2 extracellular domain (ECD) protein His-tagged (Sino Biological, Inc.) was then dotted directly onto the ACE2 in the amounts 1.0 ug, 0.1 ug, 0.05 ug, 0.01 ug, or 0 ug (−), as shown at the bottom of FIGS. 8A and 8B. The positive and negative controls were processed separately side-by-side to avoid cross contamination. The blots were probed with Anti-His HRP antibodies (1:1000).

As shown in FIG. 8A, color development can be seen for the dots which were exposed to 1.0, 0.1 and 0.05 ug of S1+S2 ECD. No color development occurred for the 0.01 ug S1+S2 ECD condition or any of the negative controls.

As shown in FIG. 8B, color development can be seen for the dots which were exposed to 1.0, 0.1, 0.05, and 0.01 ug of SARS-CoV-2 S1+S2 ECD His-tagged antigen. Faint color development can be seen for the (−) ACE2 control when high concentration of 1 ug of S1+S2 was added. No color development occurred for the any of the remaining (−) controls.

### Example 9. IgY Extraction Analysis Using Hodek and PEG 8000 Methods

The IgY may purified from immune eggs using a PEG8000 protocol. Briefly, eggs are washed, with a 1% bleach solution, rinsed and allowed to dry at ambient room temperature. The eggs are broken, allowing the white to flow through and be discarded in waste. The yolk is transferred to filter paper to remove residual white. A small incision in yolk sac is made and contents emptied into a container. Egg yolks are diluted 2-fold with PBS, the solution is pelletized and the supernatant collected. The supernatant undergoes multiple precipitation steps by PEG 8000 in order to remove lipids. For the final precipitation, the pellet is resuspended in PBS and 4M Ammonium sulfate, and centrifuged to pelletize IgY proteins. The supernatant is removed and the pellet is resuspended in PBS and dialyzed for 16-24 h in PBS. After dialyzing is complete, the extracted IgY is tested for total protein concentration using the Bradford assay and purity is checked by running a SDS-PAGE gel.

The IgY may purified from immune eggs using a Hodek protocol. Hodek et al. 2013 developed a protocol that minimizes centrifugation steps, uses only two chemicals (HCl and NaCl) and produces 97% purity of IgY. Hodek et al. 2013. “Optimized Protocol of Chicken Antibody (IgY) Purification Providing Electrophoretically Homogenous Preparations.” Int. J. Electrochem. Sci. 8:12. Briefly, egg yolks are diluted 8-fold in tap water which is then adjusted to a pH of 5 using HCl. The solution is frozen at −20° C. and then placed in a filter apparatus where frozen egg yolk dilution thaws overnight at room temperature. The water soluble fraction containing the IgY filters through the apparatus and is collected throughout the thawing process. The next day, NaCl is added to the water soluble fraction and is adjusted to a pH of 4. The IgY is precipitated during a 2 h mixing step after which the solution is centrifuged, supernatant removed and the pellet containing the purified IgY is resuspended in PBS. The IgY is tested for total protein concentration using the Bradford assay and purity is checked by running a SDS-PAGE gel.

### Example 10. ELISA Reactivity

Egg yolks from Green, Blue, Red eggs according to Table 1B, Ptx (Scourguard®) and store bought eggs were separated from the whites and placed in a test tube (n=1). An equal volume of PBS was added, the mixture shaken vigorously, then centrifuged for 10′ at 10,000 rpm. A 1 ml aliquot of the supernatant was mixed with 100 ul of 41% PEG8000 to extract lipids, the mixture vortexed for ten seconds, and the supernatant collected. The assays were run with a 1:100 dilution of each egg preparation, serially diluted 1:2 in each step, in duplicate. The duplicates were averaged and plotted below. Antigen coatings consisted of N protein (RayBiotech: 230-01104-100) from E. coli (denatured) and S1+S2 ECD (Sino Biological: Cat: 40634-V08B) from Baculovirus-Insect cells. Results are shown in FIG. 9A (S1 and S2 reactivity) and 9B (N-reactivity).

In FIG. 9A, IgY isolated from Green eggs (S1 protein produced in E. coli) exhibited highest reactivity to S1, S2 protein, followed by IgY isolated from Blue eggs (S2 protein produced in E. coli, denatured), store bought eggs, Red eggs (N protein produced in E. coli) and lastly IgY from Scourguard eggs. Surprisingly, IgY from store bought eggs exhibited binding to S1, S2 in the ELISA, similar to that seen in Western Blot infra. A further investigation revealed that store bought eggs (Simple truth cage free) came from Opal Foods in Neosho, Mo. and had received veterinary coronavirus vaccines including Newcastle-Bronchitis vaccines of the Mass, Conn, Holland and Ark types (Zoetis).

In FIG. 9B, IgY isolated from Red eggs (N protein produced in E. coli) exhibited highest reactivity to N-protein, followed by Blue eggs (S2 protein produced in E. coli, denatured), Green eggs (S1 protein produced in E. coli), and very low reactivity exhibited by IgY isolated from store bought and Scourguard® eggs.

### Example 11. Bradford Assay for IgY Quantitation

Bradford assay protocol was used to measure total protein from egg yolk IgY extractions and prepare samples for SDS-PAGE. A variation of manufacturer's protocol for Coomassie Plus (Bradford) Assay Kit, ThermoScientific was employed, using a bovine serum albumin (BSA) standard curve, and coomassie G-250 dye. Absorbance at 630 nm was measured and compared to standard curve.

### Example 12. SDS-PAGE Protocol

Unless otherwise specified, sodium dodecyl sulfate-polyacrylamide gel electrophoresis (SDS-PAGE) gels were run using BOLT™4-12% Bis-TRIS (Invitrogen) under reducing conditions using a volume (x uL) of extracted protein corresponding to ˜10 micrograms total protein.

Briefly, IgY samples were prepared using (13−x) uL mol bio water, 5 uL LDS loading dye (Bolt™ LDS Sample Buffer (4×); Novex), x uL IgY sample, 1 uL sample Reducing Agent (Bolt—Sample Reducing Agent (10×), Novex), mixed and incubated at 70 deg C. for 10 min. Gel is loaded using 10 uL sample in individual wells and a protein ladder is also added to a well. The gel is run in Electrophoresis Chamber (XCell SureLock® Mini-Cell, ThermoFisher) for 34 min at 200 V, or according to manufacturer's protocols. Gel is washed twice in 100 mL distilled water and stained using SimplyBlue SafeStain for 5-20 min, rinsed with distilled water and 20% NaCl, and photographed.

### Example 13. Plasmid Construction for Recombinant Human ACE2 Protein

This example illustrates construction of plasmid FBB_p020 encoding human ACE2. The plasmid was prepared, stocked, transformed and used to induce ACE2 protein production E. coli cultures. The high copy plasmid backbone pRAB11 was employed. Helle, Leonie, et al. “Vectors for improved Tet repressor-dependent gradual gene induction or silencing in Staphylococcus aureus.” Microbiology 157.12 (2011): 3314-3323. Table 4 shows the primers used to make and sequence the plasmid.

TABLE 4
Primers used to make and sequence the p020 plasmid

| Primer Name | Primer Sequence (5′-->3′) |
|---|---|
| DR_867 | CAGAGAGGAGGTGTATAAGGTGATGAGTTCATCC |
| | TCTTGGTTGCTATTAAGCTTG |
| | (SEQ ID NO: 29) |
| DR_868 | GTCACTtagtggtgatggtgatgatgGAAGCTGG |
| | TCTGCACGTCGTC |
| | (SEQ ID NO: 30) |
| DR_801 | catcatcaccatcaccactaaGTGACATATAGCC |
| | GCACCAATAAAAATtg |
| | (SEQ ID NO: 31) |
| DR_244 | CATCACCTTATACACCTCCTCTCTGCGG |
| | (SEQ ID NO: 32) |

Table 5 shows the DNA sequences used in the construction FBB_p020; the sequences represent one strand of the double stranded DNA fragment. Table 6 shows the amino acid sequence of the ACE2 gene.

TABLE 5
DNA Sequences of ACE2 gene and pRAB11 Backbone

| Name/ | |
|---|---|
| Seq. ID | DNA Sequence (5′-->3′) |
| Ace2/ | ATGAGTTCATCCTCTTGGTTGCTATTAAGCTTGGTTGCCGTTACGGCTGCGCAGAGCACCAT |
| FBB_DNA_ | CGAAGAGCAGGCGAAAACCTTTCTGGACAAATTCAACCACGAAGCTGAAGACCTGTTTTAT |
| 008 | CAGTCCAGCTTAGCTAGCTGGAACTATAATACGAACATTACTGAGGAGAACGTGCAGAAC |
| | ATGAATAATGCGGGTGACAAGTGGTCAGCCTTTCTGAAAGAACAGTCCACGCTGGCACAA |
| | ATGTACCCACTGCAAGAGATCCAAAACCTGACGGTCAAACTGCAGCTGCAAGCGCTGCAA |
| | CAAAACGGCAGCAGCGTTCTGTCCGAGGACAAGTCCAAACGTTTGAATACGATCCTGAAC |
| | ACGATGTCCACCATCTATTCAACCGGCAAGGTGTGCAATCCGGATAACCCGCAGGAGTGCC |
| | TGCTCCTGGAACCGGGTCTCAATGAAATCATGGCGAACAGCTTAGATTATAACGAACGTCT |
| | GTGGGCATGGGAGAGCTGGCGTAGCGAAGTGGGTAAACAGTTACGCCCTCTGTACGAGGA |
| | ATATGTTGTCCTGAAGAACGAGATGGCCCGAGCGAATCACTATGAGGACTACGGCGACTA |
| | CTGGCGCGGTGATTACGAGGTGAATGGCGTGGATGGTTATGATTACAGCCGCGGGCAGCTG |
| | ATTGAAGACGTCGAGCACACCTTCGAGGAGATCAAGCCGCTGTACGAACACCTTCACGCAT |
| | ACGTGAGAGCGAAACTGATGAACGCGTACCCGAGCTACATTTCCCCGATTGGTTGTCTGCC |
| | AGCACATCTGTTAGGCGACATGTGGGGTCGTTTTTGGACCAATCTGTATTCTTTGACCGTTC |
| | CGTTCGGCCAGAAGCCGAATATCGATGTTACCGACGCTATGGTTGACCAAGCCTGGGATGC |
| | TCAACGTATCTTTAAAGAAGCGGAAAAGTTCTTTGTTAGCGTAGGCCTGCCGAACATGACC |
| | CAGGGTTTCTGGGAGAACAGTATGCTGACCGATCCGGGAAACGTTCAGAAGGCCGTGTGTC |
| | ATCCGACCGCGTGGGATCTGGGTAAGGGCGACTTCCGCATACTGATGTGCACCAAAGTGAC |
| | CATGGATGATTTTCTGACCGCGCATCATGAGATGGGTCATATTCAGTACGACATGGCGTAC |
| | GCAGCGCAACCATTTCTGCTGCGTAATGGTGCCAACGAGGGCTTCCACGAGGCGGTGGGCG |
| | AGATTATGAGCCTGTCTGCGGCGACCCCGAAGCACCTGAAGTCAATTGGCCTGCTGAGCCC |
| | GGACTTTCAAGAAGACAACGAGACGGAGATCAATTTCTTGTTGAAGCAAGCTTTGACTATT |
| | GTGGGCACCCTGCCTTTCACCTACATGTTGGAGAAGTGGCGTTGGATGGTGTTCAAAGGTG |
| | AAATTCCGAAAGACCAGTGGATGAAAAAGTGGTGGGAAATGAAAAGAGAAATCGTAGGT |
| | GTTGTTGAACCGGTTCCGCATGATGAAACCTACTGCGACCCGGCGAGCCTGTTCCATGTTTC |
| | CAATGACTACAGCTTCATCCGTTATTACACCCGTACCTTGTACCAATTTCAGTTTCAAGAAG |
| | CGCTGTGTCAGGCCGCTAAGCACGAAGGTCCGCTGCACAAATGCGATATTAGCAACTCCAC |
| | TGAGGCCGGTCAAAAACTGTTCAACATGCTGCGCCTGGGCAAAAGCGAACCGTGGACCCT |
| | CGCGTTAGAGAATGTAGTTGGCGCAAAGAACATGAATGTGCGTCCACTGTTGAACTATTTT |
| | GAGCCGCTTTTCACCTGGCTGAAGGATCAAAACAAAAACTCCTTCGTGGGTTGGTCAACTG |
| | ATTGGTCTCCGTATGCTGATCAAAGTATTAAGGTTCGCATTTCGCTGAAGAGCGCGTTGGG |
| | TGATAAAGCTTACGAGTGGAATGATAATGAAATGTATCTGTTCCGTTCTAGCGTGGCGTAC |
| | GCAATGCGTCAGTATTTCTTGAAGGTGAAAAACCAGATGATTCTCTTCGGTGAAGAGGACG |
| | TCCGTGTCGCCAATCTGAAGCCGCGTATTTCGTTCAATTTTTTCGTGACCGCTCCGAAAAAC |
| | GTTAGCGATATCATCCCGCGTACCGAGGTGGAAAAAGCGATTCGTATGTCTCGTAGCCGCA |
| | TCAACGACGCATTTCGCCTGAACGACAATTCCTTGGAGTTCTTGGGCATCCAGCCGACATT |
| | GGGTCCCCCGAACCAGCCGCCGGTGAGCATCTGGCTGATCGTTTTTGGCGTTGTTATGGGT |
| | GTCATCGTTGTTGGCATCGTGATCCTCATTTTTACGGGCATCCGCGATCGTAAAAAGAAGA |
| | ACAAAGCGCGTTCTGGTGAGAACCCGTATGCAAGCATCGACATTAGTAAAGGTGAAAACA |
| | ACCCAGGTTTTCAAAACACCGACGACGTGCAGACCAGCTTCCATCATCACCATCACCACTAA |
| | (SEQ ID NO: 33) |
| pRAB11 | GTGACATATAGCCGCACCAATAAAAATtgataatagctgagcccgggCACTGGCCGTCGTTTTAC |
| Backbone/ | AACGTCGTGACTGGGAAAACCCTGGCGTTACCCAACTTAATCGCCTTGCAGCACATCCCCCTTTC |
| FB_DNA_ | GCCAGCTGGCGTAATAGCGAAGAGGCCCGCACCGATCGCCCTTCCCAACAGTTGCGCAGCC |
| 011 | TGAATGGCGAATGGCGCCTGATGCGGTATTTTCTCCTTACGCATCTGTGCGGTATTTCACAC |
| | CGCATATGGTGCACTCTCAGTACAATCTGCTCTGATGCCGCATAGTTAAGCCAGCCCCGAC |
| | ACCCGCCAACACCCGCTGACGCGCCCTGACGGGCTTGTCTGCTCCCGGCATCCGCTTACAG |
| | ACAAGCTGTGACCGTCTCCGGGAGCTGCATGTGTCAGAGGTTTTCACCGTCATCACCGAAA |
| | CGCGCGAGACGAAAGGGCCTCGTGATACGCCTATTTTTATAGGTTAATGTCATGATAATAA |
| | TGGTTTCTTAGACGTCAGGTGGCACTTTTCGGGGAAATGTGCGCGGAACCCCTATTTGTTTA |
| | TTTTTCTAAATACATTCAAATATGTATCCGCTCATGAGACAATAACCCTGATAAATGCTTCA |
| | ATAATATTGAAAAAGGAAGAGTATGAGTATTCAACATTTCCGTGTCGCCCTTATTCCCTTTT |
| | TTGCGGCATTTTGCCTTCCTGTTTTTGCTCACCCAGAAACGCTGGTGAAAGTAAAAGATGCT |
| | GAAGATCAGTTGGGTGCACGAGTGGGTTACATCGAACTGGATCTCAACAGCGGTAAGATC |
| | CTTGAGAGTTTTCGCCCCGAAGAACGTTTTCCAATGATGAGCACTTTTAAAGTTCTGCTATG |
| | TGGCGCGGTATTATCCCGTATTGACGCCGGGCAAGAGCAACTCGGTCGCCGCATACACTAT |
| | TCTCAGAATGACTTGGTTGAGTACTCACCAGTCACAGAAAAGCATCTTACGGATGGCATGA |
| | CAGTAAGAGAATTATGCAGTGCTGCCATAACCATGAGTGATAACACTGCGGCCAACTTACT |
| | TCTGACAACGATCGGAGGACCGAAGGAGCTAACCGCTTTTTTGCACAACATGGGGGATCAT |
| | GTAACTCGCCTTGATCGTTGGGAACCGGAGCTGAATGAAGCCATACCAAACGACGAGCGT |
| | GACACCACGATGCCTGTAGCAATGGCAACAACGTTGCGCAAACTATTAACTGGCGAACTAC |
| | TTACTCTAGCTTCCCGGCAACAATTAATAGACTGGATGGAGGCGGATAAAGTTGCAGGACC |
| | ACTTCTGCGCTCGGCCCTTCCGGCTGGCTGGTTTATTGCTGATAAATCTGGAGCCGGTGAGC |
| | GTGGGTCTCGCGGTATCATTGCAGCACTGGGGCCAGATGGTAAGCCCTCCCGTATCGTAGT |
| | TATCTACACGACGGGGAGTCAGGCAACTATGGATGAACGAAATAGACAGATCGCTGAGAT |
| | AGGTGCCTCACTGATTAAGCATTGGTAACTGTCAGACCAAGTTTACTCATATATACTTTAGA |
| | TTGATTTAAAACTTCATTTTTAATTTAAAAGGATCTAGGTGAAGATCCTTTTTGATAATCTC |
| | ATGACCAAAATCCCTTAACGTGAGTTTTCGTTCCACTGAGCGTCAGACCCCGTAGAAAAGA |
| | TCAAAGGATCTTCTTGAGATCCTTTTTTTCTGCGCGTAATCTGCTGCTTGCAAACAAAAAAA |
| | CCACCGCTACCAGCGGTGGTTTGTTTGCCGGATCAAGAGCTACCAACTCTTTTTCCGAAGGT |
| | AACTGGCTTCAGCAGAGCGCAGATACCAAATACTGTTCTTCTAGTGTAGCCGTAGTTAGGC |
| | CACCACTTCAAGAACTCTGTAGCACCGCCTACATACCTCGCTCTGCTAATCCTGTTACCAGT |
| | GGCTGCTGCCAGTGGCGATAAGTCGTGTCTTACCGGGTTGGACTCAAGACGATAGTTACCG |
| | GATAAGGCGCAGCGGTCGGGCTGAACGGGGGGTTCGTGCACACAGCCCAGCTTGGAGCGA |
| | ACGACCTACACCGAACTGAGATACCTACAGCGTGAGCTATGAGAAAGCGCCACGCTTCCC |
| | GAAGGGAGAAAGGCGGACAGGTATCCGGTAAGCGGCAGGGTCGGAACAGGAGAGCGCAC |
| | GAGGGAGCTTCCAGGGGGAAACGCCTGGTATCTTTATAGTCCTGTCGGGTTTCGCCACCTC |
| | TGACTTGAGCGTCGATTTTTGTGATGCTCGTCAGGGGGGCGGAGCCTATGGAAAAACGCCA |
| | GCAACGCGGCCTTTTTACGGTTCCTGGCCTTTTGCTGGCCTTTTGCTCACATGTTCTTTCCTG |
| | CGTTATCCCCTGATTCTGTGGATAACCGTATTACCGCCTTTGAGTGAGCTGATACCGCTCGC |
| | CGCAGCCGAACGACCGAGCGCAGCGAGTCAGTGAGCGAGGAAGCGGAAGAGCGCCCAAT |
| | ACGCAAACCGCCTCTCCCCGCGCGTTGGCCGATTCATTAATGCAGCTGGCACGACAGGTTT |
| | CCCGACTGGAAAGCGGACAGTGAGCGCAACGCAATTAATGTGAGTTAGCTCACTCATTAG |
| | GCACCCCAGGCTTTACACTTTATGCTTCCGGCTCGTATGTTGTGTGGAATTGTGAGCGGATA |
| | ACAATTTCACACAGGAAACAGCTATGACCATGATTACGCCAAGCTTCTGTAGGTTTTTAGG |
| | CATAAAACTATATGATTTACCCCTAAATCTTTAAAATGCCCCTTAAAATTCAAAATAAAGG |
| | CATTTAAAATTTAAATATTTCTTGTGATAAAGTTTGTTAAAAAGGAGTGGTTTTATGACTGT |
| | TATGTGGTTATCGATTATAGGTATGTGGTTTTGTATTGGAATGGCATTTTTTGCTATCAAGG |
| | TTATTAAAAATAAAAATTAGACCACGCATTTATGCCGAGAAAATTTATTGTGCGTTGAGAA |
| | GAACCCTTAACTAAACTTGCAGACGAATGTCGGCATAGCGTGAGCTATTAAGCCGACCATT |
| | CGACAAGTTTTGGGATTGTTAAGGGTTCCGAGGCTCAACGTCAATAAAGCAATTGGAATAA |
| | AGAAGCGAAAAAGGAGAAGTCGGTTCAGAAAAAGAAGGATATGGATCTGGAGCTGTAAT |
| | ATAAAAACCTTCTTCAACTAACGGGGCAGGTTAGTGACATTAGAAAACCGACTGTAAAAA |
| | GTACAGTCGGCATTATCTCATATTATAAAAGCCAGTCATTAGGCCTATCTGACAATTCCTGA |
| | ATAGAGTTCATAAACAATCCTGCATGATAACCATCACAAACAGAATGATGTACCTGTAAAG |
| | ATAGCGGTAAATATATTGAATTACCTTTATTAATGAATTTTCCTGCTGTAATAATGGGTAGA |
| | AGGTAATTACTATTATTATTGATATTTAAGTTAAACCCAGTAAATGAAGTCCATGGAATAA |
| | TAGAAAGAGAAAAAGCATTTTCAGGTATAGGTGTTTTGGGAAACAATTTCCCCGAACCATT |
| | ATATTTCTCTACATCAGAAAGGTATAAATCATAAAACTCTTTGAAGTCATTCTTTACAGGAG |
| | TCCAAATACCAGAGAATGTTTTAGATACACCATCAAAAATTGTATAAAGTGGCTCTAACTT |
| | ATCCCAATAACCTAACTCTCCGTCGCTATTGTAACCAGTTCTAAAAGCTGTATTTGAGTTTA |
| | TCACCCTTGTCACTAAGAAAATAAATGCAGGGTAAAATTTATATCCTTCTTGTTTTATGTTT |
| | CGGTATAAAACACTAATATCAATTTCTGTGGTTATACTAAAAGTCGTTTGTTGGTTCAAATA |
| | ATGATTAAATATCTCTTTTCTCTTCCAATTGTCTAAATCAATTTTATTAAAGTTCATTTGATA |
| | TGCCTCCTAAATTTTTATCTAAAGTGAATTTAGGAGGCTTACTTGTCTGCTTTCTTCATTAGA |
| | ATCAATCCTTTTTTAAAAGTCAATATTACTGTAACATAAATATATATTTTAAAAATATCCCA |
| | CTTTATCCAATTTTCGTTTGTTGAACTAATGGGTGCTTTAGTTGAAGAATAAAAGACCACAT |
| | TAAAAAATGTGGTCTTTTGTGTTTTTTTAAAGGATTTGAGCGTAGCGAAAAATCCTTTTCTT |
| | TCTTATCTTGATAATAAGGGTAACTATTGCCGGCGAGGCTAGTTACCCTTAAGTTATTGGTA |
| | TGACTGGTTTTAAGCGCAAAAAAAGTTGCTTTTTCGTACCTATTAATGTATCGTTTTAAATG |
| | AATAGTAAAAAACATACATAGAAAGGGGAAAAAGCAACTTTTTTTATTGTCATAGTTTGTG |
| | AAAACTAAGTTGTTTTTATGTGTTATAACATGGAAAAGTATACTGAGAAAAAACAAAGAA |
| | ATCAAGTATTTCAGAAATTTATTAAACGTCATATTGGAGAGAATCAAATGGATTTAGTTGA |
| | AGATTGCAATACATTTCTGTCTTTTGTAGCTGATAAAACTTTAGAAAAACAGAAATTATAT |
| | AAAGCTAATTCTTGTAAAAATCGATTTTGTCCTGTCTGTGCTTGGAGAAAAGCTAGGTCAG |
| | CTGTTGAATTATGCACGAGTATTTTAAAAGTTATTGTGATGACGACGATAAACGATTATCA |
| | AAAGTATAATGTTAAAATGCTTTATTATACTAACGTTATATAAACATTATACTTTCGTTATA |
| | CAAATTTTAACCCTGTTAGGAACTATAAAAAATCATGAAAATTTTAATTTGCATGTAACTG |
| | GGCAGTGTCTTAAAAAATCGACACTGAATTTGCTCAAATTTTTGTTTGTAGAATTAGAATAT |
| | ATTTATTTGGCTCATATTTGCTTTTTAAAAGCTTGCATGCCTGCAGGTCGACGGTATCGATA |
| | ACTCGACATCTTGGTTACCGTGAAGTTACCATCACGGAAAAAGGTTATGCTGCTTTTAAGA |
| | CCCACTTTCACATTTAAGTTGTTTTTCTAATCCGCATATGATCAATTCAAGGCCGAATAAGA |
| | AGGCTGGCTCTGCACCTTGGTGATCAAATAATTCGATAGCTTGTCGTAATAATGGCGGCAT |
| | ACTATCAGTAGTAGGTGTTTCCCTTTCTTCTTTAGCGACTTGATGCTCTTGATCTTCCAATAC |
| | GCAACCTAAAGTAAAATGCCCCACAGCGCTGAGTGCATATAATGCATTCTCTAGTGAAAAA |
| | CCTTGTTGGCATAAAAAGGCTAATTGATTTTCGAGAGTTTCATACTGTTTTTCTGTAGGCCG |
| | TGTACCTAAATGTACTTTTGCTCCATCGCGATGACTTAGTAAAGCACATCTAAAACTTTTAG |
| | CGTTATTACGTAAAAAATCTTGCCAGCTTTCCCCTTCTAAAGGGCAAAAGTGAGTATGGTG |
| | CCTATCTAACATCTCAATGGCTAAGGCGTCGAGCAAAGCCCGCTTATTTTTTACATGCCAAT |
| | ACAATGTAGGCTGCTCTACACCTAGCTTCTGGGCGAGTTTACGGGTTGTTAAACCTTCGATT |
| | CCGACCTCATTAAGCAGCTCTAATGCGCTGTTAATCACTTTACTTTTATCTAATCTAGACAT |
| | CATTAATTCCTCCTTTTTGTTGACATTATATCATTGATAGAGTTATTTGTCAAACTAGTTTTT |
| | TATTTGGATCCCCTCGAGTTCATGAAAAACTAAAAAAAATATTGACACTCTATCATTGATA |
| | GAGTATAATTAAAATAAGCTCTCTATCATTGATAGAGTATGATGGTACCGTTAACAGATCT |
| | GAGCCGCAGAGAGGAGGTGTATAAGGTG |
| | (SEQ ID NO: 34) |

TABLE 6
Amino Acid Sequence of Ace2 with C-terminal hexa
His tag

| Name/Seq. ID | Amino Acid Sequence (5′-->3′) |
|---|---|
| Ace2/FBB_AA_008 | MSSSSWLLLSLVAVTAAQSTIEEQAKTFLDKFN |
| | HEAEDLFYQSSLASWNYNTNITEENVQNMNNAG |
| | DKWSAFLKEQSTLAQMYPLQEIQNLTVKLQLQA |
| | LQQNGSSVLSEDKSKRLNTILNTMSTIYSTGKV |
| | CNPDNPQECLLLEPGLNEIMANSLDYNERLWAW |
| | ESWRSEVGKQLRPLYEEYVVLKNEMARANHYED |
| | YGDYWRGDYEVNGVDGYDYSRGQLIEDVEHTFE |
| | EIKPLYEHLHAYVRAKLMNAYPSYISPIGCLPA |
| | HLLGDMWGRFWTNLYSLTVPFGQKPNIDVTDAM |
| | VDQAWDAQRIFKEAEKFFVSVGLPNMTQGFWEN |
| | SMLTDPGNVQKAVCHPTAWDLGKGDFRILMCTK |
| | VTMDDFLTAHHEMGHIQYDMAYAAQPFLLRNGA |
| | NEGFHEAVGEIMSLSAATPKHLKSIGLLSPDFQ |
| | EDNETEINFLLKQALTIVGTLPFTYMLEKWRWM |
| | VFKGEIPKDQWMKKWWEMKREIVGVVEPVPHDE |
| | TYCDPASLFHVSNDYSFIRYYTRTLYQFQFQEA |
| | LCQAAKHEGPLHKCDISNSTEAGQKLFNMLRLG |
| | KSEPWTLALENVVGAKNMNVRPLLNYFEPLFTW |
| | LKDQNKNSFVGWSTDWSPYADQSIKVRISLKSA |
| | LGDKAYEWNDNEMYLFRSSVAYAMRQYFLKVKN |
| | QMILFGEEDVRVANLKPRISFNFFVTAPKNVSD |
| | IIPRTEVEKAIRMSRSRINDAFRLNDNSLEFLG |
| | IQPTLGPPNQPPVSIWLIVFGVVMGVIVVGIVI |
| | LIFTGIRDRKKKNKARSGENPYASIDISKGENN |
| | PGFQNTDDVQTSFHHHHHH* |
| | (SEQ ID NO: 35) |

PCR Fragment Generation

Genscript's codon optimization program was used to generate an E. coli codon optimized version of the human ace2 protein. The amino acid sequence was used as the input for the codon optimization. The DNA sequence generated from the codon optimization was used to design double stranded DNA fragments to be synthesized by IDT, and assembled to be used as a template for the PCR reaction to make the ace2 fragment for FBB_p010, the plasmid used as a template in the following protocol.

The following PCR reactions were performed using Q5 High Fidelity Hot Start 2× Master Mix (NEB) per the manufacturer's instructions:

FB_DNA_011 pRAB1I Backbone

DR_801/DR_244

FB_DNA_010 ACE2 Gene

DR_867/DR_868

The above PCR fragments were checked on a 1% agarose gel to confirm a clean band, and then purified using a Qiaquick PCR Purification Kit (Qiqagen) per the manufacturer's instructions.

The p024 fragment was treated with DpnI (NEB) to remove the methylated circular plasmid used as the template for the PCR, and purified again using the PCR Cleanup Kit (NEB).

The fragments were stitched together using Q5 Hot Start Master Mix (NEB) then used in a Gibson Assembly (NEB) to create a circular plasmid per the manufacturer's instructions. The assembled plasmid was then transformed into BL21 (DE3) Competent E. coli cells per the manufacturer's instructions, plated on LB (carb), and incubated overnight at 37° C.

The following day colonies were screened for fully assembled plasmids by colony PCR. Three positive colonies were chosen to start overnight cultures in LB (erythromycin) to extract the plasmids.

The next day 750 μL of culture was added to 750 μL 50% glycerol in a cryostock and stored at −80° C.

Plasmids were extracted from the remaining culture using the Zyppy Plasmid Mini Prep Kit and concentrated using the Zyppy Clean and Concentrate Kit.

The three extracted plasmids were used as the template for a high fidelity PCR reaction using Q5 Hot Start Master Mix (NEB) using primers DR_215 and DR_216 which bind to the plasmid backbone and capture the whole insertion region, purified using the Qiaquick PCR Purification Kit (Qiagen), and sent to Quintara Biosciences to be sequenced.

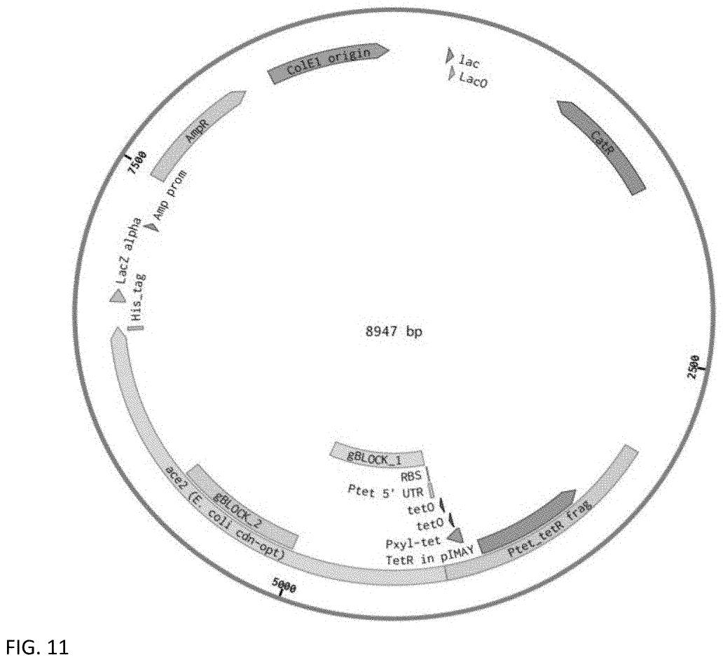

The sequencing was aligned in silico using the sequence alignment tool in the molecular biology platform Benchling. A map of the p020 plasmid for human ACE2 made in the Benchling program is shown in FIG. 11 One of the sequences that showed a perfect alignment was picked to be stocked in the plasmid database. Sanger sequencing of the constructed plasmid revealed no mutations in the Ace2 gene insertion. The plasmid was stocked and transformed into BL21 (DE3).

### Example 14. Production and Quantification of Recombinant Human ACE2 Protein for Inoculation into Chickens

This example shows production of recombinant human ACE2 protein using a plasmid based E. coli platform. A 3 L batch fermentation was performed using the strain FBB_p020, then analyzed for protein production by Western blot. The anti-His western blot was also analyzed with the ImageJ software to determine the concentration of ACE2 in the sample, which came out to 0.313 mg/mL of insoluble protein.

Plasmid FBB_p020 encoding the amino acid sequence for human ACE2 protein along with a C terminal 6×his-tag was inserted into the plasmid backbone pRAB11 behind the anhydrotetracycline (ATc) inducible promoter as disclosed herein. The cells were then grown in a 3 L batch fermentor, harvested 24 hours after induction, then analyzed for the amount of protein in the soluble and insoluble phase.

The expression plasmid was made as described herein for FBB_p020. Briefly, the backbone and insert were PCR amplified from their respective templates and circularized using the Gibson assembly kit. The circularized plasmids were transformed into BL21(DE3) and the DNA sequence of individual colonies was confirmed by Sanger sequencing. The sequence confirmed plasmids were then used to overexpress the recombinant protein.

A 5 mL LB (carb) liquid culture was started from a freezer stock of the strains and incubated overnight at 37° C. in a shaking incubator. The following day the optical density (OD₆₀₀) was taken of the overnight culture, and an appropriate volume of the culture was used to inoculate a fresh 150 mL LB (carb) to an OD₆₀₀ of 0.05 in a baffled shake flask. The shake flask was then incubated at 37° C. for about 3 hours in a shaking incubator. After about 3 hours the OD600 was checked and the appropriate volume was calculated and used to inoculate 3 L of LB (carb) to an OD₆₀₀ of 0.05-0.1. The 3 L culture was incubated at 37° C. until the OD₆₀₀ reached 0.6-0.8 (about 2 hours) at which time 900 μL of anhydrotetracycline (ATc) was added to the culture to induce protein production. Following induction, the culture was placed in a room temperature water bath and the remaining steps were carried out at room temp.

A stir bar was sterilized inside the glassware, and during production the bottle was placed on a stir plate to keep the culture mixing at a rate so the oxygen and nutrient limitation would not limit the rate of growth of the bacteria. Filter sterilized air was sparged into the tanks to provide oxygen for the entirety of the run. Additional 900 μL of anhydrotetracycline (ATc) was added at 6, 12, and 18 hours after the initial induction. The cells were harvested 24 hours post induction by centrifugation and the pellets were frozen at −20° C. and stored there until they were analyzed for protein content.

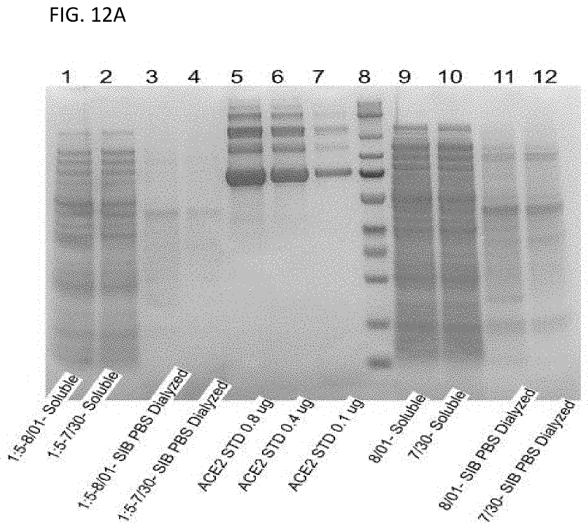

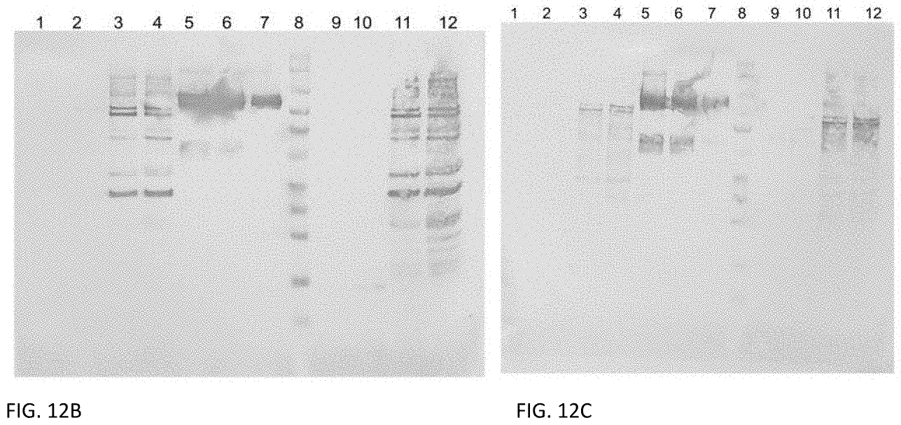

The cells were lysed using the B-Per cell lysis kit per the manufacturer's instructions. The insoluble pellet was solubilized using 2% SDS, and both the soluble and insoluble fractions were run on a Bolt™ PAGE gel per the manufacturer's instructions, and stained with SimplyBlue Coomassie stain per the manufacturer's instructions, as shown in FIG. 12A. An additional two gels were run alongside the Coomassie stained gel and following the electrophoresis step the proteins were transferred to membranes using a power blotter. The membranes were then used for analysis of the lysates by Western blot. The membranes were blocked overnight at 4° C. by soaking them in a 5% milk solution in PBS-tween (PBS-T). Following the blocking the blots were washed three times with PBS-T for 5 minutes each on a rocking table. A 1:1000 dilution of an HRP conjugated anti-his IgG antibody (Thermo) and unconjugated ACE2 IgG (Thermo) were made separately in 10 mL of PBS-T with 1% milk. Following the wash step above each membrane was soaked in one of the solutions containing the antibodies on a rocking table for 90 minutes at room temperature. Following the 90 minute incubation, the membranes were washed again 3 times with PBST for 10 minutes each wash. The membrane that was incubated with the anti-His antibody and developed using a Dab substrate kit (Thermo) per the manufacturer's instructions. Following the 3× wash with PBS-T, the membrane that was probed with the anti-ACE2 IgG was incubated with a PBS-T solution with 1% milk and a 1:2500 dilution of a goat anti-mouse IgG (Thermo) for 60 minutes mixing on a rocking table at room temp. Following the 60 min room temp incubation, the membrane was washed again with PBS-T three more times for 10 minutes a piece. The membrane was then developed using the Dab substrate kit per the manufacturer's instructions.

Following confirmation that the majority of the protein produced was located in the insoluble fraction, the whole pellet from the 3 L batch fermentation was processed. The pellet was processed and analyzed by Coomassie staned gel and Western blot, as shown in FIGS. 12A-B. Recombinant human ACE2 protein was quantified compared to human ACE2 standard using ImageJ software.

FIG. 12A shows a photo of a Coomassie stained gel with ACE2 standards and both soluble and insoluble fractions of cell lysates from FBB_p020 production batches. Lane 1 and 2 shows two lots of soluble fraction, lanes 3 and 4 show 2 lots of solubilized inclusion bodies (SIB) PBS dialyzed, lanes 5, 6, 7 show ACE2 standards loaded at 0.8 ug, 0.4 ug, 0.1 ug, respectively, Lane 8 shows Thermo Scientific Spectra Multicolor Broad Range Protein Ladder with bands at top to bottom ˜260 kDa, ˜140 kDa, ˜100 kDa, ˜70 kDa, ˜50 kDa, ˜40 kDa, ˜35 kDa, ˜25 kDa, ˜15 kDa, and ˜10 kDa. Lane 9 and 10 show soluble fractions, lanes 11 and 12 show SIB PBS dialyzed.

FIG. 12B shows a photo of a Western blot using anti-His tag probed membrane. The antibodies used to probe and develop the blot above were: primary=anti-His antibody, secondary=anti-mouse HRP conjugated. Lanes 1-12 are the same as shown for FIG. 12A.

FIG. 12C photo shows a Western blot using anti-ACE2 probed membrane. The antibodies used in the western blot above are; primary=anti-ACE2 (anti-ACE2 polyclonal Antibody (PA5-20045, Invitrogen) developed using immunogen synthetic peptide corresponding to amino acids near N-terminus of human ACE2 purified by antigen affinity chromatography), secondary=anti-rabbit IgG. The lanes in the blots are the same as the Coomassie stained gel.

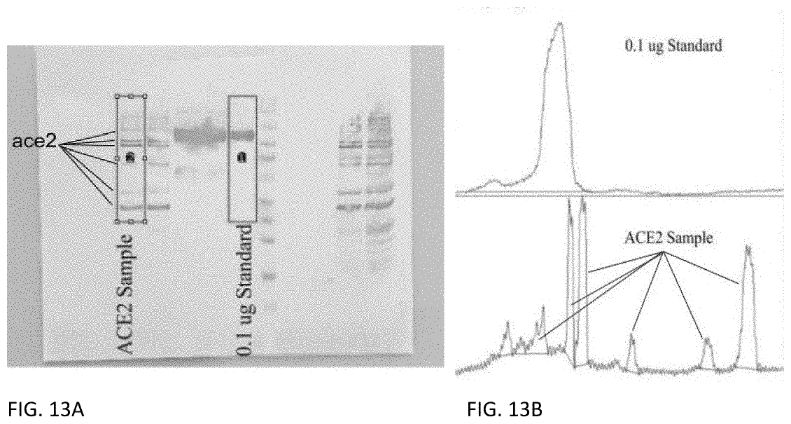

FIG. 13A-B show the Western blot (A) and histogram (B) produced by each box in FIG. 13A using ImageJ software. The standard peak in FIG. 13B (top) was quantified by drawing a line under the peak and measuring the area which corresponds to the band on the Western blot. The sample was measured by taking a histogram of the entire lane and comparing the intensity of the bands against the standard. The areas of the peaks were used to quantify the amount of ACE2 using ImageJ software as shown in Table 7. Total recombinant human ACE2 protein was calculated as 0.313 mg/mL from solubilized inclusion bodies.

TABLE 7
Band Intensity and Concentration Calculations from ImageJ Analysis

| | | | | | | | | total ug | |
|---|---|---|---|---|---|---|---|---|---|
| | | Volume | Total | Ratio | ug | | Total | for 400 uL | |
| | | in Gel | Ace2 | (ug per | per | | uL of | straight | |
| Band | Intensity | (uL) | (ug) | intensity) | band | ug/uL | sample | sample | ug/mL |
| ACE2 | 16565.028 | n/a | 0.1 | 6.04E−06 | 0.100 | n/a | n/a | n/a | n/a |
| STD-0.1 ug | | | | | | | | | |
| Sample | 671.799 | 0.2 | ? | 6.04E−06 | 0.004 | 0.020 | 400 | 8.11 | 20.3 |
| Band-1 | | | | | | | | | |
| Sample | 323.678 | 0.2 | ? | 6.04E−06 | 0.002 | 0.010 | 400 | 3.91 | 9.8 |
| Band-2 | | | | | | | | | |
| Sample | 1374.527 | 0.2 | ? | 6.04E−06 | 0.008 | 0.041 | 400 | 16.60 | 41.5 |
| Band-3 | | | | | | | | | |
| Sample | 2905.456 | 0.2 | ? | 6.04E−06 | 0.018 | 0.088 | 400 | 35.08 | 87.7 |
| Band-4 | | | | | | | | | |
| Sample | 4342.92 | 0.2 | ? | 6.04E−06 | 0.026 | 0.131 | 400 | 52.43 | 131.1 |
| Band-5 | | | | | | | | | |
| Sample | 768.92 | 0.2 | ? | 6.04E−06 | 0.005 | 0.023 | 400 | 9.28 | 23.2 |
| Band-6 | | | | | | | | | |
| Total | n/a | 0.2 | n/a | n/a | 0.063 | 0.314 | 400 | 125.41 | 313.5 |

A total of 25 chickens were to be inoculated and each chicken required at least 200 ug of protein in a 0.5 mL injection volume. The protein was mixed with an equal amount of Freund's Adjuvant which decreased the concentration by half. Therefore the target protein concentration had to be at least 800 ug/mL to deliver 200 ug per chicken. It was decided to increase the 800 ug/mL by 20% to 1000 ug/mL to account for any margin of error in the quantitative Western blot. The starting protein concentration was taken from the total protein concentration in Table 7. The starting protein solution was diluted in a large volume of PBS, therefore 31.9 mL of the protein suspension was spun down, supernatant removed and the protein pellet was resuspended in 10 ml of PBS for the final inoculum. Table 8 shows the calculations for diluting the recombinant human ACE2 proteins for chicken inoculations. Chickens were inoculated as described herein.

TABLE 8
Final Calculation for Chicken Inoculation

| Target | Resuspension | Starting | Starting Protein |
|---|---|---|---|
| Concentration | Final | Protein | Concentration |
| ug/mL | Volume (mL) | Volume (mL) | (ug/mL) |
| 1000 | 10 | 31.9 | 313.5 |

### Example 15. Spray Dried Egg Powder

Spray dried whole egg powder formulations were prepared as follows. Shell eggs were collected from immunized chickens, washed and broken. Whole immune egg was recovered and used as follows. Amounts of each material were based on weight of whole egg, assuming 26 wt % solids and 74 wt % liquids. Formulation is shown in Table 9.

TABLE 9
Formulation for Spray dried Whole Egg Powder

| | Parts by | | | | | |
|---|---|---|---|---|---|---|
| Material | weight (pbw) | | Range | | Purpose | Notes |
| Whole egg | 100 | pbw | 90-110 | pbw | Antibody | Assuming 26% |
| | | | | | source | Solids + 74% |
| | | | | | | Liquids |
| trehalose | 26 | pbw | 10-50 | pbw | stabilizer | 1:1 w/w of |
| | | | | | | trehalose:egg |
| | | | | | | solids |
| Silicon | 0.52 | pbw | 0.2-0.7 | pbw | glidant | 1% of solids |
| dioxide | | | | | | |
| Benzyl | 0.26 | pbw | 0.1-0.5 | pbw | preservative | 0.5 wt % of |
| alcohol | | | | | | solids |
| Distilled | 158 | pbw | 50-250 | pbw | diluent | 75:25 g water:g |
| water | | | | | | total solids |

All components in Table 9 were mixed with an immersion blender until just combined. The formulation was run through a GB22 Yamato spray dryer while being continuously stirred using a magnetic stir bar and plate to provide spray dried whole egg powder.

### Example 16. Orally Disintegrable Tablet Formulations

Dissolvable tablet formulations were prepared as follows. The active pharmaceutical ingredient (API) was spray dried whole egg powder of example 13. A 1:1:1 mixture by weight of spray dried whole egg powder/FIRMAPRESS® pharmaceutical grade excipient powder/dextrose monohydrate was blended with mint flavoring. FIRMAPRESS® contains microcrystalline cellulose as a solid diluent, magnesium stearate as a lubricant, silicon dioxide as a glidant, and dicalcium phosphate as a filler. Specifically, 5.0 gram of API+5.0 gram of FIRMAPRESS®+5.0 gram of Dextrose monohydrate were weighed and homogenized into one mixture. Then 12 drops of mint extract were added and homogenized. Two batches were generated using this formulation: one using baby blue FIRMAPRESS® and the other using white FIRMAPRESS®. A TDP 5 desktop tablet press (LFA Machines Oxford LTD) was employed to prepare tablets. Twenty-four tablets of each color were generated with an average weight of 515 mg per tablet. The tablets were firm (not easily crushed with applied pressure between fingers) and tasted subtly sweet with hints of mint. The API is a very compressible material and will likely form suitable tablets with any weight ratio equal to or exceeding 1:0.5 (API: Excipients), or 1:0.5-1:10, or 1:1 to 1:5, or 1:1 to 1:3.

Formulations:

The API is adjusted based on specific IgY in each spray-dried batch. For example, if 1 egg contained 2 mg of anti-RBD antibodies, and that egg+trehalose generated 25 grams of powder, 1 gram of powder would contain 80 ug of anti-RBD antibodies. This 1 egg could be used to generate (using a 1:1:1.5 ratio): 100 (875 mg) tablets, each with 20 ug of anti-RBD antibodies or 50 (1750 mg) tablets, each with 40 ug of anti-RBD antibodies. When, using a 1:1:1 ratio of API/, 100 (750 mg) tablets, each with 20 ug of anti-RBD antibodies or 50 (1500 mg) tablets, each with 40 ug of anti-RBD antibodies. The formulation is determined based on the amount of specific IgY within each batch and the target dose for each protein. Tablets were stored at room temperature in airtight bags.

In vitro reactivity assays are performed by dissolving 1 gram of tablet sample in 9 mL of PBS, mixing with equal volume of chloroform and extracting by mixing at 20 deg C. while stirring for 30 min. The mixture is centrifuged at 2,000 g for 10 min, water soluble fraction supernatant containing the IgY is collected and analyzed using specific IgY ELISA, and total IgY ELISA.

### Example 17. IgY Water Soluble Fraction from Raw Egg Yolk

An IgY-rich water soluble fraction from raw egg yolk was prepared for the processing of a 50-egg batch, but can be scaled up (or down). The protocol was adapted from Hodek et al., 2013, “Optimized Protocol of Chicken Antibody (IgY) Purification Providing Electrophoretically Homogenous Preparations.” Int. J. Electrochem. Sci. 8:113-124. The method eliminates the need for precipitation, centrifugation, and the resuspension of pelletized proteins.

A modified IgY extraction method was developed to expedite processing and production. Shell eggs are washed in 1% bleach solution, broken, and egg yolk separated from the white. The egg yolk is broken and diluted with tap water at room temperature, and diluted at either 1:4 or 1:7 with water, and adjusted to pH 5.0 with 0.5 M HCl to provide diluted yolks. The diluted yolks were frozen in dry ice ethanol bath (−72 deg C.) for about 30 min. until frozen solid. The frozen material is then thawed through funnels and paper filtration using two coffee filters to collect the filtrate. The resulting IgY WSF is a transparent IgY-rich solution that is used for product development.

### Example 18. Nasal Spray Solution from IgY Water Soluble Fraction

A functional nasal spray containing anti-COVID-19 antibodies from an IgY-rich water soluble fraction (WSF) of example 17 was prepared. The nasal spray is designed to promote passive immunity for 8-12 hours when administered. The number of functional nasal sprays that can be generated is dependent on the concentration and volume of the prepared WSF. The functional nasal sprays generated according to this SOP were 15 mL each.

A trehalose-based nasal spray solution was prepared as follows. Equipment and glassware were sanitized with 70% ethanol. 236.6 mL of WSF containing the IgY was added to a 500 mL beaker with a magnetic stir bar stir bar was added to the beaker. The solution was stirred magnetically while adding 2.1 g of trehalose, 2.1 g of NaCl, and 2.1 g of Baking Soda. The solution was stirred until the solutes have completely dissolved (about one minute). 0.3% (0.7 mL) of benzyl alcohol was added to the solution with stirring until completely dissolved. Test and record the pH of the formulation. Add flavoring as desired. Generate the functional nasal spray by aliquoting 15-mL fractions into nasal spray bottles. Cap each bottle and mark with an expiration date.

### Example 19. Nasal Spray Solution from Purified IgY with Pelleted Extraction

A method for obtaining purified IgY was performed by the method of Hodek et al., 2013, “Optimized Protocol of Chicken Antibody (IgY) Purification Providing Electrophoretically Homogenous Preparations.” Int. J. Electrochem. Sci. 8:113-124.

An IgY-rich water soluble fraction from raw egg yolk was prepared by the method of example 15 for the processing of a 50-egg batch. Briefly, shell eggs were washed in 1% bleach solution, broken, and the egg yolk was separated from the albumen (egg white), and the egg yolks were pooled into a 500 mL graduated beaker. The yolk volume of the pooled 50-yolk batch was measured and recorded. The pooled yolk was transferred to a 4 L pitcher, diluted to 1:7 with tap water and adjusted to pH 5.0 with 0.5 M HCl, to form a diluted egg yolk solution. The egg yolk solution was frozen in an ethanol/dry ice bath for about 30 min until solid. The frozen egg yolk was allowed to thaw at room temperature with gravity paper filtration to form a IgY-rich WSF.

Sodium chloride was added to the WSF to make a 8.8% NaCl (w/vol) solution with magnetic stirring at room temperature and adjusted to pH 4.0 with addition of 0.5 M HCl. The mixture was stirred for 2 h. Mixture was centrifuged at 3,200 rcf at 4 deg C. for 40 min. The supernatant was discarded and the pellets containing IgY were retained.

A trehalose based solution was prepared using 750 mL distilled water, 6.66 g trehalose, 6.66 g NaHCO₃ baking soda, and 6.66 g NaCl. The mixture was magnetically stirred until dissolved. 2.25 mL benzyl alcohol was added (0.3%) and solution was stirred until completely dissolved. 100 mL trehalose solution was added to each pellet containing IgY. Resuspended pellets were pooled, stirred and pH tested. Flavoring was added to the solution, which was aliquoted into 15 mL bottles.

### Example 20. ELISA Analysis: Inhibition of hACE2:RBD Binding by Isolated IgY Antibodies or Spray Dried Eggs

The SARS-CoV-2 primary mechanism leading to infection involves binding of Spike protein to human ACE2 receptors that are expressed on cells within the upper respiratory tract. Quinlan et al., The SARS-CoV-2 Receptor-Binding Domain Elicits a Potent Neutralizing Response without Antibody-Dependent Enhancement. Microbiology; 2020. doi:10.1101/2020.04.10.036418.

The ability of Sample material such as isolated IgY antibodies or spray dried immune egg yolk to inhibit SARS-CoV-2 spike protein RBD:ACE2 binding was tested using two different commercial ELISA assays.

A commercial SARS-CoV-2 Inhibitor Screening Kit (ACRO EP-105, ACROBiosystems Inc.) was employed according to manufacturer's protocol with minor variations. Briefly, ELISA plates were coated with SARS-CoV-2 S protein RBD. 50 uL antibody test material or reference material was added to the wells of coated plate; then 50 uL biotinylated human ACE2 was added to coated plate. Streptavidin-HRP was added, followed by TMB (3,3′,5,5′-tetramethylbenzidine) substrate, quench, and plates were read at 450 nm using UV/Vis microplate spectrophotometer. The ability of the isolated IgY or immune egg to inhibit S protein RBD:ACE2 binding was determined by comparing OD450 readings among experimental groups.

Spray-dried immune eggs from various color groups (i.e., different inoculations) and isolated IgY derived therefrom were reconstituted in distilled water and diluted to various concentrations to determine whether the sample materials could inhibit Human ACE2 binding to SARS-CoV-2 RBD protein. Spray-dried egg samples and isolated IgY samples were shown to inhibit biotinylated Human ACE2 from binding to SARS-CoV-2 Spike protein RBD, as shown in following examples.

A commercial GenScript SARS-CoV-2 Surrogate Virus Neutralization Test (sVNT) C-Pass™ Kit (GenScript Biotech Corporation) was employed according to manufacturer's protocol for detection of neutralizing antibodies. The SARS-CoV-2 sVNT Kit is a blocking ELISA detection tool, which mimics the virus neutralization process. The kit contains Horseradish peroxidase (HRP) conjugated recombinant SARS-CoV-2 RBD fragment (HRP-RBD) and the human ACE2 receptor protein (hACE2). The protein-protein interaction between HRP-RBD and hACE2 can be blocked by neutralizing antibodies against SARS-CoV-2 RBD. Briefly, ELISA plates are pre-coated with ACE2 protein. The Test Samples or Controls are pre-incubated with HRP-RBD to allow neutralizing antibodies to bind to the HRP-RBD. The mixture is added to the capture plates. The unbound HRP-RBD as well as any HRP-RBD bound to non-neutralizing antibody is captured on the plate, while the neutralizing antibodies-HRP-RBD complexes remain in supernatant and get removed during washing. After wash steps, TMB solution is added, making the color blue. By adding the Stop solution, the reaction is quenched and the color turns yellow. Absorbance of the final solution is read at 450 nm in a UV/Vis microtiter spectrophotometer plate reader. The absorbance of the sample is inversely dependent on the titer of the anti-SARS-CoV-2 neutralizing antibodies.

### Example 21. Inhibition of RBD:hACE2 Binding by Anti-RBD IgY Antibodies

Recombinant COVID-19 Spike protein receptor binding domain (RBD), His-tagged, was obtained from Creative Biomart® Spike-190V. Briefly, the protein was obtained by use of a DNA sequence encoding the SARS-CoV-2 (2019-CoV) RBD which was fused to His-tag at C-terminus and expressed in human HEK293 cells. The recombinant protein includes 234 amino acids comprising SARS-CoV-2 (2019-nCoV) Spike Protein (RBD) (YP_009724390.1; Arg319-Phe541) (SEQ ID NO: 36).

Chickens were inoculated with 125 micrograms of the SARS-CoV-2 S1 Subunit RBD protein in PBS with Freund's complete adjuvant on Day 0, and boosted on a biweekly schedule: Day 14, Day 28, and Day 42 using the SARS-CoV-2 S1 Subunit RBD protein in PBS with Freund's incomplete adjuvant.

To test IgY present in egg yolk over time, eggs from Day 0 (prior to inoculation), Day 14, Day 21, Day 28, Day 35, and Day 50 were collected and processed. Eggs were also collected from chickens not inoculated with a target protein (non-targeted) and processed for comparison.

The eggs were washed, broken and the egg yolks separated from the egg whites to undergo an IgY extraction process starting from a pool of 24 or 30 egg yolks for each collection date.

Briefly, egg yolks were diluted 8 fold in water and adjusted to pH 5 using HCl. The solution was frozen at −20° C. until completely frozen, then placed on a filtration apparatus using a cellulose filter where the frozen egg yolk is allowed to thaw overnight at room temperature. The water-soluble fraction containing the IgY antibodies is found in the filtrate. Sodium chloride is added to filtrate and solution adjusted to pH 4. After mixing for 2 h, the IgY is precipitate is collected by centrifugation and resuspended in PBS. Hodek et al., 2013 Int J Electrochem Sci 8:12. The resulting extracted IgY samples were used for testing.

Chicken blood samples from Day 15 and Day 29 were also collected and the serum was isolated. Control blood samples were also collected from chickens not inoculated with a target protein (non-targeted) and processed for comparison. The resulting serum samples were used for testing.

Commercial ELISA kits ACRO EP-105 and GenScript sVNT inhibition screening kits were performed according to manufacturer instructions, with slight adjustments, as described in Example 20.

Table 10 shows inhibition of RBD:ACE2 Binding by anti-RBD IgY Extracted from Egg Yolks at Day 0-Day 21 Post-RBD Inoculation Compared to IgY Extracted from Non-Targeted Egg Yolks (EP-105 Kit).

TABLE 10
Average percent inhibition ± standard deviations of the anti-RBD
IgY samples from Day 0 to Day 21 following initial RBD inoculation and
the non-targeted IgY sample, tested in the EP-105 ACRO inhibition assay.

| | Extracted | Extracted | Extracted | Extracted |
|---|---|---|---|---|
| | anti-RBD | anti-RBD | anti-RBD | Non-Targeted |
| | IgY (Day 0) | IgY (Day 14) | IgY (Day 21) | IgY |
| | Concentration of Total IgY | | | |
| Dilution | 52.1 mg/mL | 48.4 mg/mL | 69.2 mg/mL | 52.5 mg/mL |
| Factor | Inhibition (%) ± Standard Deviation (%) | | | |
| Stock | 21.74 ± 1.59 | 75.83 ± 1.51 | 99.80 ± 0.02 | 21.57 ± 0.33 |
| 1:2 | 11.13 ± 0.61 | 54.63 ± 0.31 | 99.67 ± 0.04 | 15.63 ± 2.69 |
| 1:5 | 10.72 ± 3.48 | 40.98 ± 5.12 | 98.26 ± 0.20 | 9.94 ± 1.98 |
| 1:10 | 10.59 ± 2.08 | 28.00 ± 3.14 | 96.10 ± 0.77 | 6.56 ± 1.41 |
| 1:100 | 7.98 ± 3.18 | 9.87 ± 2.32 | 34.26 ± 1.51 | 3.65 ± 0.27 |
| 1:1,000 | 4.41 ± 1.04 | 3.73 ± 0.75 | 5.55 ± 1.08 | 0.95 ± 0.79 |
| 1:10,000 | 0.39 ± 0.22 | 0.64 ± 1.08 | 2.73 ± 0.31 | 2.48 ± 0.59 |
| 0 | 0.00 ± 1.04 | 0.00 ± 1.43 | 0.37 ± 0.39 | 0.55 ± 1.63 |

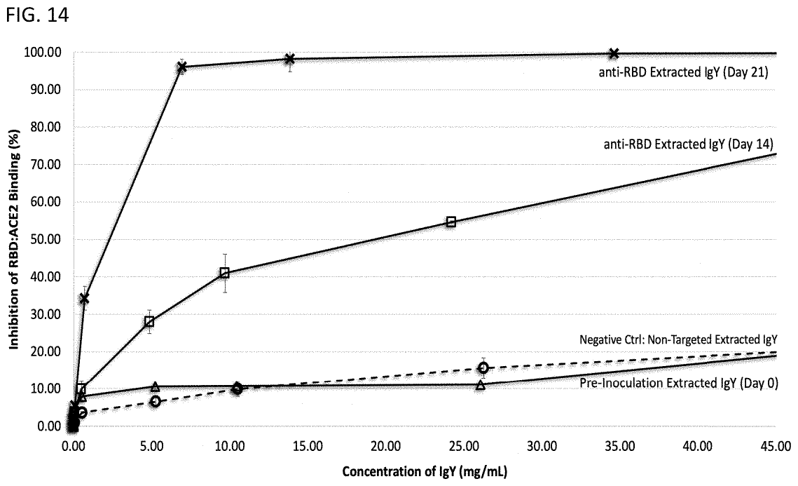

The contents of Table 10 are plotted in FIG. 14 showing a graph of percent inhibition of RBD:ACE2 binding versus IgY concentration (mg/mL) of anti-RBD IgY Test samples extracted from chicken egg yolks in the first three weeks post-inoculation compared to negative control extracted non-targeted IgY, by ELISA using ACRO Biosystems EP-105 Kit. A significant increase in titer from Day 0 to Day 21 was observed.

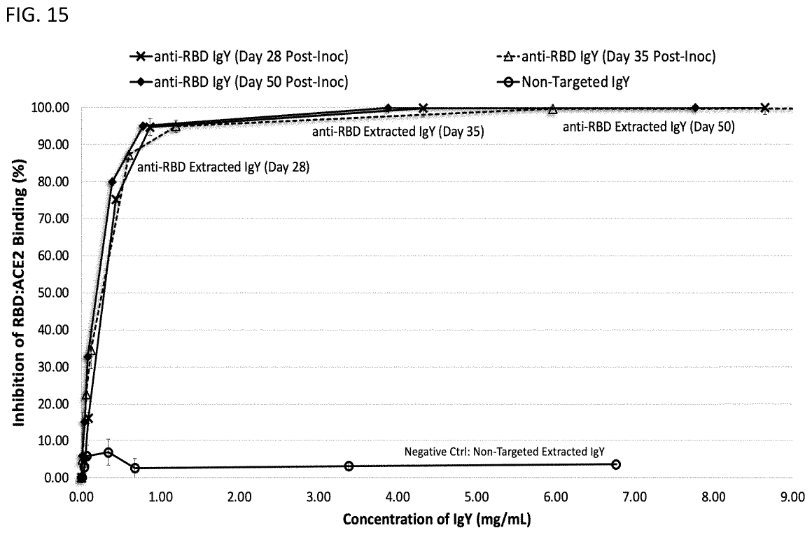

Extracted anti-RBD IgY test samples from Day 28, Day 35, and Day 50 were tested in the EP-105 ACRO ELISA inhibition assay compared to Negative Control extracted IgY from non-targeted egg yolks. FIG. 15 shows a graph of percent inhibition of RBD:hACE2 binding versus anti-RBD IgY concentration (mg/mL) at Day 28, Day 35, and Day 50 post-inoculation, compared to negative control extracted non-targeted IgY, by ELISA using ACRO Biosystems EP-105 Kit. Note the change in the x-axis from FIG. 14 to FIG. 15 to accommodate the increase in RBD-specific IgY titer. A significant increase in titer occurred from Day 0 until Day 28 and titer was maintained through at least day 50, as shown in FIG. 15.

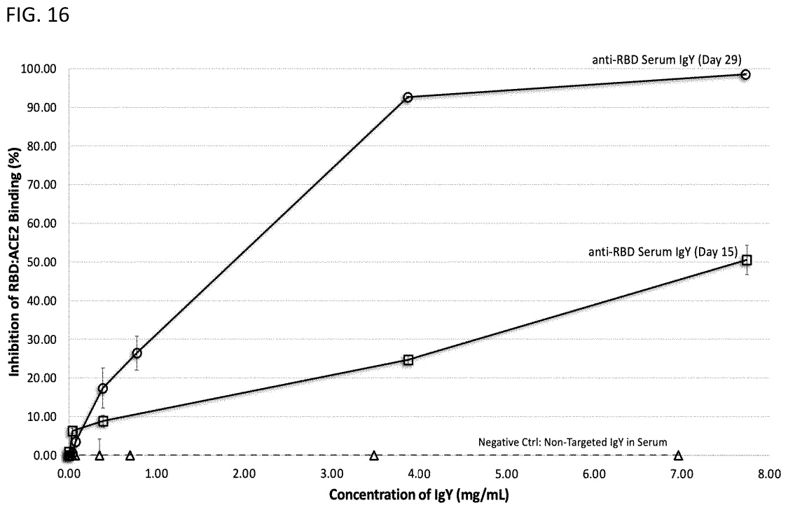

Inhibition of RBD:ACE2 binding by anti-RBD IgY from inoculated Chicken serum was compared to negative control non-targeted IgY in non-inoculated chicken serum by ELISA (EP-105 Kit) at day 15 and day 29. Results are shown in FIG. 16 showing the percent inhibition of RBD:ACE2 binding vs. anti-RBD IgY concentration (mg/mL) in serum extracted from inoculated chickens at Days 15 and 29 post-inoculation, tested with the ACRO Biosystems ELISA EP-105 Kit. An increase in serum titer is seen between Day 15 and Day 29.

### Example 22. Increase in IgY Titer Using Protein-Based Inoculation

In this example, hen chickens were inoculated with SARS-CoV-2 S1 Subunit RBD protein. Hen chickens were inoculated with 125 μg of SARS-CoV-2 S1 Subunit RBD protein in Freund's Complete Adjuvant on Day 0 and boostered with the same quantity of protein in Freund's Incomplete Adjuvant on Days 14, 28 and 42. Hens produced escalating titers of specific IgY targeted to RBD in egg and serum.

IgY antibodies were extracted from raw egg yolks and whole blood samples collected at weekly time points following inoculation. The IgY extractions and serum samples were tested using an indirect ELISA format to determine whether the sample materials contained active anti-RBD IgY capable of binding to the SARS-CoV-2 RBD protein.

Within a month of first inoculating chickens with the SARS-CoV-2 S1 Subunit RBD protein, polyclonal antibodies against the full-length RBD antigen were recovered and qualitative binding activity against the RBD protein measured. The specific anti-RBD IgY antibodies bind RBD protein in vitro and were used to block the RBD protein:ACE2 receptor interaction, for example as shown in example 21.

Prior to inoculation, two groups of 25 Leghorn chickens each were isolated from one another. Chickens in one group received an initial inoculation (Day 0) of 500 μL containing ˜110 μg of SARS-CoV-2 S1 Subunit RBD protein purified from HEK cells (Creative Biomart) in Freund's Complete Adjuvant. This same group received three separate booster inoculations (Day 14, Day 28 and Day 42) of 500 μL containing 125 μg of the same SARS-CoV-2 RBD protein purified from HEK cells (Creative Biomart) in Freund's Incomplete Adjuvant. Eggs collected on Days 0, 14, 21, 28, 35, and 50 following the first inoculation were processed and tested. Blood samples were collected from the same group of chickens on Days 15 and 29 following the first inoculation. The other group of 25 Leghorn chickens served as a control group (Non-Targeted) and were not inoculated with any protein or adjuvant. Materials to prepare inoculum are shown in Table 11.

TABLE 11
Materials for inoculum

| Item | Description | Manufacturer | Part # |
|---|---|---|---|
| Recombinant COVID-19 | Derived from HEK cells, | Creative Biomart | Spike-190V |
| Spike protein receptor | Lot#: PLN5042003, | | |
| binding domain | reported at 5.8 mg/mL by | | |
| (RBD), His-tagged | manufacturer, suspended | | |
| | in PBS, pH = 7.4 | | |
| Freund's Adjuvant, | Lot#: SLCD6299 | Sigma | F5881-10X10ML |
| Complete | | | |
| Freund's Adjuvant, | Lot#: SLCD0721 | Sigma | F5506-10ML |
| Incomplete | | | |

RBD recombinant protein (Creative Biomart Cat #Spike-190V) was concentration-verified by NanoDrop™ A280. The volumes of protein resuspension, PBS and Freund's adjuvant were calculated as shown in Table 12, measured, and combined into a single formulation containing two distinct liquid layers. A water-in-oil emulsion was formed between the aqueous protein solution and Freund's adjuvant by forcibly mixing the formulation between two syringes. After the emulsion was properly formed into one cohesive formulation, it was administered to the chickens via an intramuscular inoculation.

TABLE 12
Inoculation Mixtures for each 25-chicken injection

| Inoculum Component | Day 0 | Day 14 | Day 28 | Day 42 |
|---|---|---|---|---|
| PBS (mL) | 6.17 | 6.08 | 6.17 | 6.17 |
| 5.8 mg/mL RBD (mL) | 0.58 | 0.67 | 0.58 | 0.58 |
| Freund's Complete | 6.75 | — | — | — |
| Adjuvant (mL) | | | | |
| Freund's Incomplete | — | 6.75 | 6.75 | 6.75 |
| Adjuvant (mL) | | | | |
| Total Volume (mL) | 13.5 | 13.5 | 13.5 | 13.5 |

Egg and blood samples were collected and processed as follows. Eggs were collected on Days 0, 14, 21, 28, 35 and 50 following the first inoculation. Eggs were stored at 4° C., cleaned, broken, yolks separated and processed using the Hodek IgY extraction method. Briefly, the egg yolks were diluted 8-fold in water and adjusted to pH of 5 with HCl. The solution was frozen at −20° C., placed in a filter apparatus and allowed to thaw overnight at room temperature. The filtrate containing the water-soluble fraction was collected. The next day, NaCl was added to the water soluble fraction and adjusted to a pH 4. The IgY was allowed to precipitate during a 2 h mixing step, then centrifuged, supernatant removed and the pellet containing the purified IgY was resuspended in PBS.

The IgY extractions were analyzed using a ThermoScientific™ NanoDrop™ OneC Microvolume UV-Vis Spectrophotometer to determine the total IgY concentration within each sample by A280. IgY antibodies sourced from raw egg yolks and serum isolated from chicken blood were tested in an anti-RBD IgY:RBD indirect binding assay to qualitatively measure RBD binding activity. In brief, RBD was coated with a concentration of 0.5 μg/mL onto a 96-well microplate and incubated for 12-16 hours. After blocking with BLOKHEN (Aves Labs), Hodek extracted IgY and serum samples were added to the RBD-coated wells in a dilution series. After a 1-hour incubation followed by washing, goat anti-Chicken IgY-HRP secondary antibody solution was added to the wells. After another 1-hour incubation followed by washing, TMB substrate was added, causing a colorimetric reaction. Sulfuric acid was added to stop the reaction and the plate was read at 450 nm to measure the Optical Density (OD). Resulting OD measurements were compared across samples to qualitatively assess the change in RBD-reactivity over time following RBD-inoculation.

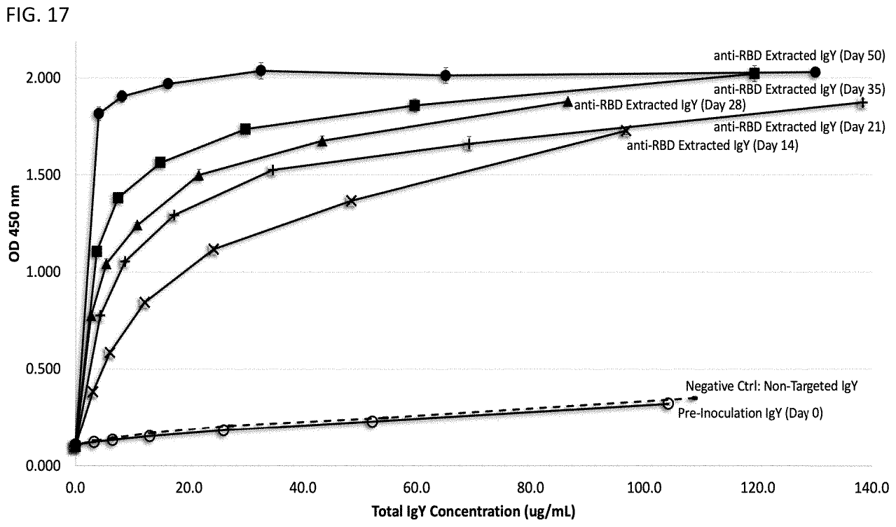

Results. Total egg yolk IgY was fairly consistent through the experimental time period. Total egg yolk IgY was 52.1 mg/mL (day 0), 48.4 mg/mL (day 14), 69.2 mg/mL (day 21), 43.3 mg/mL (day 28), 59.7 mg/mL (day 35), and 65.1 mg/mL (day 50). Extracted non-targeted egg yolk exhibited 54.4 mg/mL total IgY. In contrast, FIG. 17 shows ELISA specific reactivity of anti-RBD IgY isolated from raw egg yolk against coated RBD protein, collected at varying time points following RBD inoculation, compared to IgY antibodies sourced from non-targeted chicken eggs. OD at 450 nm was measured and plotted against the concentration of total IgY measured by ThermoScientific™ NanoDrop™ OneC Microvolume UV-Vis Spectrophotometer. Each sample was plated in duplicate and the averages and standard deviations were calculated. Eggs collected pre-inoculation (Day 0) and non-targeted IgY antibodies show little to no binding activity. The RBD binding activity for each sample dilution series shows a steady increase in RBD-specific antibody titer from Day 14 to Day 50.

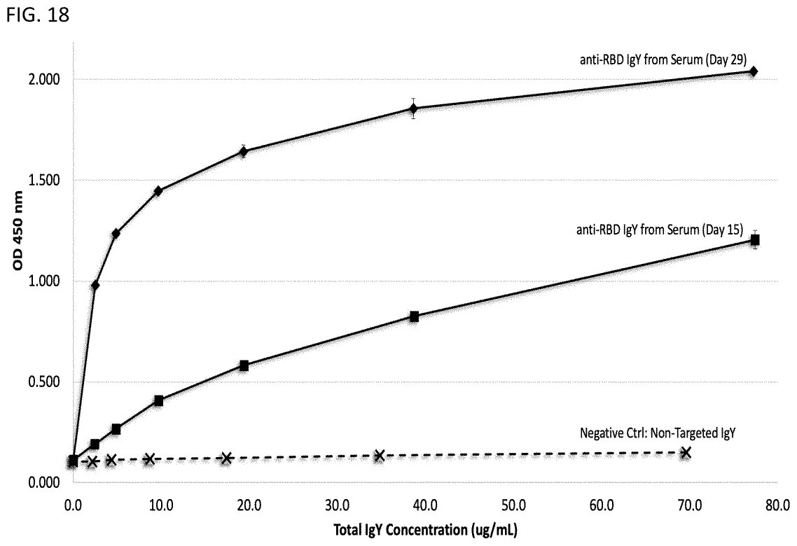

FIG. 18 shows reactivity of anti-RBD IgY from serum of inoculated chickens against coated RBD protein. Blood was collected at day 15 and 29 following initial RBD inoculation. Reactivity of serum sourced from non-targeted chicken blood was used as negative control. Samples were run in duplicate. Error bars indicate standard deviation. Serum IgY antibodies exhibited ELISA reactivity similar to the egg yolk IgY. Serum IgY exhibited escalating specific ELISA reactivity to the inoculated target protein (RBD) in vitro from day 15 to day 29. The inoculation platform allows for time-sensitive, highly-scalable polyclonal antibody production against antigens of interest such as RBD.

### Example 23. Antiviral Activity of Anti-RBD IgY Against SARS-CoV-2 Selected Variants from UK, South Africa, California, New York, and Brazil

In this example, anti-RBD IgY was tested for its ability to bind to three SARS-CoV-2 mutant RBDs present in the variants from the UK, South Africa and Brazil and the Spike S1 protein from the South African variant using an indirect ELISA compared to IgY antibodies from uninoculated chickens (non-targeted).

Competitive ELISA was performed to determine the ability of anti-RBD IgY to inhibit the RBD/S1 mutant:ACE2 binding interaction. A neutralization assay format was employed to determine whether the anti-RBD IgY could inhibit the binding interaction between each mutant protein and human ACE2.

Therapeutics and vaccines are under rapid development to address the SARS-CoV-2 global pandemic. One of the greatest concerns for government and health officials at present is the emergence of variants. Of particular concern are the variants from the UK (B.1.1.7.), South Africa (B.1.351) and Brazil (P.1) variants.

The SARS-CoV-2 Spike S1 RBD (receptor-binding domain) docks with human ACE2 present on epithelial cells at the back of the throat in order to cause infection. Quinlan et al. The SARS-CoV-2 Receptor-Binding Domain Elicits a Potent Neutralizing Response without Antibody-Dependent Enhancement. Microbiology; 2020. doi:10.1101/2020.04.10.036418.

This study demonstrates that polyclonal anti-RBD IgY antibodies generated through the inoculation of chickens with Wuhan RBD protein shown in Example 21 (SEQ ID NO: 36) and processing of collected egg yolks demonstrate high binding affinity to four SARS-CoV-2 Spike/RBD variant proteins. The variant proteins were chosen based on current government and health official concerns regarding the variants originating from the UK (B.1.1.7.), South Africa (B.1.351), and Brazil (P.1). The mutations tested are present in all or some of these three variants and are the primary cause for increased transmissibility and/or reduced efficacy with current therapeutics or vaccines.

Four SARS-CoV-2 RBD/Spike S1 mutant proteins shown in Table 13 were commercially obtained and used in indirect and competitive assay formats for testing anti-RBD IgY antiviral activity with SARS-CoV-2 variants. The RBD [N501Y] mutation is present in the variants from the UK (B.1.1.7.), South Africa (B.1.351) and Brazil (P.1). The RBD [E484K] mutation is present in the B.1.351 and P.1 variants. The RBD [K417N] mutation is present in the B.1.351 variant. The Spike S1 [K417N, E484K, N501Y, D614G] protein is from the B.351 variant. The recombinant proteins include the following amino acid sequences: SARS-CoV-2 RBD [N501Y] mutant protein comprises amino acid sequence of SEQ ID NO: 37; SARS-CoV-2 RBD [E484K] mutant protein comprises amino acid sequence of SEQ ID NO: 38; SARS-CoV-2 RBD [K417N] mutant protein comprises amino acid sequence of SEQ ID NO: 39; and SARS-CoV-2 Spike S1(K417N, E484K, N501Y, D614G) comprises the amino acid sequence of SEQ ID NO: 40. Each of the recombinant proteins further comprise a His tag as shown in Table 13.

TABLE 13
SARS-CoV-2 variant mutant proteins

| Item | Description | Manufacturer | Part # |
|---|---|---|---|
| SARS-CoV-2 (2019- | Derived from HEK cells, | SinoBiological | 40592-V08H82 |
| nCoV) Spike | (YP_009724390.1) (Arg319- | | |
| RBD(N501Y)-His | Phe541(N501Y)) >90% purity | | |
| Recombinant Protein | | | |
| SARS-CoV-2 (2019- | Derived from HEK cells, | SinoBiological | 40592-V08H84 |
| nCoV) Spike | (YP_009724390.1) (Arg319- | | |
| RBD(E484K)-His | Phe541(E484K)) >95% purity | | |
| Recombinant Protein | | | |
| SARS-CoV-2 (2019- | Derived from HEK cells, | SinoBiological | 40592-V08H59 |
| nCoV) Spike | (YP_009724390.1) (Arg319- | | |
| RBD(K417N)-His | Phe541(K417N)) >85% purity | | |
| Recombinant Protein | | | |
| SARS-CoV-2 (2019- | Derived from HEK cells, | SinoBiological | 40592-V08H10 |
| nCoV) Spike | (YP_009724390.1) (Met1- | | |
| S1(K417N, E484K, | Arg685(K417N, E484K, | | |
| N501Y, D614G)-His | N501Y, D614G)) >90% purity | | |
| Recombinant Protein | | | |

### Chicken Inoculation and Sample Preparation

Prior to inoculation, two groups of 25 Leghorn chickens each were isolated from one another. Chickens in one group received an initial inoculation (Day 0) of 500 μL containing ˜110 μg of SARS-CoV-2 S1 Subunit RBD protein purified from HEK cells (Creative Biomart) (SEQ ID NO: 36) in Freund's Complete Adjuvant. This same group received three separate booster inoculations (Day 14, Day 28 and Day 42) of 500 μL containing 125 μg of the same SARS-CoV-2 RBD protein purified from HEK cells (Creative Biomart) in Freund's Incomplete Adjuvant (Tech.004). Eggs collected on Day 50 following the first inoculation were processed and tested. The other group of 25 Leghorn chickens served as a control group (Non-Targeted) and were not inoculated with any protein or adjuvant. Eggs were collected and processed from this group for comparison. IgY antibodies from both groups were extracted from raw egg yolks by Hodek protocol described herein and used for testing.

### Indirect Binding Assay of Mutant Proteins to Anti-RBD IgY Antibodies

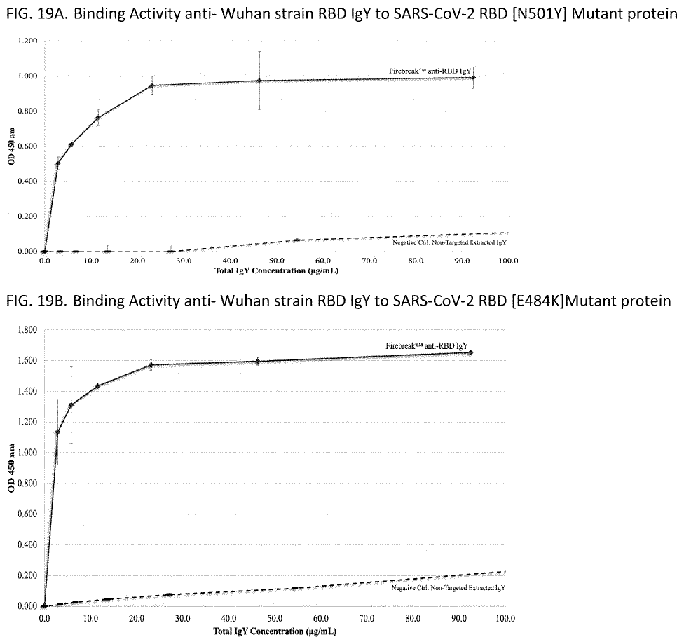

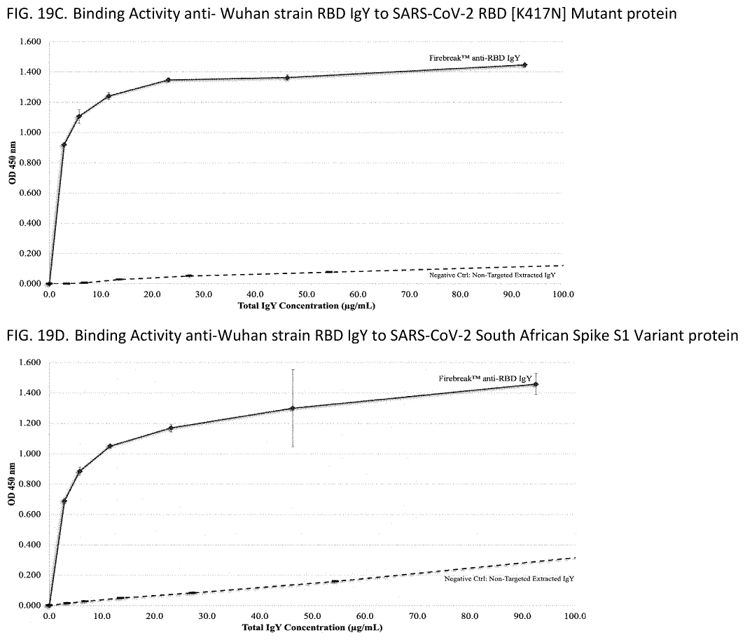

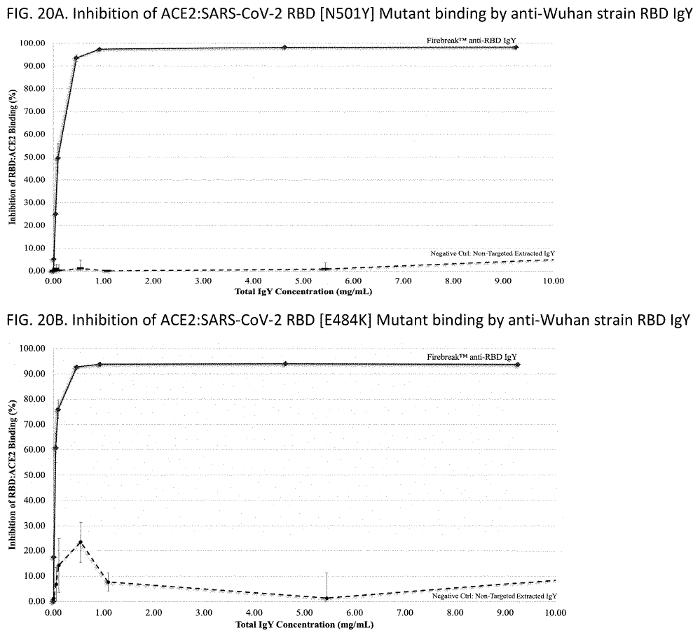

The extracted IgY antibodies were tested in an anti-RBD IgY:RBD indirect binding assay to qualitatively measure RBD binding activity. In brief, the mutant RBD proteins of Table 13 were coated with a concentration of 0.5 μg/mL, and the mutant Spike S1 at 1.0 μg/mL, onto a 96-well microplate and incubated for 12-16 hours. After blocking with BLOKHEN (Aves Labs), Hodek-extracted IgY samples were added to the RBD-coated (or S1-coated) wells in a dilution series. After a 1-hour incubation followed by washing, Goat anti-Chicken IgY-HRP secondary antibody solution was added to the wells. After another 1-hour incubation followed by washing, TMB substrate was added, causing a colorimetric reaction. Sulfuric acid was added to stop the reaction and the plate was read at 450 nm to measure the Optical Density (OD). OD450 was plotted against concentration of total IgY measured by ThermoScientific™ NanoDrop™ OneC Microvolume UV-Vis Spectrophotometer. Resulting OD measurements were compared across samples to qualitatively assess the mutant RBD- or mutant Spike S1-reactivity in Wuhan RBD-inoculated chickens as compared to uninoculated chickens (Non-Targeted). Each sample was run in duplicate. Results are shown in FIG. 19A-D. The extracted anti-SARS-CoV-2 RBD IgY demonstrated high binding affinity for each of the mutant proteins, as compared to IgY antibodies extracted from eggs of uninoculated chickens (non-targeted). Specifically, the extracted anti-Wuhan SARS-CoV-2 RBD IgY bound with high affinity to each of recombinant proteins SARS-CoV-2 RBD [N501Y] mutant protein; SARS-CoV-2 RBD [E484K] mutant protein; SARS-CoV-2 RBD [K417N] mutant protein; and SARS-CoV-2 Spike S1(K417N, E484K, N501Y, D614G).

### ACRO Biosystems SARS-CoV-2 Inhibitor Screening Kit

anti-RBD IgY antibodies were also tested for their ability to prevent each mutant protein from binding to the human ACE2 protein in a competitive assay format. A commercial ELISA ACRO Biosystems SARS—CoV-Inhibitor Screening Kit (EP-105) was employed.

Before performing the ACRO's SARS-CoV-2 inhibition assay, each mutant protein of Table 13 was tested in an indirect assay to determine its ability to bind to human ACE2. As the EP-105 kit was designed using the Wuhan strain RBD, measuring this reactivity was performed to confirm the ability of each mutant to perform in the competitive assay format. All mutants exhibited confirmed reactivity to human ACE2 (data not shown) and assay parameters were chosen based on the findings (Table 14A).

TABLE 14A
EP-105 Competitive Assay Parameters for Each Mutant Protein

| Mutant | ACE2 Concentration | TMB Reaction Time |
|---|---|---|
| RBD [N501Y] | 0.12 μg/mL | 7.5 minutes |
| RBD [E484K] | 0.25 μg/mL | 30 minutes |
| RBD [K417N] | 0.25 μg/mL | 30 minutes |
| Spike S1 [K417N, E484K, | 0.25 μg/mL | 15 minutes |
| N501Y, D614G] | | |

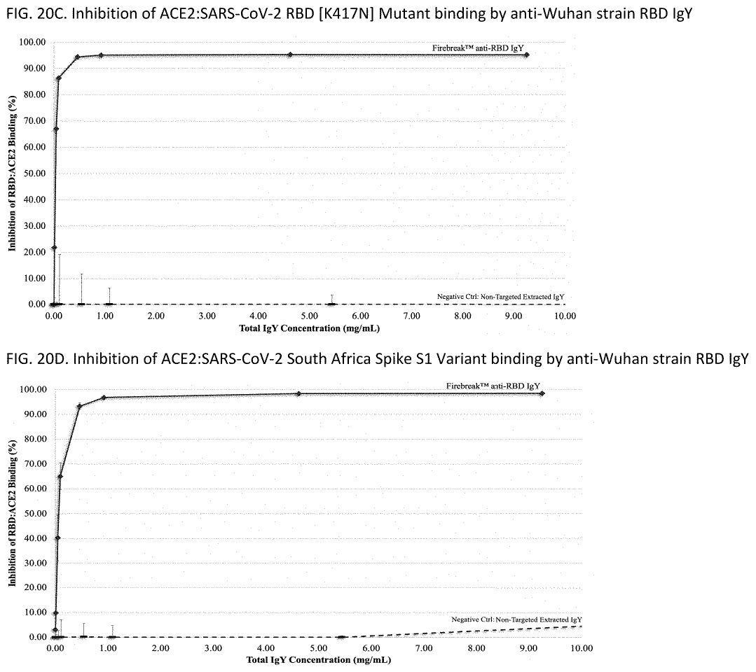

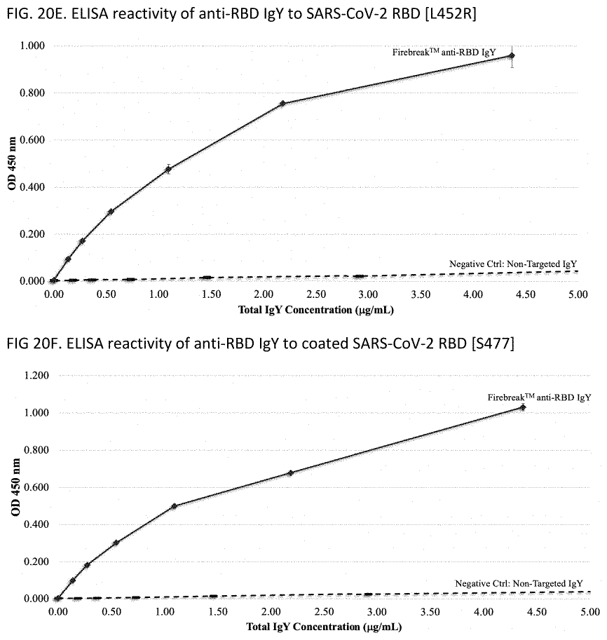

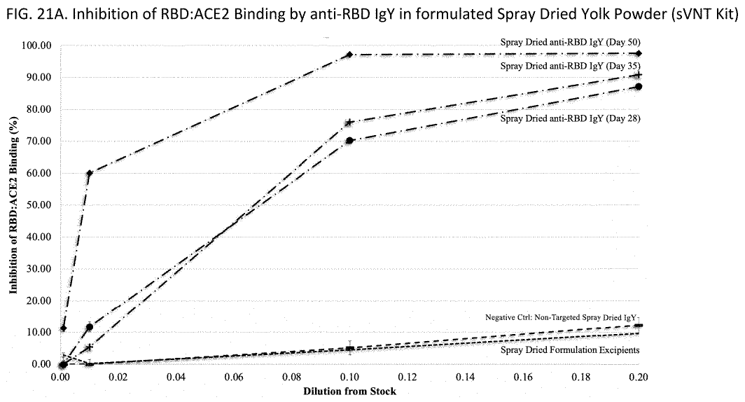

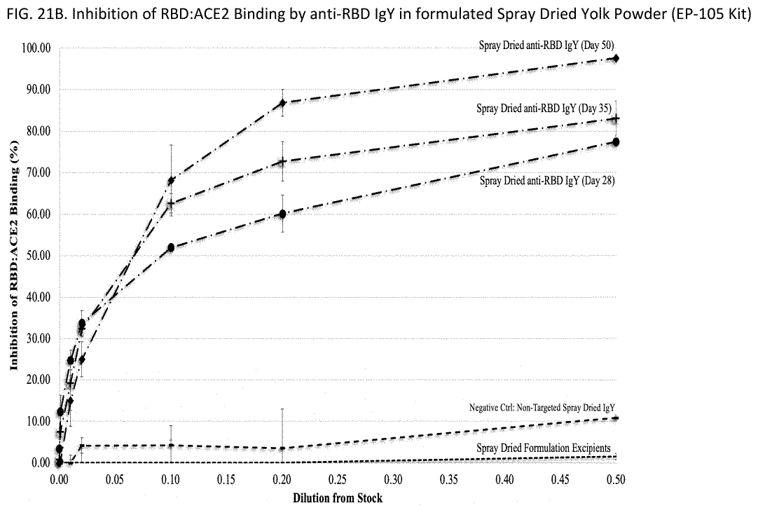

The ACRO EP-105 inhibition screening kit was performed according to manufacturer instructions, with minor variations shown in Table 14. In brief, separate plates were coated with one each of the recombinant SARS-CoV-2 RBD or S1 mutant proteins of Table 13. anti-RBD IgY test samples and biotinylated ACE2 were applied together to the RBD-coated microplate. The biotinylated ACE2 was then probed for by a Streptavidin-HRP conjugated secondary molecule followed by TMB substrate. The absorbance was then measured at 450 nm, and this value corresponds to the binding activity of the RBD and ACE2. The percentage of inhibition was then calculated for each well relative to the absorbance value generated in a Positive Control well without IgY to allow for full RBD:ACE2 binding. Competitive ELISA results are shown in FIGS. 20A-D.

FIG. 20A shows a graph of competitive ELISA showing inhibition of ACE2:SARS-CoV-2 RBD [N501Y] mutant binding by anti-Wuhan strain RBD IgY. The percent inhibition averages as compared to positive control wells without IgY antibody, of the anti-RBD IgY and the non-targeted IgY samples inhibiting the RBD [N501Y] Mutant:ACE2 interaction, was tested in the EP-105 ACRO inhibition assay. Error bars indicate standard deviation. Each sample dilution was tested in duplicate. Very low inhibition was exhibited by negative control non-targeted extracted IgY at all tested concentrations. Greater than about 97% inhibition of ACE2:SARS-CoV-2 RBD [N501Y] mutant binding by anti-Wuhan strain RBD IgY was demonstrated at total IgY concentrations above 1 mg/mL.

FIG. 20B shows a graph of competitive ELISA showing inhibition of ACE2:SARS-CoV-2 RBD [E484K] mutant binding by anti-Wuhan strain RBD IgY. The percent inhibition averages as compared to positive control wells without IgY antibody, of the anti-RBD IgY and the non-targeted IgY samples inhibiting the RBD [E484K] Mutant:ACE2 interaction, was tested in the EP-105 ACRO inhibition assay. Error bars indicate standard deviation. Each sample dilution was tested in duplicate. Very low inhibition was exhibited by negative control non-targeted extracted IgY. Greater than about 92% inhibition of ACE2:SARS-CoV-2 RBD [E484K] mutant binding by anti-Wuhan strain RBD IgY was demonstrated at total IgY concentrations above 1 mg/mL.

FIG. 20C shows a graph of competitive ELISA showing inhibition of ACE2:SARS-CoV-2 RBD [K417N] mutant binding by anti-Wuhan strain RBD IgY. The percent inhibition averages as compared to positive control wells without IgY antibody, of the anti-RBD IgY and the non-targeted IgY samples inhibiting the RBD [K417N] Mutant:ACE2 interaction, was tested in the EP-105 ACRO inhibition assay. Error bars indicate standard deviation. Each sample dilution was tested in duplicate. Very low to no inhibition was exhibited by negative control non-targeted extracted IgY. Greater than about 95% inhibition of ACE2:SARS-CoV-2 RBD [K417N] mutant binding by anti-Wuhan strain RBD IgY was demonstrated at total IgY concentrations above 1 mg/mL.

FIG. 20D shows a graph of competitive ELISA showing inhibition of ACE2:SARS-CoV-2 South Africa Spike S1 variant [K417N, E484K, N501Y, D614G] binding by anti-Wuhan strain RBD IgY. The percent inhibition averages as compared to positive control wells without IgY antibody, of the anti-RBD IgY and the non-targeted IgY samples inhibiting the SARS-CoV-2 S1 Variant:ACE2 interaction, was tested in the EP-105 ACRO inhibition assay. Error bars indicate standard deviation. Each sample dilution was tested in duplicate. Very low to no inhibition was exhibited by negative control non-targeted extracted IgY. Greater than about 96% inhibition of ACE2:SARS-CoV-2 S1 variant [K417N, E484K, N501Y, D614G] binding by anti-Wuhan strain RBD IgY was demonstrated at total IgY concentrations above 1 mg/mL.

Additional Mutant Testing was performed against SARS-CoV-2-mutants RBD [L452R] and RBD [S477N] as shown in Table 14B. Hodek-extracted anti-RBD IgY antibodies from eggs collected 50 days following the initial inoculation (Wuhan RBD Protein-based inoculation) was tested against additional mutants causing concern among the general population (FIGS. 9 and 10). All mutants tested to date and their corresponding variants are listed in Table 14C.

TABLE 14B
Mutant Proteins used in Indirect and Competitive Assay Formats

| Item | Description | Manufacturer | Part # |
|---|---|---|---|
| SARS-CoV-2 (2019- | Derived from HEK cells, | SinoBiological | 40592-V08H28 |
| nCoV) Spike | (YP_009724390.1) (Arg319- | | |
| RBD(L452R)-His | Phe541(L452R) | | |
| Recombinant Protein | | | |
| SARS-CoV-2 (2019- | Derived from HEK cells, | SinoBiological | 40592-V08H46 |
| nCoV) Spike | (YP_009724390.1) (Arg319- | | |
| RBD(S477N)-His | Phe541(S477N) | | |
| Recombinant Protein | | | |

FIG. 20E shows a graph of ELISA reactivity to coated SARS-CoV-2 RBD [L452R] Mutant in indirect binding assay for IgY antibodies extracted from chicken eggs 50 days after initial Wuhan RBD inoculation as compared to IgY antibodies sourced from non-targeted chicken eggs. OD at 450 nm was measured and plotted against the concentration of total IgY measured by the NanoDrop™ One© Instrument. Each sample was plated in duplicate and the averages and standard deviations are shown.

FIG. 20F shows a graph of ELISA reactivity to coated SARS-CoV-2 RBD [S477N] mutant in an indirect binding assay for anti-RBD IgY antibodies extracted from chicken eggs following Wuhan RBD inoculation as compared to IgY antibodies sourced from non-targeted chicken eggs. OD at 450 nm was measured and plotted against the concentration of total IgY measured by the NanoDrop™ One© Instrument. Each sample was plated in duplicate and the averages and standard deviations are shown.

TABLE 14C
Summary of Polyclonal anti-RBD IgY Positive
Reactivity to SARS-CoV-2 Mutants

| Variant Origin | Mutations Present (with +Reactivity to IgY) |
|---|---|
| UK (B.1.1.7) | RBD [N501Y] |
| South Africa (B.1.351) | S1 [RBD [K417N, E484K, N501Y] D614G] |
| Brazil/Japan (P.1) | RBD [E484K, N501Y] |
| New York (B.1.526) | RBD [S477N, E484K] |
| New York (B.1.524) | RBD [E484K] |
| California (B.1.427) | RBD [L452R] |
| California (B.1.429) | RBD [L452R] |

Table 14C displays clinically relevant SARS-CoV-2 variants and their Spike protein mutations (mainly RBD region) to which present anti-RBD IgY has successfully demonstrated positive binding reactivity and/or inhibition of ACE2 docking. The anti-SARS-CoV-2 RBD IgY provided herein from chicken inoculations with SARS-CoV-2 RBD exhibits binding reactivity to RBD variants comprising mutations present in UK (B.1.1.7), South Africa (B.1.351), Brazil/Japan (P.1), New York (B.1.526), New York (B.1.524), California (B.1.427), and California (B.1.429) variant strains. The anti-SARS-CoV-2 RBD IgY antibodies provided herein were able to prevent over 90% of Spike S1/RBD:ACE2 binding activity for each mutant protein. These in vitro results demonstrate that the present anti-RBD IgY antibodies have broad spectrum anti-SARS-CoV-2 antiviral activity against each of the SARS-CoV-2 variants tested. The results support use of the present anti-RBD IgY antibodies for prevention of SARS-CoV-2 transmission and infectivity as evidenced by their ability to bind to the ACE2 protein—the protein present on human epithelial cells at the back of the throat, which is targeted by SARS-CoV-2 to cause host infection.

### Example 24. Inhibition of RBD:ACE2 Binding by Anti-RBD IgY Antibodies in Spray Dried Powder

In this example egg yolks comprising anti-RBD IgY antibodies from chickens immunized with recombinant SARS-CoV-2 RBD protein were processed through a spray dryer and tested for binding active. The spray dried yolk powder was tested in two competitive neutralization formats to determine whether the SARS-CoV-2 specific antibodies within the sample could inhibit the SARS-CoV-2 RBD protein from binding to human ACE2. The ACE2 receptor is the primary docking site for the virus to initiate host infection. With neutralizing capabilities, the spray dried yolk powder was found to be appropriate for use in generating directly compressible dissolvable tablets for the goal of passive mucosal immunity to the SARS-CoV-2 virus.

Chickens were inoculated and boosted with recombinant SARS-CoV-2 S1 Subunit RBD protein purified from HEK cells (Creative Biomart) including 234 amino acids comprising SARS-CoV-2 (2019-nCoV) Spike Protein (RBD) (YP_009724390.1; Arg319-Phe541) (SEQ ID NO: 36) on a biweekly schedule: Days 0, 14, 28, and 42. Eggs from days 28, 35 and 50 were collected and processed. Eggs were also collected from un inoculated chickens (Non-Targeted) and processed for comparison. The eggs were washed, broken and the egg yolks separated from the egg whites to undergo a spray drying process.

The spray dried egg powder was tested in two ELISA neutralization assay formats. The ACRO Biosystems Inhibitor Screening Kit (EP-105) and GenScript SARS-CoV-2 Surrogate Virus Neutralization Test (sVNT)C-Pass™ Kit (GenScript Biotech Corporation) were employed according to manufacturer's protocols for detection of neutralizing antibodies.

These ELISA kits were designed to characterize the ability of test samples to inhibit the SARS-CoV-2 RBD:ACE2 binding event. The quantity of spray dried egg yolk powder found in a single dissolvable tablet (˜200 mg) was resuspended in 1.6 mL of diluent and tested in both assays to quantify the ability of the anti-RBD IgY within the powder to inhibit RBD:ACE2 binding.

Specifically, 200 mg of spray dried yolk powder (0.50 yolk, 0.49 trehalose, 0.01 silicon dioxide, and 0.005 benzyl alcohol) containing anti-RBD IgY was reconstituted in 1.6 mL distilled water to generate a stock solution which was used to create a dilution series of samples for testing. The same stock solution was generated for the Non-targeted negative control eggs. For quality control purposes, a spray dry formulation excluding egg yolk was also processed and tested for comparison. Specifically, 100 mg of spray dried excipients (0.98 trehalose, 0.02 silicon dioxide, and 0.01 benzyl alcohol) was reconstituted in 1.6 mL of distilled water.

The two ELISA neutralization assays, ELISA ACRO EP-105 and GenScript sVNT inhibition screening kits, were performed according to manufacturer's instructions, with slight adjustments described herein above.

In brief, the ACRO protocol calls for test samples and Biotinylated ACE2 to be applied together into an RBD-coated microplate. The Biotinylated ACE2 is then probed for by a Streptavidin-HRP conjugated secondary molecule. The absorbance is then measured at 450 nm, and this value corresponds to the binding activity of RBD and ACE2. The percentage of inhibition is then calculated for each well relative to the absorbance value generated in a Positive Control well. The GenScript protocol conversely involves an ACE2 pre-coated plate, and requires test samples to pre-incubate with RBD-HRP conjugated protein. This (test sample+RBD) mixture is applied to the immobilized ACE2 and the amount of binding is measured by absorbance reading at 450 nm, whereby HRP-RBD binding to ACE2 is detected. The percentage of inhibition is then calculated relative to the kit's Negative Control absorbance value.

In the GenScript sVNT assay, neutralizing anti-RBD IgY antibodies present in spray dried yolk powder from RBD-inoculated chickens exhibit >90% percent inhibition of RBD:ACE2 binding, as shown in FIG. 21A. Escalating specific antibody titers are observed from day 28 to day 35 to day 50. In comparison, the spray dried non-targeted IgY and spray dried excipient materials displayed a complete absence of neutralizing antibodies, by the sVNT kit standards.

In the ACRO EP-105 inhibition assay, neutralizing anti-RBD IgY antibodies present in spray dried yolk powder from RBD-inoculated chickens exhibit >95% percent inhibition of RBD:ACE2 binding, as shown in FIG. 21B. Escalating specific antibody titers are observed from day 28 to day 35 to day 50. In comparison, the spray dried non-targeted IgY and spray dried excipient materials displayed little to no RBD:ACE binding inhibition indicating an absence of neutralizing antibodies.

Chickens inoculated and boosted with recombinant SARS-CoV-2 RBD protein generate increasing anti-RBD IgY titer within their eggs over time. The anti-RBD IgY antibodies are viable within a spray dried formulation of the egg yolk to neutralize the RBD:ACE2 binding event in vitro in a competitive assay format. The formulated spray dried egg yolk powder from chicken eggs collected 50 days following initial RBD inoculation was able to inhibit >97% of RBD:ACE2 binding down to a powder concentration of 12 mg/mL. A single dissolvable tablet contains 200 mg of spray dried egg yolk powder.

The ability of raw egg yolk to be spray dried and retain viable anti-RBD specific IgY antibodies at significant titer allows for the development of prophylactic and therapeutic protection with anti-RBD IgY as the active pharmaceutical ingredient (API). Spray dried egg powder is directly compressible with proper excipients and has been used to generate a dissolvable tablet using dextrose monohydrate and Firmapress (LFA Tablet Presses) as flow agents. In this form and with the evidence of this study, the spray dried egg powder containing anti-RBD IgY may offer protection against SARS-CoV-2 infection at the mucosal surface and within the upper respiratory tract. The resuspension volume of H₂O used to reconstitute the spray dried powder is comparable to the volume of saliva in the mouth, suggesting that the antibody concentrations within the spray dried stock formulation is sufficient to inhibit the binding event between SARS-CoV-2 RBD and Human ACE2 in vivo, resulting in reduced transmission and infection of SARS-CoV-2.

### Example 25. Inhibition of RBD:ACE2 Binding by Anti-RBD IgY in a Cell-Based Pseudovirus Assay

A commercial service (Sino Biological, Wayne Pa.) was commissioned to evaluate samples of anti-SARS-CoV-2 RBD IgY polyclonal antibodies in a cell-based pseudovirus neutralization assay. A Covid-19 pseudovirus using HIV lentivirus vector packaging system was. Producer cell line 293T-ACE2 (human HEK293T(ACE2) stable cell line comprising a human ACE2 transgene) was employed. Pseudovirus Neutralization Assay Services brochure. Sino Biological, Inc. 2020.

A pseudovirus is a recombinant viral particle consisting of a surrogate virus core surrounded by a lipid envelope with the surface glycoproteins of another virus, such as SARS-CoV-2. The genes inside the pseudovirus are usually altered or modified so that they are unable to produce the surface protein on their own. As a result, an additional plasmid or stable cell line expressing the surface proteins is needed to make the pseudovirus.

Pseudoviruses are capable of infecting susceptible cells from various species with high titer and resistance to sera complement, but they only replicate for 1 round in the infected host cells. Compared with wild-type viruses, pseudoviruses are considered to be safer and easier to manipulate experimentally for neutralization assay. Viruses such as SARS-CoV-2 are highly contagious and pathogenic. The pseudovirus-based neutralization assay is recognized to be a suitable platform for safely and rapidly assessing and screening for neutralization antibodies or serum neutralization activities.

Two types of SARS-CoV-2 anti-RBD IgY antibody samples were generated for testing in the cell-base pseudovirus assay. Affinity-purified anti-RBD-IgY and Hodek-extracted anti-RBD IgY antibodies were prepared and shipped to Sino Biological Inc. for testing in the cell-based pseudovirus neutralization assay.

To generate Hodek-extracted anti-RBD IgY antibodies, eggs were collected from inoculated chickens at time points from ˜35 days to ˜49 days following initial RBD inoculation with recombinant SARS-CoV-2 (2019-CoV) RBD protein (Creative Biomart Spike-190V) which was fused to His-tag at C-terminus and expressed in human HEK293 cells. The recombinant protein includes a 234 amino acid sequence comprising SARS-CoV-2 (2019-nCoV) Spike Protein (RBD) (YP_009724390.1; Arg319-Phe541) (SEQ ID NO: 36). Immune eggs were collected, washed, broken and yolks were separated and subjected to Hodek IgY extraction process, as described in example 9. Pellets containing IgY extractions were generated, and resuspended in 5 mL of PBS, dialyzed, and shipped to Sino Biological for testing (Samples 2, 3). The Hodek extracted anti-RBD IgY contained approximately 5-10% of specific anti-RBD IgY antibodies.

To generate the affinity purified anti-RBD IgY antibodies, several dozen eggs collected from chickens innoculated with recombinant SARS-CoV-2 (2019-nCoV) Spike Protein (RBD) (YP_009724390.1; Arg319-Phe541) (SEQ ID NO: 36) were collected approximately 35 days following initial inoculation and shipped to Aves Labs, Inc. Aves Labs was also provided with HEK-produced RBD protein (Creative Biomart Spike-190V) for coating an affinity purified column. IgY antibodies were extracted from the eggs and subjected to affinity chromatography to obtain the affinity purified RBD-specific IgY antibodies. The affinity purified anti-RBD antibodies were shown to be specific and active by a simple indirect ELISA using the provided RBD protein coated on a 96-well plate. The affinity purified anti-RBD IgY antibodies were shipped to Sino Biological Inc. for testing (sample 1).

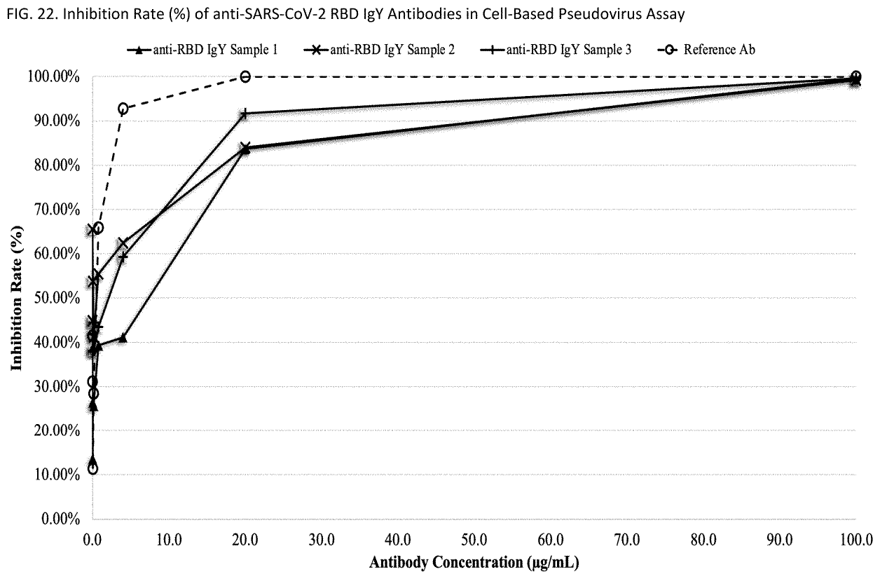

In the Pseudovirus Neutralization assay, the three different samples of anti-SARS-CoV-2 RBD IgY antibodies and a Reference antibody were serially diluted across a 96-well plate, a known quantity of pseudovirus was added, and incubated to allow antibody binding. A set quantity of 293T-ACE2 cells was added and plates were incubated for 48 h. After the 48 h incubation period, luciferase substrate was added to each well and RLU values were read. Antibody neutralization activity or inhibitory rate was calculated based on RLU, relative light unit, values measuring luciferase activity. The inhibitory rate was calculated as follows: Inhibition Rate (%)=1−(Average RLU of test group−Average of negative control)/(Average RLU of positive control−Average RLU of negative control). The inhibitory rate was used for accessing antibody and neutralization activities. Results are shown in Table 15 and FIG. 22.

TABLE 15
Pseudovirus Neutralization Assay Results and IC₅₀ values

| | Concentration (μg/mL) - Inhibition Rate (%) | | | | | | | | |
|---|---|---|---|---|---|---|---|---|---|
| Sample | 100 | 20 | 4.0 | 0.8 | 0.16 | 0.032 | 0.0064 | 0.0013 | IC50 |
| Sample 1 | 99.46% | 83.66% | 41.15% | 39.29% | 25.61% | 26.41% | 13.56% | 38.74% | 5.592 |
| Sample 2 | 99.18% | 83.96% | 62.49% | 55.40% | 41.16% | 53.77% | 65.54% | 44.88% | 0.435 |
| Sample 3 | 99.56% | 91.67% | 59.29% | 43.44% | 38.13% | 44.43% | 38.00% | 41.77% | 1.557 |
| Reference Ab | 100.0% | 99.95% | 92.79% | 65.94% | 28.42% | 11.38% | 31.16% | 41.46% | 0.404 |

The Reference antibody was SARS-CoV/SARS-CoV-2 Spike antibody, chimeric Mab (Sino Biological Inc., #40150-D001) produced using recombinant SARS-CoV spike RBD protein (Cat. #40150-V08B2). The Reference antibody is a recombinant chimeric monoclonal antibody combining the constant domains of the human IgG1 molecule with mouse variable regions. The immunogen for the reference antibody was recombinant receptor binding domain (RBD) of SARS-CoV (isolate:WH20) spike (AAX16192.1) (Arg306-Phe527) expressed with a C-terminal polyhistidine tag using expression host baculovirus insect cells. The reference antibody has cross-reactivity in ELISA with SARS-CoV Spike S1 protein (Cat #40150-V08B1); SARS-CoV-2 (2019-nCoV) Spike S1 protein (Cat #40591-V08H); and SARS-CoV-2(2019-nCoV) Spike RBD protein (Cat #40592-V08B).

Results in the pseudovirus-based neutralization assay show the IC₅₀ value of Sample 2 anti-SARS-CoV-2 RBD IgY antibodies (0.435 μg/mL) was comparable to the positive control monoclonal reference antibody (0.404 μg/mL), as shown in Table 15. Samples 1 and 2 anti-SARS-CoV-2 RBD IgY antibodies exhibited IC₅₀ values of 5.592 μg/mL and 1.557 μg/mL, respectively, in the pseudovirus neutralization assay. FIG. 22 shows a graph of the Table 15 data as inhibition rate (%) v. anti-RBD IgY concentration (μg/mL) for the three antibody samples compared to a Sino Biological positive control antibody. The results demonstrate that the anti-SARS-CoV-2 RBD IgY antibodies—whether affinity purified or contained within a total IgY extraction—are capable of neutralizing >99% of the pseudovirus activity in the Sino Biological cell-based neutralization assay. The present example shows the Hodek-extracted anti-SARS-CoV-2 RBD IgY antibodies exhibited a comparable IC₅₀ compared to MAb reference antibody in the COVID-19 pseudovirus-based neutralization assay. These results support use of the anti-RBD IgY antibodies to prevent transmission and infection of the SARS-CoV-2 virus in human subjects.

### Example 26. Production of Anti-Human ACE2 IgY Antibodies and Inhibition of the SARS-CoV-2 RBD:ACE2 Interaction

The purpose of this experiment is to demonstrate the SARS-CoV-2 neutralizing effects of anti-ACE2 IgY antibodies promoted by the inoculation of a recombinant purified ACE2 protein extracellular domain inoculum. ACE2 proteins were sourced from two suppliers for the first inoculation. White Leghorn chickens were inoculated with rACE2 protein in Freund's Complete Adjuvant. The chickens also received subsequent inoculations (booster inoculations). Following inoculation, blood samples were collected and serum was used to qualitatively measure specific antibody titers using antigen-specific indirect ELISA binding assays. The blood samples were also tested in a GenScript SARS-CoV-2 Surrogate Virus Neutralization Test (sVNT) C-Pass™ Kit to quantitatively determine the neutralizing capacity of the IgY antibodies targeted to ACE2 protein. The specific antibodies were successfully produced and detected, giving indication of potential oral-based therapeutics in which ACE2 IgY antibodies inhibit the binding capabilities of SARS-CoV-2.

Human Angiotensin-Converting Enzyme 2 (ACE2) belongs to the angiotensin-converting enzyme family of dipeptidyl carboxydipeptidases and has considerable homology to human angiotensin 1 converting enzyme. This secreted protein catalyzes the cleavage of angiotensin I into angiotensin 1-9, and angiotensin II into the vasodilator angiotensin 1-7. The organ- and cell-specific expression of this gene suggests that it may play a role in the regulation of cardiovascular and renal function, as well as fertility. In addition, the encoded protein is a functional receptor for the spike glycoprotein of the human coronaviruses SARS and HCoV-NL63. Human ACE2 amino acid sequence may have Accession Q9BYF1 (SEQ ID NO: 43).

Creative Biomart ACE2-736H recombinant human ACE2, His-tagged, was employed in development of an immunogen. Specifically, recombinant Human Angiotensin-Converting Enzyme 2/ACE-2 was produced by transfected human HEK293 cells, and is a secreted protein with amino acid sequence (Gln18-Ser740) of human ACE-2 fused with a polyhistidine tag at the C-terminus (SEQ ID NO: 42).

RayBiotech human ACE 2 230-30165 recombinant human ACE 2 comprises amino acid sequence of Gln18-Ser740 (Extracellular domain) of Accession Q9BYF1 and a C-terminal His-tag. The protein was expressed in human embryonic kidney 293 (HEK293) cells, and purified by His-tag affinity purification by immobilized metal ion chromatography (IMAC). RayBiotech human ACE 2 was also employed as an immunogen.

Freund's Adjuvant was used as a water-in-oil emulsion including non-metabolizable oils (paraffin oil and mannide monooleate). If it also contains killed Mycobacterium tuberculosis it is known as Complete Freund's Adjuvant (e.g., Sigma F5881). Without the bacteria it is Incomplete Freund's Adjuvant (e.g., Sigma F5506). Each ml of F 5881 contains 1 mg of heat-killed and dried Mycobacterium tuberculosis (strain H37Ra, ATCC 25177), 0.85 ml paraffin oil and 0.15 ml of mannide monooleate. Each ml of F 5506 contains 0.85 ml of paraffin oil and 0.15 ml of mannide monooleate.

Recombinant ACE2 protein from either Creative Biomart (Cat #ACE2-736H) or RayBiotech (Cat #: 230-30165) was concentration-verified by Thermofisher NanoDrop™ One© Protein A280 application. A group of 5 chickens was selected for ACE2 (supplied by Creative Biomart) inoculation and another group of 5 chickens for ACE2 (supplied by RayBiotech) inoculation. The volumes of protein resuspension, PBS and Freund's adjuvant were calculated, measured, and combined into a single formulation containing two distinct liquid layers having target protein concentration of 500 micrograms/mL. To prepare the CBio group inoculum, 591 μL was combined with 1.159 mL of 1×PBS for a final protein mixture. The mixture was then combined with an equal volume of Freund's Complete Adjuvant. To prepare the RBio group inoculation, 313 μL was combined with 1.438 mL of 1×PBS for a final protein mixture. The final protein mixture was then combined with an equal volume of Freund's Complete Adjuvant to create each inoculum.

The inoculum was shipped overnight to the farming facility on ice packs. Immediately before injection, a water-in-oil emulsion was formed between the aqueous protein solution and Freund's adjuvant by forcibly mixing the formulation between two syringes using a three-way stop cock. After the emulsion was properly formed (i.e., the two distinct liquid layers had formed into one homogenous formulation), it was administered to the chickens via two intramuscular injections (a 250 μL injection in each breast for a total volume of 500 μL per chicken). The first group of chickens received an initial inoculation (Day 0) of 500 μL containing ˜125 μg of ACE2 protein purified from HEK-293 cells (Creative Biomart) in Freund's Complete Adjuvant. The second group of chickens received an initial inoculation (Day 0) of 500 μL containing ˜125 μg of ACE2 protein purified from HEK-293 cells (RayBiotech) in Freund's Complete Adjuvant.

Processing of Antisera. Whole blood from both test groups was collected 17 days following the initial inoculation. The blood had not been treated with any anti-coagulants and could therefore separate the serum from fibrinogen and other clotting factors. The serum samples were diluted with 1×PBS and analyzed for estimated total IgY concentration using NanoDrop One© Protein A280 application.

Detection of Specific Anti-ACE2 IgY Antibodies by Indirect ELISA

Briefly, ACE2 protein at a concentration of 1.0 μg/mL was coated to a 96-well microtiter plate and incubated at 4° C. overnight. Two plates were used for testing—one coated with ACE2 from Creative Biomart and the other from RayBiotech). After incubation, the plates were washed and blocked with a 10% BlokHen® Solution for 2 h. After blocking, the plates were washed, and isolated serum from inoculated and uninoculated chickens were plated in duplicate in a dilution series and incubated for 1 h. After the primary incubation period, the plates were washed and a goat anti-chicken HRP-conjugated antibody was applied to probe for any IgY antibodies bound to the ACE2 protein coated on the plates. The plates were developed with TMB and the reaction terminated with 1.0 M H₂SO₄. Absorbance at 450 nm was measured and recorded in a plate reader.

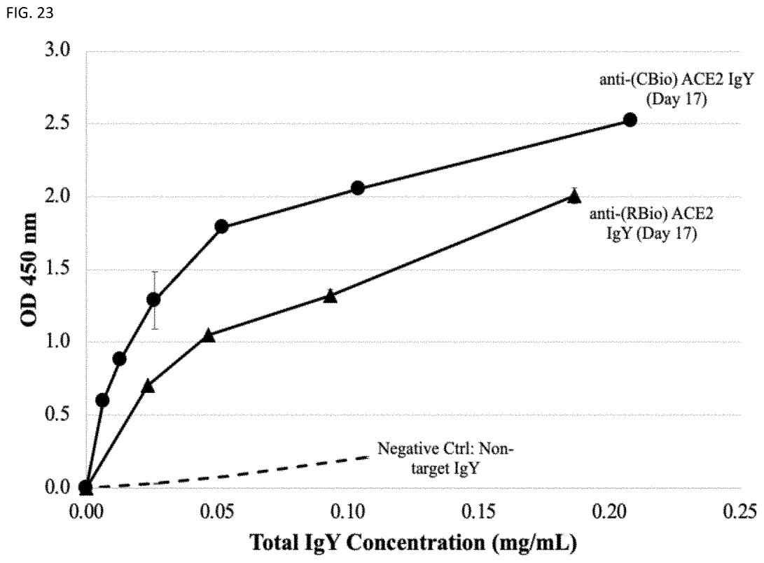

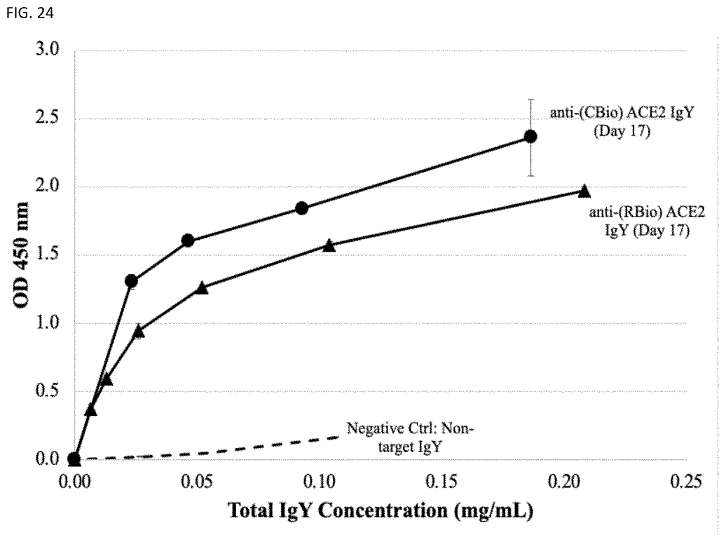

Reactivity of anti-ACE2 IgY from serum against coated Creative Biomart-Sourced ACE2 Protein in the indirect ELISA is shown in FIG. 23. Reactivity of anti-ACE2 IgY from serum against coated RayBiotech-Sourced ACE2 Protein in the indirect ELISA is shown in FIG. 24. Serum from uninoculated (non-targeted) chickens was used as a negative control. The OD values were plotted against total IgY concentration (mg/mL) to show a dose-dependence in specific antibody concentration. The Creative Biotech-ACE2 inoculated chicken serum exhibited higher reactivity in both FIG. 23 and FIG. 24.

The serum anti-ACE2 IgY was also evaluated in GenScript SARS-CoV-2 Surrogate Virus Neutralization Test (sVNT) C-Pass' Kit assay according to modified manufacturer's protocols to assess the neutralizing capability of anti-ACE2 IgY. The modified protocol calls for anti-ACE2 IgY Test Samples to incubate with ACE2-coated microplate for 30 minutes at 37° C. After this incubation period, HRP-conjugated RBD protein is applied to the microplate wells at a 1:1 volumetric ratio. This mixture of HRP-RBD and anti-ACE2 IgY within the ACE2-coated microplate is incubated for 15 minutes at 37° C. After this step, the plate is washed, and TMB substrate is applied to visualize any HRP-RBD binding activity to ACE2. The reaction was terminated with a provided STOP Solution and the absorbance of the microplate is read at 450 nm. Inhibition of the binding event of RBD-HRP to ACE2 is indicated by little to no color change, and the percentage of inhibition is calculated relative to the provided Negative Control's absorbance value.

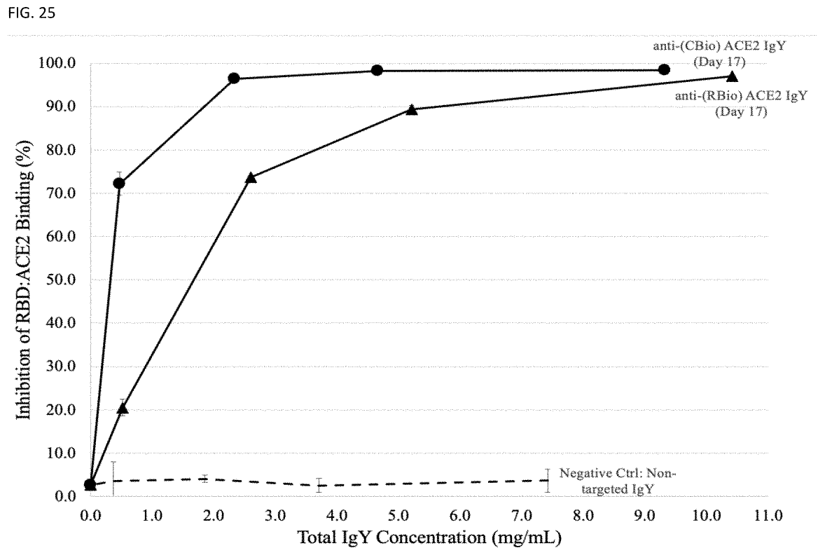

FIG. 25 shows percent inhibition of RBD:ACE2 binding by anti-ACE2 IgY antibodies in serum from recombinant ACE2-inoculated chickens, compared to serum from uninoculated chickens (non-targeted IgY). In the neutralization assay format anti-(Creative Biotech) ACE2 IgY antibodies displayed higher percentages of inhibition compared to anti-(RayBio) ACE2 IgY antibodies.

At 35 days following the initial inoculation of the two test groups, both groups of chickens received a 500 μL boost inoculation containing 125 μg of recombinant ACE2 protein purified from HEK-293 cells (Creative Biomart) (SEQ ID NO: 1) in Freund's incomplete adjuvant.

The reactivity of anti-ACE2 IgY antibodies to the coated ACE2 protein indicates the present inoculation technique elicits the production of specific antibodies in serum of the target host (White Leghorn chickens). Using this inoculation strategy, antibodies to specific targets may be produced in yolks of eggs. In this case, the anti-ACE2 IgY antibodies in sera exhibited reactivity to ACE2 protein after a single inoculation. The anti-ACE2 polyclonal IgY antibodies also prevented SARS-CoV-2 from binding to ACE2 in a neutralizing ELISA assay format. The anti-ACE2 IgY may be useful alone or in combination with anti-SARS-CoV-2 RBD IgY for preparation of compositions for treating or preventing a coronavirus infection, for example, for preventing or decreasing SARS-CoV-2 viral transmission.

### Example 27. Production of Anti-Human ACE2 IgY Antibodies and Inhibition of the SARS-CoV-2 RBD:ACE2 Interaction

The purpose of this experiment is to demonstrate the SARS-CoV-2 neutralizing effects of anti-ACE2 IgY antibodies promoted by the inoculation of a recombinant purified ACE2 protein extracellular domain inoculum. ACE2 proteins were sourced from two suppliers for the first inoculation. White Leghorn chickens were inoculated with rACE2 protein in Freund's Complete Adjuvant. The chickens also received subsequent inoculations (booster inoculations). Following inoculation, blood samples were collected and serum was used to qualitatively measure specific antibody titers using antigen-specific indirect ELISA binding assays. The serum may be obtained by centifuging blood samples to remove red blood cells at 3,000 rcf at 4° C. for 15-20 minutes to obtain the serum layer. The blood samples were also tested in a GenScript SARS-CoV-2 Surrogate Virus Neutralization Test (sVNT) C-Pass™ Kit to quantitatively determine the neutralizing capacity of the IgY antibodies targeted to ACE2 protein. The specific antibodies were successfully produced and detected, giving indication of potential oral-based therapeutics in which ACE2 IgY antibodies inhibit the binding capabilities of SARS-CoV-2.

Human Angiotensin-Converting Enzyme 2 (ACE2) belongs to the angiotensin-converting enzyme family of dipeptidyl carboxydipeptidases and has considerable homology to human angiotensin 1 converting enzyme. This secreted protein catalyzes the cleavage of angiotensin I into angiotensin 1-9, and angiotensin II into the vasodilator angiotensin 1-7. The organ- and cell-specific expression of this gene suggests that it may play a role in the regulation of cardiovascular and renal function, as well as fertility. In addition, the encoded protein is a functional receptor for the spike glycoprotein of the human coronaviruses SARS and HCoV-NL63. Human ACE2 amino acid sequence may have Accession Q9BYF1 (SEQ ID NO: 78).

Creative Biomart ACE2-736H recombinant human ACE2, His-tagged, was employed. Specifically, recombinant Human Angiotensin-Converting Enzyme 2/ACE-2 was produced by transfected human HEK293 cells, and is a secreted protein with sequence (Gln18-Ser740) of human ACE-2 fused with a polyhistidine tag at the C-terminus (SEQ ID NO: 77).

RayBiotech human ACE 2 230-30165 recombinant human ACE 2 comprises Gln18-Ser740 (Extracellular domain) of Accession Q9BYF1 and a C-terminal His-tag. The protein was expressed in human embryonic kidney 293 (HEK293) cells, and purified by His-tag affinity purification by immobilized metal ion chromatography (IMAC).

Freund's Adjuvant is used as a water-in-oil emulsion. It is prepared from non-metabolizable oils (paraffin oil and mannide monooleate). If it also contains killed Mycobacterium tuberculosis it is known as Complete Freund's Adjuvant (e.g., Sigma F5881). Without the bacteria it is Incomplete Freund's Adjuvant (e.g., Sigma F5506). Each ml of F 5881 contains 1 mg of heat-killed and dried Mycobacterium tuberculosis (strain H37Ra, ATCC 25177), 0.85 ml paraffin oil and 0.15 ml of mannide monooleate. Each ml of F 5506 contains 0.85 ml of paraffin oil and 0.15 ml of mannide monooleate.

Recombinant ACE2 protein from either Creative Biomart (Cat #ACE2-736H) or RayBiotech (Cat #: 230-30165) was concentration-verified by Thermofisher NanoDrop™ One© Protein A280 application. A group of 5 chickens was selected for ACE2 (supplied by Creative Biomart) inoculation and another group of 5 chickens for ACE2 (supplied by RayBiotech) inoculation. The volumes of protein resuspension, PBS and Freund's adjuvant were calculated, measured, and combined into a single formulation containing two distinct liquid layers having target protein concentration of 500 micrograms/mL. To prepare the CBio group inoculum, 591 μL was combined with 1.159 mL of 1×PBS for a final protein mixture. The mixture was then combined with an equal volume of Freund's Complete Adjuvant. To prepare the RBio group inoculation, 313 μL was combined with 1.438 mL of 1×PBS for a final protein mixture. The final protein mixture was then combined with an equal volume of Freund's Complete Adjuvant to create each inoculum.

The inoculum was shipped overnight to the farming facility on ice packs. Immediately before injection, a water-in-oil emulsion was formed between the aqueous protein solution and Freund's adjuvant by forcibly mixing the formulation between two syringes using a three-way stop cock. After the emulsion was properly formed (i.e., the two distinct liquid layers had formed into one homogenous formulation), it was administered to the chickens via two intramuscular injections (a 250 μL injection in each breast for a total volume of 500 μL per chicken). The first group of chickens received an initial inoculation (Day 0) of 500 μL containing ˜125 μg of ACE2 protein purified from HEK-293 cells (Creative Biomart) in Freund's Complete Adjuvant. The second group of chickens received an initial inoculation (Day 0) of 500 μL containing ˜125 μg of ACE2 protein purified from HEK-293 cells (RayBiotech) in Freund's Complete Adjuvant.

Processing of Antisera. Whole blood from both test groups was collected 17 days following the initial inoculation. The blood had not been treated with any anti-coagulants and could therefore separate the serum from fibrinogen and other clotting factors. The serum samples were diluted with 1×PBS and analyzed for estimated total IgY concentration using NanoDrop One© Protein A280 application.

Detection of Specific Anti-ACE2 IgY Antibodies by Indirect ELISA

Briefly, ACE2 protein at a concentration of 1.0 μg/mL was coated to a 96-well microtiter plate and incubated at 4° C. overnight. Two plates were used for testing—one coated with ACE2 from Creative Biomart and the other from RayBiotech). After incubation, the plates were washed and blocked with a 10% BlokHen® Solution for 2 h. After blocking, the plates were washed, and isolated serum from inoculated and uninoculated chickens were plated in duplicate in a dilution series and incubated for 1 h. After the primary incubation period, the plates were washed and a goat anti-chicken HRP-conjugated antibody was applied to probe for any IgY antibodies bound to the ACE2 protein coated on the plates. The plates were developed with TMB and the reaction terminated with 1.0 M H₂SO₄. Absorbance at 450 nm was measured and recorded in a plate reader.

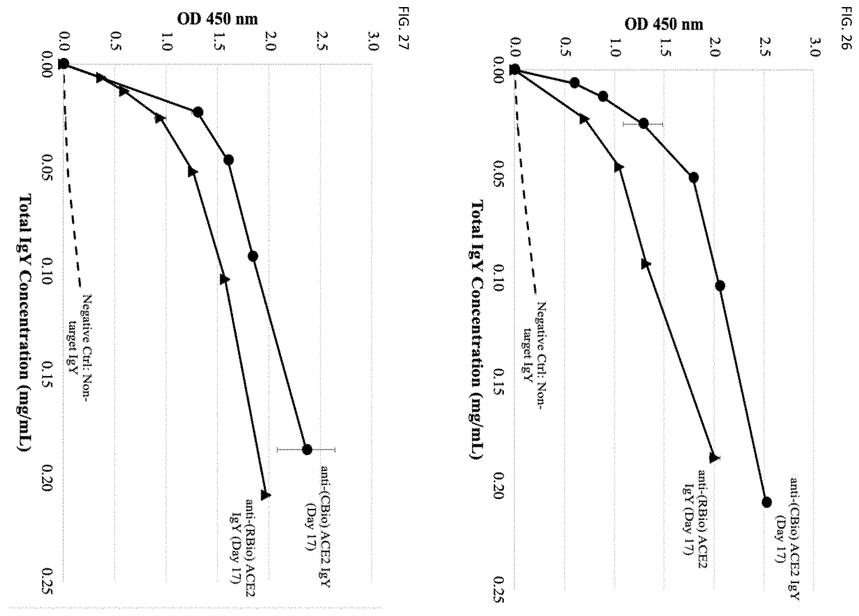

Reactivity of anti-ACE2 IgY from serum against coated Creative Biomart-Sourced ACE2 Protein in the indirect ELISA is shown in FIG. 1. Reactivity of anti-ACE2 IgY from serum against coated RayBiotech-Sourced ACE2 Protein in the indirect ELISA is shown in FIG. 2. Serum from uninoculated (non-targeted) chickens was used as a negative control. The OD values were plotted against total IgY concentration (mg/mL) to show a dose-dependence in specific antibody concentration. The Creative Biotech-ACE2 inoculated chicken serum exhibited higher reactivity in both FIG. 26 and FIG. 27.

The serum anti-ACE2 IgY was also evaluated in GenScript SARS-CoV-2 Surrogate Virus Neutralization Test (sVNT) C-Pass' Kit assay according to modified manufacturer's protocols to assess the neutralizing capability of anti-ACE2 IgY. The modified protocol calls for anti-ACE2 IgY Test Samples to incubate with ACE2-coated microplate for 30 minutes at 37° C. After this incubation period, HRP-conjugated RBD protein is applied to the microplate wells at a 1:1 volumetric ratio. This mixture of HRP-RBD and anti-ACE2 IgY within the ACE2-coated microplate is incubated for 15 minutes at 37° C. After this step, the plate is washed, and TMB substrate is applied to visualize any HRP-RBD binding activity to ACE2. The reaction was terminated with a provided STOP Solution and the absorbance of the microplate is read at 450 nm. Inhibition of the binding event of RBD-HRP to ACE2 is indicated by little to no color change, and the percentage of inhibition is calculated relative to the provided Negative Control's absorbance value.

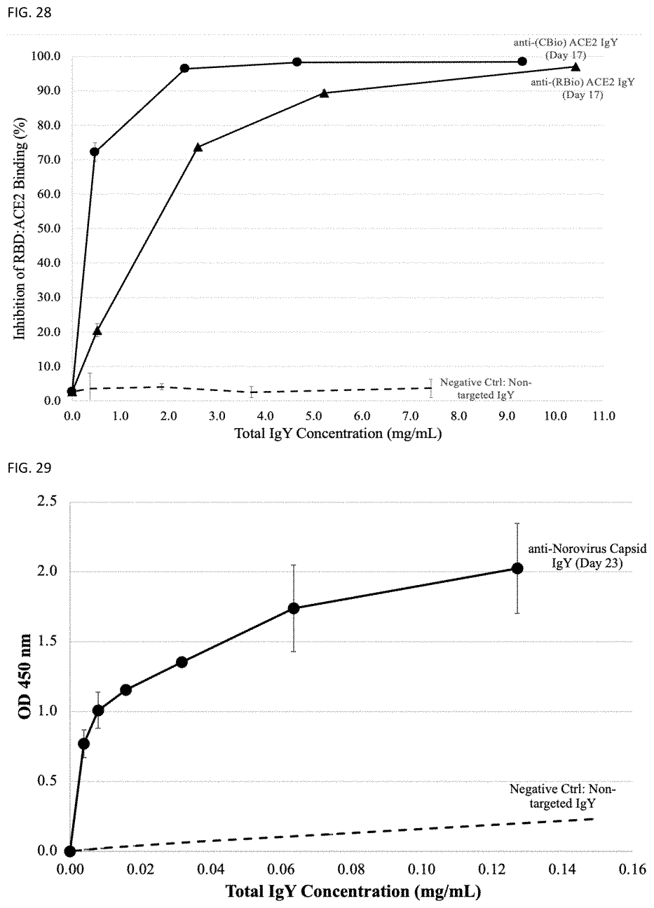

FIG. 28 shows percent inhibition of RBD:ACE2 binding by anti-ACE2 IgY antibodies in serum from recombinant ACE2-inoculated chickens, compared to serum from uninoculated chickens (non-targeted IgY). In the neutralization assay format anti-(Creative Biotech) ACE2 IgY antibodies displayed higher percentages of inhibition compared to anti-(RayBio) ACE2 IgY antibodies.

At 35 days following the initial inoculation of the two test groups, both groups of chickens received a 500 μL boost inoculation containing 125 μg of recombinant ACE2 protein purified from HEK-293 cells (Creative Biomart) (SEQ ID NO: 77) in Freund's incomplete adjuvant.

The reactivity of anti-ACE2 IgY antibodies to the coated ACE2 protein indicates the present inoculation technique elicits the production of specific antibodies in serum of the target host (White Leghorn chickens). Using this inoculation strategy, antibodies to specific targets may be produced in yolks of eggs. In this case, the anti-ACE2 IgY antibodies in sera exhibited reactivity to ACE2 protein after a single inoculation. The anti-ACE2 polyclonal IgY antibodies also prevented SARS-CoV-2 from binding to ACE2 in a neutralizing ELISA assay format. The anti-ACE2 IgY may be useful alone or in combination with anti-SARS-CoV-2 RBD IgY for preparation of compositions for treating or preventing a coronavirus infection, for example, for preventing or decreasing SARS-CoV-2 viral transmission.

### Example 28. Generation and Detection of Anti-Norovirus IgY Antibodies

The purpose of this experiment was to demonstrate that chickens inoculated with Norovirus Group-1 Capsid Recombinant protein produce specific IgY targeted to the capsid protein in order to develop Norovirus-specific IgY antibodies that could be used in a therapeutic or prophylactic composition. An indirect ELISA assay was used to qualitatively assess the presence of anti-Norovirus Group-1 Capsid IgY titer within the inoculated chickens.

A group of 5 White Leghorn hen chickens were inoculated with 125 μg of Norovirus Group-1 Capsid Recombinant protein (ProSpec NRV-213) in Freund's Complete Adjuvant/PBS on Day 0 and boosted with the same quantity of protein in Freund's Incomplete Adjuvant/PBS on Day 17.

The recombinant Norovirus Group-1 capsid, E. Coli derived, is a positive sense RNA virus with 7.5 kb nucleotides, encoding a major structural protein VP1 with 50-55 kDa and a VP2 protein. The full length of VP1 capsid protein is derived from the group 1 Norwalk virus. Norovirus Group-1 Capsid protein Accession Q83884 (SEQ ID NO: 79). The protein is fused to a 6 His tag at N-terminal and purified by chromatography techniques

A target concentration of Norovirus Group-1 Capsid Recombinant protein of 125 μg per chicken was chosen for inoculation. The volumes of protein resuspension, 10×PBS and Freund's adjuvant were calculated, measured, and combined into a single formulation (Note: At this point, the single formulation contained two distinct liquid layers). The inoculum was shipped overnight to the farming facility on ice packs. Immediately before injection, a water-in-oil emulsion was formed between the aqueous protein solution and Freund's adjuvant by forcibly mixing the formulation between two syringes using a three-way stop cock. After the emulsion was properly formed and the two distinct liquid layers had formed into one homogenized formulation, it was administered to the chickens via two intramuscular inoculations: one 250 μL injection in each breast for a total inoculation volume of 500 μL per chicken.

Whole blood was collected at 23 days following the initial inoculation. The whole blood was not treated with any anti-coagulants and could therefore separate the serum from fibrinogen and other clotting factors. The serum samples were diluted with 1×PBS and analyzed for estimated total IgY concentrations using NanoDrop One© Protein A280 application.

IgY antibodies from serum were tested in an indirect ELISA assay designed to detect the presence of specific antibodies. The Norovirus capsid protein (ProSpec NRV-213) was coated to a 96-well microtiter plate at 1.0 μg/mL and incubated at 4° C. overnight. After incubation, the plate was blocked with a 10% BlokHen solution for 2 h. After blocking, the plate was washed and the serum samples were plated in duplicate in a dilution series and incubated for 1 h. A goat anti-chicken HRP-conjugated antibody was applied to probe for any IgY antibodies bound to the capsid protein coated on the plate. The plate was developed with TMB and the reaction terminated with 1.0 M H₂SO₄. Absorbance at 450 nm was measured and recorded in a plate reader.

FIG. 29 shows a graph of ELISA reactivity plotted as OD450 v. total IgY concentration between the coated norovirus capsid protein and anti-norovirus capsid protein IgY antibodies present in serum 23 days post the initial inoculation. Serum from uninoculated (non-targeted) chickens was used as a negative control. Each sample was plated in duplicate and the averages and standard deviations are shown in the graph. Specific reactivity was demonstrated. The presence of specific antibodies within the blood coupled with the natural processes of chicken passive immunity indicate the specific antibodies are already present in the laid eggs

The anti-norovirus group −1 capsid protein IgY may be used in production of an anti-Norovirus Group-1 Capsid therapeutic compositions. The compositions may be useful for the treating or preventing norovirus community acquired infections, particularly in the cases of cruise ships, military personnel, and prisons, or wherever a large amount of people gather and Norovirus can spread easily.

### Example 29. Preparation of Specific Anti-Staphylococcus aureus and Staphylococcal Protein A Antibodies

The purpose of this experiment was production of specific IgY antibodies in chickens inoculated with S. aureus fixed whole cells and SpA protein and to demonstrate ELISA reactivity in polyclonal IgY antibodies within chicken serum and isolated from raw egg yolks. The specific IgY antibodies—extracted from raw egg yolks and isolated from blood samples—are tested using ELISA indirect assays to qualitatively measure the binding of anti-S. aureus and anti-SpA IgY to S. aureus and SpA, respectively.

Preparation of Formalin-Fixed S. aureus Whole Cells for Chicken Inoculation

Briefly, for each inoculation cycle, S. aureus cells were grown and expanded in liquid culture media overnight. After ˜16 h of growth, the cells were harvested via centrifugation and washed 3× in PBS. The cell pellet was resuspended in PBS and dilution plating was performed to calculate the CFU/mL. Next, 5 mL of the cell suspension was fixed in a 1% formalin solution for ˜16 h at 4° C. After the cells were fixed, they were harvested via centrifugation and washed 3× in PBS. The resulting cell pellet was resuspended in 5 mL of PBS to equal the amount of cell suspension removed from the original suspension. As the two volumes are equal, the CFU/mL calculated from the non-fixed PBS cell suspension should also be equal to the formalin-fixed cell suspension.

A target concentration of 1.00E+9 CFU/mL (before addition of Freund's adjuvant) was chosen for inoculation based on literature values. The CFU count from the cell suspension was used to determine the volume of formalin-fixed cells needed to meet the target concentration for inoculation. A group of 13 Leghorn chickens was selected for formalin-fixed S. aureus whole cell inoculation. The quantities of formalin-fixed cell suspension, PBS and Freund's adjuvant were calculated, measured, and combined into a single formulation containing two distinct liquid layers. The inoculum was shipped overnight to the farming facility on ice packs. Immediately before injection, a water-in-oil emulsion was formed between the aqueous protein solution and Freund's adjuvant by forcibly mixing the formulation between two syringes using a three-way stop cock. After the emulsion was properly formed (i.e., the two distinct liquid layers had formed into one homogenized formulation), it was administered to the chickens via four intramuscular inoculations (two 250-μL injections in each breast for a total inoculation volume of 1 mL per chicken). A group of chickens were inoculated with 1.00E+9 CFU/mL formalin-fixed Staphylococcus aureus (S. aureus) cells in Freund's Complete Adjuvant on Day 0 and boosted with the same CFU/mL in Freund's Incomplete Adjuvant on Days 14, 35 and 49.

The isolated biomolecule inoculum was prepared as follows. In brief, for each inoculation cycle, lyophilized Staphylococcal protein A (SpA) was purchased from Sigma Aldrich, and rehydrated to 1 mg/mL in mol bio water according to the supplier's specifications. A target amount of 125 μg protein per chicken was chosen for inoculation. The Spa protein may comprise the amino acid sequence of SEQ ID NO: 119. The volumes of protein resuspension, 10×PBS and Freund's adjuvant were calculated, measured, and combined into a single formulation having two distinct liquid layers. The inoculum was shipped overnight to the farming facility on ice packs. Immediately before injection, a water-in-oil emulsion was formed between the aqueous protein solution and Freund's adjuvant by forcibly mixing the formulation between two syringes using a three-way stop cock. After the emulsion was properly formed (i.e., the two distinct liquid layers had formed into one homogenized formulation), it was administered to the chickens via two intramuscular inoculations (one 250-μL injection in each breast for a total inoculation volume of 500 μL per chicken). Chicken inoculations were performed as initial (Day 0, Freund's complete adjuvant) and booster inoculations (Days 12, 38 and 49, Freund's incomplete adjuvant). There were a total of 12 chickens to be inoculated and each chicken required 125 μg of protein in a 500-μL injection volume.

### Processing of Antisera

Whole blood was collected from both chicken groups (S. aureus 502a whole cell- and Staphylococcal protein A-based inoculations) at 35 days following the initial inoculation. The blood had not been treated with any anti-coagulants and could therefore separate the serum from fibrinogen and other clotting factors. The serum samples were diluted with 1×PBS and analyzed for estimated total IgY concentrations using NanoDrop One© Protein A280 application.

Processing of IgY from Egg Yolks

Eggs collected 18 days after the initial inoculations were selected for processing. Collected eggs from S. aureus and SpA inoculated chickens were pooled and processed for detection of S. aureus-specific antibodies. IgY antibodies were extracted from 12 whole eggs collected 18 days following initial inoculation and pooled together for processing. Briefly, the eggs were washed, cracked, and the yolk separated from the albumen. The yolks were then diluted 8-fold with tap water and the pH adjusted to 5.0 using HCl. The yolk solution was then frozen for 12-16 h. Once frozen, the solution was thawed over a filtration apparatus to remove the lipidic components of the yolk and the water-soluble fraction was collected. The volume of the water-soluble fraction was measured, 8.8% (w/v) NaCl was added, and the pH adjusted to 4.0 using HCl. Using centrifugation, the IgY antibodies were pelleted. The pellet was resuspended in 6 mL of PBS and dialyzed for 12-16 h. The dialyzed extraction was analyzed using the NanoDrop™ One© Protein A280 application to determine the total IgY concentration within the sample.

Antibodies from serum or extracted from yolk via centrifugation/precipitation extraction processes were used to qualitatively demonstrate specific IgY titer in antigen-specific indirect ELISA assays. An indirect ELISA assay was used to detect the presence of specific anti-S. aureus (whole cell) antibodies.

Formalin-fixed cells at a concentration of 5.50E+08 CFU/mL were coated to a 96-well microtiter plate and incubated at 4° C. overnight. After incubation, the plate was blocked with a 5% BSA solution for 2 h. After blocking, the plate was washed, samples-isolated serum and extracted IgY antibodies from inoculated chickens-were plated in a dilution series and incubated for 1 h. A goat anti-chicken HRP-conjugated antibody was applied to probe for any IgY antibodies bound to the whole cells coated on the plate. The plate was developed with TMB and the reaction terminated with 1.0 M H₂SO₄. Absorbance at 450 nm was measured and recorded in a plate reader.

An indirect ELISA assay was used to detect the presence of specific anti-Staphylococcal protein A antibodies. The same steps as described above were used to perform the assay with the exception of the coating material. For the SpA-specific binding assay, SpA protein was coated to a 96-well microtiter plate at a concentration of 1.0 μg/mL.

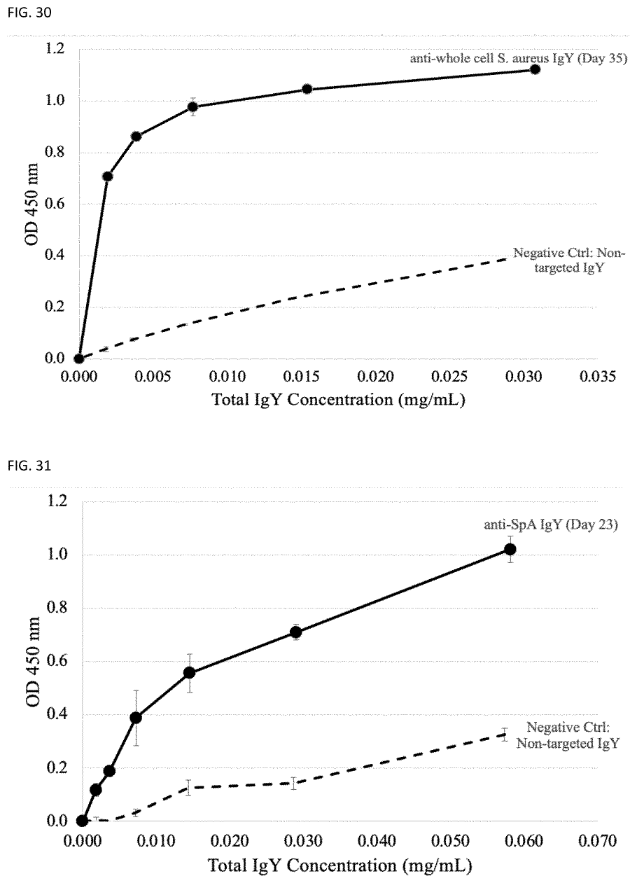

A graph of ELISA reactivity of anti-whole cell S. aureus IgY antibodies from serum collected 35 days post initial inoculation to coated formalin-fixed S. aureus cells is shown in FIG. 30. The OD (absorbance at 450 nm) values were plotted against total IgY concentration (mg/mL) to show a dose-dependence in specific antibody concentration. Serum from uninoculated (non-targeted) chickens was used as a negative control. Each sample was plated in duplicate and the averages and standard deviations are shown. Specific reactivity to coated formalin-fixed S. aureus cells was demonstrated.

A graph of ELISA reactivity of anti-Staphylococcal protein A (Spa) IgY from serum collected 35 days post initial inoculation against coated Spa is shown in FIG. 31. The OD (absorbance at 450 nm) values were plotted against total IgY concentration (mg/mL) to show a dose-dependence in specific antibody concentration. Serum from uninoculated (non-targeted) chickens was used as a negative control. Each sample was plated in duplicate and the averages and standard deviations are shown. Specific reactivity to coated Spa protein was demonstrated.

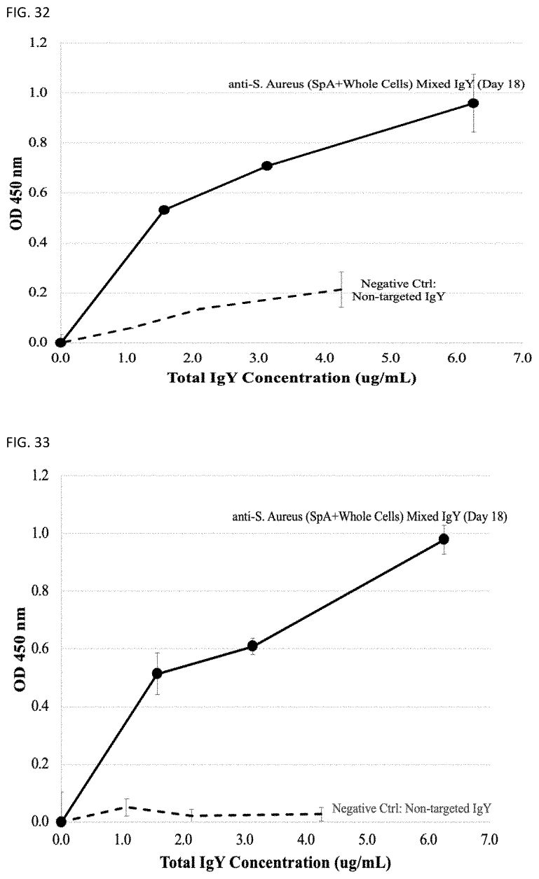

A graph of ELISA reactivity of a mixture of anti-SpA and anti-formalin fixed whole cell S. aureus IgY extracted from raw egg yolks against coated formalin-fixed S. aureus cells is shown in FIG. 32. The OD (absorbance at 450 nm) values were plotted against total IgY concentration (mg/mL) to show a dose-dependence in specific antibody concentration. IgY was extracted from eggs that were harvested 18 days after initial inoculation from a mixture of SpA and whole cell S. aureus inoculated chickens. Extracted IgY from non-targeted chickens was run for comparison. Each sample was plated in duplicate and the averages and standard deviations are shown. Specific reactivity to coated formalin-fixed S. aureus cells was demonstrated.

A graph of ELISA reactivity of a mixture of anti-SpA and anti-formalin fixed whole cell S. aureus IgY extracted from raw egg yolks against coated Spa protein is shown in FIG. 33. The OD (absorbance at 450 nm) values were plotted against total IgY concentration (mg/mL) to show a dose-dependence in specific antibody concentration. IgY was extracted from eggs that were harvested 18 days after initial inoculation from a mixture of SpA and whole cell S. aureus inoculated chickens. Extracted IgY from non-targeted chickens was run for comparison. Each sample was plated in duplicate and the averages and standard deviations are shown. Specific reactivity to coated Spa protein was demonstrated.

Results of indirect ELISA assays showed the presence of specific antibodies within the serum and isolated egg yolk anti-S. aureus-whole cell IgY and anti-SpA protein IgY samples. IgY antibodies from chickens inoculated with formalin-fixed S. aureus-whole cells and SpA protein displayed significantly greater binding to S. aureus and SpA than IgY antibodies from uninoculated chickens, by measure of absorbance at 450 nm. Antibodies specific to S. aureus cells and SpA protein offer a potential therapeutic to inhibit the colonization or binding capabilities of S. aureus and the virulent SpA protein.

### Example 30. Generation of Anti-Vibrio cholerae and Choleragen (Cholera Toxin) IgY Antibodies

The purpose of this experiment was to produce antibodies (mainly IgY) specific to V. cholerae and its toxin using a chicken host for the purposes of generating a low-cost, high-volume enteric therapeutic for humans. In this experiment, White Leghorn chickens were inoculated with choleragen and formalin-fixed V. cholerae cells.

Formalin-fixation is a technique which allows for the microbes and their surface proteins to be presented to the immune system while inhibiting cellular activity. The chicken's immune system recognizes the microbes and their surface proteins as antigen targets, inducing the production of specific polyclonal antibodies; while the microbes remain incapable of colonization.

A group of 13 chickens was inoculated with 2.00E+10 CFU/mL formalin-fixed Vibrio cholerae cells in Freund's Complete Adjuvant on Day 0 and boostered with the same concentration of cells in Freund's Incomplete Adjuvant on Days 23 and 37. Vibrio cholera ATCC® 14035™ were Formalin fixed and resuspended in PBS as follows. Lyophilized V. cholerae cells were ordered from ATCC (Cat #14035). Upon arrival, the cells were rehydrated and propagated according to the supplier's specifications. Briefly, for each inoculation cycle, V. cholerae cells were grown and expanded in liquid culture media overnight. After 16 h of growth, the cells were harvested via centrifugation and washed 3× in PBS. The cell pellet was resuspended in PBS and dilution plating was performed to calculate the CFU/mL. Next, 5 mL of the cell suspension was fixed in a 1% formalin solution for 16 h at 4° C. After the cells were fixed, they were harvested via centrifugation and washed 3× in PBS. The resulting cell pellet was resuspended in 5 mL of PBS to equal the amount of cell suspension removed from the original suspension. As the two volumes are equal, the CFU/mL calculated from the non-fixed PBS cell suspension was equal to the formalin-fixed cell suspension. A target concentration of 2.00E+10 CFU/mL (before addition of Freund's) was chosen for inoculation based on literature values. Hirai et al., 2010 Acta Medica Okayama, vol. 64, no. 3, 163-170. The CFU count from the cell suspension was used to determine the volume of formalin-fixed cells needed to meet the target concentration for inoculation. A group of 13 Leghorn chickens was selected for formalin-fixed V. cholerae whole cell inoculation. The quantities of formalin-fixed cell suspension, PBS and Freund's adjuvant were calculated, measured, and combined into a single formulation containing two distinct liquid layers. The inoculum was shipped overnight to the farming facility on ice packs. Immediately before injection, a water-in-oil emulsion was formed between the aqueous protein solution and Freund's adjuvant by forcibly mixing the formulation between two syringes using a three-way stop cock. After the emulsion was properly formed (i.e., the two distinct liquid layers had formed into one homogenized formulation), it was administered to the chickens via four intramuscular inoculations (two 250 μL injections in each breast for a total inoculation volume of 1 mL per chicken).

Preparation of V. cholerae Toxin Protein for Inoculation

Lyophilized isolated native V. cholerae toxin (composed of the A and B subunits, AB5, ˜85 kDa) was ordered from Sigma Aldrich (Cat C8052). Upon arrival, the toxin was rehydrated according to the supplier's specifications. A target concentration of toxin of 125 μg per chicken was chosen for inoculation. A group of 12 Leghorn chickens was selected for V. cholerae toxin (choleragen) inoculation. The volumes of protein resuspension, PBS and Freund's adjuvant were calculated, measured, and combined into a single formulation (Note: At this point, the single formulation contained two distinct liquid layers). The inoculum was shipped overnight to the farming facility on ice packs. Immediately prior to injection, a water-in-oil emulsion was formed between the aqueous protein solution and Freund's adjuvant by forcibly mixing the formulation between two syringes. After the emulsion was properly formed (i.e., the two distinct liquid layers had formed into one homogenous formulation), it was administered to the chickens via two intramuscular inoculations (one 250 μL injection in each breast for a total inoculation volume of 500 μL per chicken). A group of 12 chickens was inoculated with 125 μg of Vibrio cholerae choleragen (Cholera toxin-full alpha and beta subunit complex) in Freund's Complete Adjuvant on Day 0 and boosted with the same quantity of protein in Freund's Incomplete Adjuvant on Days 14, 37 and 48.

Following inoculation, chicken serum was isolated from whole blood samples and used to qualitatively measure specific antibody titer using antigen-specific indirect binding ELISA assays. IgY antibodies targeted to V. cholerae and choleragen were successfully produced and detected, giving indication of potential therapeutics in which antibodies inhibit the colonization or binding capabilities of virulent microorganisms and their toxins.

Processing of Antisera

Whole blood from chickens inoculated with choleragen was collected at 21 days post-inoculation (7 days post-1^{st} booster). Whole blood from chickens inoculated with 1% formalin-fixed V. cholerae cells was collected at 23 days post-inoculation. The blood had not been treated with any anti-coagulants and could therefore the serum could be separated from fibrinogen and other clotting factors. The serum samples were diluted with 1×PBS and analyzed for estimated total IgY concentrations using NanoDrop One© Protein A280 application.

Detection of anti-Whole Cell V. cholerae and anti-Choleragen IgY Antibodies was performed using indirect ELISA assay to detect the presence of anti-V. cholerae (whole cell) antibodies. Formalin-fixed V. cholerae cells at a concentration of 5.50E+08 CFU/mL were coated to a 96-well microtiter plate and incubated at 4° C. overnight. After incubation, the plate was washed and blocked with a 10% BlokHen® Solution for 2 h. After blocking, the plate was washed, and isolated serum from the fixed V. cholerae whole cell inoculated chickens were plated in a dilution series and incubated for 1 h. After the primary incubation period, the plate was washed and goat anti-chicken HRP-conjugated antibody was applied to probe for any IgY antibodies bound to the whole cells coated on the plate. The plate was developed with TMB and the reaction terminated with 1.0 M H₂SO₄. Absorbance at 450 nm was measured and recorded in a plate reader. FIG. 35 shows a graph of ELISA reactivity to coated formalin-fixed V. cholerae cells by anti-whole cell V. cholerae IgY in serum collected at 23 days post first inoculation and anti-choleragen IgY antibodies present in serum 21 days following the initial inoculation. Serum from uninoculated (non-targeted) chickens was used as a negative control. Each sample was plated in duplicate and the averages and standard deviations are plotted in the graph. Results show that anti-whole cell V. cholerae IgY in serum exhibited specific binding to coated fixed V. cholerae cells, but anti-choleragen IgY in serum did not exhibit specific binding to coated V. cholerae whole cells.

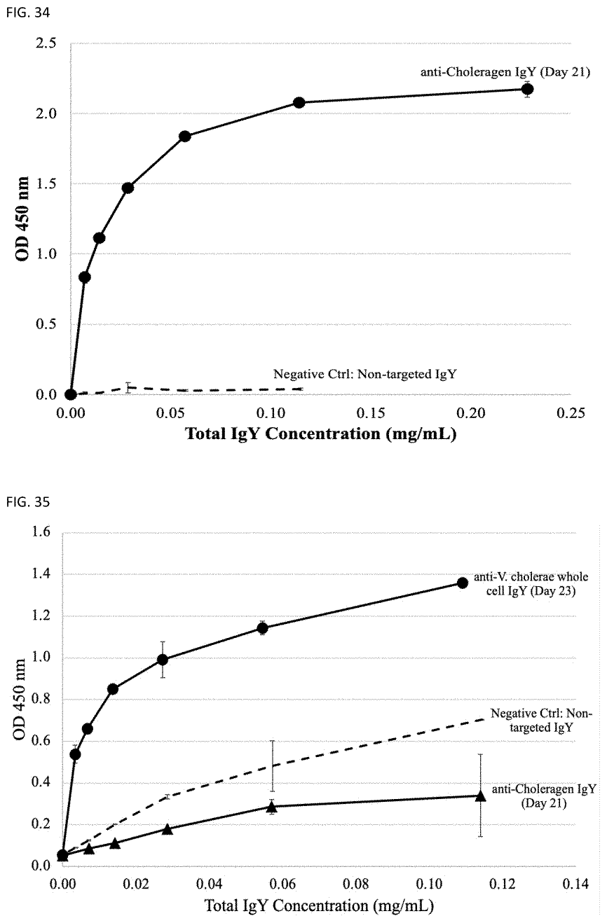

An indirect ELISA assay was used to detect the presence of anti-choleragen antibodies. The same steps as described above were used to perform the assay with the exception of the coating material. For the choleragen-specific binding assay, choleragen protein was coated to a 96-well microtiter plate at a concentration of 1.0 μg/mL. FIG. 34 shows a graph of ELISA reactivity of anti-choleragen IgY antibodies present in serum 21 days following the initial inoculation to coated choleragen. Serum from uninoculated (non-targeted) chickens was used as a negative control. Each sample was plated in duplicate and the averages and standard deviations were are plotted. Specific IgY reactivity to Choleragen was demonstrated. Inoculation of chickens with choleragen was successful in inducing specific antibody production in the target host. The presence of specific antibodies within the blood coupled with the natural processes of chicken passive immunity indicate specific antibodies are already present in the laid eggs.

In conclusion, these findings suggest that formalin-fixed whole cell V. cholerae and choleragen (cholera toxin) intramuscular inoculations induce an immunogenic response in the White Leghorn chickens resulting in the production of specific polyclonal antibodies. Specific IgY may be useful in the production of an anti-V. cholerae and anti-choleragen oral composition for enteric use. These supplements could be used for the relief of symptoms caused by choleragen, and the prevention of V. cholerae colonization and derived illnesses in developing countries.

### Example 31. Production of Anti-SARS-CoV-2 IgY (Neutralizing) Antibodies Following Plasmid DNA Inoculation

The purpose of this example was to demonstrate that chickens inoculated with anti-SARS-CoV-2 DNA plasmid vaccines produce specific IgY targeted to the antigen encoded by the plasmid in order to develop egg-based SARS-CoV-2-specific IgY antibodies that could be used as a potential therapeutic. SARS-CoV-2 neutralizing effects of anti-ACE2 IgY antibodies promoted by the inoculation of a plasmid DNA inoculum were demonstrated.

Most chicken inoculations are done using proteins mixed with adjuvants, but proteins can be difficult to produce and extremely expensive to purchase. Plasmid vaccines can be developed much more quickly and are easily produced by growing cultures of E. coli containing the plasmid. The plasmid vectors have been designed to produce proteins by placing the gene of interest behind a promoter sequence that promotes expression in eukaryotic cells.

anti-SARS-CoV-2 DNA plasmid vaccines were developed using the pCI_Neo vector (SEQ ID NO: 83) to express proteins in eukaryotic cells. A chicken codon optimized ACE2 nucleotide sequence encoding human ACE2 (SEQ ID NO: 78) was cloned on the pCI_Neo plasmid to form pCI_Neo-ACE2 plasmid (FBB_p071) which uses the human cytomegalovirus (CMV) promoter region to produce constitutive expression of the gene of interest. The codon-optimized target DNA sequence was inserted between the T7 promoter sequence TAATACGACTCACTATAGG (SEQ ID NO: 84) and the SV40 terminator sequence TGATAAGATACATTGATGAGTTTGGACAAACCACAACTAGAATGCAGTGAAAAA AATGCTTTATTTGTGAAATTTGTGATGCTATTGCTTTATTTGTAACCATTATAAGC TGCAATAAAC (SEQ ID NO: 85) to obtain a plasmid DNA eukaryotic expression vector. The new expression plasmid was assembled using Gibson assembly. The assembled plasmid was transformed into electrocompetent E. coli cells. The growth and extraction of large quantities of plasmid was performed using Gigaprep kits (Qiagen). The sequence confirmed purified plasmid pCI_Neo-ACE2 plasmid (FBB_p071) were used for inoculation into the chicken host.

An inoculum was prepared containing a mixture of phosphate buffered saline (PBS), the plasmid DNA, and an adjuvant. One adjuvant was Class B oligonucleotide ODN 1826 (Invivogen tlrl-1826)(referred to as CpG). Additionally, a plasmid adjuvant in a separate plasmid or within the sequence of the plasmid DNA of interest was employed in certain inoculations. CpG is a synthetic oligodeoxynucleotides of the following sequence: TCCATGACGTTCCTGACGTT (SEQ ID NO: 93). This sequence is interpreted, by the host, as a signal of prokaryote invasion and therefore initiates immune system defense mechanisms. CpG has been used to enhance the immune response induced by DNA vaccines. The plasmid adjuvants used for the DNA-based inoculations encode sequences of the following cytokines: interferon gamma (IFNγ), heat shock protein from M. tuberculosis (HSP70), interleukin-2 from Gallus gallus (IL-2), and chicken granulocyte-macrophage colony stimulating factor (chGMCSF). The role of cytokines as plasmid adjuvants is to drive an immune response directed towards the plasmid of interest with which they were simultaneously inoculated. The first inoculation, and all subsequent injections (boosters), use the same materials at the same concentrations.

Briefly, the plasmid DNA is cultured in E. coli, purified, and the resulting concentration is measured by NanoDrop. Next, the amount of plasmid of interest DNA, PBS, plasmid adjuvant DNA, and CpG needed for the inoculation was calculated. After the formulation components are combined, the solution is mixed and the chickens were injected intramuscularly.

A group of 5 chickens was selected for the plasmid DNA ACE2 inoculation. The volumes of plasmid suspension, PBS and CpG adjuvant were calculated, measured, and combined into a single formulation. The inoculum was shipped overnight to the farming facility on ice packs. Immediately before injection, the tube containing the inoculum was invented a few times to ensure the solution was homogenous. After the solution was gently mixed, it was administered to the chickens via two intramuscular injections (a 250 μL injection in each breast for a total volume of 500 μL per chicken). The chickens received an initial inoculation (Day 0) of 500 μL containing ˜300 μg of ACE2 plasmid with 20 μg of CpG. The group received booster inoculations on Day 8, 19 and 34.

Plasmid adjuvants, pCI_Neo-IL2, pCI_Neo-IFNγ and pCI_Neo-chGMCSF, were prepared in the same manner as pCI_Neo-ACE2. Each plasmid adjuvant was separately paired with pCI_Neo-ACE2 and CpG and inoculated into a group of 5 chickens. The volumes of plasmid suspensions, PBS and CpG were calculated, measured, and combined into a single formulation. The inoculum was shipped overnight to the farming facility on ice packs. Immediately before injection, the tube containing the inoculum was invented a few times to ensure the solution was homogenous. After the solution was gently mixed, it was administered to the chickens via two intramuscular injections (a 250 μL injection in each breast for a total volume of 500 μL per chicken). The chickens received an initial inoculation (Day 0) of 500 μL containing a total of 600 μg plasmid DNA (300 μg of ACE2 plasmid+300 μg adjuvant plasmid) and 20 μg of CpG. The three groups received booster inoculations on Days 12 and 24.

Processing of Antisera. Whole blood from test groups was collected 27 and 28 days following the initial inoculation. The blood had not been treated with any anti-coagulants and could therefore separate the serum from fibrinogen and other clotting factors. The serum samples were diluted with 1×PBS and analyzed for estimated total IgY concentration using NanoDrop One© Protein A280 application.

Processing of IgY from Egg Yolks. IgY antibodies were extracted from 5 whole eggs collected 16, 23, 30 and 40 days following initial inoculation and pooled together for processing. Briefly, the eggs were washed, cracked, and the yolk separated from the albumen. The yolks were then diluted 8-fold with tap water and the pH adjusted to 5.0 using HCl. The yolk solution was then frozen for 12-16 h. Once frozen, the solution was thawed over a filtration apparatus to remove the lipidic components of the yolk and the water-soluble fraction was collected. The volume of the water-soluble fraction was measured, 8.8% (w/v) NaCl was added, and the pH adjusted to 4.0 using HCl. Using centrifugation, the IgY antibodies were pelleted. The pellet was resuspended in 2.5 mL of PBS and dialyzed for 12-16 h. The dialyzed extraction was analyzed using the NanoDrop™ One© Protein A280 application (SOP.017) to determine the total IgY concentration within the sample.

Detection of Specific Anti-ACE2 IgY Antibodies by Indirect Assay

An ELISA indirect assay was used to detect the presence of specific anti-ACE2 antibodies. ACE2 protein from Creative Biomart, at a concentration of 1.0 μg/mL, was coated to a 96-well microtiter plate and incubated at 4° C. overnight. After incubation, the plates were washed and blocked with a 10% BlokHen® Solution for 2 h. After blocking, the plates were washed and isolated serum from inoculated and uninoculated chickens were plated, in duplicate, in a dilution series and incubated for 1 h. After the primary incubation period, the plates were washed and a goat anti-chicken HRP-conjugated antibody was applied to probe for any IgY antibodies bound to the ACE2 protein coated on the plates. The plates were developed with TMB and the reaction terminated with 1.0 M H₂SO₄. Absorbance at 450 nm was measured and recorded in a plate reader.

A modified GenScript SARS-CoV-2 Surrogate Virus Neutralization Test (sVNT) C-Pass™ Kit protocol was performed to assess the neutralizing capability of anti-ACE2 IgY from the plasmid DNA inoculated chickens. The modified GenScript protocol calls for anti-ACE2 IgY Test Samples to incubate with an ACE2-coated microplate for 30 minutes at 37° C. After this incubation period, HRP-conjugated RBD protein is applied to the microplate wells at a 1:1 volumetric ratio. This mixture of HRP-RBD and anti-ACE2 IgY within the ACE2-coated microplate is incubated for 15 minutes at 37° C. After this step, the plate is washed, and TMB substrate is applied to visualize any HRP-RBD binding activity to ACE2. The reaction is terminated with a provided STOP Solution and the absorbance of the microplate is read at 450 nm. Inhibition of the binding event of RBD-HRP to ACE2 is indicated by little to no color change, and the percentage of inhibition is calculated relative to the provided Negative Control's absorbance value.

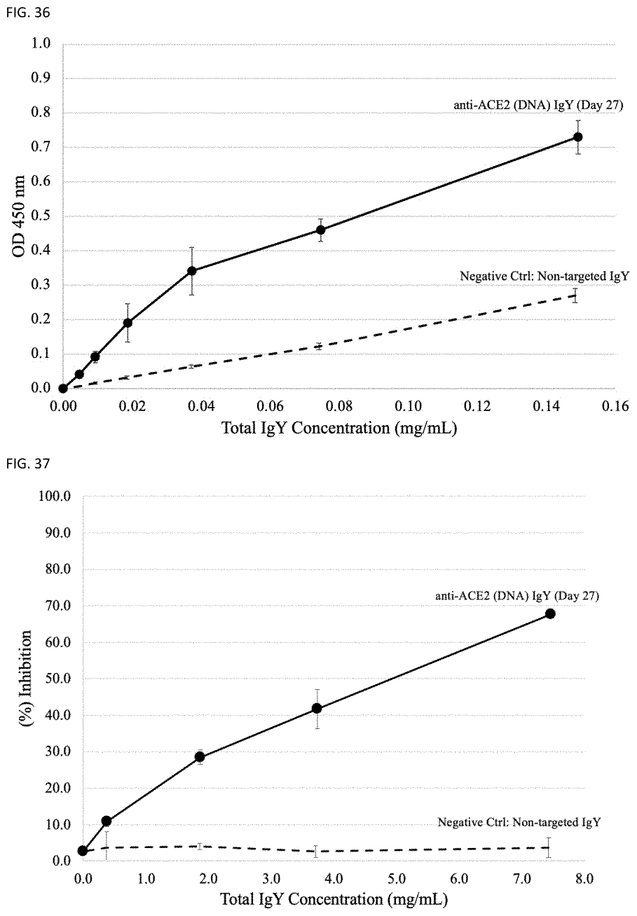

FIG. 36 shows a graph of ELISA reactivity of anti-ACE2 IgY antibodies present in serum 27 days post the initial plasmid DNA inoculation (ACE2 DNA+CpG) to coated ACE2 protein (Creative Biomart) v total IgY concentration. Serum from uninoculated (non-targeted) chickens was used as a negative control. Each sample was plated in duplicate and the averages and standard deviations are plotted.

FIG. 37 shows a graph of average percent inhibition of RBD:ACE2 binding by anti-ACE2 IgY from Chicken Serum collected at Day 27 post initial plasmid DNA-based inoculation compared to IgY Extracted from non-targeted chicken serum (sVNT Kit). The percent inhibition of RBD:ACE2 binding by anti-ACE2 IgY antibodies in serum from plasmid DNA-inoculated chickens is plotted v total IgY concentration (mg/mL), as compared to serum from uninoculated chickens (non-targeted IgY), tested within the GenScript surrogate Virus Neutralization Test (sVNT) Kit.

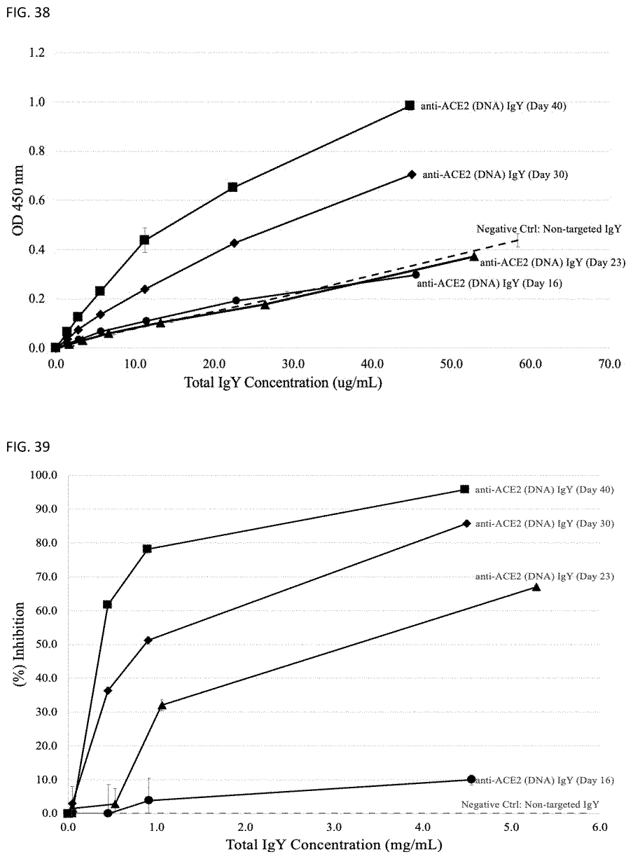

FIG. 38 shows a graph of ELISA reactivity of anti-ACE2 IgY extracted from raw egg yolks against collected at Day 16, 23, 30, and 40 post initial inoculation against coated ACE2 Protein. The optical density at 450 nm (OD)±the standard deviation (std dev) of the extracted IgY dilution series from eggs sampled at 16, 23, 30, and 40 days post inoculation (300 ug plasmid ACE2 DNA+20 ug CpG per chicken per innoculation) against coated ACE2 protein. The plates were read at 450 nm, and the OD values were plotted against total IgY concentration (μg/mL), measured by NanoDrop to show a dose-dependence in specific antibody concentration.

FIG. 39 shows a graph of average percent inhibition of RBD:ACE2 binding by anti-ACE2 IgY antibodies in extracted IgY from raw egg yolks from plasmid DNA inoculated chickens at Day 16, 23, 30 and 40 post initial inoculation (ACE2 DNA+CpG adjuvant), as compared to negative non-targeted IgY control samples, tested within the GenScript surrogate Virus Neutralization Test (sVNT) Kit. The same samples were employed in FIG. 38. At day 30, anti-ACE2 IgY exhibits >80% inhibition of RBD:ACE2 binding. At day 40, anti-ACE2 IgY exhibits >90% inhibition of RBD:ACE2 binding.

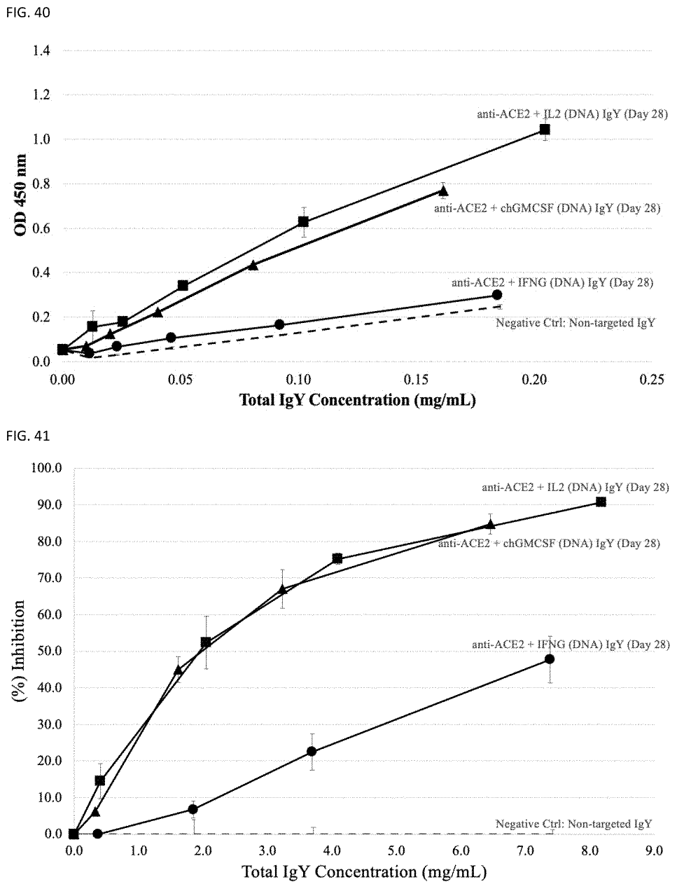

FIG. 40 shows a graph of ELISA reactivity plotted as optical density 450 nm (OD)±the standard deviation (std dev) of a chicken serum dilution series from pCI_Neo-ACE2 and plasmid adjuvant co-inoculated chickens plated against ACE2 protein. The plasmid adjuvants are as follows: pCI_Neo-IL2, pCl_Neo-IFNγ, and pCl_Neo-chGMCSF. Serum from uninoculated (non-targeted) chickens was used as a negative control. The plates were read at 450 nm and the OD values were plotted against total IgY concentration (mg/mL), measured by NanoDrop, to show a dose-dependent response.

FIG. 41 shows a graph of ELISA inhibition of RBD:ACE2 Binding by anti-ACE2 IgY Serum collected at day 28 post first inoculation from Chickens co-inoculated with pCl_Neo-ACE2 and Plasmid Adjuvants. The average percent inhibition values and standard deviations of the anti-ACE IgY and negative control samples tested in the GenScript surrogate Virus Neutralization Test (sVNT) Kit. Greater than 80% inhibition was exhibited in anti-ACE2 IgY serum from chickens co-inoculated with either the 1L2 plasmid adjuvant or the chGNCSF plasmid adjuvant compared to the IFNgamma plasmid adjuvant.

1. An antiviral composition comprising an effective amount of anti-SARS-CoV-2-S-protein immunoglobulin Y (IgY) antibodies, and a pharmaceutically acceptable carrier.

2. The antiviral composition according to claim 1, further comprising an effective amount of anti-human ACE-2 IgY antibodies.

3. The antiviral composition according to claim 1, further comprising an effective amount of anti-SARS-CoV-2N-protein-specific polyclonal IgY antibodies.

4. The antiviral composition according to claim 1, wherein the anti-SARS-CoV-2-S-protein immunoglobulin Y (IgY) antibodies bind to a SARS-CoV-2 S protein RBD domain.

5. An antiviral composition comprising an effective amount of anti-SARS-CoV-2-RBD-protein immunoglobulin Y (IgY) antibodies, and a pharmaceutically acceptable carrier.

6. An antiviral composition comprising an effective amount of anti-ACE-2-immunoglobulin Y (IgY) antibodies, and a pharmaceutically acceptable carrier.

7. An antiviral composition comprising an effective amount of anti-SARS-CoV-2-RBD-protein immunoglobulin Y (IgY) antibodies, an effective amount of anti-human ACE-2 IgY antibodies, and a pharmaceutically acceptable carrier.

8. The antiviral composition according to claim 1, wherein the SARS-CoV-2-S-protein comprises an amino acid sequence selected from the group consisting of SEQ ID NO: 1, 6, 7, 10, 11, 12, 13, 14, 15, 16, 17, 18, 26, 27, 36, 37, 38, 39, 40, 41, 86, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

9. The antiviral composition according to claim 2, wherein the human ACE2 protein comprises an amino acid sequence selected from the group consisting of SEQ ID NO: 28, 35, 42, 43, 77, and 78, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

10. The antiviral composition according to claim 3, wherein the SARS-CoV-2-N-protein comprises an amino acid sequence selected from the group consisting of SEQ ID NO: 4, 8, 9, 24, 25, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

11. The antiviral composition according to claim 5, wherein the SARS-CoV-2-RBD-protein comprises an amino acid sequence selected from the group consisting of SEQ ID NO: 15, 36, 37, 38, 39, 122, 123 or a fragment thereof comprising from 10 to 200, 10 to 100, 20 to 50, or at least 10, 20, 30, 40, 50, 60, 70, 80, 100, 150, or 200 contiguous amino acid residues thereof, or a substantially similar protein thereof.

12.-13. (canceled)

14. The antiviral composition according to claim 1, further comprising anti-TMPRSS2 IgY antibodies.

15. The antiviral composition according to claim 1, further comprising a flavoring, sweetener, stabilizer, pH regulator, preservative, antibody matrix, or vitamin.

16. The antiviral composition according to claim 1, wherein the IgY antibodies are in the form of isolated IgY antibodies, whole immune egg, immune egg yolk, defatted immune egg yolk, whole immune egg powder, immune egg yolk powder, defatted immune egg yolk powder, egg extract, serum, or serum extract.

17. The antiviral composition according to claim 16, wherein the isolated IgY antibodies are in a purified and or concentrated form.

18. The antiviral composition according to claim 1, wherein the IgY antibodies comprise polyclonal IgY antibodies.

19. The antiviral composition according to claim 1, wherein the IgY antibodies comprise neutralizing polyclonal IgY antibodies.

20. The antiviral composition according to claim 19, wherein the neutralizing anti-SARS-CoV-2-S-protein immunoglobulin Y (IgY) antibodies are derived from eggs of hens inoculated with an immunogen selected from the group consisting of a recombinant SARS-CoV-2 RBD-protein or a fragment thereof, a recombinant SARS-CoV-2 S-protein or a fragment thereof, a recombinant SARS-CoV-2 S-ECD protein or a fragment thereof, a recombinant SARS-CoV-2 S1-protein or a fragment thereof, and a recombinant SARS-CoV-2 S2-protein or a fragment thereof, or a substantially similar protein.

21. (canceled)

22. A dosage form comprising the antiviral composition according to claim 1, in the form of a spray, mouth spray, nasal spray, inhalation aerosol, lozenge, troche, gel, mucoadhesive gel, film, liquid, powder, capsule, tablet, caplet, mouth wash, mouth rinse, mouth gargle, inhalable powder, suppository, inhalable fluid, and injectable fluid.

23. A method of reducing viral replication in a cell comprising treating a coronavirus-infected cell with an effective amount of a composition according to claim 1.

24. A method for the treatment or prevention of a SARS-CoV-2 viral infection in a subject in need thereof comprising administering to or exposing the subject to an effective amount of a composition according to claim 1.

25. (canceled)

26. A method for reducing severity or duration of symptoms of a SARS-CoV-2 coronavirus infection in a subject in need thereof, comprising administering a composition according to claim 1.

27. The method according to claim 26, wherein the symptoms are selected from the group consisting of fever, cough, muscle aches, lethargy, diarrhea, vomiting, headache, stomachache, shortness of breath, muscle pain, sputum production, diarrhea, sore throat, complete or partial loss of smell, and complete or partial loss of taste.

28.-37. (canceled)

38. A kit for preventing or decreasing transmission of a SARS-CoV-2 virus, comprising in at least one container, the antiviral composition according to claim 1, and optionally at least a second container comprising a diluent, a sheet of instructions, and/or an applicator.

39. (canceled)

40. A pharmaceutical composition comprising an effective amount of isolated anti-coronavirus IgY antibodies, immune egg, or immune egg yolk derived from eggs of poultry vaccinated with a coronavirus vaccine, a recombinant polynucleotide encoding a SARS-CoV-2 protein, or a recombinant SARS-CoV-2 protein, or fragment thereof, and a pharmaceutically acceptable excipient or carrier.

41. The composition according to claim 40, wherein the anti-coronavirus IgY antibodies comprise anti-SARS-CoV-2 coronavirus protein IgY antibodies.

42. The composition according to claim 40, wherein the SARS-CoV-2 protein is selected from the group consisting of an S-protein, S1-protein, S2-protein, RBD-protein, S1-S2-ECD-protein, S-RBD-protein, N-protein, or M-SARS-CoV-2 protein.

43. The composition according to claim 42, wherein the SARS-CoV-2-S-protein comprises an amino acid sequence selected from the group consisting of SEQ ID NO: 1, 6, 7, 10, 11, 12, 13, 14, 15, 16, 17, 18, 26, 27, 36, 37, 38, 39, 40, 41, 86 or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

44. The composition according to claim 42, wherein the SARS-CoV-2-RBD-protein comprises an amino acid sequence selected from the group consisting of SEQ ID NO: 15, 36, 37, 38, 39, 122, 123 or a fragment thereof comprising from 10 to 200, 10 to 100, 20 to 50, or at least 10, 20, 30, 40, 50, 60, 70, 80, 100, 150, or 200 contiguous amino acid residues thereof, or a substantially similar protein thereof.

45. The composition according to claim 42, wherein the SARS-CoV-2-N-protein comprises an amino acid sequence selected from the group consisting of SEQ ID NO: 4, 8, 9, 24, 25, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

46. The composition according to claim 40, wherein the coronavirus vaccine is a poultry, bovine, porcine, canine, human, feline, or ferret coronavirus vaccine.

47. The composition according to claim 40, further comprising an effective amount of isolated anti-human ACE2 IgY antibodies, immune egg, or immune egg yolk derived from eggs of poultry vaccinated with a recombinant polynucleotide encoding a human ACE2 protein and/or a recombinant or synthetic human ACE2 protein.

48. The antiviral composition according to claim 47, wherein the human ACE2 protein comprises an amino acid sequence selected from the group consisting of SEQ ID NO: 28, 35, 42, 43, 77, and 78, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

49.-50. (canceled)

51. A method for producing immunoglobulin Y (IgY) polyclonal antibodies comprising
identifying a target pathogen and/or target biomolecule;
selecting a first immunogen derived from the target pathogen and/or biomolecule;
preparing a first inoculant comprising the first immunogen, a first adjuvant, and a first vehicle or carrier;
inoculating a host avian with the first inoculant;
reinoculating the host avian with a second inoculant comprising a second immunogen, a second adjuvant, and a second vehicle or carrier;
collecting eggs and/or blood from the host avian; and
processing the eggs or blood to obtain isolated IgY antibodies.

52. The method according to claim 51, wherein the second inoculant is prepared comprising
selecting a second immunogen derived from the target pathogen or target biomolecule; and
preparing the second inoculant comprising the second immunogen, the second adjuvant, and the second vehicle.

53. The method according to claim 51, wherein the first and second immunogens are selected from the group consisting of a fixed, attenuated, or inactivated whole cell immunogen, a protein immunogen, and a plasmid DNA encoding a protein immunogen.

54. The method according to claim 53, wherein the first and second immunogens are different.

55. The method according to claim 51, wherein the first immunogen is a protein immunogen selected from the group consisting of an isolated protein, synthetic protein, or a recombinant protein.

56. The method according to claim 55, wherein the second immunogen is a plasmid DNA immunogen encoding the protein immunogen, or fragment thereof, or a substantially similar protein.

57. (canceled)

58. The method according to claim 51, wherein the target pathogen or target biomolecule is selected from the group consisting of coronavirus, norovirus, zika virus such as PRV ABC59, rhinovirus, herpes virus, influenza virus, smallpox virus, Ebola virus, rotavirus, calicivirus, cytomegalovirus, astrovirus, adenovirus, enteric adenovirus, Staphylococcus aureus, Vibrio cholerae such as Vibrio O1, Vibrio O139, Non-O1 Vibrios, Vibrio parahaemolyticus, Campylobacter jejuni, Salmonella spp. such as Salmonella typhimurium, Salmonella enterica serovar Typhi, bacillus spp. such as Bacillus cereus, Bacillus anthracis, Shigella dystenteriae, Plasmodium falciparum, Plesiomonas shigelloides, Escherichia coli [including (EPEC) enteropathogenic E. coli, (ETEC) enterotoxigenic E. coli, (EaggEC) enteroaggregative E. coli, (EIEC) enteroinvasive E. coli, and (EHEC) haemorrhagic E. coli], Yersinia enterocolitica, Aeromonas hydrophila, Clostridium perfringens, Clostridium dificile, enterohepatic Helicobacter (including Helicobacter pylori), Staphylococcus aureus, Klebsiella spp., Mycobacterium tuberculosis, Streptococcus pyogenes, Salmonella enterica serotypes Paratyphi A and B, Enterobacter spp. such as Enterobacter cloacae or Enterobacter sakazakii, Aeromonas spp. such as A. caviae, A. veronii biovar sobria, Proteus spp. such as P. mirabilis or P. vulgaris, Citrobacter spp. such as C. freundii, Serratia spp. such as S. marcescens, S. rubidaea, Cryptosporidium spp., venom, toxin such as cholera toxin, adhesion element, prion protein, and prion-like protein, or a receptor-binding domain therefor.

59. The method of claim 58, wherein the coronavirus is selected from the group consisting of a SARS-CoV-2 virus, a SARS-CoV virus, and a MERS virus.

60. The method according to claim 51, wherein the target biomolecule is a human ACE2 protein, or fragment thereof, or a substantially similar protein.

61. The method according to claim 51, wherein the first and optionally the second adjuvant is selected from the group consisting of Freund's Complete Adjuvant (FCA), Freund's Incomplete Adjuvant, mineral adjuvants, such as aluminum compounds, aluminum hydroxide, ALUM, potassium alum, potassium aluminum sulfate, aluminum hydroxy phosphate sulfate, aluminum phosphate, calcium phosphate hydroxide, bacterial adjuvants such as muramyl dipeptides, flagellin, monophosphoryl lipid A, killed Bordetella pertussis, Mycobacterium bovis, toxoids, lipopolysaccharide, aluminum monostearate, mannide monooleate, vegetable oil, paraffin oil, water, polysorbate 80, polysorbate 20, octoxynol-10, octylphenol ethoxylate, block copolymer, CRL-89-41, squalene, oil in water emulsion comprising squalene, Titermax Classical adjuvant (SIGMA-ALDRICH), lipid based immunostimulant complexes (ISCOMS) mix of cholesterol, dioleoyl phosphatidyl choline, 3-O-desacyl-4′monophosphoryl lipid A, Quillaja saponins, Quil A, Lipid A derivatives, cholera toxin derivatives, diphtheria toxoid, heat shock protein (HSP) derivatives, lipopolysaccharide (LPS) derivatives, synthetic peptide matrixes, GMDP, oil-based adjuvant such as Xtend®III (Grand Laboratories, Inc., Larchwood, Iowa) immunostimulants (U.S. Pat. No. 5,876,735), interleukins such as IL-1, IL-2, IL-6, IL-8, IL-12, IL-15, IL-18, cytokines such as interferon gamma, chGMCSF, Flt3 ligand, class B oligodeoxynucleotide (ODN) CpG, phosphorothioate-linked oligodeoxynucleotide, and a plasmid adjuvant DNA encoding a cytokine, interleukin, or heat shock protein.

62. The method according to claim 51, wherein the first and optionally the second vehicle or carrier comprises one or more components selected from the group consisting of water, phosphate buffered saline, sodium chloride, sucrose, lactose, trehalose, dextrose, microcrystalline cellulose, potassium phosphate, sodium phosphate, magnesium stearate, sodium bicarbonate, sodium carbonate, 2-phenoxyethanol, protamine sulfate, urea, citric acid, sodium metabisulfite, monosodium glutamate, ethlenediamine tetraacetic acid (EDTA), optionally wherein the vehicle or carrier comprises a preservative.

63. The method according to claim 62, wherein the preservative is selected from the group consisting of neomycin, neomycin sulfate, polymixin B, thimerosal, formaldehyde, and phenol.

64. The method according to claim 55, wherein the protein immunogen is selected from the group consisting of SARS-CoV-2 RBD-protein, SARS-CoV-2 S-protein, SARS-CoV-2 S2-protein, SARS-CoV-2 S1-protein, SARS-CoV-2 N-protein, human ACE2 protein, norovirus capsid protein, Plasmodium falciparum circumsporozoite protein, Cryptosporidium protein such as C. parvum P23, a Clostridium difficile protein FliC, FliD, Cwp84, or Toxin B (TcdB), Staphylococcal protein A, CD20 protein, venom, rhinovirus VP4 protein, influenza VP1 capsid protein, prion protein, prion-like protein, herpes simplex virus glycoprotein gD, herpes simplex virus glycoprotein gD, rotavirus VP4 capsid protein, rotavirus VP7 surface glycoporotein, rotavirus NSP4 viral enterotoxin, zika virus NS-1 protein, Smallpox virus vaccinia complement protein (VCP), Bacillus anchracis lethal factor, Bacillus anchracis edema factor, Bacillus anchracis protective antigen (pagA), Ebola virus glycoprotein, Staphylococcus aureus SpA, cholera toxin subunit A, cholera toxin subunit B, and cholera toxin AB5, or a substantially similar protein.

65. The method according to claim 51, wherein the host avian is a chicken.

66. The method according to claim 51, wherein the first or second immunogen is a plasmid DNA encoding a protein of the target pathogen or biomolecule.

67. The method according to claim 66, wherein the plasmid DNA comprises a eukaryotic expression vector.

68. The method according to claim 67, wherein the eukaryotic expression vector is selected from the group consisting of pCI-neo mammalian expression vector, pVIVO2-mcs vector, pVAX1 vector, pIRES Vector, and a pcDNA 3.1 Mammalian Expression Vector, optionally wherein the pCI-neo mammalian expression vector comprises the sequence of SEQ ID NO: 83.

69. (canceled)

70. The method according to claim 68, wherein the plasmid DNA encodes a protein selected from the group consisting of a SARS-CoV-2 S-protein, a SARS-CoV-2 RBD-protein, and a human ACE2 protein, or a fragment thereof, or a substantially similar protein.

71. The method according to claim 70, wherein the plasmid DNA encodes a SARS-CoV-2 S-protein comprising an amino acid sequence selected from the group consisting of SEQ ID NO: 1, 6, 7, 10, 11, 12, 13, 14, 15, 16, 17, 18, 36, 37, 38, 39, 40, 41, and 86, or a fragment thereof comprising from 50 to 1000, or from 100 to 500, or at least 10, 20, 30, 40, 50, 60, 70, 80, 100, 150, or 200 contiguous amino acid residues thereof, or a substantially similar protein.

72. The method of claim 70, wherein the plasmid DNA encodes a SARS-CoV-2-RBD-protein comprising an amino acid sequence selected from the group consisting of SEQ ID NO: 15, 36, 37, 38, 39, 122, and 123 or a fragment thereof comprising from 10 to 200, 10 to 100, 20 to 50, or at least 10, 20, 30, 40, 50, 60, 70, 80, 100, 150, or 200 contiguous amino acid residues thereof, or a substantially similar protein thereof.

73. The method according to claim 70, wherein the plasmid DNA encodes a human ACE2 protein comprising an amino acid sequence selected from the group consisting of SEQ ID NO: 28, 35, 42, 43, 77, and 78, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

74. The method according to claim 61, wherein the plasmid adjuvant comprises a eukaryotic expression vector and encodes a cytokine, interleukin, or heat shock protein.

75. The method according to claim 74, wherein the plasmid adjuvant encodes a cytokine, interleukin, and/or heat shock protein selected from the group consisting of interferon gamma (IFNγ), heat shock protein from M. tuberculosis (HSP70), interleukin-2 from Gallus gallus (IL-2), IL-6, IL-8, IL-15, chicken granulocyte-macrophage colony stimulating factor (chGMCSF), cytokine Flt3 ligand, CCL19.

76. The method according to claim 74, wherein the eukaryotic expression vector of the plasmid adjuvant is selected from the group consisting of pCI-neo mammalian expression vector, pVIVO2-mcs vector, pVAX1 vector, pIRES Vector, and a pcDNA 3.1 mammalian expression vector.

77. A method for preparing a plasmid DNA immunogen, comprising
a) selecting a target protein amino acid sequence or a DNA sequence encoding the target protein amino acid sequence;
b) optimizing the codons of a DNA sequence encoding the amino acid sequence of the target protein for expression in an avian to obtain a codon-optimized target DNA sequence, optionally wherein the avian is Gallus gallus; and
c) cloning the codon-optimized target DNA sequence into a eukaryotic expression vector to obtain the plasmid DNA immunogen.

78. The method of claim 77, wherein the target protein sequence is selected from the group consisting of a SARS-CoV-2 S-protein, SARS-CoV-2 S1-protein, SARS-CoV-2 RBD-protein, SARS-CoV-2 N-protein, human ACE2 protein, norovirus capsid protein, Plasmodium falciparum circumsporozoite protein, Cryptosporidium protein such as C. parvum P23, a Clostridium difficile protein, for example, FliC, FliD, Cwp84, or Toxin B (TcdB), Staphylococcal protein A, CD20 protein, venom, rhinovirus VP4 protein, influenza VP1 capsid protein, prion protein, prion-like protein, herpes simplex virus glycoprotein gD, herpes simplex virus glycoprotein gD, rotavirus VP4 capsid protein, rotavirus VP7 surface glycoporotein, rotavirus NSP4 viral enterotoxin, zika virus NS-1 protein, Smallpox virus vaccinia complement protein (VCP), Bacillus anchracis lethal factor, Bacillus anchracis edema factor, Bacillus anchracis protective antigen (pagA), Ebola virus glycoprotein, Staphylococcus aureus SpA, cholera toxin subunit A, cholera toxin subunit B, or cholera toxin AB5, or a fragment thereof, or substantially similar protein.

79. The method of claim 78, wherein the SARS-CoV-2-S-protein comprises the amino acid sequence selected from the group consisting of SEQ ID NO: 1, 6, 7, 10, 11, 12, 13, 14, 15, 16, 17, 18, 36, 37, 38, 39, 40, 41, 86, or a fragment thereof comprising from 50 to 1000, or from 100 to 500, or at least 10, 20, 30, 40, 50, 60, 70, 80, 100, 150, or 200 contiguous amino acid residues thereof, or a substantially similar protein.

80. The method of claim 78, wherein the human ACE2 protein comprises an amino acid sequence of SEQ ID NO: 28, 35, 42, 43, 77, 78, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

81. The method of claim 78, wherein the SARS-CoV-2-N-protein comprises an amino acid sequence selected from the group consisting of SEQ ID NO: 4, 8, 9, 24, 25, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

82. The method of claim 78, wherein the SARS-CoV-2-RBD-protein comprises an amino acid sequence selected from the group consisting of SEQ ID NO: 15, 36, 37, 38, 39, 122, 123 or a fragment thereof comprising from 10 to 200, 10 to 100, 20 to 50, or at least 10, 20, 30, 40, 50, 60, 70, 80, 100, 150, or 200 contiguous amino acid residues thereof, or a substantially similar protein thereof.

83. The method according to claim 77, wherein the eukaryotic expression vector is selected from the group consisting of pCI-neo mammalian expression vector, pVIVO2-mcs vector, pVAX1 vector, pIRES Vector, and a pcDNA 3.1 mammalian expression vector.

84. An oral composition comprising an effective amount of anti-SARS-CoV-2-S-immunoglobulin Y (IgY) antibodies, an effective amount of anti-human ACE-2 IgY antibodies, and a pharmaceutically acceptable carrier.

85. The oral composition comprising according to claim 84, wherein the anti-SARS-CoV-2-S-immunoglobulin Y (IgY) antibodies comprise anti-SARS-CoV-2-RBD-immunoglobulin Y (IgY) antibodies.

86. The oral composition according to claim 84, further comprising a flavoring, sweetener, stabilizer, pH regulator, preservative, antibody matrix, or vitamin.

87. The oral composition according to claim 86, wherein the antibody matrix comprises an enteric coating.

88. A dosage form comprising the oral composition according to claim 84, in the form of a spray, lozenge, troche, gel, mucoadhesive gel, film, liquid, powder, capsule, tablet, caplet, mouth rinse, or powder.

89. The antiviral composition of claim 8, wherein the SARS-CoV-2 S-protein or fragment thereof is a substantially similar protein comprising one or more amino acid mutations selected from the group consisting of orfΔ3b, deletion 69-70, M129I, deletion 144, P337S, F338K, V341I, F342L, A344S, A348S, A352S, N354D, S359N, V367F, N379S, A372S, A372T, F377L, K378R, K378N, P384L, T385A, T393P, V395I, D405V, E406Q, R408I, Q409E, Q414A, Q414E, Q414R, K417N, A435S, W436R, N439K, N440K, K444R, V445F, G446V, G446S, P499R, L452R, Y453F, F456L, F456E, K458R, K458Q, E471Q, I472V, G476S, S477N, S477I, S477R, T478I, P479S, N481D, G482S, V483A, V483I, E484K, G485S, F486S, F490S, S494P, N501Y, V503F, Y505C, Y508H, A520S, A520V, P521S, P521R, A522V, A522S, A570D, D614G, P681H, R683A, R685A, I692V, T716I, F817P, A829T, A892P, A899P, A942P, S982A, K986P, V987P, and D1118H, compared to SEQ ID NO: 1.

90. The antiviral composition according to claim 20, wherein the neutralizing IgY antibodies derived from eggs of hens inoculated with a SARS-CoV-2 S-protein or a fragment thereof are capable of neutralizing a SARS-CoV-2 viral variant comprising one or more amino acid mutations.

91. The antiviral composition according to claim 90, wherein the one or more amino acid mutations is selected from the group consisting of orfΔ3b, deletion 69-70, M129I, deletion 144, P337S, F338K, V341I, F342L, A344S, A348S, A352S, N354D, S359N, V367F, N379S, A372S, A372T, F377L, K378R, K378N, P384L, T385A, T393P, V395I, D405V, E406Q, R408I, Q409E, Q414A, Q414E, Q414R, K417N, A435S, W436R, N439K, N440K, K444R, V445F, G446V, G446S, P499R, L452R, Y453F, F456L, F456E, K458R, K458Q, E471Q, I472V, G476S, S477N, S477I, S477R, T478I, P479S, N481D, G482S, V483A, V483I, E484K, G485S, F486S, F490S, S494P, N501Y, V503F, Y505C, Y508H, A520S, A520V, P521S, P521R, A522V, A522S, A570D, D614G, P681H, R683A, R685A, I692V, T716I, F817P, A829T, A892P, A899P, A942P, S982A, K986P, V987P, and D1118H, compared to SEQ ID NO: 1.

92. The antiviral composition according the claim 90, wherein the SARS-CoV-2-S-protein or fragment thereof comprises an amino acid sequence selected from the group consisting of SEQ ID NO: 1, 6, 7, 10, 11, 12, 13, 14, 15, 16, 17, 18, 26, 27, 36, 37, 38, 39, 40, 41, 86, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

93. The method according to claim 55, wherein the first immunogen is a SARS-CoV-2 S-protein, a human ACE2 protein, a fragment thereof, or a substantially similar protein.

94. The method according to claim 93, wherein the SARS-CoV-2 S-protein, fragment thereof, or substantially similar protein, is selected from the group consisting of SARS-CoV-2 RBD protein, SARS-CoV-2 S1-protein, a SARS-CoV-2 S2-protein, a fragment thereof, or a substantially similar protein thereof.

95. The method of claim 94, wherein the SARS-CoV-2-S-protein or fragment thereof comprises an amino acid sequence selected from the group consisting of SEQ ID NO: 1, 6, 7, 10, 11, 12, 13, 14, 15, 16, 17, 18, 26, 27, 36, 37, 38, 39, 40, 41, 86, or a fragment thereof comprising from 50 to 500, or from 100 to 300, or at least 10, 20, 30, 40, 50, 60, 70, 80, 100, 150, or 200 contiguous amino acid residues thereof, or a substantially similar protein.

96. The method according to claim 93, wherein the SARS-CoV-2 S-protein or fragment thereof, comprises a mutation selected from the group consisting of orfΔ3b, deletion 69-70, M129I, deletion 144, P337S, F338K, V341I, F342L, A344S, A348S, A352S, N354D, S359N, V367F, N379S, A372S, A372T, F377L, K378R, K378N, P384L, T385A, T393P, V395I, D405V, E406Q, R408I, Q409E, Q414A, Q414E, Q414R, K417N, A435S, W436R, N439K, N440K, K444R, V445F, G446V, G446S, P499R, L452R, Y453F, F456L, F456E, K458R, K458Q, E471Q, I472V, G476S, S477N, S477I, S477R, T478I, P479S, N481D, G482S, V483A, V483I, E484K, G485S, F486S, F490S, S494P, N501Y, V503F, Y505C, Y508H, A520S, A520V, P521 S, P521R, A522V, A522S, A570D, D614G, P681H, R683A, R685A, 1692V, T716I, F817P, A829T, A892P, A899P, A942P, S982A, K986P, V987P, and DI 118H, compared to SEQ ID NO: 1.

97. The composition according to claim 9, wherein the human ACE2 protein or fragment thereof comprises the amino acid sequence (Gln18-Ser740) of SEQ ID NO: 78, or a substantially similar protein.

98. The method of claim 73, wherein the plasmid DNA encodes a human ACE2 protein or fragment thereof comprising the amino acid sequence (Gln18-Ser740) of SEQ ID NO: 78, or a substantially similar protein.

99. The dosage form according to claim 22, comprising a pharmaceutically acceptable carrier or excipient selected from the group consisting of lactose, mannitol, sorbitol, microcrystalline cellulose, sucrose, sodium citrate, dextrose, dextrose monohydrate, dicalcium phosphate, phosphate buffer, agar-agar, calcium carbonate, sodium carbonate, silicates, alginic acid, corn starch, potato tapioca starch, primogel, magnesium stearate, calcium stearate, talc, solid polyethylene glycols, sodium lauryl, colloidal silicon dioxide, mannitol, sucrose, trehalose, glycine, arginine, dextran, acetyl alcohol, glyceryl monostearate, kaolin, bentonite clay, wax, and paraffin.


SEQUENCE LISTING (127 sequences)

SEQ ID NO: 1 - protein, 1273 aa, Artificial Sequence
  Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 222 aa, Artificial Sequence
  Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 75 aa, Artificial Sequence
  Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 419 aa, Artificial Sequence
  Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 222 aa, Artificial Sequence; identical to SEQ ID NO: 2
  Severe acute respiratory syndrome coronavirus 2

SEQ ID NO: 6 - protein, 1279 aa, Artificial Sequence
  Severe acute respiratory syndrome coronavirus 2
MHHHHHHFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1279 aa, Artificial Sequence
  Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTHHHHHH

SEQ ID NO: 8 - protein, 425 aa, Artificial Sequence
  Severe acute respiratory syndrome coronavirus 2
MHHHHHHSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 425 aa, Artificial Sequence
  Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAHHHHHH

SEQ ID NO: 10 - protein, 685 aa, Artificial Sequence
  Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 11 - protein, 588 aa, Artificial Sequence
  Severe acute respiratory syndrome coronavirus 2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 205 aa, Artificial Sequence
  Severe acute respiratory syndrome coronavirus 2
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 13 - protein, 22 aa, Artificial Sequence
  Severe acute respiratory syndrome coronavirus 2
CASYQTQTNSPRRARSVASQSI

SEQ ID NO: 14 - protein, 27 aa, Artificial Sequence
  Severe acute respiratory syndrome coronavirus 2
GAGICASYQTQTNSPRRARSVASQSII

SEQ ID NO: 15 - protein, 59 aa, Artificial Sequence
  Severe acute respiratory syndrome coronavirus 2
YLYRLFRKSNLKPFERDISTEIYQAGSTPCNGVEGFNCYFPLQSYGFQPTNGVGYQPYR

SEQ ID NO: 16 - protein, 266 aa, Artificial Sequence
  Severe acute respiratory syndrome coronavirus 2
TRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTI

SEQ ID NO: 17 - protein, 223 aa, Artificial Sequence
  Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 18 - protein, 666 aa, Artificial Sequence
  Severe acute respiratory syndrome coronavirus 2
TRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 19 - DNA, 24 bp, Artificial Sequence
  Primer sequence
caagagcagcatcaccgccattgc

SEQ ID NO: 20 - DNA, 30 bp, Artificial Sequence
  Primer sequence
ccgacctcattaagcagctctaatgcgctg

SEQ ID NO: 21 - DNA, 29 bp, Artificial Sequence
  Primer sequence
ggtgtgaaataccgcacagatgcgtaagg

SEQ ID NO: 22 - DNA, 28 bp, Artificial Sequence
  Primer sequence
gcaatcatttcatctgtgagcaaaggtg

SEQ ID NO: 23 - DNA, 26 bp, Artificial Sequence
  Primer sequence
gatccatcaaaaccaagcaagaggtc

SEQ ID NO: 24 - DNA, 1278 bp, Artificial Sequence
  N gene with N-term 6x His-tag (p001) FBB_DNA_012
atgcatcatcaccatcaccactctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 25 - DNA, 1278 bp, Artificial Sequence
  N gene with C-term 6x His-tag (p002) FBB_DNA_013
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcccatcatcaccatcaccactaa

SEQ ID NO: 26 - DNA, 3840 bp, Artificial Sequence
  S gene with N-term 6x His-tag (p003) FBB_DNA_014
atgcatcatcaccatcaccactttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 27 - DNA, 3840 bp, Artificial Sequence
  S gene with C-term 6x His-tag (p004) FBB_DNA_15
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacacatcatcaccatcaccactaa

SEQ ID NO: 28 - protein, 805 aa, Artificial Sequence [sequence omitted]

SEQ ID NO: 29 - DNA, 55 bp, Artificial Sequence
  Primer sequence
cagagaggaggtgtataaggtgatgagttcatcctcttggttgctattaagcttg

SEQ ID NO: 30 - DNA, 47 bp, Artificial Sequence
  Primer sequence
gtcacttagtggtgatggtgatgatggaagctggtctgcacgtcgtc

SEQ ID NO: 31 - DNA, 50 bp, Artificial Sequence
  Primer sequence
catcatcaccatcaccactaagtgacatatagccgcaccaataaaaattg

SEQ ID NO: 32 - DNA, 28 bp, Artificial Sequence
  Primer sequence
catcaccttatacacctcctctctgcgg

SEQ ID NO: 33 - DNA, 2436 bp, Artificial Sequence
  Ace2/ FBB_DNA_008
atgagttcatcctcttggttgctattaagcttggttgccgttacggctgcgcagagcaccatcgaagagcaggcgaaaacctttctggacaaattcaaccacgaagctgaagacctgttttatcagtccagcttagctagctggaactataatacgaacattactgaggagaacgtgcagaacatgaataatgcgggtgacaagtggtcagcctttctgaaagaacagtccacgctggcacaaatgtacccactgcaagagatccaaaacctgacggtcaaactgcagctgcaagcgctgcaacaaaacggcagcagcgttctgtccgaggacaagtccaaacgtttgaatacgatcctgaacacgatgtccaccatctattcaaccggcaaggtgtgcaatccggataacccgcaggagtgcctgctcctggaaccgggtctcaatgaaatcatggcgaacagcttagattataacgaacgtctgtgggcatgggagagctggcgtagcgaagtgggtaaacagttacgccctctgtacgaggaatatgttgtcctgaagaacgagatggcccgagcgaatcactatgaggactacggcgactactggcgcggtgattacgaggtgaatggcgtggatggttatgattacagccgcgggcagctgattgaagacgtcgagcacaccttcgaggagatcaagccgctgtacgaacaccttcacgcatacgtgagagcgaaactgatgaacgcgtacccgagctacatttccccgattggttgtctgccagcacatctgttaggcgacatgtggggtcgtttttggaccaatctgtattctttgaccgttccgttcggccagaagccgaatatcgatgttaccgacgctatggttgaccaagcctgggatgctcaacgtatctttaaagaagcggaaaagttctttgttagcgtaggcctgccgaacatgacccagggtttctgggagaacagtatgctgaccgatccgggaaacgttcagaaggccgtgtgtcatccgaccgcgtgggatctgggtaagggcgacttccgcatactgatgtgcaccaaagtgaccatggatgattttctgaccgcgcatcatgagatgggtcatattcagtacgacatggcgtacgcagcgcaaccatttctgctgcgtaatggtgccaacgagggcttccacgaggcggtgggcgagattatgagcctgtctgcggcgaccccgaagcacctgaagtcaattggcctgctgagcccggactttcaagaagacaacgagacggagatcaatttcttgttgaagcaagctttgactattgtgggcaccctgcctttcacctacatgttggagaagtggcgttggatggtgttcaaaggtgaaattccgaaagaccagtggatgaaaaagtggtgggaaatgaaaagagaaatcgtaggtgttgttgaaccggttccgcatgatgaaacctactgcgacccggcgagcctgttccatgtttccaatgactacagcttcatccgttattacacccgtaccttgtaccaatttcagtttcaagaagcgctgtgtcaggccgctaagcacgaaggtccgctgcacaaatgcgatattagcaactccactgaggccggtcaaaaactgttcaacatgctgcgcctgggcaaaagcgaaccgtggaccctcgcgttagagaatgtagttggcgcaaagaacatgaatgtgcgtccactgttgaactattttgagccgcttttcacctggctgaaggatcaaaacaaaaactccttcgtgggttggtcaactgattggtctccgtatgctgatcaaagtattaaggttcgcatttcgctgaagagcgcgttgggtgataaagcttacgagtggaatgataatgaaatgtatctgttccgttctagcgtggcgtacgcaatgcgtcagtatttcttgaaggtgaaaaaccagatgattctcttcggtgaagaggacgtccgtgtcgccaatctgaagccgcgtatttcgttcaattttttcgtgaccgctccgaaaaacgttagcgatatcatcccgcgtaccgaggtggaaaaagcgattcgtatgtctcgtagccgcatcaacgacgcatttcgcctgaacgacaattccttggagttcttgggcatccagccgacattgggtcccccgaaccagccgccggtgagcatctggctgatcgtttttggcgttgttatgggtgtcatcgttgttggcatcgtgatcctcatttttacgggcatccgcgatcgtaaaaagaagaacaaagcgcgttctggtgagaacccgtatgcaagcatcgacattagtaaaggtgaaaacaacccaggttttcaaaacaccgacgacgtgcagaccagcttccatcatcaccatcaccactaa

SEQ ID NO: 34 - DNA, 5741 bp, Artificial Sequence
  pRAB11 Backbone / FB_DNA_011
gtgacatatagccgcaccaataaaaattgataatagctgagcccgggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcggacagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttctgtaggtttttaggcataaaactatatgatttacccctaaatctttaaaatgccccttaaaattcaaaataaaggcatttaaaatttaaatatttcttgtgataaagtttgttaaaaaggagtggttttatgactgttatgtggttatcgattataggtatgtggttttgtattggaatggcattttttgctatcaaggttattaaaaataaaaattagaccacgcatttatgccgagaaaatttattgtgcgttgagaagaacccttaactaaacttgcagacgaatgtcggcatagcgtgagctattaagccgaccattcgacaagttttgggattgttaagggttccgaggctcaacgtcaataaagcaattggaataaagaagcgaaaaaggagaagtcggttcagaaaaagaaggatatggatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgataataagggtaactattgccggcgaggctagttacccttaagttattggtatgactggttttaagcgcaaaaaaagttgctttttcgtacctattaatgtatcgttttaaatgaatagtaaaaaacatacatagaaaggggaaaaagcaactttttttattgtcatagtttgtgaaaactaagttgtttttatgtgttataacatggaaaagtatactgagaaaaaacaaagaaatcaagtatttcagaaatttattaaacgtcatattggagagaatcaaatggatttagttgaagattgcaatacatttctgtcttttgtagctgataaaactttagaaaaacagaaattatataaagctaattcttgtaaaaatcgattttgtcctgtctgtgcttggagaaaagctaggtcagctgttgaattatgcacgagtattttaaaagttattgtgatgacgacgataaacgattatcaaaagtataatgttaaaatgctttattatactaacgttatataaacattatactttcgttatacaaattttaaccctgttaggaactataaaaaatcatgaaaattttaatttgcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtg

SEQ ID NO: 35 - protein, 811 aa, Artificial Sequence
  Ace2/ FBB_AA_008
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSFHHHHHH

SEQ ID NO: 36 - protein, 223 aa, Artificial Sequence; identical to SEQ ID NO: 17
  Severe acute respiratory syndrome coronavirus 2

SEQ ID NO: 37 - protein, 223 aa, Artificial Sequence
  Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCYGVEGFNCYFPLQSYGFQPTNGVGYQPYRVVVLSFELLHAPATVCGPKKSTNLVKNKCVNF

SEQ ID NO: 38 - protein, 223 aa, Artificial Sequence
  Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKCVNF

SEQ ID NO: 39 - protein, 223 aa, Artificial Sequence
  Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 40 - protein, 685 aa, Artificial Sequence
  Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAR

SEQ ID NO: 41 - protein, 1273 aa, Artificial Sequence; identical to SEQ ID NO: 1
  Severe acute respiratory syndrome coronavirus 2

SEQ ID NO: 42 - protein, 731 aa, Artificial Sequence
  Human Angiotensin converting enzyme 2 (Gln18-Ser740) fused with polyhistidine tag at C-terminus
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSVDHHHHHH

SEQ ID NO: 43 - protein, 805 aa, Artificial Sequence [sequence omitted]

SEQ ID NO: 44 - DNA, 40 bp, Artificial Sequence
  Primer sequence
gtgtataaggtgatgtctgataatggaccccaaaatcagc

SEQ ID NO: 45 - DNA, 39 bp, Artificial Sequence
  Primer sequence
agtggtgatggtgatgatgggcctgagttgagtcagcac

SEQ ID NO: 46 - DNA, 40 bp, Artificial Sequence
  Primer sequence
tcaggcccatcatcaccatcaccactaagtgacatatagc

SEQ ID NO: 47 - DNA, 38 bp, Artificial Sequence
  Primer sequence
cattatcagacatcaccttatacacctcctctctgcgg

SEQ ID NO: 48 - DNA, 40 bp, Artificial Sequence
  Primer sequence
catcatcaccatcaccactctgataatggaccccaaaatc

SEQ ID NO: 49 - DNA, 40 bp, Artificial Sequence
  Primer sequence
tggtgcggctatatgtcacttaggcctgagttgagtcagc

SEQ ID NO: 50 - DNA, 40 bp, Artificial Sequence
  Primer sequence
agtggtgatggtgatgatgcatcaccttatacacctcctc

SEQ ID NO: 51 - DNA, 40 bp, Artificial Sequence
  Primer sequence
tgctgactcaactcaggcctaagtgacatatagccgcacc

SEQ ID NO: 52 - DNA, 53 bp, Artificial Sequence
  Primer sequence
cagagaggaggtgtataaggtgatgtttgtttttcttgttttattgccactag

SEQ ID NO: 53 - DNA, 52 bp, Artificial Sequence
  Primer sequence
cacttagtggtgatggtgatgatgtgtgtaatgtaatttgactcctttgagc

SEQ ID NO: 54 - DNA, 51 bp, Artificial Sequence
  Primer sequence
acatcatcaccatcaccactaagtgacatatagccgcaccaataaaaattg

SEQ ID NO: 55 - DNA, 48 bp, Artificial Sequence
  Primer sequence
gcaataaaacaagaaaaacaaacatcaccttatacacctcctctctgc

SEQ ID NO: 56 - DNA, 56 bp, Artificial Sequence
  Primer sequence
aggtgatgcatcatcaccatcaccactttgtttttcttgttttattgccactagtc

SEQ ID NO: 57 - DNA, 44 bp, Artificial Sequence
  Primer sequence
ggctatatgtcacttatgtgtaatgtaatttgactcctttgagc

SEQ ID NO: 58 - DNA, 23 bp, Artificial Sequence
  Primer sequence
ggtgatgaagtcagacaaatcgc

SEQ ID NO: 59 - DNA, 56 bp, Artificial Sequence
  Primer sequence
tacattacacataagtgacatatagccgcaccaataaaaattgataatagctgagc

SEQ ID NO: 60 - DNA, 30 bp, Artificial Sequence; identical to SEQ ID NO: 20
  Primer sequence

SEQ ID NO: 61 - DNA, 29 bp, Artificial Sequence
  Primer sequence
ggtgtgaaataccgcacagatgcgtaagg

SEQ ID NO: 62 - DNA, 24 bp, Artificial Sequence
  Primer sequence
caagagcagcatcaccgccattgc

SEQ ID NO: 63 - DNA, 28 bp, Artificial Sequence
  Primer sequence
gcaatcatttcatctgtgagcaaaggtg

SEQ ID NO: 64 - DNA, 26 bp, Artificial Sequence
  Primer sequence
gatccatcaaaaccaagcaagaggtc

SEQ ID NO: 65 - DNA, 25 bp, Artificial Sequence
  Primer sequence
cacacgcctattaatttagtgcgtg

SEQ ID NO: 66 - DNA, 24 bp, Artificial Sequence
  Primer sequence
cgcactaaattaataggcgtgtgc

SEQ ID NO: 67 - DNA, 23 bp, Artificial Sequence
  Primer sequence
ggtgatgaagtcagacaaatcgc

SEQ ID NO: 68 - DNA, 25 bp, Artificial Sequence
  Primer sequence
tcaggatgttaactgcacagaagtc

SEQ ID NO: 69 - DNA, 26 bp, Artificial Sequence
  Primer sequence
gatccatcaaaaccaagcaagaggtc

SEQ ID NO: 70 - DNA, 30 bp, Artificial Sequence
  Primer sequence
ctgaagtgcaaattgataggttgatcacag

SEQ ID NO: 71 - DNA, 29 bp, Artificial Sequence
  Primer sequence
caacacagtttatgatcctttgcaacctg

SEQ ID NO: 72 - DNA, 25 bp, Artificial Sequence
  Primer sequence
ccatcatgacaaatggcaggagcag

SEQ ID NO: 73 - DNA, 26 bp, Artificial Sequence
  Primer sequence
ccacaaacagttgctggtgcatgtag

SEQ ID NO: 74 - DNA, 28 bp, Artificial Sequence
  Primer sequence
gcaatcatttcatctgtgagcaaaggtg

SEQ ID NO: 75 - DNA, 48 bp, Artificial Sequence
  Primer sequence
cttagtggtgatggtgatgatgtttgtatagttcatccatgccatgtg

SEQ ID NO: 76 - DNA, 51 bp, Artificial Sequence; identical to SEQ ID NO: 54
  Primer sequence

SEQ ID NO: 77 - protein, 731 aa, Artificial Sequence; identical to SEQ ID NO: 42
  Human Angiotensin converting enzyme 2 (Gln18-Ser740) fused with polyhistidine tag at C-terminus

SEQ ID NO: 78 - protein, 805 aa, Artificial Sequence [sequence omitted]

SEQ ID NO: 79 - protein, 530 aa, Artificial Sequence
  Norovirus Group-1 Capsid protein Accession Q83884
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 80 - protein, 89 aa, Artificial Sequence
  E. coli Shiga toxin 1 B subunit
MKKTLLIAASLSFFSASALATPDCVTGKVEYTKYNDDDTFTVKVGDKELFTNRWNLQSLLLSAQITGMTVTIKTNACHNGGGFSEVIFR

SEQ ID NO: 81 - protein, 89 aa, Artificial Sequence
  E. coli Shiga toxin 2 subunit B
MKKMFMAVLFALASVNAMAADCAKGKIEFSKYNEDDTFTVKVDGKEYWTSRWNLQPLLQSAQLTGMTVTIKSSTCESGSGFAEVQFNND

SEQ ID NO: 82 - protein, 424 aa, Artificial Sequence
  P. falciparum circumsporozoite protein
MMRKLAILSVSSFLFVEALFQEYQCYGSSSNTRVLNELNYDNAGINLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 83 - DNA, 5472 bp, Artificial Sequence
  PCI-NEO EXPRESSION VECTOR
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 84 - DNA, 19 bp, Artificial Sequence
  T7 Promoter
taatacgactcactatagg

SEQ ID NO: 85 - DNA, 120 bp, Artificial Sequence
  SV40_PA_Terminator
tgataagatacattgatgagtttggacaaaccacaactagaatgcagtgaaaaaaatgctttatttgtgaaatttgtgatgctattgctttatttgtaaccattataagctgcaataaac

SEQ ID NO: 86 - protein, 1273 aa, Artificial Sequence; identical to SEQ ID NO: 1
  SARS-CoV-2 S-protein

SEQ ID NO: 87 - protein, 61 aa, Artificial Sequence
  Short neurotoxin 2, recombinant venom peptide (Snouted cobra)
MICHNQQSSQPPTIKTCPGETNCYKKRWRDHRGTIIERGCGCPSVKKGVGIYCCKTNKCNR

SEQ ID NO: 88 - protein, 22 aa, Artificial Sequence
  Waglerin-1, recombinant venom peptide (temple pit viper)
GGKPDLRPCHPPCHYIPRPKPR

SEQ ID NO: 89 - protein, 55 aa, Artificial Sequence
  Kunitz-type kappaPI-theraphotoxin-Hs1a, recombinant venom peptide
IDTCRLPSDRGRCKASFERWYFNGRTCAKFIYGGCGGNGNKFPTQEACMKRCAKA

SEQ ID NO: 90 - protein, 253 aa, Artificial Sequence
  prion protein may have accession UniProtKB - P04156 (PRIO_HUMAN)
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 91 - protein, 229 aa, Artificial Sequence
  Prion protein
MGSSHHHHHHSSGLVPRGSHMKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGS

SEQ ID NO: 92 - protein, 42 aa, Artificial Sequence [sequence omitted]

SEQ ID NO: 93 - DNA, 20 bp, Artificial Sequence
  Synthetic oligodeoxynucleotides
tccatgacgttcctgacgtt

SEQ ID NO: 94 - protein, 758 aa, Artificial Sequence
  UniProtKB - P10636 (TAU_HUMAN), Microtubule-associated protein tau
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 95 - protein, 758 aa, Artificial Sequence
  UniProtKB - A0A0G2JPD5 (A0A0G2JPD5_HUMAN), HUMAN Microtubule-associated protein
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 96 - protein, 227 aa, Artificial Sequence
  M. Tuberculosis ESAT-6 Antigen protein
MRYLIATAVLVAVVLVGWPAAGAPPSCAGLGGTVQAGQICHVHASGPKYMLDMTFPVDYPDQQALTDYITQNRDGFVNVAQGSPLRDQPYQMDATSEQHSSGQPPQATRSVVLKFFQDLGGAHPSTWYKAFNYNLATSQPITFDTLFVPGTTPLDSIYPIVQRELARQTGFGAAILPSTGLDPAHYQNFAITDDSLIFYFAQGELLPSFVGACQAQVPRSAIPPLAI

SEQ ID NO: 97 - protein, 180 aa, Artificial Sequence
  Clostridium difficile FliC protein
MRVNTNVSALIANNQMGRNVNGQSKSMEKLSSGVRIKRAADDAAGLAISEKMRAQIKGLDQAGRNVQDGISVVQTAEGSLEETGNILQRMRTLSLQSANEINNTEEREKIADELTQLKDEIERISSSTEFNGKKLLDGTSSTIRLQVGASYGTNVSGTSNNNNEIKIQLVNTASIMASAG

SEQ ID NO: 98 - protein, 110 aa, Artificial Sequence
  Clostridium difficile FliC protein
ITTASIGSMKAGGTTGTDAAKTMVSSLDAALKSLNSSRAKLGAQQNRLESTQNNLNNTLENVTAAESRIRDTDVASEMVNLSKMNILVQASQSMLAQANQQPQGVLQLLG

SEQ ID NO: 99 - protein, 507 aa, Artificial Sequence
  Clostridium difficile FliD protein
MSSISPVRVTGLSGNFDMEGIIEASMIRDKEKVNKAKQDQQIVKWKQEIYRDIIKESKNLYDKYLNGDSPNSITNKKAYSATRITSSDESIIVAKGSAGAEKINYQFAVSQMAEPAKVTIKLNSSDPIVQQFPPNASGASSLNIGGVNIPISEQDTTSTIVSKINSLCADNDIRASYSEMTGELIISRKQTGSSSDIDLKVIGNDSLAGQIASDNGITFTTDASGTKSAVVYGKNLEADVTDDQGRVTHISKEQNSFKIDNIDYNVNSKGSAKLVSVTDTEEATKNMKAFVDDYNALMDKVYGLVTTKKSKDYPPLTDEQKDDMTTEEIEKWEKKAKEGILRNDDELRAFVEDIQSMFFGDADTIIALRKLGISEHENYNKKGQISFNADTFSKALIDDSDKVYKALAGYSSNYDDKGMFEKLKKIVFEYSGSSASKLTKKAGMENSSSASQNVYSKQIAEQERNISRLVEKMNDKEKRLYAKYSALESLLNKYSSQMNYFSQAQGN

SEQ ID NO: 100 - protein, 610 aa, Artificial Sequence
  Clostridium difficile Cwp84 protein
MRKYKSKKLSKLLALLTVCFLIVSTIPVSAENHKTLDGVETAEYSESYLQYLEDVKNGDTAKYNGVIPFPHEMEGTTLRNKGRSSLPSAYKSSVAYNPMDLGLTTPAKNQGSLNTCWSFSGMSTLEAYLKLKGYGTYDLSEEHLRWWATGGKYGWNLDDMSGSSNVTAIGYLTAWAGPKLEKDIPYNLKSEAQGATKPSNMDTAPTQFNVTDVVRLNKDKETVKNAIMQYGSVTSGYAHYSTYFNKDETAYNCTNKRAPLNHAVAIVGWDDNYSKDNFASDVKPESNGAWLVKSSWGEFNSMKGFFWISYEDKTLLTDTDNYAMKSVSKPDSDKKMYQLEYAGLSKIMSNKVTAANVFDFSRDSEKLDSVMFETDSVGAKYEVYYAPVVNGVPQNNSMTKLASGTVSYSGYINVPTNSYSLPKGKGAIVVVIDNTANPNREKSTLAYETNIDAYYLYEAKANLGESYILQNNKFEDINTYSEFSPCNFVIKAITKTSSGQATSGESLTGADRYETAVKVSQKGWTSSQNAVLVNGDAIVDALTATPFTAAIDSPILLTGKDNLDSKTKAELQRLGTKKVYLIGGENSLSKNVQTQLSNMGISVERISGSD

SEQ ID NO: 101 - protein, 2366 aa, Artificial Sequence
  C. difficile Toxin B, TcdB
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 102 - protein, 111 aa, Artificial Sequence
  Cryptosporidium parvum P23 protein
MGCSSSKPETKVAENKSAADANKQRELAEKKAQLAKAVKNPAPISNQAQQKPEEPKKSEPAPNNPPAADAPAAQAPAAPAEPAPQDKPADAPAAEAPAAEPAAQQDKPADS

SEQ ID NO: 103 - protein, 297 aa, Artificial Sequence
  CD20 protein
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 104 - protein, 68 aa, Artificial Sequence
  Rhinovirus VP2 protein
GAQVSTQKSGSHENQNILTNGSNQTFTVINYYKDAASTSSAGQSLSMDPSKFTEPVKDLMLKGAPALN

SEQ ID NO: 105 - protein, 530 aa, Artificial Sequence; identical to SEQ ID NO: 79
  Influenza virus VP1 capsid protein

SEQ ID NO: 106 - protein, 253 aa, Artificial Sequence; identical to SEQ ID NO: 90
  Prion protein

SEQ ID NO: 107 - protein, 374 aa, Artificial Sequence
  Herpes Simplex virus 1 glycoprotein gD
MMTRLHFWWCGIFAVLKYLVCTSSLTTTPKTTTVYVKGFNIPPLRYNYTQARIVPKIPQAMDPKITAEVRYVTSMDSCGMVALISEPDIDATIRTIQLSQKKTYNATISWFKVTQGCEYPMFLMDMRLCDPKREFGICALRSPSYWLEPLTKYMFLTDDELGLIMMAPAQFNQGQYRRVITIDGSMFYTDFMVQLSPTPCWFAKPDRYEEILHEWCRNVKTIGLDGARDYHYYWVPYNPQPHHKAVLLYWYRTHGREPPVRFQEAIRYDRPAIPSGSEDSKRSNDSRGESSGPNWIDIENYTPKNNVPIIISDDDVPTAPPKGMNNQSVVIPAIVLSCLIIALILGVIYYILRVKRSRSTAYQQLPIIHTTHHP

SEQ ID NO: 108 - protein, 393 aa, Artificial Sequence
  Herpes Simplex virus 2 glycoprotein gD
MGRLTSGVGTAALLVVAVGLRVVCAKYALADPSLKMADPNRFRGKNLPVLDRLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 109 - protein, 775 aa, Artificial Sequence
  Rotavirus Vp4 capsid protein
MASLIYRQLLTNSYTVELSDEINTIGSEKSQNITINPGPFAQTNYAPVTWSHGEVNDSTTIEPVLDGPYQPTSFKPPSDYWILLNPTNQQVVLEGTNKTDIWIALLLVEPNVTNQSRQYTLFGETKQITIENNTNKWKFFEMFRSNVSSEFQHKRTLTSDTKLAGFLKHYNSVWTFHGETPHATTDYSSTSNLSEVETTIHVEFYIISRSQESKCVEYINTGLPPMQNTRNIVPVALSSRSVTYQRAQVSEDIIISKTSLWKEMQYNRDIIIRFKFNNSIIKLGGLGYKWSEISFKAANYQYNYLRDGEQVTAHTTCSVNGVNNFSYNGGLLPTHFSISRYEVIKENSYVYVDYWDDSQAFRNMVYVRSLAANLNSVKCSGGNYNFQMPVGAWPVMSGGAVSLHFAGVTLSTQFTDFVSLNSLRFRFSLTVEEPPFSILRTRVSGLYGLPASNPNSGHEYYEIAGRFSLISLVPSNDDYQTPIMNSITVRQDLERQLGDLREEFNSLSQEIAITQLIDLALLPLDMFSMFSGIKSTIDAAKSMATKVMKKFKRSGLATSISELTGSLSNAASSVSRSSSIRSNISSISEWTDVSEQIAGSSDSVRNISTQTSAISRRLRLREITTQTEGMNDIDISAAVLKTKIDRSTHIRPDTLPDIITESSEKFIPKRAYRVLKDDEVMEADVDGKFFAYKVDTFEEVPFDVDKFVDLVTDSPVISAIIDFKTLKNLNDNYGITRSQALDLIRSDPRVLRDFINQNNPIIKNRIEQLILQCRL

SEQ ID NO: 110 - protein, 297 aa, Artificial Sequence
  Rotavirus Vp7 surface glycoprotein
MDYIIYRITFVIVVLSVLSNAQNYGINLPITGSMDTAYANSTQDNNFLFSTLCLYYPSEAPTQISDTEWKDTLSQLFLTKGWPTGSVYFNEYSNVLEFSIDPKLYCDYNVVLIRFVSGEELDISELADLILNEWLCNPMDITLYYYQQTGEANKWISMGSSCTVKVCPLNTQTLGIGCQTTNTATFETVADSEKLAIIDVVDSVNHKLNITSTTCTIRNCNKLGPRENVAIIQVGGSNILDITADPTTSPQTERMMRVNWKKWWQVFYTVVDYINQIVQVMSKRSRSLDSSSFYYRV

SEQ ID NO: 111 - protein, 175 aa, Artificial Sequence
  Rotavirus NSP4 viral enterotoxin
MDKLADLNYTLSVITSMNDTLHSIIEDPGMAYFPYIASVLTVLFTLHKASIPTMKIALKASKCSYKVIKYCVVTIINTLLKLAGYKEQVTTKDEIEQQMDRIVKEMRRQLEMIDKLTTREIEQIELLKRIHDNLITRPVNVIDMSMEFNQKNIKTLDEWESRKNPYEPSEVTASM

SEQ ID NO: 112 - protein, 352 aa, Artificial Sequence
  Zika virus ZIKV non-structural-1 (NS1) protein
DVGCSVDFSKKETRCGTGVFIYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLEHRAWNSFLVEDHGFGVFHTSVWLKVREDYSLECDPAVIGTAVKGREAAHSDLGYWIESEKNDTWRLKRAHLIEMKTCEWPKSHTLWTDGVEESDLIIPKSLAGPLSHHNTREGYRTQVKGPWHSEELEIRFEECPGTKVYVEETCGTRGPSLRSTTASGRVIEEWCCRECTMPPLSFRAKDGCWYGMEIRPRKEPESNLVRSMVTA

SEQ ID NO: 113 - protein, 263 aa, Artificial Sequence
  Smallpox vaccinia complement protein (VCP)
MKVESVTFLTLLGIGCVLSCCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 114 - protein, 263 aa, Artificial Sequence; identical to SEQ ID NO: 113
  Smallpox SPICE protein

SEQ ID NO: 115 - protein, 809 aa, Artificial Sequence
  Bacillus anthracis (Anthrax) Lethal Factor
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLARYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 116 - protein, 800 aa, Artificial Sequence
  Bacillus anthracis (Anthrax) Edema Factor
MTRNKFIPNKFSIISFSVLLFAISSSQAIEVNAMNEHYTESDIKRNHKTEKNKTEKEKFKDSINNLVKTEFTNETLDKIQQTQGLLKKIPKDVLEIYSELGGEIYFTDIDLVEHKELQDLSEEEKNSMNSRGEKVPFASRFVFEKKRETPKLIINIKDYAINSEQSKEVYYEIGKGISLDIISKDKSLDPEFLNLIKSLSDDSDSSDLLFSQKFKEKLELNNKSIDINFIKENLTEFQHAFSLAFSYYFAPDHRTVLELYAPDMFEYMNKLEKGGFEKISESLKKEGVEKDRIDVLKGEKALKASGLVPEHADAFKKIARELNTYILFRPVNKLATNLIKSGVATKGLNVHVKSSDWGPVAGYIPFDQDLSKKHGQQLAVEKGNLENKKSITEHEGEIGKIPLKLDHLRIEELKENGIILKGKKEIDNGKKYYLLESNNQVYEFRISDENNEVQYKTKEGKITVLGEKFNWRNIEVMAKNVEGVLKPLTADYDLFALAPSLTEIKKQIPQKEWDKVVNTPNSLEKQKGVTNLLIKYGIERKPDSTKGTLSNWQKQMLDRLNEAVKYTGYTGGDVVNHGTEQDNEEFPEKDNEIFIINPEGEFILTKNWEMTGRFIEKNITGKDYLYYFNRSYNKIAPGNKAYIEWTDPITKAKINTIPTSAEFIKNLSSIRRSSNVGVYKDSGDKDEFAKKESVKKIAGYLSDYYNSANHIFSQEKKRKISIFRGIQAYNEIENVLKSKQIAPEYKNYFQYLKERITNQVQLLLTHQKSNIEFKLLYKQLNFTENETDNFEVFQKIIDEK

SEQ ID NO: 117 - protein, 764 aa, Artificial Sequence
  Bacillus anthracis (Anthrax) Protective Antigen (pagA)
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 118 - protein, 676 aa, Artificial Sequence
  Ebola glycoprotein
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 119 - protein, 516 aa, Artificial Sequence
  Staphylococcus Aureus SpA
LKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 120 - protein, 258 aa, Artificial Sequence
  Cholra toxin subunit A
MVKIIFVFFIFLSSFSYANDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 121 - protein, 124 aa, Artificial Sequence
  Cholera toxin subunit B
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 122 - protein, 223 aa, Artificial Sequence
  SARS-CoV-2 RBD [L452R]
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 123 - protein, 223 aa, Artificial Sequence
  SARS-CoV-2 RBD [S477N]
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRVVVLSFELLHAPATVCGPKKSTNLVKNKCVNF

SEQ ID NO: 124 - protein, 144 aa, Artificial Sequence
  Granulocyte-macrophage colony-stimulating factor (chicken) (ch GMCSF) encoded by plasmid adjuvant
MLAQLTILLALGVLCSPAPTTTYSCCYKVYTILEEITSHLESTAATAGLSSVPMDIRDKTCLRNNLKTFIESLKTNGTEEESGIVFQLNRVHECERLFSNITPTPQVPDKECRTAQVSREKFKEALKTFFIYLSDVLPEEKDCI

SEQ ID NO: 125 - protein, 164 aa, Artificial Sequence
  IFN-gamma encoded by plasmid adjuvant
MTCQTYNLFVLSVIMIYYGHTASSLNLVQLQDDIDKLKADFNSSHSDVADGGPIIVEKLKNWTERNEKRIILSQIVSMYLEMLENTDKSKPHIKHISEELYTLKNNLPDGVKKVKDIMDLAKLPMNDLRIQRKAANELFSILQKLVDPPSFKRKRSQSQRRCNC

SEQ ID NO: 126 - protein, 143 aa, Artificial Sequence
  interleukin-2 (IL-2) (O73883 Uniprot) encoded by plasmid adjuvant
MMCKVLIFGCISVAMLMTTAYGASLSSAKRKPLQTLIKDLEILENIKNKIHLELYTPTETQECTQQTLQCYLGEVVTLKKETEDDTEIKEEFVTAIQNIEKNLKSLTGLNHTGSECKICEANNKKKFPDFLHELTNFVRYLQK

SEQ ID NO: 127 - protein, 253 aa, Artificial Sequence
  cHSP70 encoded by plasmid adjuvant
EVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGGEPGGAHPGSADD